SEQUENCE LISTING (58 sequences)

SEQ ID NO: 1 - protein, 18 aa, Artificial Sequence
  Synthetic Construct
MLRLLLALNLFPSIQVTG

SEQ ID NO: 2 - protein, 9 aa, Artificial Sequence
  Synthetic Construct
CDCRGDCFC

SEQ ID NO: 3 - protein, 8 aa, Artificial Sequence
  Synthetic Construct
DYKDDDDK

SEQ ID NO: 4 - protein, 39 aa, Artificial Sequence
  Synthetic Construct
IEVMYPPPYLDNEKSNGTIIHVKGKHLCPSPLFPGPSKP

SEQ ID NO: 5 - protein, 29 aa, Artificial Sequence
  Synthetic Construct
FWVLVVVGGVLACYSLLVTVAFIIFWVRS

SEQ ID NO: 6 - protein, 7 aa, Artificial Sequence
  Synthetic Construct
QLFHLQK

SEQ ID NO: 7 - protein, 32 aa, Artificial Sequence
  Synthetic Construct
LKKRGNHSTGLCLLVMFFMVLVALVGLGLGMF

SEQ ID NO: 8 - protein, 509 aa, Artificial Sequence
  Synthetic Construct
MALPVTALLLPLALLLHAARPQVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSSGGGGSGGGGSGGGGSEVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - protein, 22 aa, Artificial Sequence
  Synthetic Construct
GSGATNFSLLKQAGDVEENPGP

SEQ ID NO: 10 - protein, 649 aa, Artificial Sequence
  Synthetic Construct
MALPVTALLLPLALLLHAARPQVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSSGGGGSGGGGSGGGGSEVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMLRLLLALNLFPSIQVTGCDCRGDCFCGGGGSGGGGSGGGGSDYKDDDDKIEVMYPPPYLDNEKSNGTIIHVKGKHLCPSPLFPGPSKPFWVLVVVGGVLACYSLLVTVAFIIFWVRS

SEQ ID NO: 11 - protein, 602 aa, Artificial Sequence
  Synthetic Construct
MALPVTALLLPLALLLHAARPQVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSSGGGGSGGGGSGGGGSEVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPLKKRGNHSTGLCLLVMFFMVLVALVGLGLGMFQLFHLQKDYKDDDDKGGGGSGGGGSGGGGSCDCRGDCFC

SEQ ID NO: 12 - protein, 239 aa, Artificial Sequence
  Synthetic Construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 13 - protein, 35 aa, Artificial Sequence
  Synthetic Construct
PPRASALPAPPTGSALPDPQTASALPDPPAASALP

SEQ ID NO: 14 - protein, 21 aa, Artificial Sequence
  Synthetic Construct
AALAVISFLLGLGLGVACVLA

SEQ ID NO: 15 - protein, 2 aa, Artificial Sequence
  Synthetic Construct
DN

SEQ ID NO: 16 - protein, 12 aa, Artificial Sequence
  Synthetic Construct
WNTTKQEHFPDN

SEQ ID NO: 17 - protein, 98 aa, Artificial Sequence
  Synthetic Construct
PSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 18 - protein, 46 aa, Artificial Sequence
  Synthetic Construct
LLPSWAITLISVNGIFVICCLTYCFAPRCRERRRNERLRRESVRPV

SEQ ID NO: 19 - protein, 888 aa, Artificial Sequence
  Synthetic Construct
MALPVTALLLPLALLLHAARPQVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSSGGGGSGGGGSGGGGSEVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMLRLLLALNLFPSIQVTGCDCRGDCFCGGGGSGGGGSGGGGSDYKDDDDKIEVMYPPPYLDNEKSNGTIIHVKGKHLCPSPLFPGPSKPFWVLVVVGGVLACYSLLVTVAFIIFWVRSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 20 - protein, 879 aa, Artificial Sequence
  Synthetic Construct
MALPVTALLLPLALLLHAARPQVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSSGGGGSGGGGSGGGGSEVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMLRLLLALNLFPSIQVTGGGGGSGGGGSGGGGSDYKDDDDKIEVMYPPPYLDNEKSNGTIIHVKGKHLCPSPLFPGPSKPFWVLVVVGGVLACYSLLVTVAFIIFWVRSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 21 - protein, 880 aa, Artificial Sequence
  Synthetic Construct
MALPVTALLLPLALLLHAARPQVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSSGGGGSGGGGSGGGGSEVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMLRLLLALNLFPSIQVTGCDCRGDCFCGGGGSGGGGSGGGGSIEVMYPPPYLDNEKSNGTIIHVKGKHLCPSPLFPGPSKPFWVLVVVGGVLACYSLLVTVAFIIFWVRSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 22 - protein, 643 aa, Artificial Sequence
  Synthetic Construct
MALPVTALLLPLALLLHAARPQVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSSGGGGSGGGGSGGGGSEVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMPMGSLQPLATLYLLGMLVASCLGCDCRGDCFCGGGGSGGGGSGGGGSDYKDDDDKPPRASALPAPPTGSALPDPQTASALPDPPAASALPAALAVISFLLGLGLGVACVLA

SEQ ID NO: 23 - protein, 635 aa, Artificial Sequence
  Synthetic Construct
MALPVTALLLPLALLLHAARPQVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSSGGGGSGGGGSGGGGSEVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMGHTRRQGTSPSKCPYLNFFQLLVLAGLSHFCSGCDCRGDCFCGGGGSDYKDDDDKDNLLPSWAITLISVNGIFVICCLTYCFAPRCRERRRNERLRRESVRPV

SEQ ID NO: 24 - protein, 645 aa, Artificial Sequence
  Synthetic Construct
MALPVTALLLPLALLLHAARPQVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSSGGGGSGGGGSGGGGSEVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMGHTRRQGTSPSKCPYLNFFQLLVLAGLSHFCSGCDCRGDCFCGGGGSDYKDDDDKWNTTKQEHFPDNLLPSWAITLISVNGIFVICCLTYCFAPRCRERRRNERLRRESVRPV

SEQ ID NO: 25 - protein, 731 aa, Artificial Sequence
  Synthetic Construct
MALPVTALLLPLALLLHAARPQVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSSGGGGSGGGGSGGGGSEVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMGHTRRQGTSPSKCPYLNFFQLLVLAGLSHFCSGCDCRGDCFCGGGGSDYKDDDDKPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 26 - protein, 635 aa, Artificial Sequence
  Synthetic Construct
MALPVTALLLPLALLLHAARPQVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSSGGGGSGGGGSGGGGSEVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMPMGSLQPLATLYLLGMLVASCLGCDCRGDCFCGGGGSGGGGSGGGGSPPRASALPAPPTGSALPDPQTASALPDPPAASALPAALAVISFLLGLGLGVACVLA

SEQ ID NO: 27 - protein, 125 aa, Artificial Sequence
  Synthetic Construct
QVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSS

SEQ ID NO: 28 - protein, 125 aa, Artificial Sequence
  Synthetic Construct
EVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSS

SEQ ID NO: 29 - protein, 10 aa, Artificial Sequence
  Synthetic Construct
FSGYGVSTMA

SEQ ID NO: 30 - protein, 17 aa, Artificial Sequence
  Synthetic Construct
AITVGSGNTYYADSVKG

SEQ ID NO: 31 - protein, 16 aa, Artificial Sequence
  Synthetic Construct
GYLSGGSWDVPGRYNY

SEQ ID NO: 32 - protein, 10 aa, Artificial Sequence
  Synthetic Construct
GNTYSSNYMG

SEQ ID NO: 33 - protein, 17 aa, Artificial Sequence
  Synthetic Construct
VIYTRGGHTYYVDSVRG

SEQ ID NO: 34 - protein, 16 aa, Artificial Sequence
  Synthetic Construct
SSRHRLGLNNPRDYDY

SEQ ID NO: 35 - protein, 487 aa, Artificial Sequence
  Synthetic Construct
MQSGTHWRVLGLCLLSVGVWGQQVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSSGGGGSGGGGSGGGGSEVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSSGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 36 - protein, 613 aa, Artificial Sequence
  Synthetic Construct
MQSGTHWRVLGLCLLSVGVWGQQVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSSGGGGSGGGGSGGGGSEVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSSGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGSGATNFSLLKQAGDVEENPGPMPMGSLQPLATLYLLGMLVASCLGCDCRGDCFCGGGGSGGGGSGGGGSPPRASALPAPPTGSALPDPQTASALPDPPAASALPAALAVISFLLGLGLGVACVLA

SEQ ID NO: 37 - protein, 621 aa, Artificial Sequence
  Synthetic Construct
MQSGTHWRVLGLCLLSVGVWGQQVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSSGGGGSGGGGSGGGGSEVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSSGGGGSGGGGSGGGGSDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGSGATNFSLLKQAGDVEENPGPMPMGSLQPLATLYLLGMLVASCLGCDCRGDCFCGGGGSGGGGSGGGGSDYKDDDDKPPRASALPAPPTGSALPDPQTASALPDPPAASALPAALAVISFLLGLGLGVACVLA

SEQ ID NO: 38 - protein, 6 aa, Artificial Sequence
  Synthetic Construct
CNGRCG

SEQ ID NO: 39 - protein, 617 aa, Artificial Sequence
  Synthetic Construct
MALPVTALLLPLALLLHAARPQVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSSGGGGSGGGGSGGGGSEVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMPMGSLQPLATLYLLGMLVASCLGCNGRCGPPRASALPAPPTGSALPDPQTASALPDPPAASALPAALAVISFLLGLGLGVACVLA

SEQ ID NO: 40 - protein, 625 aa, Artificial Sequence
  Synthetic Construct
MALPVTALLLPLALLLHAARPQVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSSGGGGSGGGGSGGGGSEVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMPMGSLQPLATLYLLGMLVASCLGCNGRCGDYKDDDDKPPRASALPAPPTGSALPDPQTASALPDPPAASALPAALAVISFLLGLGLGVACVLA

SEQ ID NO: 41 - protein, 369 aa, Artificial Sequence
  Synthetic Construct
MALPVTALLLPLALLLHAARPQVQLVESGGGVVQPGGSLRLSCAASGLSFSTYTVAWFRQAPGKGLEGVAAIPYTSQHMVYTDSVKGRFTISRDNSKNTLYLQMNSLRAEDTAVYYCATDRRPGTSMLAINGYNRWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - protein, 492 aa, Artificial Sequence
  Synthetic Construct
MALPVTALLLPLALLLHAARPQVQLVESGGGVVQPGGSLRLSCAASGLSFSTYTVAWFRQAPGKGLEGVAAIPYTSQHMVYTDSVKGRFTISRDNSKNTLYLQMNSLRAEDTAVYYCATDRRPGTSMLAINGYNRWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMPMGSLQPLATLYLLGMLVASCLGCNGRCGGGGGSGGGGSGGGGSPPRASALPAPPTGSALPDPQTASALPDPPAASALPAALAVISFLLGLGLGVACVLA

SEQ ID NO: 43 - protein, 500 aa, Artificial Sequence
  Synthetic Construct
MALPVTALLLPLALLLHAARPQVQLVESGGGVVQPGGSLRLSCAASGLSFSTYTVAWFRQAPGKGLEGVAAIPYTSQHMVYTDSVKGRFTISRDNSKNTLYLQMNSLRAEDTAVYYCATDRRPGTSMLAINGYNRWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMPMGSLQPLATLYLLGMLVASCLGCNGRCGGGGGSGGGGSGGGGSDYKDDDDKPPRASALPAPPTGSALPDPQTASALPDPPAASALPAALAVISFLLGLGLGVACVLA

SEQ ID NO: 44 - protein, 368 aa, Artificial Sequence
  Synthetic Construct
MALPVTALLLPLALLLHAARPQVQLVESGGGSAQSGGSLRLSCAISEFTYKNTCVGWFRQAPGKEREGVAAIDSDGNTNYVDSVKGRFTISQGNAKNTVYLQMNSLKPEDTAMYYCAAGAYCGRLLLWIGNYAYWGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - protein, 491 aa, Artificial Sequence
  Synthetic Construct
MALPVTALLLPLALLLHAARPQVQLVESGGGSAQSGGSLRLSCAISEFTYKNTCVGWFRQAPGKEREGVAAIDSDGNTNYVDSVKGRFTISQGNAKNTVYLQMNSLKPEDTAMYYCAAGAYCGRLLLWIGNYAYWGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMPMGSLQPLATLYLLGMLVASCLGCNGRCGGGGGSGGGGSGGGGSPPRASALPAPPTGSALPDPQTASALPDPPAASALPAALAVISFLLGLGLGVACVLA

SEQ ID NO: 46 - protein, 499 aa, Artificial Sequence
  Synthetic Construct
MALPVTALLLPLALLLHAARPQVQLVESGGGSAQSGGSLRLSCAISEFTYKNTCVGWFRQAPGKEREGVAAIDSDGNTNYVDSVKGRFTISQGNAKNTVYLQMNSLKPEDTAMYYCAAGAYCGRLLLWIGNYAYWGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMPMGSLQPLATLYLLGMLVASCLGCNGRCGGGGGSGGGGSGGGGSDYKDDDDKPPRASALPAPPTGSALPDPQTASALPDPPAASALPAALAVISFLLGLGLGVACVLA

SEQ ID NO: 47 - protein, 45 aa, Artificial Sequence
  Synthetic Construct
TTTPAPRPPTPAPTIASQPLSLRPEACRPAAGGAVHTRGLDFACD

SEQ ID NO: 48 - protein, 21 aa, Artificial Sequence
  Synthetic Construct
IYIWAPLAGTCGVLLLSLVIT

SEQ ID NO: 49 - protein, 533 aa, Artificial Sequence
  Synthetic Construct
MALPVTALLLPLALLLHAARPCDCRGDCFCGGGGSGGGGSGGGGSQVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSSGGGGSGGGGSGGGGSEVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 533 aa, Artificial Sequence
  Synthetic Construct
MALPVTALLLPLALLLHAARPQVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSSGGGGSGGGGSGGGGSEVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSSGGGGSGGGGSGGGGSCDCRGDCFCTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - protein, 729 aa, Artificial Sequence
  Synthetic Construct
MALPVTALLLPLALLLHAARPQVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSSGGGGSGGGGSGGGGSEVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSS

SEQ ID NO: 52 - protein, 739 aa, Artificial Sequence
  Synthetic Construct
MALPVTALLLPLALLLHAARPQVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSSGGGGSGGGGSGGGGSEVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMLRLLLALNLFPSIQVTGCDCRGDCFCGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSS

SEQ ID NO: 53 - protein, 745 aa, Artificial Sequence
  Synthetic Construct
MALPVTALLLPLALLLHAARPQVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSSGGGGSGGGGSGGGGSEVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMPMGSLQPLATLYLLGMLVASCLGCDCRGDCFCGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSS

SEQ ID NO: 54 - protein, 736 aa, Artificial Sequence
  Synthetic Construct
MALPVTALLLPLALLLHAARPQVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSSGGGGSGGGGSGGGGSEVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMLRLLLALNLFPSIQVTGCNGRCGGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSS

SEQ ID NO: 55 - protein, 852 aa, Artificial Sequence
  Synthetic Construct
MALPVTALLLPLALLLHAARPQVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSSGGGGSGGGGSGGGGSEVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSGSGATNFSLLKQAGDVEENPGPMPMGSLQPLATLYLLGMLVASCLGCNGRCGGGGGSGGGGSGGGGSPPRASALPAPPTGSALPDPQTASALPDPPAASALPAALAVISFLLGLGLGVACVLA

SEQ ID NO: 56 - protein, 860 aa, Artificial Sequence
  Synthetic Construct
MALPVTALLLPLALLLHAARPQVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSSGGGGSGGGGSGGGGSEVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSGSGATNFSLLKQAGDVEENPGPMPMGSLQPLATLYLLGMLVASCLGCNGRCGGGGGSGGGGSGGGGSDYKDDDDKPPRASALPAPPTGSALPDPQTASALPDPPAASALPAALAVISFLLGLGLGVACVLA

SEQ ID NO: 57 - protein, 863 aa, Artificial Sequence
  Synthetic Construct
MALPVTALLLPLALLLHAARPQVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSSGGGGSGGGGSGGGGSEVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMPMGSLQPLATLYLLGMLVASCLGCNGRCGGGGGSCNGRCGGGGGSGGGGSGGGGSPPRASALPAPPTGSALPDPQTASALPDPPAASALPAALAVISFLLGLGLGVACVLAGSGEGRGSLLTCGDVEENPGPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSS

SEQ ID NO: 58 - protein, 871 aa, Artificial Sequence
  Synthetic Construct
MALPVTALLLPLALLLHAARPQVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSSGGGGSGGGGSGGGGSEVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMPMGSLQPLATLYLLGMLVASCLGCNGRCGGGGGSCNGRCGGGGGSGGGGSGGGGSDYKDDDDKPPRASALPAPPTGSALPDPQTASALPDPPAASALPAALAVISFLLGLGLGVACVLAGSGEGRGSLLTCGDVEENPGPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSS

(19) United States
(12) Patent Application Publication
(10) Pub. No.: US 2024/0150456 A1
(43) Pub. Date: May 9, 2024

(54) ENGINEERED IMMUNE CELLS AND USES THEREOF

(71) Applicant: Nanjing Legend Biotech Co., Ltd., Nanjing (CN)

(72) Inventors: Fei XU, Nanjing (CN); Ruidong Hao, Nanjing (CN); Wang Zhang, Nanjing (CN); Shu Wu, Nanjing (CN)

(21) Appl. No.: 18/282,696

(22) PCT Filed: Apr. 2, 2022
(86) PCT No.: PCT/CN2022/085044

§ 371 Date: Sep. 18, 2023

(30) Foreign Application Priority Data
Apr. 2, 2021 (WO) PCT/CN2021/085325

(51) Int. Cl. C07K 16/28; A61K 39/00; A61P 35/00; C07K 14/705; C07K 14/71; C12N 5/0783

(52) U.S. Cl. CPC C07K 16/28; A61K 39/4631; A61K 39/4637; A61K 39/464402; A61P 35/00; C07K 14/70521; C07K 14/71; C12N 5/0636; C07K 2319/03; C12N 2510/00

(57) ABSTRACT

The present disclosure provides immune effector cells engineered to express a functional exogenous receptor such as a CAR armored with a tumor homing peptide. The immune effector cells according to the present disclosure have enhanced tumor infiltration and anti-tumor efficacy.

### CROSS REFERENCE TO RELATED APPLICATION

This application claims priority benefits of International Application No. PCT/CN2021/085325, filed Apr. 2, 2021, entitled “ENGINEERED IMMUNE CELLS AND USES THEREOF”, the contents of which is incorporated herein by reference in its entirety.

### SEQUENCE STATEMENT

The content of the following submission on ASCII text file is incorporated herein by reference in its entirety: a computer readable form (CRF) of the Sequence Listing (file name: 761422003840SEQLIST.TXT, date recorded: Mar. 30, 2022, size: 185,862 bytes).

### FIELD OF THE INVENTION

The present disclosure relates to genetically engineered immune cells for therapeutic and related applications. In particular, the present disclosure relates to immune effector cells comprising a functional exogenous receptor and an exogenous tumor homing peptide and uses thereof.

### BACKGROUND OF THE INVENTION

Adoptive cell therapy, also known as cellular immunotherapy, is a form of treatment that uses cells of the immune system to treat diseases, for example, to eliminate cancer. Cellular immunotherapies can be deployed in different ways such as Tumor-Infiltrating Lymphocyte (TIL) Therapy, Engineered T Cell Receptor (TCR) Therapy, Chimeric Antigen Receptor (CAR) T Cell Therapy, and Natural Killer (NK) Cell Therapy.

Chimeric antigen receptors (CARs) are synthetic receptors that recognize their cognate target ligands and induce T cell intrinsic signaling. Although profound progress in hematologic cancer has been achieved, various barriers still restrict the application of CAR-T in solid tumors. Key challenges include tumor trafficking, infiltration into tumor, activation and persistence in tumors, and tumor heterogeneity. See, Rafiq S, Hackett C S, Brentjens R J. Engineering strategies to overcome the current roadblocks in CAR T cell therapy. Nat Rev Clin Oncol. 2020 March; 17(3):147-167. doi: 10.1038/s41571-019-0297-y. Epub 2019 Dec. 17. PMID: 31848460; PMCID: PMC7223338. Thus, new designs to tackle these key challenges are urgently needed to improve the clinical efficacy of CAR-T or other cellular therapies in solid tumors.

All publications, patents and patent applications referred to herein are incorporated by reference in their entirety to the same extent as if each individual publication, patent or patent application was specifically and individually indicated to be incorporated by reference in its entirety. Citation or identification of any document in this application is not an admission that such document is available as prior art to the present disclosure.

### SUMMARY OF THE INVENTION

The present application in some aspects provides an immune effector cell expressing: (a) a functional exogenous receptor, and (b) an exogenous tumor homing peptide (THP).

In some embodiments of the immune effector cell, the functional exogenous receptor is selected from the group consisting of a chimeric antigen receptor (CAR), an engineered T cell receptor (TCR), a chimeric TCR (cTCR), a T cell antigen coupler (TAC), a TAC-like chimeric receptor, and combinations thereof. In some embodiments, the functional exogenous receptor is a CAR. In some embodiments, the functional exogenous receptor is a TCR. In some embodiments, the functional exogenous receptor is a cTCR. In some embodiments, the functional exogenous receptor is a TAC. In some embodiments, the functional exogenous receptor is a TAC-like chimeric receptor.

In some embodiments of the immune effector cell, the functional exogenous receptor is a CAR and wherein the CAR comprises (i) an extracellular antigen binding domain, (ii) a transmembrane domain, and (iii) an intracellular signaling domain. In some embodiments, the transmembrane domain is derived from a molecule selected from the group consisting of CD7, CD8, CD8α, CD4, CD28, CD137 (4-1BB), CD80, CD86, CD152, FasL, TR2 and PD1. In some embodiments, the intracellular signaling domain comprises a primary intracellular signaling domain of an immune effector cell. In some embodiments, the primary intracellular signaling domain is from CD3. In some embodiments, the intracellular signaling domain comprises a co-stimulatory signaling domain. In some embodiments, the co-stimulatory signaling domain is derived from a co-stimulatory molecule selected from the group consisting of CD27, CD28, CD137 (4-1BB), OX40, CD30, CD40, CD3, LFA-1, ICOS, CD2, CD7, LIGHT, NKG2C, B7-H3, ligands of CD83 and combinations thereof. In some embodiments, the immune effector cell further comprises a hinge domain located between the C-terminus of the extracellular antigen binding domain and the N-terminus of the transmembrane domain. In some embodiments, the hinge domain is from CD8 or CD28. In some embodiments, the immune effector cell further comprises a signal peptide located at the N-terminus of the extracellular antigen binding domain. In some embodiments, the signal peptide is from CD8 or CD28.

In some embodiments of the immune effector cell, the immune effector cell is selected from the group consisting of T cells, natural killer (NK) cells, B cells, neutrophils, eosinophils, monocytes, macrophages, dendritic cells, peripheral blood mononuclear cells (PBMC), stem cells from which lymphoid cells may be differentiated, and combinations thereof. In some embodiments, the immune effector cell is a T cell.

In some embodiments of the immune effector cell, the CAR is an anti-DLL3 CAR. In some embodiments, the anti-DLL3 CAR comprises a first V_{H}H antibody moiety that comprises a CDR1 comprising the amino acid sequence of SEQ ID NO: 29 or a variant thereof comprising up to about 3 amino acid substitutions, a CDR2 comprising the amino acid sequence of SEQ ID NO: 30 or a variant thereof comprising up to about 3 amino acid substitutions, and a CDR3 comprising the amino acid sequence of SEQ ID NO: 31 or a variant thereof comprising up to about 3 amino acid substitutions, and a second V_{H}H antibody moiety that comprises a CDR1 comprising the amino acid sequence of SEQ ID NO: 32 or a variant thereof comprising up to about 3 amino acid substitutions, a CDR2 comprising the amino acid sequence of SEQ ID NO: 33 or a variant thereof comprising up to about 3 amino acid substitutions, and a CDR3 comprising the amino acid sequence of SEQ ID NO: 34 or a variant thereof comprising up to about 3 amino acid substitutions. In some embodiments, the anti-DLL3 CAR comprises a first V_{H}H antibody moiety comprising the amino acid sequences of SEQ ID NO: 27 or an amino acid sequence having at least 75%, 80%, 90%, 91%, 92%, 93%, 94%, 95%, 96%, 97%, 98%, or 99% identity to the amino acid sequence of SEQ ID NO: 27, and a second V_{H}H antibody moiety comprising the amino acid sequences of SEQ ID NO: 28 or an amino acid sequence having at least 75%, 80%, 90%, 91%, 92%, 93%, 94%, 95%, 96%, 97%, 98%, or 99% identity to the amino acid sequence of SEQ ID NO: 28. In some embodiments, the anti-DLL3 CAR comprises the amino acid sequence of SEQ ID NO: 8 or an amino acid sequence having at least 75%, 80%, 90%, 91%, 92%, 93%, 94%, 95%, 96%, 97%, 98%, or 99% identity to the amino acid sequence of SEQ ID NO: 8.

In some embodiments of the immune effector cell, the CAR is an anti-MSLN CAR. In some embodiments, the anti-MSLN CAR comprises the amino acid sequence of SEQ ID NO: 41 or an amino acid sequence having at least 75%, 80%, 90%, 91%, 92%, 93%, 94%, 95%, 96%, 97%, 98%, or 99% identity to the amino acid sequence of SEQ ID NO: 41.

In some embodiments of the immune effector cell, the CAR is an anti-GPC2 CAR. In some embodiments, the anti-GPC2 CAR comprises the amino acid sequence of SEQ ID NO: 44 or an amino acid sequence having at least 75%, 80%, 90%, 91%, 92%, 93%, 94%, 95%, 96%, 97%, 98%, or 99% identity to the amino acid sequence of SEQ ID NO: 44.

In some embodiments of the immune effector cell, the CAR is multispecific.

In some embodiments of the immune effector cell, the THP is selected from the group consisting of arginine-glycine-aspartic (RGD)-based peptides, asparagine-glycine-arginine (NGR)-based peptides, and combinations thereof. In some embodiments, the THP comprises the RGD-4C peptide having the amino acid sequence of SEQ ID NO: 2. In some embodiments, the THP comprises the NGR peptide having the amino acid sequence of SEQ ID NO: 38.

In some embodiments of the immune effector cell, the THP is fused with a transmembrane domain and/or a hinge domain. In some embodiments, the transmembrane domain and/or the hinge domain is from CD8, CD8α, CD7, CD80, CD28, TR2, or FasL. In some embodiments, the transmembrane domain and/or the hinge domain is from CD7. In some embodiments, the transmembrane domain comprises the amino acid sequence of SEQ ID NO: 14, and the hinge domain comprises the amino acid sequence of SEQ ID NO: 13. In some embodiments, the transmembrane domain and/or the hinge domain is from TR2.

In some embodiments of the immune effector cell, the THP is fused with a TGF-β dominant-negative receptor (TGF-β DNR). In some embodiments, the immune effector cell comprises a polypeptide comprising: i) the amino acid sequence of SEQ ID NO: 55 or an amino acid sequence having at least 75%, 80%, 90%, 91%, 92%, 93%, 94%, 95%, 96%, 97%, 98%, or 99% identity to the amino acid sequence of SEQ ID NO: 55; ii) the amino acid sequence of SEQ ID NO: 57 or an amino acid sequence having at least 75%, 80%, 90%, 91%, 92%, 93%, 94%, 95%, 96%, 97%, 98%, or 99% identity to the amino acid sequence of SEQ ID NO: 57; iii) the amino acid sequence of SEQ ID NO: 54 or an amino acid sequence having at least 75%, 80%, 90%, 91%, 92%, 93%, 94%, 95%, 96%, 97%, 98%, or 99% identity to the amino acid sequence of SEQ ID NO: 54; iv) the amino acid sequence of SEQ ID NO: 56 or an amino acid sequence having at least 75%, 80%, 90%, 91%, 92%, 93%, 94%, 95%, 96%, 97%, 98%, or 99% identity to the amino acid sequence of SEQ ID NO: 56; v) the amino acid sequence of SEQ ID NO: 58 or an amino acid sequence having at least 75%, 80%, 90%, 91%, 92%, 93%, 94%, 95%, 96%, 97%, 98%, or 99% identity to the amino acid sequence of SEQ ID NO: 58; vi) the amino acid sequence of SEQ ID NO: 53 or an amino acid sequence having at least 75%, 80%, 90%, 91%, 92%, 93%, 94%, 95%, 96%, 97%, 98%, or 99% identity to the amino acid sequence of SEQ ID NO: 53; or vii) the amino acid sequence of SEQ ID NO: 52 or an amino acid sequence having at least 75%, 80%, 90%, 91%, 92%, 93%, 94%, 95%, 96%, 97%, 98%, or 99% identity to the amino acid sequence of SEQ ID NO: 52.

In some embodiments of the immune effector cell, the THP is fused with a tag sequence. In some embodiments, the THP is fused with a peptide linker. In some embodiments, the THP is fused with a tag sequence and a peptide linker. In some embodiments, the tag sequence is a Flag tag comprising the amino acid sequence of SEQ ID NO: 3, and the peptide linker is a (G4S)₃, (G4S)₂, or G4S linker. In some embodiments, the THP is fused with a glycosylphosphatidylinositol (GPI) linkage.

In some embodiments of the immune effector cell, the THP is fused with an intracellular domain. In some embodiments, the THP is not fused with an intracellular domain.

In some embodiments of the immune effector cell, the functional exogenous receptor is a cTCR. In some embodiments, the cTCR is an anti-DLL3 cTCR. In some embodiments, the anti-DLL3 cTCR comprises the amino acid sequence of SEQ ID NO: 35 or an amino acid sequence having at least 75%, 80%, 90%, 91%, 92%, 93%, 94%, 95%, 96%, 97%, 98%, or 99% identity to the amino acid sequence of SEQ ID NO: 35. In some embodiments, the anti-DLL3 cTCR comprises the amino acid sequence of SEQ ID NO: 36 or an amino acid sequence having at least 75%, 80%, 90%, 91%, 92%, 93%, 94%, 95%, 96%, 97%, 98%, or 99% identity to the amino acid sequence of SEQ ID NO: 36.

In some embodiments of the immune effector cell, the immune effector cell comprises a polypeptide having the amino acid sequence set forth in any one of SEQ ID NOs: 10, 11, 19, 21-26, 35-37, 39-40, 42, 43, 45, 46, 49, 50, and 52-58.

In other aspects, provided herein is a polypeptide comprising: (a) a functional exogenous receptor, and (b) an exogenous THP.

In some embodiments of the polypeptide, the functional exogenous receptor is selected from the group consisting of a chimeric antigen receptor (CAR), an engineered T cell receptor (TCR), a chimeric TCR (cTCR), a T cell antigen coupler (TAC), a TAC-like chimeric receptor, and combinations thereof. In some embodiments, the functional exogenous receptor is a CAR. In some embodiments, the functional exogenous receptor is a TCR. In some embodiments, the functional exogenous receptor is a cTCR. In some embodiments, the functional exogenous receptor is a TAC. In some embodiments, the functional exogenous receptor is a TAC-like chimeric receptor.

In some embodiments, the functional exogenous receptor is a CAR and wherein the CAR comprises (i) an extracellular antigen binding domain, (ii) a transmembrane domain, and (iii) an intracellular signaling domain. In some embodiments, the transmembrane domain is derived from a molecule selected from the group consisting of CD7, CD8, CD8α, CD4, CD28, CD137 (4-1BB), CD80, CD86, CD152, FasL, TR2, and PD1. In some embodiments, the intracellular signaling domain comprises a primary intracellular signaling domain of an immune effector cell. In some embodiments, the primary intracellular signaling domain is from CD3. In some embodiments, the intracellular signaling domain comprises a co-stimulatory signaling domain. In some embodiments, the co-stimulatory signaling domain is derived from a co-stimulatory molecule selected from the group consisting of CD27, CD28, CD137 (4-1BB), OX40, CD30, CD40, CD3, LFA-1, ICOS, CD2, CD7, LIGHT, NKG2C, B7-H3, ligands of CD83 and combinations thereof. In some embodiments, the polypeptide further comprises a hinge domain located between the C-terminus of the extracellular antigen binding domain and the N-terminus of the transmembrane domain. In some embodiments, the hinge domain is from CD28 or CD8. In some embodiments, the polypeptide further comprises a signal peptide located at the N-terminus of the extracellular antigen binding domain. In some embodiments, the signal peptide is from CD28 or CD8.

In some embodiments of the polypeptide, the CAR is an anti-DLL3 CAR. In some embodiments, the anti-DLL3 CAR comprises a first V_{H}H antibody moiety that comprises a CDR1 comprising the amino acid sequence of SEQ ID NO: 29 or a variant thereof comprising up to about 3 amino acid substitutions, a CDR2 comprising the amino acid sequence of SEQ ID NO: 30 or a variant thereof comprising up to about 3 amino acid substitutions, and a CDR3 comprising the amino acid sequence of SEQ ID NO: 31 or a variant thereof comprising up to about 3 amino acid substitutions, and a second V_{H}H antibody moiety that comprises a CDR1 comprising the amino acid sequence of SEQ ID NO: 32 or a variant thereof comprising up to about 3 amino acid substitutions, a CDR2 comprising the amino acid sequence of SEQ ID NO:33 or a variant thereof comprising up to about 3 amino acid substitutions, and a CDR3 comprising the amino acid sequence of SEQ ID NO: 34 or a variant thereof comprising up to about 3 amino acid substitutions. In some embodiments, the anti-DLL3 CAR comprises a first V_{H}H antibody moiety comprising the amino acid sequences of SEQ ID NO: 27 or an amino acid sequence having at least 75%, 80%, 90%, 91%, 92%, 93%, 94%, 95%, 96%, 97%, 98%, or 99% identity to the amino acid sequence of SEQ ID NO: 27, and a second V_{H}H antibody moiety comprising the amino acid sequences of SEQ ID NO: 28 or an amino acid sequence having at least 75%, 80%, 90%, 91%, 92%, 93%, 94%, 95%, 96%, 97%, 98%, or 99% identity to the amino acid sequence of SEQ ID NO: 28. In some embodiments, the anti-DLL3 CAR comprises the amino acid sequence of SEQ ID NO: 8 or an amino acid sequence having at least 75%, 80%, 90%, 91%, 92%, 93%, 94%, 95%, 96%, 97%, 98%, or 99% identity to the amino acid sequence of SEQ ID NO: 8.

In some embodiments of the polypeptide, the CAR is an anti-MSLN CAR. In some embodiments, the anti-MSLN CAR comprises the amino acid sequence of SEQ ID NO: 41 or an amino acid sequence having at least 75%, 80%, 90%, 91%, 92%, 93%, 94%, 95%, 96%, 97%, 98%, or 99% identity to the amino acid sequence of SEQ ID NO: 41.

In some embodiments of the polypeptide, the CAR is an anti-GPC2 CAR. In some embodiments, the anti-GPC2 CAR comprises the amino acid sequence of SEQ ID NO: 44 or an amino acid sequence having at least 75%, 80%, 90%, 91%, 92%, 93%, 94%, 95%, 96%, 97%, 98%, or 99% identity to the amino acid sequence of SEQ ID NO: 44.

In some embodiments of the polypeptide, the CAR is multispecific.

In some embodiments of the polypeptide, the exogenous THP is selected from the group consisting of RGD-based peptides, NGR-based peptides, and combinations thereof. In some embodiments, the THP comprises the RGD-4C peptide having the amino acid sequence of SEQ ID NO: 2. In some embodiments, the THP comprises the NGR peptide having the amino acid sequence of SEQ ID NO: 38.

In some embodiments of the polypeptide, the THP is fused with a transmembrane domain and/or a hinge domain. In some embodiments, the transmembrane domain and/or the hinge domain is from CD8, CD8α, CD7, CD80, CD28, TR2, or FasL. In some embodiments, the transmembrane domain and/or the hinge domain is from CD7. In some embodiments, the transmembrane domain comprises the amino acid sequence of SEQ ID NO: 14, and the hinge domain comprises the amino acid sequence of SEQ ID NO: 13. In some embodiments, the transmembrane domain and/or the hinge domain is from TR2.

In some embodiments of the polypeptide, the THP is fused with a TGF-β DNR. In some embodiments, the polypeptide comprises: i) the amino acid sequence of SEQ ID NO: 55 or an amino acid sequence having at least 75%, 80%, 90%, 91%, 92%, 93%, 94%, 95%, 96%, 97%, 98%, or 99% identity to the amino acid sequence of SEQ ID NO: 55; ii) the amino acid sequence of SEQ ID NO: 57 or an amino acid sequence having at least 75%, 80%, 90%, 91%, 92%, 93%, 94%, 95%, 96%, 97%, 98%, or 99% identity to the amino acid sequence of SEQ ID NO: 57; iii) the amino acid sequence of SEQ ID NO: 54 or an amino acid sequence having at least 75%, 80%, 90%, 91%, 92%, 93%, 94%, 95%, 96%, 97%, 98%, or 99% identity to the amino acid sequence of SEQ ID NO: 54; iv) the amino acid sequence of SEQ ID NO: 56 or an amino acid sequence having at least 75%, 80%, 90%, 91%, 92%, 93%, 94%, 95%, 96%, 97%, 98%, or 99% identity to the amino acid sequence of SEQ ID NO: 56; v) the amino acid sequence of SEQ ID NO: 58 or an amino acid sequence having at least 75%, 80%, 90%, 91%, 92%, 93%, 94%, 95%, 96%, 97%, 98%, or 99% identity to the amino acid sequence of SEQ ID NO: 58; vi) the amino acid sequence of SEQ ID NO: 53 or an amino acid sequence having at least 75%, 80%, 90%, 91%, 92%, 93%, 94%, 95%, 96%, 97%, 98%, or 99% identity to the amino acid sequence of SEQ ID NO: 53; or vii) the amino acid sequence of SEQ ID NO: 52 or an amino acid sequence having at least 75%, 80%, 90%, 91%, 92%, 93%, 94%, 95%, 96%, 97%, 98%, or 99% identity to the amino acid sequence of SEQ ID NO: 52.

In some embodiments of the polypeptide, the THP is fused with a tag sequence. In some embodiments, the THP is fused with a peptide linker. In some embodiments, the THP is fused with a tag sequence and a peptide linker. In some embodiments, the tag sequence is a Flag tag comprising the amino acid sequence of SEQ ID NO: 3, and the peptide linker is a (G4S)₃, (G45)₂, or G4S linker. In some embodiments, the THP is fused with a glycosylphosphatidylinositol (GPI) linkage.

In some embodiments of the polypeptide, the THP is fused with an intracellular domain. In some embodiments of the polypeptide, the THP is not fused with an intracellular domain.

In some embodiments of the polypeptide, the polypeptide further comprises a self-cleaving peptide between the functional exogenous receptor and the THP. In some embodiments, the self-cleaving peptide is P2A. In some embodiments, the self-cleaving peptide is T2A.

In some embodiments of the polypeptide, the functional exogenous receptor is at the N terminus or C terminus of the exogenous tumor homing peptide. In some embodiments, the polypeptide comprises the amino acid sequence set forth in any one of SEQ ID NOs: 10, 11, 19, 21-26, 35, 36, 37, 39-40, 42, 43, 45, 46, 49, 50, and 52-58.

In further aspects, provided herein is an isolated nucleic acid comprising a nucleic acid sequence encoding the polypeptide of any one of the preceding embodiments.

In additional aspects, provided herein is a vector comprising the isolated nucleic acid of the preceding embodiment.

In other aspects, provided herein is a host cell comprising the vector of the preceding embodiment.

In still further aspects, provided herein is a method of making an immune effector cell of any one of the preceding embodiments comprising introducing into an immune effector cell: (i) the nucleic acid of the preceding embodiment or the vector of the preceding embodiment; or (ii) a composition comprising two nucleic acids each encoding: (a) a functional exogenous receptor, and (b) an exogenous THP. In some embodiments, provided herein is an immune effector cell produced according to the method of the preceding embodiment.

In other aspects, provided herein is a pharmaceutical composition, comprising the immune effector cell of any one of the preceding embodiments, the polypeptide of any one of the preceding embodiments, the nucleic acid of the preceding embodiment, or the vector of the preceding embodiment, and a pharmaceutically acceptable carrier.

In other aspects, provided herein is a method of treating a disease or disorder in a subject, comprising administering to the subject an effective amount of the pharmaceutical composition of the preceding embodiment. In some embodiments, the disease or disorder is selected from the group consisting of cancer, infectious disease, inflammatory disease, autoimmune disease, and the combinations thereof. In some embodiments, the cancer is a solid tumor cancer or hematological cancer. In some embodiments, the cancer is small-cell lung cancer (SCLC), ovarian cancer (OC) or neuroblastoma (NBL).

### BRIEF DESCRIPTION OF THE DRAWINGS

The following detailed description, given by way of example, but not intended to limit the disclosure solely to the specific embodiments described, may best be understood in conjunction with the accompanying drawings.

FIG. 1 shows the expression of membrane bound RGD (mbRGD) and an anti-DLL3 CAR on T cells; the mbRGD comprises either the transmembrane domain of CD28 or the transmembrane domains of FasL; mbRGD expression is detected by anti-Flag staining and FACS, and CAR expression is determined by anti-DLL3 single domain antibody (sdAb) staining and FACS.

FIG. 2 shows the influence of different components of mbRGD on the expression on T cells: mbRGD expression is detected by eGFP, and CAR expression is determined by anti-DLL3 sdAb staining and FACS.

FIG. 3 shows the expression of mbRGD and CAR on T cells; the mbRGD comprises the transmembrane of CD80; mbRGD expression is detected by anti-Flag staining and FACS, and CAR expression is determined by anti-DLL3 sdAb staining and FACS.

FIG. 4 shows the expression of mbRGD and CAR on T cells; the mbRGD is comprises the transmembrane of CD7; mbRGD expression is detected by anti-Flag staining and FACS, and CAR expression is determined by anti-DLL3 sdAb staining and FACS.

FIGS. 5A-5B shows the representative images (FIG. 5A) and quantification (FIG. 5B) of adhesion of mbRGD armored DLL3-CAR-T cells or conventional DLL3-CAR-T cells to HUVEC cells.

FIGS. 6A-6B show the cytolytic effect by mbRGD armored DLL3-CAR-T cells or un-armored DLL3-CAR-T cells against SHP77 (FIG. 6A) or HUVEC (FIG. 6B) cells.

FIGS. 7A-7B show IFN-γ secretion by mbRGD armored DLL3-CAR-T cells or un-armored DLL3-CAR-T cells co-incubated with SHP77 (FIG. 7A) or HUVEC (FIG. 7B) cells.

FIGS. 8A-8B show the antitumor efficacy of mbRGD armored DLL3-CAR-T cells or conventional DLL3-CAR-T cells in xenografted mice model; tumor growth curve (FIG. 8A) and CAR-T expansion in peripheral blood (FIG. 8B).

FIGS. 9A-8B shows tumor infiltration of mbRGD armored DLL3-CAR-T cells or conventional DLL3-CAR-T cells in xenografted mice model. Tumor infiltration was detected by IHC staining of sdAb. Results shown are pooled results from 3 mice each group.

FIGS. 10A-10B show the representative images (FIG. 10A) and quantification (FIG. 10B) of adhesion of different expression levels of mbRGD armored DLL3-CAR-T cells or conventional DLL3-CAR-T cells to HUVEC cells.

FIG. 11 shows the expression of mbRGD and 6-T cell receptor fusion construct (TRuC) on T cells; the mbRGD is based on the transmembrane of CD7; mbRGD expression is detected by anti-Flag staining and FACS, and 6-TRuC expression is determined by anti-DLL3 sdAb staining and FACS.

FIGS. 12A-12B show the representative images (FIG. 12A) and quantification (FIG. 12B) of adhesion of mbRGD armored DLL3-ε-TRuC-T cells or conventional DLL3-ε-TRuC-T cells to HUVEC cells.

FIGS. 13A-13B show the cytolytic effect by mbRGD armored DLL3-ε-TRuC-T cells or un-armored DLL3-ε-TRuC-T cells against SHP77 (FIG. 13A) or HUVEC (FIG. 13B) cells.

FIGS. 14A-14B show IFN-γ secretion by mbRGD armored DLL3-ε-TRuC-T cells or un-armored DLL3-ε-TRuC-T cells co-incubated with SHP77 (FIG. 14A) or HUVEC (FIG. 14B) cells.

FIG. 15 shows the expression of mbNGR and DLL3-CAR on T cells; the mbNGR is based on the transmembrane of CD7; mbNGR expression is detected by anti-Flag staining and FACS, and CAR expression is determined by anti-sdAb staining and FACS.

FIGS. 16A-16B show the representative images (FIG. 16A) and quantification (FIG. 16B) of adhesion of mbNGR armored DLL3-CAR-T cells or conventional DLL3-CAR-T cells to HUVEC cells.

FIGS. 17A-17B show the cytolytic effect by mbNGR armored DLL3-CAR-T cells or un-armored DLL3-CAR-T cells against SHP77 (FIG. 17A) or HUVEC (FIG. 17B) cells.

FIGS. 18A-18B show IFN-γ secretion by mbNGR armored DLL3-CAR-T cells or un-armored DLL3-CAR-T cells co-incubated with SHP77 (FIG. 18A) or HUVEC (FIG. 18B) cells.

FIG. 19 shows the expression of mbNGR and MSLN-CAR on T cells; the mbNGR is based on the transmembrane of CD7; mbNGR expression is detected by anti-Flag staining and FACS, and CAR expression is determined by anti-sdAb staining and FACS.

FIGS. 20A-20B show the representative images (FIG. 20A) and quantification (FIG. 20B) of adhesion of mbNGR armored MSLN-CAR-T cells or conventional MSLN-CAR-T cells to HUVEC cells.

FIGS. 21A-21B show the cytolytic effect by mbNGR armored MSLN-CAR-T cells or un-armored MSLN-CAR-T cells against OVCAR3 (FIG. 21A) or HUVEC (FIG. 21B) cells.

FIGS. 22A-22B show IFN-γ secretion by mbNGR armored MSLN-CAR-T cells or un-armored MSLN-CAR-T cells co-incubated with OVCAR3 (FIG. 22A) or HUVEC (FIG. 22B) cells.

FIG. 23 shows the expression of mbNGR and GPC2-CAR on T cells; the mbNGR is based on the transmembrane of CD7; mbNGR expression is detected by anti-Flag staining and FACS, and CAR expression is determined by anti-sdAb staining and FACS.

FIGS. 24A-24B show the representative images (FIG. 24A) and quantification (FIG. 24B) of adhesion of mbNGR armored GPC2-CAR-T cells or conventional GPC2-CAR-T cells to HUVEC cells.

FIGS. 25A-25B show the cytolytic effect by mbNGR armored GPC2-CAR-T cells or un-armored GPC2-CAR-T cells against SH-SY5Y (FIG. 25A) or HUVEC (FIG. 25B) cells.

FIGS. 26A-26B show IFN-γ secretion by mbNGR armored GPC2-CAR-T cells or un-armored GPC2-CAR-T cells co-incubated with SH-SY5Y (FIG. 26A) or HUVEC (FIG. 26B) cells.

FIG. 27 shows the expression of CAR on RGD embedding CAR-T cells and mbRGD armored CAR-T cells; CAR expression is determined by anti-sdAb staining and FACS.

FIGS. 28A-28B show the representative images (FIG. 28A) and quantification (FIG. 28B) of adhesion of RGD embedding CAR-T cells, mbRGD armored CAR-T cells or conventional CAR-T cells to HUVEC cells.

FIGS. 29A-29B show the cytolytic effect by RGD embedding CAR-T cells, mbRGD armored CAR-T cells or conventional CAR-T cells against SHP-77 cells (FIG. 29A) or HUVEC cells (FIG. 29B).

FIGS. 30A-30B show IFN-γ secretion by RGD embedding CAR-T cells, mbRGD armored CAR-T cells or conventional CAR-T cells co-incubated with SHP-77 (FIG. 30A) or HUVEC (FIG. 30B) cells.

FIGS. 31A-31C show the expression of DLL3-CAR/mbRGD/mbNGR/TGFβRII on mbRGD (FIG. 31A)/mbNGR (FIGS. 31B & 31C) combined TGF-β DNR armored CAR-T cells. CAR expression is determined by anti-sdAb staining and FACS, mbRGD/mbNGR expression is detected by anti-Flag staining and FACS, and TGF-β DNR expression is detected by anti-TGF-βRII staining and FACS.

FIGS. 32A-32F show the representative images (FIG. 32A, C, E) and quantification (FIG. 32B, D, F) of adhesion of mbRGD/mbNGR combined TGF-β DNR armored CAR-T cells, mbRGD/mbNGR armored CAR-T cells, TGFβ DNR armored CAR-T cells or conventional CAR-T cells to HUVEC cells.

FIGS. 33A-33C show the expression of dnTGF-βRII on mbRGD (FIG. 33A)/mbNGR (FIGS. 33B & 33C) combined TGF-β DNR armored CAR-T cells prevents TGF-β signal induction through SMAD2.

FIGS. 34A-34F show the cytolytic effect by mbRGD/mbNGR combined TGF-β DNR armored CAR-T cells, mbRGD/mbNGR armored CAR-T cells, TGFβ DNR armored CAR-T cells or conventional CAR-T cells against SHP-77 (FIGS. 34A, 34C, and 34E) or HUVEC (FIGS. 34B, 34D, and 34F) cells.

FIGS. 35A-35F show IFN-γ secretion by mbRGD/mbNGR combined TGF-β DNR armored CAR-T cells, mbRGD/mbNGR armored CAR-T cells, TGF-β DNR armored CAR-T cells or conventional CAR-T cells co-incubated with SHP-77 (FIGS. 35A, 35C, and 35E) or HUVEC (FIGS. 35B, 35D, and 35F) cells.

FIG. 36 shows tumor infiltration of mbRGD/mbNGR combined TGF-β DNR armored CAR-T cells or TGF-β DNR armored CAR-T cells in xenografted mice model. Tumor infiltration were detected by IHC staining of anti-sdAb. Results shown are pooled results from 3 mice each group.

### DETAILED DESCRIPTION OF THE INVENTION

The present disclosure is based, in part, on the surprising discovery that armoring CAR T cells with a tumor homing peptide can bring improvements such as enhanced adhesion of the T cells to neo-vasculature, and increased anti-tumor efficacy.

### 1. Definitions

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dilbel eds., 2d ed. 2010). Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

All embodiments provided throughout this application are non-limiting embodiments which are given for illustration purposes only and are not intended to limit the disclosure in any way. Different technical features, technical solutions, and/or embodiments that are discussed in the same or different aspects/parts of the present application can be combined to form new features, solutions, or embodiments, which also fall into the scope of the present disclosure.

It is understood that wherever embodiments are described herein with the term “comprising”, otherwise analogous embodiments described in terms of “consisting of” and/or “consisting essentially of” are also provided.

The terms “about” and “approximately” mean within 20%, within 15%, within 10%, within 9%, within 8%, within 7%, within 6%, within 5%, within 4%, within 3%, within 2%, within 1%, or less of a given value or range.

An “antigen” is a structure to which an antibody can selectively bind. A target antigen may be a polypeptide, carbohydrate, nucleic acid, lipid, hapten, or other naturally occurring or synthetic compound. In some embodiments, the target antigen is a polypeptide. In certain embodiments, an antigen is associated with a cell, for example, is present on or in a cell.

The terms “antibody” and “antibody moiety” are interchangeable in the context of the present application, and they are used in their broadest sense and encompass various antibody structures, including but not limited to monoclonal antibodies, polyclonal antibodies, multispecific antibodies (e.g., bispecific antibodies), full-length antibodies and antigen-binding fragments thereof, so long as they exhibit the desired antigen-binding activity. The terms include conventional four-chain antibodies, and single-domain antibodies, such as heavy-chain only antibodies or fragments thereof, e.g., V_{H}H.

A full-length four-chain antibody comprises two heavy chains and two light chains. The variable regions of the light and heavy chains are responsible for antigen binding. The variable domains of the heavy chain and light chain may be referred to as “VH” and “VL”, respectively. The variable regions in both chains generally contain three highly variable loops called the complementarity determining regions (CDRs) (light chain (LC) CDRs including LC-CDR1, LC-CDR2, and LC-CDR3, heavy chain (HC) CDRs including HC-CDR1, HC-CDR2, and HC-CDR3). CDR boundaries for the antibodies and antigen-binding fragments disclosed herein may be defined or identified by the conventions of Kabat, Chothia, or Al-Lazikani (Al-Lazikani, 1997, J. Mol. Biol., 273:927-948; Chothia 1985, J. Mol Biol., 186: 651-663; Chothia 1987, J. Mol. Biol., 196: 901-917; Chothia 1989, Nature, 342:877-883; Kabat 1987, Sequences of Proteins of Immunological Interest, Fourth Edition. US Govt. Printing Off. No. 165-492; Kabat 1991, Sequences of Proteins of Immunological Interest, Fifth Edition. NIH Publication No. 91-3242). The three CDRs of the heavy or light chains are interposed between flanking stretches known as framework regions (FRs), which are more highly conserved than the CDRs and form a scaffold to support the hypervariable loops. The constant regions of the heavy and light chains are not involved in antigen binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and μ heavy chains, respectively. Several of the major antibody classes are divided into subclasses such as lgG1 (γ1 heavy chain), lgG2 (γ2 heavy chain), lgG3 (γ3 heavy chain), lgG4 (γ4 heavy chain), IgA1 (α1 heavy chain), or lgA2 (α2 heavy chain).

The term “heavy chain-only antibody” or “HCAb” refers to a functional antibody, which comprises heavy chains, but lacks the light chains usually found in 4-chain antibodies. Camelid animals (such as camels, llamas, or alpacas) are known to produce HCAbs.

The term “single-domain antibody” or “sdAb” refers to a single antigen-binding polypeptide having three complementary determining regions (CDRs). The sdAb alone is capable of binding to the antigen without pairing with a corresponding CDR-containing polypeptide. In some cases, single-domain antibodies are engineered from camelid HCAbs, and their heavy chain variable domains are referred herein as “V_{H}Hs” (Variable domain of the heavy chain of the Heavy chain antibody). Camelid sdAb is one of the smallest known antigen-binding antibody fragments (see, e.g., Hamers-Casterman et al., Nature 363:446-8 (1993); Greenberg et al., Nature 374:168-73 (1995); Hassanzadeh-Ghassabeh et al., Nanomedicine (Lond), 8:1013-26 (2013)). A basic V_{H}H has the following structure from the N-terminus to the C-terminus: FR1-CDR1-FR2-CDR2-FR3-CDR3-FR4, in which FR1 to FR4 refer to framework regions 1 to 4, respectively, and in which CDR1 to CDR3 refer to the complementarity determining regions 1 to 3.

The term “antigen-binding fragment” as used herein refers to an antibody fragment including, for example, a diabody, a Fab, a Fab′, a F(ab′)₂, an Fv fragment, a disulfide stabilized Fv fragment (dsFv), a (dsFv)₂, a bispecific dsFv (dsFv-dsFv′), a disulfide stabilized diabody (ds diabody), a single-chain Fv (scFv), an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment (e.g., a parent scFv) binds. In some embodiments, an antigen-binding fragment may comprise one or more CDRs from a particular human antibody grafted to a framework region from one or more different human antibodies.

“Fv” is the minimum antibody fragment, which contains a complete antigen-recognition and -binding site. This fragment consists of a dimer of one heavy- and one light-chain variable region domain in tight, non-covalent association. From the folding of these two domains emanate six hypervariable loops (3 loops each from the heavy and light chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv,” also abbreviated as “sFv” or “scFv,” are antibody fragments that comprise the V_{H} and V_{L} antibody domains connected into a single polypeptide chain. In some embodiments, the scFv polypeptide further comprises a polypeptide linker between the VH and VL domains, which enables the scFv to form the desired structure for antigen binding. For a review of scFv, see Phickthun in The Pharmacology of Monoclonal Antibodies, vol. 113, Rosenburg and Moore eds., Springer-Verlag, New York, pp. 269-315 (1994).

The term “diabodies” refers to small antibody fragments prepared by constructing scFv fragments (see preceding paragraph) typically with short linkers (such as about 5 to about 10 residues) between the VH and VL domains such that inter-chain but not intra-chain pairing of the V domains is achieved, resulting in a bivalent fragment, i.e., fragment having two antigen-binding sites. Bispecific diabodies are heterodimers of two “crossover” scFv fragments in which the VH and VL domains of the two antibodies are present on different polypeptide chains. Diabodies are described more fully in, for example, EP 404,097; WO 93/11161; and Hollinger et al., Proc. Natl. Acad. Sci. USA, 90:6444-6448 (1993).

As used herein, the term “CDR” or “complementarity determining region” is intended to mean the non-contiguous antigen combining sites found within the variable region of both heavy and light chain polypeptides. These particular regions have been described by Kabat et al., J. Biol. Chem. 252:6609-6616 (1977); Kabat et al., U.S. Dept. of Health and Human Services, “Sequences of proteins of immunological interest” (1991); Chothia et al., J. Mol. Biol. 196:901-917 (1987); Al-Lazikani B. et al., J. Mol. Biol., 273: 927-948 (1997); MacCallum et al., J. Mol. Biol. 262:732-745 (1996); Abhinandan and Martin, Mol. Immunol., 45: 3832-3839 (2008); Lefranc M. P. et al., Dev. Comp. Immunol., 27: 55-77 (2003); and Honegger and Pluckthun, J. Mol. Biol., 309:657-670 (2001), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues, which encompass the CDRs as defined by each of the above-cited references, are set forth in the Table below as a comparison. CDR prediction algorithms and interfaces are known in the art, including, for example, Abhinandan and Martin, Mol. Immunol., 45: 3832-3839 (2008); Ehrenmann F. et al., Nucleic Acids Res., 38: D301-D307 (2010); and Adolf-Bryfogle J. et al., Nucleic Acids Res., 43: D432-D438 (2015). The contents of the references cited in this paragraph are incorporated herein by reference in their entireties for use in the present disclosure and for possible inclusion in one or more claims herein. Unless otherwise defined, the CDR sequences provided herein are based on Kabat definition.

CDR DEFINITIONS

| | Kabat¹ | Chothia² | MacCallum³ | IMGT⁴ | AHo⁵ |
|---|---|---|---|---|---|
| V_{H} CDR1 | 31-35 | 26-32 | 30-35 | 27-38 | 25-40 |
| V_{H} CDR2 | 50-65 | 53-55 | 47-58 | 56-65 | 58-77 |
| V_{H} CDR3 | 95-102 | 96-101 | 93-101 | 105-117 | 109-137 |
| V_{L} CDR1 | 24-34 | 26-32 | 30-36 | 27-38 | 25-40 |
| V_{L} CDR2 | 50-56 | 50-52 | 46-55 | 56-65 | 58-77 |
| V_{L} CDR3 | 89-97 | 91-96 | 89-96 | 105-117 | 109-137 |

¹Residue numbering follows the nomenclature of Kabat et al., supra
²Residue numbering follows the nomenclature of Chothia et al., supra
³Residue numbering follows the nomenclature of MacCallum et al., supra
⁴Residue numbering follows the nomenclature of Lefranc et al., supra
⁵Residue numbering follows the nomenclature of Honegger and Plückthun, supra

“Framework” or “FR” residues are those variable-domain residues other than the CDR residues as herein defined.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a HVR of the recipient are replaced by residues from a HVR of a non-human species (donor antibody) such as mouse, rat, rabbit, or nonhuman primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance. In general, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, e.g., Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is one that possesses an amino acid sequence, which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol. 227:381 (1991); Marks et al., J. Mol. Biol. 222:581 (1991). Also available for the preparation of human monoclonal antibodies are methods described in Cole et al., Monoclonal Antibodies and Cancer Therapy, Alan R. Liss, 77 (1985); Boerner et al., J. Immunol. 147(1):86-95 (1991). See also van Dijk and van de Winkel, Curr. Opin. Pharmacol. 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA 103:3557-3562 (2006) regarding human antibodies generated via a human B-cell hybridoma technology.

As use herein, the term “binds”, “specifically bind to” or is “specific for” refers to measurable and reproducible interactions such as binding between a target and an antibody, which is determinative of the presence of the target in the presence of a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (Kd) of ≤1 μM, ≤100 nM, ≤10 nM, ≤1 nM, or ≤0.1 nM. In certain embodiments, an antibody specifically binds to an epitope on a protein that is conserved among the protein from different species. In another embodiment, specific binding can include, but does not require exclusive binding.

The term “specificity” refers to selective recognition of an antigen binding protein (such as a chimeric receptor or an antibody construct) for a particular epitope of an antigen. Natural antibodies, for example, are monospecific. The term “multispecific” as used herein denotes that an antigen binding protein has two or more antigen-binding sites of which at least two bind different antigens or epitopes. “Bispecific” as used herein denotes that an antigen binding protein has two different antigen-binding specificities. The term “monospecific” as used herein denotes an antigen binding protein that has one or more binding sites each of which bind the same antigen or epitope.

The term “functional exogenous receptor” as used herein, refers to an exogenous receptor (e.g., TCR such as a recombinant or engineered TCR, cTCR, a T cell antigen coupler (TAC), a TAC-like chimeric receptor, or CAR) that retains its biological activity after being introduced into an immune effector cell such as a T cell. The biological activity include but are not limited to the ability of the exogenous receptor in specifically binding to a molecule, properly transducing downstream signals, such as inducing cellular proliferation, cytokine production and/or performance of regulatory or cytolytic effector functions.

The term “chimeric antigen receptor” or “CAR” as used herein refers to an artificially constructed hybrid protein or polypeptide containing a binding moiety (e.g. an antibody) linked to immune cell (e.g. T cell) signaling or activation domains. CARs can be synthetic receptors that retarget T cells to tumor surface antigens (Sadelain et al., Nat. Rev. Cancer 3(1):35-45 (2003); Sadelain et al., Cancer Discovery 3(4):388-398 (2013)). CARs can provide both antigen binding and immune cell activation functions onto an immune cell such as a T cell. CARs have the ability to redirect T-cell specificity and reactivity toward a selected target in a non-MHC-restricted manner, exploiting the antigen-binding properties of monoclonal antibodies. The non-MHC-restricted antigen recognition can give T-cells expressing CARs the ability to recognize an antigen independent of antigen processing, thus bypassing a mechanism of tumor escape. “CAR-T cell” refers to a T cell that expresses a CAR.

“T-cell receptor” or “TCR” as used herein refers to an endogenous or modified T-cell receptor comprising an extracellular antigen binding domain that binds to a specific antigenic peptide bound in an MEW molecule. In some embodiments, the TCR comprises a TCRα polypeptide chain and a TCRβ polypeptide chain. In some embodiments, the TCR comprises a TCRγ polypeptide chain and a TCRδ polypeptide chain. In some embodiments, the TCR specifically binds a tumor antigen. “TCR-T” refers to a T cell that expresses a recombinant TCR.

“T-cell antigen coupler receptor” or “TAC receptor” as used herein refers to an engineered receptor comprising an extracellular antigen binding domain that binds to a specific antigen and a T-cell receptor (TCR) binding domain, a transmembrane domain, and an intracellular domain of a co-receptor molecule. The TAC receptor co-opts the endogenous TCR of a T cell that expressed the TAC receptor to elicit antigen-specific T-cell response against a target cell.

The term “recombinant or engineered TCR” as used herein is included as a kind of functional exogenous receptor provided herein, and refers to peptide expressed into an immune cell. The functions of recombinant or engineered TCR may include for example redirecting immune activity of the immune cell against a desired type of cells, such as cancer and infected cells having specific markers at their surface. It can replace or be-co-expressed with the endogenous TCR. In some embodiments, such recombinant TCR are single-chain TCRs comprising an open reading frame where the variable Vα and Vβ domains are paired with a protein linker. This involves the molecular cloning of the TCR genes known to be specific for an antigen of choice. These chains are then introduced into T cells usually by means of a retroviral vector. Consequently, expression of the cloned TCRα and TCRβ genes endows the transduced T cell with a functional specificity determined by the pairing of these new genes. A component of a recombinant or engineered TCR is any functional subunit of a TCR, such as a recombined TCRα and TCRβ, which is encoded by an exogenous polynucleotide sequence introduced into the cell.

“Chimeric T cell receptor” or “cTCR” as used herein refers to an engineered receptor comprising an extracellular antigen-binding domain that binds to a specific antigen, a transmembrane domain of a first subunit of the TCR complex or a portion thereof, and an intracellular signaling domain of a second subunit of the TCR complex or a portion thereof, wherein the first or second subunit of the TCR complex is a TCRα chain, TCRβ chain, TCRγ chain, TCRδ chain, CD3ε, CD3δ, or CD3γ. The transmembrane domain and the intracellular signaling domain of a cTCR may be derived from the same subunit of the TCR complex, or from different subunits of the TCR complex. The intracellular domain may be the full-length intracellular signaling domain or a portion of the intracellular domain of a naturally occurring TCR subunit. In some embodiments, the cTCR comprises the extracellular domain of the TCR subunit or a portion thereof. In some embodiments, the cTCR does not comprise the extracellular domain of the TCR subunit. An “εTCR”, which is used interchangeably with “ε-TRuC”, refers to a cTCR comprising an extracellular domain of CD3ε.

In some embodiments, the functional exogenous receptor provided herein is a cTCR, which has both antigen-binding and T-cell activating functions. For example, a cTCR can comprise: (a) an extracellular ligand binding domain comprising an antigen-binding fragment (e.g., sdAb, scFv) that specifically recognizes one or more epitopes of a tumor antigen (e.g., DLL3); (b) an optional linker; (c) an optional extracellular domain of a first TCR subunit (e.g., CD3ε) or a portion thereof; (d) a transmembrane domain comprising a transmembrane domain of a second TCR subunit (e.g., CDRε); and (e) an intracellular signaling domain comprising an intracellular signaling domain of a third TCR subunit (e.g., CDRε); wherein the first, second, and third TCR subunit are all selected from the group consisting of TCRα, TCRβ, TCRγ, TCRδ, CD3ε, CD3γ, and CD3δ. In some embodiments, the first, second, and third TCR subunits are the same (e.g., all CDR). In some embodiments, the first, second, and third TCR subunits are different. In some embodiments, the cTCR further comprises a hinge domain located between the C-terminus of the extracellular ligand binding domain and the N-terminus of the transmembrane domain. In some embodiments, the hinge domain is derived from CD8α. In some embodiments, the cTCR further comprises a signal peptide located at the N-terminus of the cTCR, such as a signal peptide derived from CD8α.

In some embodiments, the functional exogenous receptor is a T cell antigen coupler (TAC), e.g., comprising: (a) an extracellular ligand binding domain comprising an antigen-binding fragment (e.g., sdAb, scFv) that specifically recognizes one or more epitopes of a tumor antigen (e.g., DLL3); (b) an optional first linker; (c) an extracellular TCR binding domain that specifically recognizes the extracellular domain of a TCR subunit (e.g., CDRε); (d) an optional second linker; (e) an optional extracellular domain of a first TCR co-receptor (e.g., CD4) or a portion thereof; (f) a transmembrane domain comprising a transmembrane domain of a second TCR co-receptor (e.g., CD4); and (g) an optional intracellular signaling domain comprising an intracellular signaling domain of a third TCR co-receptor (e.g., CD4); wherein the TCR subunit is selected from the group consisting of TCRα, TCRβ, TCRγ, TCRδ, CD3ε, CD3γ, and CD3δ; and wherein the first, second, and third TCR co-receptors are all selected from the group consisting of CD4, CD8, and CD28. In some embodiments, the first, second, and third TCR co-receptors are the same. In some embodiments, the first, second, and third TCR co-receptors are different. In some embodiments, the TAC further comprises a hinge domain located between the C-terminus of the extracellular ligand binding domain and the N-terminus of the transmembrane domain. In some embodiments, the hinge domain is derived from CD8α. In some embodiments, the TAC further comprises a signal peptide located at the N-terminus of the TAC, such as a signal peptide derived from CD8α. In some embodiments, the extracellular ligand binding domain is at N-terminal of the extracellular TCR binding domain. In some embodiments, the extracellular ligand binding domain is at C-terminal of the extracellular TCR binding domain.

In some embodiments, the functional exogenous receptor is a TAC-like chimeric receptor, e.g., comprising: (a) an extracellular ligand binding domain comprising an antigen-binding fragment (e.g., sdAb, scFv) that specifically recognizes one or more epitopes of a tumor antigen (e.g., DLL3); (b) an optional first linker; (c) an extracellular TCR binding domain that specifically recognizes the extracellular domain of a first TCR subunit (e.g., TCRα); (d) an optional second linker; (e) an optional extracellular domain of a second TCR subunit (e.g., CD3ε) or a portion thereof; (f) a transmembrane domain comprising a transmembrane domain of a third TCR subunit (e.g., CD3ε); and (g) an optional intracellular signaling domain comprising an intracellular signaling domain of a fourth TCR subunit (e.g., CD3ε); wherein the first, second, third, and fourth TCR subunits are all selected from the group consisting of TCRα, TCRβ, TCRγ, TCRδ, CD3ε, CD3γ, and CD3δ. In some embodiments, the second, third, and fourth TCR subunits are the same. In some embodiments, the first, second, third, and fourth TCR subunits are the same. In some embodiments, the first, second, third, and fourth TCR subunits are different. In some embodiments, the second, third, and fourth TCR subunits are the same, but different from the first TCR subunit. In some embodiments, the extracellular ligand binding domain is at N-terminal of the extracellular TCR binding domain. In some embodiments, the extracellular ligand binding domain is at C-terminal of the extracellular TCR binding domain. In some embodiments, the TAC-like chimeric receptor further comprises a hinge domain located between the C-terminus of the extracellular ligand binding domain and the N-terminus of the transmembrane domain. In some embodiments, the hinge domain is derived from CD8α. In some embodiments, the TAC-like chimeric receptor further comprises a signal peptide located at the N-terminus of the TAC-like chimeric receptor, such as a signal peptide derived from CD8α.

“Tumor homing peptide (THP)” is a kind of peptides specifically targeting tumor stroma, especially tumor vasculature, possibly via specific ligands expressed on tumor vessels. Arginine-glycine-aspartic (RGD) peptides and asparagine-glycine-arginine (NGR) peptides are two of the well-known THPs. For the purpose of the present application, “tumor homing peptide” refers to any of the peptides that have a function of “homing” to tumor vasculature or tumor stroma.

“Percent (%) amino acid sequence identity” with respect to a polypeptide sequence are defined as the percentage of amino acid residues in a candidate sequence that are identical with the amino acid residues in the specific polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full-length of the sequences being compared. For example, polypeptides having at least 70%, 85%, 90%, 95%, 98% or 99% identity to specific polypeptides described herein and preferably exhibiting substantially the same functions, as well as polynucleotide encoding such polypeptides, are contemplated.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. It is understood that, because the polypeptides of this disclosure may be based upon antibodies or other members of the immunoglobulin superfamily, in certain embodiments, a “polypeptide” can occur as a single chain or as two or more associated chains.

“Polynucleotide” or “nucleic acid,” as used interchangeably herein, refers to polymers of nucleotides of any length and includes DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. “Oligonucleotide,” as used herein, refers to short, generally single-stranded, synthetic polynucleotides that are generally, but not necessarily, fewer than about 200 nucleotides in length. The terms “oligonucleotide” and “polynucleotide” are not mutually exclusive.

An “isolated nucleic acid” is a nucleic acid, for example, an RNA, DNA, or a mixed nucleic acids, which is substantially separated from other genome DNA sequences as well as proteins or complexes such as ribosomes and polymerases, which naturally accompany a native sequence. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. The term embraces nucleic acid sequences that have been removed from their naturally occurring environment, and includes recombinant or cloned DNA isolates and chemically synthesized analogues or analogues biologically synthesized by heterologous systems.

The term “express” refers to translation of a nucleic acid into a protein. Proteins may be expressed and remain intracellular, become a component of the cell surface membrane, or be secreted into extracellular matrix or medium.

As used herein, the term “exogenous” is intended to mean that the referenced molecule or other material is introduced into, or non-native to, the cell, tissue, organism, or system. The molecule can be introduced, for example, by introduction of an encoding nucleic acid into the host genetic material such as by integration into a host chromosome or as non-chromosomal genetic material such as a plasmid.

The term “fuse with” or “fused with”, for example, in the context of a polypeptide fused with another polypeptide, means the two entities are connected via a peptide bond or via a peptide linker. Similarly for a nucleic acid fusion. For example, if a RGD-4C sequence is fused with a hinge domain, they can be directly connected via a peptide bond or there can be other peptide(s) between and connecting them such as a tag sequence and/or a (GxS)n linker, wherein x and n, independently can be an integer between 1 and 20, preferably between 3 and 12, such as, for example 3, 4, 5, 6, 7, 8, 9, 10, 11, 12, or more.

As used herein, the term “operatively linked,” and similar phrases (e.g., genetically fused), when used in reference to nucleic acids or amino acids, refer to the operational linkage of nucleic acid sequences or amino acid sequence, respectively, placed in functional relationships with each other. For example, an operatively linked promoter, enhancer elements, open reading frame, 5′ and 3′ UTR, and terminator sequences result in the accurate production of a nucleic acid molecule (e.g., RNA). In some embodiments, operatively linked nucleic acid elements result in the transcription of an open reading frame and ultimately the production of a polypeptide (i.e., expression of the open reading frame). As another example, an operatively linked peptide is one in which the functional domains are placed with appropriate distance from each other to impart the intended function of each domain.

The term “vector” refers to a substance that is used to carry or include a nucleic acid sequence, including for example, a nucleic acid sequence encoding a binding molecule (e.g., an antibody) as described herein, in order to introduce a nucleic acid sequence into a host cell. Vectors applicable for use include, for example, expression vectors, plasmids, phage vectors, viral vectors, episomes, and artificial chromosomes, which can include selection sequences or markers operable for stable integration into a host cell's chromosome. Expression control sequences can include constitutive and inducible promoters, transcription enhancers, transcription terminators, and the like, which are well known in the art. When two or more nucleic acid molecules are to be co-expressed (e.g., both an antibody heavy and light chain or an antibody VH and VL), both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, immunoblotting for expression of gene products, or other suitable analytical methods to test the expression of an introduced nucleic acid sequence or its corresponding gene product.

The term “host cell” as used herein refers to a particular subject cell that may be transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

As used herein, the term “autologous” is meant to refer to any material derived from the same individual to whom it is later to be re-introduced into the individual.

“Allogeneic” refers to a graft derived from a different individual of the same species.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

“Excipient” means a pharmaceutically-acceptable material, composition, or vehicle, such as a liquid or solid filler, diluent, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. In some embodiments, pharmaceutically acceptable excipients are nontoxic to the cell or mammal being exposed thereto at the dosages and concentrations employed. Pharmaceutically acceptable carriers or excipients have preferably met the required standards of toxicological and manufacturing testing and/or are included on the Inactive Ingredient Guide prepared by the U.S. Food and Drug administration.

The term “effective amount” or “therapeutically effective amount” as used herein refers to the amount of engineered immune effector cells or a therapeutic molecule comprising an agent and the engineered immune effector cells or pharmaceutical composition provided herein which is sufficient to result in the desired outcome.

The terms “subject” and “patient” may be used interchangeably. As used herein, in certain embodiments, a subject is a mammal, such as a non-primate or a primate (e.g., human). In specific embodiments, the subject is a human. In one embodiment, the subject is a mammal, e.g., a human, diagnosed with a disease or disorder. In another embodiment, the subject is a mammal, e.g., a human, at risk of developing a disease or disorder.

“Administer” or “administration” refers to the act of injecting or otherwise physically delivering a substance as it exists outside the body into a patient, such as by mucosal, intradermal, intravenous, intramuscular delivery, and/or any other method of physical delivery described herein or known in the art.

As used herein, the terms “treat,” “treatment” and “treating” refer to the reduction or amelioration of the progression, severity, and/or duration of a disease or condition resulting from the administration of one or more therapies. Treating may be determined by assessing whether there has been a decrease, alleviation and/or mitigation of one or more symptoms associated with the underlying disorder such that an improvement is observed with the patient, despite that the patient may still be afflicted with the underlying disorder. The term “treating” includes both managing and ameliorating the disease. The terms “manage,” “managing,” and “management” refer to the beneficial effects that a subject derives from a therapy which does not necessarily result in a cure of the disease.

### 2. Engineered Immune Effector Cells

One aspect of the present disclosure provides genetically engineered immune effector cells which comprise one or more functional exogenous engineered receptors (such as a CAR, TCR, cTCR, TAC receptor, or TAC-like chimeric receptor) and a tumor homing peptide (“THP”; such as an arginine-glycine-aspartate (RGD) peptide or an asparagine-glycine-arginine (NGR) peptide). In some embodiments, the THP is a RGD peptide. In some embodiments, the THP is a NGR peptide. In some embodiments, the engineered receptor comprises an extracellular domain that specifically binds to an antigen (e.g., a tumor antigen), a transmembrane domain, and an intracellular signaling domain. In some embodiments, the intracellular signaling domain comprises a primary intracellular signaling domain and/or a co-stimulatory domain. In some embodiments, the intracellular signaling domain comprises an intracellular signaling domain of a TCR co-receptor. In some embodiments, the engineered receptor is encoded by a heterologous nucleic acid operably linked to a promoter (such as a constitutive promoter or an inducible promoter). The engineered receptor may enhance the function of the modified therapeutic cells, such as by targeting the modified therapeutic cells (e.g., modified immune cells), by transducing signals, and/or by enhancing cytotoxicity of the modified therapeutic cells (e.g., modified immune cells).

The tumor homing peptide can be viewed as an armor to the engineered receptor such that the armored engineered receptor, such as an armored CAR, has enhanced properties compared with the engineered receptor without the armor. The THP is preferably membrane bound. The enhanced properties include such as increased trafficking and infiltration into tumor, and increased cytotoxicity efficacy.

To be concise, the THP in the present disclosure is not a part of the CAR per se or a part of any other functional exogenous receptor, i.e. the tumor homing peptide is not contained within the composition of the CAR. The functional exogenous receptor and the tumor homing peptide can be expressed from two or more different nucleic acids, or alternatively they can be expressed from one single nucleic acid. In the latter situation, the THP is still not within the composition of the CAR; and it is preferably that the resultant one single polypeptide is subsequently cleaved to produce the two separate entities, i.e. the functional exogenous receptor and the THP. Without being bound by any theory, but the approach that includes a THP within the composition of a CAR might generate more cytotoxicity than what is desired and it might also be associated with other side effects such as affecting the cytotoxicity of CAR-T cells against target cells or endothelial cells.

Additionally, the THP according to the present disclosure is genetically engineered to the immune effector cell, such as by expressing on the membrane of the cell a nucleic acid encoding the THP, rather than by using a chemical approach, such as chemical conjugation or chemically attaching the THP to the cells via a chemical coupler. Without being bound by any theory, chemically attaching a THP molecule to a cell (such as the surface of a cell) has increased complexity of therapeutic cell manufacturing, among other disadvantages.

One of the major obstacles in CAR-T therapy for solid tumors is the lack of trafficking of CAR-T into tumors. Abnormal vessel growth and function are hallmarks of cancer, and non-adhesive and abnormal tumor vessels contribute to poor tumor infiltration. Common features of tumor vasculature such as tortuousness, leakiness and lack of proper pericyte coverage, contribute to tumor progression (Potente M, Gerhardt H, Carmeliet P. Basic and therapeutic aspects of angiogenesis. Cell. 2011 Sep. 16; 146(6):873-87. doi: 10.1016/j.ce11.2011.08.039. PMID: 21925313). In addition, endothelial cells from neo-vasculature poorly express leukocyte adhesion molecules such as intracellular adhesion molecule-1/2 (ICAM-1/2) (Griffioen A W, Damen C A, Martinotti S, Blijham G H, Groenewegen G. Endothelial intercellular adhesion molecule-1 expression is suppressed in human malignancies: the role of angiogenic factors. Cancer Res. 1996 Mar. 1; 56(5):1111-17. PMID: 8640769) and vascular cell adhesion molecule 1 (VCAM-1) (Piali L, Fichtel A, Terpe H J, Imhof B A, Gisler R H. Endothelial vascular cell adhesion molecule 1 expression is suppressed by melanoma and carcinoma. J Exp Med. 1995 Feb. 1; 181(2):811-6. doi: 10.1084/jem.181.2.811. PMID: 7530765; PMCID: PMC2191895), which phenotype is also referred to as endothelial cell anergy in various types of cancers (Zhang J, Endres S, Kobold S. Enhancing tumor T cell infiltration to enable cancer immunotherapy. Immunotherapy. 2019 February; 11(3):201-213. doi: 10.2217/imt-2018-0111. PMID: 30730277). All of these factors make it difficult for the T cells to traffick and infiltrate into tumor.

To address the aforementioned and other issues, in the present disclosure THPs were utilized to guide T cells homing to tumor neovasculature, for example, to enhance infiltration of T cells into tumor and thereby improving the clinical efficacy of CAR-T to solid tumors. Specifically, membrane bound form of RGD peptides (mbRGD) and NGR (mbNGR) were utilized to guide T cells homing to tumor. The mbRGD comprises an extracellular RGD domain, preferably a transmembrane region and a hinge domain, with or without an intracellular domain. The mbNGR comprises an extracellular NGR domain, preferably a transmembrane region and a hinge domain, with or without an intracellular domain. After genetically engineered to T cells, mbRGD and mbNGR could improve T cell's adhesion to tumor neo-vasculature. Furthermore, the mbRGD and mbNGR armored CAR-T cells demonstrated enhanced anti-tumor efficacy in vitro and in vivo.

According to one aspect of the disclosure, there is provided an engineered immune effector cell expressing (a) a functional exogenous receptor, and (b) an exogenous tumor homing THP.

Such engineered immune effector cell can be generated by using any appropriate molecular cloning and genetic engineering approaches and means. For example, the engineered immune effector cell can be generated by introducing into an immune effector cell one or more nucleic acids encoding the two components, i.e. the functional exogenous receptor and THP. Said two components can be translated as one polypeptide, or alternatively, they can be translated as two polypeptides.

Therefore, one aspect of the present disclosure provides a composition comprising (a) a functional exogenous receptor, and (b) an exogenous THP; or one or more nucleic acids encoding a functional exogenous receptor and a THP.

In some embodiments, the composition comprises a functional exogenous receptor and a THP. In some embodiments, the composition comprises or consists of one polypeptide comprising a functional exogenous receptor and a THP. In some embodiments, the polypeptide comprises a self-cleaving peptide linker between the functional exogenous receptor and the THP. In some embodiments, the composition comprises or consists of two polypeptides each comprising a functional exogenous receptor and a THP respectively.

In some embodiments, the composition comprises one or more (e.g. one or two) nucleic acids encoding a functional exogenous receptor and a THP. In some embodiments, the composition comprises one nucleic acid encoding a functional exogenous receptor and a THP. In some embodiments, the composition comprises two nucleic acids each encoding a functional exogenous receptor and a THP respectively.

In some embodiments, the functional exogenous receptor is selected from the group consisting of a chimeric antigen receptor (CAR), an engineered T cell receptor (TCR), a chimeric TCR (cTCR), a T cell antigen coupler (TAC), a TAC-like chimeric receptor, and combinations thereof.

In some embodiments, the functional exogenous receptor is a CAR and wherein the CAR comprises (i) an extracellular antigen binding domain, (ii) a transmembrane domain, and (iii) an intracellular signaling domain.

In some embodiments, the transmembrane domain is derived from a molecule selected from the group consisting of CD7, CD8, CD8α, CD4, CD28, CD137 (4-1BB), CD80, CD86, CD152, FasL, TR2 and PD1.

In some embodiments, the intracellular signaling domain comprises a primary intracellular signaling domain of an immune effector cell. In some embodiments, the primary intracellular signaling domain is from CD3.

In some embodiments, the intracellular signaling domain comprises a co-stimulatory signaling domain. In some embodiments, the co-stimulatory signaling domain is derived from a co-stimulatory molecule selected from the group consisting of CD27, CD28, CD137 (4-1BB), OX40, CD30, CD40, CD3, LFA-1, ICOS, CD2, CD7, LIGHT, NKG2C, B7-H3, ligands of CD83 and combinations thereof.

In some embodiments, the CAR further comprises a hinge domain located between the C-terminus of the extracellular antigen binding domain and the N-terminus of the transmembrane domain. In some embodiments, the hinge domain is from CD8. In some embodiments, the hinge domain is from CD7. In some embodiments, the hinge domain is from CD28.

In some embodiments, the CAR further comprises a signal peptide located at the N-terminus of the extracellular antigen binding domain. In some embodiments, the signal peptide is from CD8.

In some embodiments, the immune effector cell is selected from the group consisting of T cells, natural killer (NK) cells, B cells, neutrophils, eosinophils, monocytes, macrophages, dendritic cells, peripheral blood mononuclear cells (PBMC), stem cells from which lymphoid cells may be differentiated, and combinations thereof. In some embodiments, the immune effector cell is a T cell.

In some embodiments, the CAR is an anti-DLL3, an anti-MSLN CAR, or an anti-GPC2 CAR.

In some embodiments, the CAR is an anti-DLL3 CAR. In some embodiments, the anti-DLL3 CAR comprises a first V_{H}H antibody moiety that comprises a CDR1 comprising the amino acid sequence of SEQ ID NO: 29 or a variant thereof comprising up to about 3 amino acid substitutions, a CDR2 comprising the amino acid sequence of SEQ ID NO:30 or a variant thereof comprising up to about 3 amino acid substitutions, and a CDR3 comprising the amino acid sequence of SEQ ID NO: 31 or a variant thereof comprising up to about 3 amino acid substitutions, and a second V_{H}H antibody moiety that comprises a CDR1 comprising the amino acid sequence of SEQ ID NO: 32 or a variant thereof comprising up to about 3 amino acid substitutions, a CDR2 comprising the amino acid sequence of SEQ ID NO: 33 or a variant thereof comprising up to about 3 amino acid substitutions, and a CDR3 comprising the amino acid sequence of SEQ ID NO: 34 or a variant thereof comprising up to about 3 amino acid substitutions.

In some embodiments, the anti-DLL3 CAR comprises a first V_{H}H antibody moiety comprising the amino acid sequences of SEQ ID NO: 27 or an amino acid sequence having at least 75%, 80%, 90%, 91%, 92%, 93%, 94%, 95%, 96%, 97%, 98%, or 99% identity to the amino acid sequence of SEQ ID NO: 27, and a second V_{H}H antibody moiety comprising the amino acid sequences of SEQ ID NO: 28 or an amino acid sequence having at least 75%, 80%, 90%, 91%, 92%, 93%, 94%, 95%, 96%, 97%, 98%, or 99% identity to the amino acid sequence of SEQ ID NO: 28.

In some embodiments, the anti-DLL3 CAR comprises the amino acid sequence of SEQ ID NO: 8 or an amino acid sequence having at least 75%, 80%, 90%, 91%, 92%, 93%, 94%, 95%, 96%, 97%, 98%, or 99% identity to the amino acid sequence of SEQ ID NO: 8.

In some embodiments, the CAR is an anti-MSLN CAR. In some embodiments, the anti-MSLN CAR comprises the amino acid sequence of SEQ ID NO: 41 or an amino acid sequence having at least 75%, 80%, 90%, 91%, 92%, 93%, 94%, 95%, 96%, 97%, 98%, or 99% identity to the amino acid sequence of SEQ ID NO: 41.

In some embodiments, the CAR is an anti-GPC2 CAR. In some embodiments, the anti-GPC2 CAR comprises the amino acid sequence of SEQ ID NO: 44 or an amino acid sequence having at least 75%, 80%, 90%, 91%, 92%, 93%, 94%, 95%, 96%, 97%, 98%, or 99% identity to the amino acid sequence of SEQ ID NO: 44.

In some embodiments, the CAR is a multispecific CAR. In some embodiments, the CAR is a bispecific CAR.

In some embodiments, the exogenous THP is selected from the group consisting of RGD-based peptides, NGR-based peptides, and combinations thereof.

In some embodiments, the THP comprises the RGD-4C peptide having at least 90% sequence identity with the amino acid sequence of SEQ ID NO: 2. In some embodiments, the THP comprises the RGD-4C peptide having the amino acid sequence of SEQ ID NO: 2. In some embodiments, the THP comprises the NGR peptide having at least 90% sequence identity with the amino acid sequence of SEQ ID NO: 38. In some embodiments, the THP comprises the NGR peptide having the amino acid sequence of SEQ ID NO: 38.

In some embodiments, the THP is fused with a transmembrane domain or a hinge domain. In some embodiments, the THP is fused with a transmembrane domain. In some embodiments, the THP is fused with a hinge domain. In some embodiments, the THP is fused with a transmembrane domain and a hinge domain. In some embodiments, the transmembrane domain is from CD7, CD8, CD8α, CD80, CD28, TR2, or FasL. In some embodiments, the hinge domain is from CD7, CD8, CD8α CD80, CD28, TR2, or FasL. In some embodiments, the transmembrane domain is from CD7. In some embodiments, the transmembrane domain comprises the amino acid sequence of SEQ ID NO: 14. In some embodiments, the hinge domain is from CD7. In some embodiments, the hinge domain comprises the amino acid sequence of SEQ ID NO: 13. In some embodiments, the transmembrane domain comprises the amino acid sequence of SEQ ID NO: 14, and the hinge domain comprises the amino acid sequence of SEQ ID NO: 13. In some embodiments, the transmembrane domain is from CD28. In some embodiments, the transmembrane domain comprises the amino acid sequence of SEQ ID NO: 5. In some embodiments, the hinge domain is from CD28. In some embodiments, the hinge domain comprises the amino acid sequence of SEQ ID NO: 4. In some embodiments, the transmembrane domain comprises the amino acid sequence of SEQ ID NO: 5, and the hinge domain comprises the amino acid sequence of SEQ ID NO: 4. In some embodiments, the transmembrane domain is from CD8α. In some embodiments, the transmembrane domain comprises the amino acid sequence of SEQ ID NO: 48. In some embodiments, the hinge domain is from CD8α. In some embodiments, the hinge domain comprises the amino acid sequence of SEQ ID NO: 47. In some embodiments, the transmembrane domain comprises the amino acid sequence of SEQ ID NO: 48, and the hinge domain comprises the amino acid sequence of SEQ ID NO: 47.

In some embodiments, the THP is fused with TGF-β. In some embodiments, the THP is directly fused with TGF-β. In some embodiments, the THP is indirectly fused with TGF-β. In some embodiments, the TGF-β is a TGF-β dominant-negative receptor (TGF-β DNR), such as dnTGF-βRII).

In some embodiments, the THP is fused with a tag sequence or a peptide linker. In some embodiments, the THP is fused with a tag sequence. In some embodiments, the tag sequence is a Flag tag comprising the amino acid sequence of SEQ ID NO: 3. In some embodiments, the THP is fused with a peptide linker. In some embodiments, and the peptide linker is a G4S linker. In some embodiments, the peptide linker is a (G45)₂ linker. In some embodiments, the peptide linker is a (G4S)₃ linker. In some embodiments, the THP is fused with a tag sequence and a peptide linker. In some embodiments, the tag sequence is a Flag tag comprising the amino acid sequence of SEQ ID NO: 3, and the peptide linker is a (G45)₃ linker. In some embodiments, the THP is fused with a membrane anchoring sequence. In some embodiments, the membrane anchoring sequence is a glycosylphosphatidylinositol (GPI)-anchoring peptide sequence. In some embodiments, the GPI-anchoring peptide sequence is attached to a GPI linker.

In some embodiments, the THP is not fused with an intracellular domain. In some embodiments, the THP is fused with an intracellular domain.

In some embodiments, the immune effector cell comprises an amino acid having the amino acid sequence set forth in any one of SEQ ID NOs: 10, 11, 19, 21-26, 35-37, 39-40, 42, 43, 45, 46, 49, 50, and 52-58.

In some embodiments, there is provided an immune effector cell comprising a functional exogenous receptor and an exogenous THP. In some embodiments, the functional exogenous receptor is selected from the group consisting of a CAR, a TCR, a cTCR, a TAC, a TAC-like chimeric receptor, and combinations thereof. In some embodiments, the functional exogenous receptor is a multispecific exogenous receptor. In some embodiments, functional exogenous receptor specifically binds DLL3, MSLN, GPC2, or a combination thereof. In some embodiments, the THP is a RGD-based THP, a NGR-based THP, or a combination thereof. In some embodiments, the THP is fused with a transmembrane domain. In some embodiments, the THP is fused with a transmembrane domain and a hinge domain. In some embodiments, the THP is fused with a tag sequence, a peptide linker, and/or a membrane anchoring sequence. In some embodiments, the THP is fused with a TGF-β dominant-negative receptor (TGF-β DNR, e.g., dnTGF-βRII) optionally via a peptide linker. In some embodiments, the immune effector cell is selected from the group consisting of T cells, natural killer (NK) cells, B cells, neutrophils, eosinophils, monocytes, macrophages, dendritic cells, peripheral blood mononuclear cells (PBMC), stem cells from which lymphoid cells may be differentiated, and combinations thereof.

In some embodiments, there is provided an immune effector cell comprising an anti-DLL3 functional exogenous receptor and an exogenous RGD, wherein the immune effector cell comprises a polypeptide comprising, from N-terminus to C-terminus: a RGD-4C (SEQ ID NO: 2), a (G45)₃ linker, an anti-DLL3 single domain antibody (sdAb), a hinge domain (SEQ ID NO: 47) and a transmembrane domain from CD8α (SEQ ID NO: 48), 4-1BB, and an intracellular signaling domain from CD3. In some embodiments, the immune effector cell comprises a polypeptide comprising the amino acid sequence of SEQ ID NO: 49 or a variant thereof having, at least about 95% (for example at least about any of 96%, 97%, 98%, or 99%) sequence identity to SEQ ID NO: 49. In some embodiments, the immune effector cell comprises a polypeptide comprising the amino acid sequence of SEQ ID NO: 49. In some embodiments, the immune effector cell is selected from the group consisting of T cells, natural killer (NK) cells, B cells, neutrophils, eosinophils, monocytes, macrophages, dendritic cells, peripheral blood mononuclear cells (PBMC), stem cells from which lymphoid cells may be differentiated, and combinations thereof.

In some embodiments, there is provided an immune effector cell comprising an anti-DLL3 functional exogenous receptor and an exogenous RGD, wherein the immune effector cell comprises a polypeptide comprising, from N-terminus to C-terminus: an anti-DLL3 sdAb, a (G4S)₃ linker, a RGD-4C (SEQ ID NO: 2), a hinge domain (SEQ ID NO: 47) and a transmembrane domain (SEQ ID NO: 48) from CD8α, 4-1BB, and an intracellular signaling domain from CD3. In some embodiments, the immune effector cell comprises a polypeptide comprising the amino acid sequence of SEQ ID NO: 50 or a variant thereof having, at least about 95% (for example at least about any of 96%, 97%, 98%, or 99%) sequence identity to SEQ ID NO: 50. In some embodiments, the immune effector cell comprises a polypeptide comprising the amino acid sequence of SEQ ID NO: 50. In some embodiments, the immune effector cell is selected from the group consisting of T cells, natural killer (NK) cells, B cells, neutrophils, eosinophils, monocytes, macrophages, dendritic cells, peripheral blood mononuclear cells (PBMC), stem cells from which lymphoid cells may be differentiated, and combinations thereof.

In some embodiments, there is provided an immune effector cell comprising a CAR comprising (i) an extracellular antigen binding domain, (ii) a transmembrane domain, and (iii) an intracellular signaling domain, and an exogenous THP. In some embodiments, the CAR is a multispecific CAR. In some embodiments, the CAR is an anti-DLL3 CAR, an anti-MSLN CAR, or an anti-GPC2 CAR. In some embodiments, the extracellular antigen binding domain of the CAR comprises a tandem V_{H}H antibody moiety. In some embodiments, the transmembrane domain of the CAR is derived from a molecule selected from the group consisting of CD7, CD8, CD8α, CD4, CD28, CD137 (4-1BB), CD80, CD86, CD152, FasL, TR2 and PD1. In some embodiments, the transmembrane domain of the CAR is derived from CD7. In some embodiments, the transmembrane domain of the CAR is derived from CD8. In some embodiments, the transmembrane domain of the CAR is derived from TR2. In some embodiments, the intracellular signaling domain of the CAR comprises a primary intracellular signaling domain of an immune effector cell. In some embodiments, the intracellular signaling domain of the CAR is from CD3. In some embodiments, the intracellular signaling domain of the CAR comprises a co-stimulatory signaling domain. In some embodiments, the co-stimulatory signaling domain is derived from a co-stimulatory molecule selected from the group consisting of CD27, CD28, CD137 (4-1BB), OX40, CD30, CD40, CD3, LFA-1, ICOS, CD2, CD7, LIGHT, NKG2C, B7-H3, ligands of CD83 and combinations thereof. In some embodiments, the CAR further comprises a hinge domain located between the C-terminus of the extracellular antigen binding domain and the N-terminus of the transmembrane domain. In some embodiments, the hinge domain is from CD8. In some embodiments, the CAR further comprises a signal peptide located at the N-terminus of the extracellular antigen binding domain. In some embodiments, the signal peptide is from CD8. In some embodiments, the THP is a RGD-based THP, a NGR-based THP, or a combination thereof. In some embodiments, the THP is fused with a transmembrane domain. In some embodiments, the THP is fused with a transmembrane domain and a hinge domain. In some embodiments, the THP is fused with a tag sequence, a peptide linker, and/or a membrane anchoring sequence. In some embodiments, the THP is fused with a TGF-β dominant-negative receptor (TGF-β DNR, e.g., dnTGF-βRII), optionally via a peptide linker. In some embodiments, the immune effector cell is selected from the group consisting of T cells, natural killer (NK) cells, B cells, neutrophils, eosinophils, monocytes, macrophages, dendritic cells, peripheral blood mononuclear cells (PBMC), stem cells from which lymphoid cells may be differentiated, and combinations thereof.

In some embodiments, there is provided an immune effector cell comprising a CAR and a membrane-bound THP fused with a GPI-anchoring peptide sequence. In some embodiments, the CAR is a multispecific CAR. In some embodiments, the CAR is an anti-DLL3 CAR, an anti-MSLN CAR, or an anti-GPC2 CAR. In some embodiments, the THP is a RGD-based THP, a NGR-based THP, or a combination thereof. In some embodiments, the immune effector cell is selected from the group consisting of T cells, natural killer (NK) cells, B cells, neutrophils, eosinophils, monocytes, macrophages, dendritic cells, peripheral blood mononuclear cells (PBMC), stem cells from which lymphoid cells may be differentiated, and combinations thereof.

In some embodiments, there is provided an immune effector cell comprising an anti-DLL3 CAR comprising (i) an extracellular DLL3 binding domain, (ii) a transmembrane domain, and (iii) an intracellular signaling domain, and an exogenous THP. In some embodiments, the extracellular antigen binding domain of the anti-DLL3 CAR comprises a tandem V_{H}H antibody moiety. In some embodiments, the transmembrane domain of the anti-DLL3 CAR is derived from a molecule selected from the group consisting of CD7, CD8, CD8α, CD4, CD28, CD137 (4-1BB), CD80, CD86, CD152, FasL, TR2 and PD1. In some embodiments, the transmembrane domain of the anti-DLL3 CAR is derived from CD7. In some embodiments, the transmembrane domain of the anti-DLL3 CAR is derived from CD8. In some embodiments, the transmembrane domain of the anti-DLL3 CAR is derived from TR2. In some embodiments, the intracellular signaling domain of the anti-DLL3 CAR comprises a primary intracellular signaling domain of an immune effector cell. In some embodiments, the intracellular signaling domain of the anti-DLL3 CAR is from CD3. In some embodiments, the intracellular signaling domain of the anti-DLL3 CAR comprises a co-stimulatory signaling domain. In some embodiments, the co-stimulatory signaling domain is derived from a co-stimulatory molecule selected from the group consisting of CD27, CD28, CD137 (4-1BB), OX40, CD30, CD40, CD3, LFA-1, ICOS, CD2, CD7, LIGHT, NKG2C, B7-H3, ligands of CD83 and combinations thereof. In some embodiments, the anti-DLL3 CAR further comprises a hinge domain located between the C-terminus of the extracellular antigen binding domain and the N-terminus of the transmembrane domain. In some embodiments, the hinge domain is from CD8. In some embodiments, the anti-DLL3 CAR further comprises a signal peptide located at the N-terminus of the extracellular antigen binding domain. In some embodiments, the signal peptide is from CD8. In some embodiments, the THP is a RGD-based THP, a NGR-based THP, or a combination thereof. In some embodiments, the THP is fused with a transmembrane domain. In some embodiments, the THP is fused with a transmembrane domain and a hinge domain. In some embodiments, the THP is fused with a tag sequence, a peptide linker, and/or a membrane anchoring sequence. In some embodiments, the THP is fused with a TGF-β dominant-negative receptor (TGF-β DNR, e.g., dnTGF-βRII) optionally via a peptide linker. In some embodiments, the immune effector cell is selected from the group consisting of T cells, natural killer (NK) cells, B cells, neutrophils, eosinophils, monocytes, macrophages, dendritic cells, peripheral blood mononuclear cells (PBMC), stem cells from which lymphoid cells may be differentiated, and combinations thereof.

In some embodiments, there is provided an immune effector cell comprising an anti-DLL3 CAR comprising (i) an extracellular DLL3 binding domain, (ii) a CD7 transmembrane domain, and (iii) an intracellular signaling domain, and an exogenous THP. In some embodiments, the extracellular antigen binding domain of the anti-DLL3 CAR comprises a tandem V_{H}H antibody moiety. In some embodiments, the intracellular signaling domain of the anti-DLL3 CAR comprises a primary intracellular signaling domain of an immune effector cell. In some embodiments, the intracellular signaling domain of the anti-DLL3 CAR is from CD3. In some embodiments, the intracellular signaling domain of the anti-DLL3 CAR comprises a co-stimulatory signaling domain. In some embodiments, the co-stimulatory signaling domain is derived from a co-stimulatory molecule selected from the group consisting of CD27, CD28, CD137 (4-1BB), OX40, CD30, CD40, CD3, LFA-1, ICOS, CD2, CD7, LIGHT, NKG2C, B7-H3, ligands of CD83 and combinations thereof. In some embodiments, the THP is a RGD-based THP, a NGR-based THP, or a combination thereof. In some embodiments, the THP is fused with a transmembrane domain. In some embodiments, the THP is fused with a transmembrane domain and a hinge domain. In some embodiments, the THP is fused with a tag sequence, a peptide linker, and/or a membrane anchoring sequence. In some embodiments, the THP is fused with a TGF-β dominant-negative receptor (TGF-β DNR, e.g., dnTGF-βRII) optionally via a peptide linker. In some embodiments, the immune effector cell is selected from the group consisting of T cells, natural killer (NK) cells, B cells, neutrophils, eosinophils, monocytes, macrophages, dendritic cells, peripheral blood mononuclear cells (PBMC), stem cells from which lymphoid cells may be differentiated, and combinations thereof.

In some embodiments, there is provided an immune effector cell comprising an anti-DLL3 CAR comprising (i) an extracellular DLL3 binding domain, (ii) a TR2 transmembrane domain, and (iii) an intracellular signaling domain, and an exogenous THP. In some embodiments, the extracellular antigen binding domain of the anti-DLL3 CAR comprises a tandem V_{H}H antibody moiety. In some embodiments, the intracellular signaling domain of the anti-DLL3 CAR comprises a primary intracellular signaling domain of an immune effector cell. In some embodiments, the intracellular signaling domain of the anti-DLL3 CAR is from CD3. In some embodiments, the intracellular signaling domain of the anti-DLL3 CAR comprises a co-stimulatory signaling domain. In some embodiments, the co-stimulatory signaling domain is derived from a co-stimulatory molecule selected from the group consisting of CD27, CD28, CD137 (4-1BB), OX40, CD30, CD40, CD3, LFA-1, ICOS, CD2, CD7, LIGHT, NKG2C, B7-H3, ligands of CD83 and combinations thereof. In some embodiments, the THP is a RGD-based THP, a NGR-based THP, or a combination thereof. In some embodiments, the THP is fused with a transmembrane domain. In some embodiments, the THP is fused with a transmembrane domain and a hinge domain. In some embodiments, the THP is fused with a tag sequence, a peptide linker, and/or a membrane anchoring sequence. In some embodiments, the THP is fused with a TGF-β dominant-negative receptor (TGF-β DNR, e.g., dnTGF-βRII) optionally via a peptide linker. In some embodiments, the immune effector cell is selected from the group consisting of T cells, natural killer (NK) cells, B cells, neutrophils, eosinophils, monocytes, macrophages, dendritic cells, peripheral blood mononuclear cells (PBMC), stem cells from which lymphoid cells may be differentiated, and combinations thereof.

In some embodiments, there is provided an immune effector cell comprising an anti-DLL3 CAR comprising (i) an extracellular DLL3 binding domain, (ii) a CD8 transmembrane domain, and (iii) an intracellular signaling domain, and an exogenous THP. In some embodiments, the extracellular antigen binding domain of the anti-DLL3 CAR comprises a tandem V_{H}H antibody moiety. In some embodiments, the intracellular signaling domain of the anti-DLL3 CAR comprises a primary intracellular signaling domain of an immune effector cell. In some embodiments, the intracellular signaling domain of the anti-DLL3 CAR is from CD3. In some embodiments, the intracellular signaling domain of the anti-DLL3 CAR comprises a co-stimulatory signaling domain. In some embodiments, the co-stimulatory signaling domain is derived from a co-stimulatory molecule selected from the group consisting of CD27, CD28, CD137 (4-1BB), OX40, CD30, CD40, CD3, LFA-1, ICOS, CD2, CD7, LIGHT, NKG2C, B7-H3, ligands of CD83 and combinations thereof. In some embodiments, the THP is a RGD-based THP, a NGR-based THP, or a combination thereof. In some embodiments, the THP is fused with a transmembrane domain. In some embodiments, the THP is fused with a transmembrane domain and a hinge domain. In some embodiments, the THP is fused with a tag sequence, a peptide linker, and/or a membrane anchoring sequence. In some embodiments, the THP is fused with a TGF-β dominant-negative receptor (TGF-β DNR, e.g, dnTGF-βRII) optionally via a peptide linker. In some embodiments, the immune effector cell is selected from the group consisting of T cells, natural killer (NK) cells, B cells, neutrophils, eosinophils, monocytes, macrophages, dendritic cells, peripheral blood mononuclear cells (PBMC), stem cells from which lymphoid cells may be differentiated, and combinations thereof.

In some embodiments, there is provided an immune effector cell comprising an anti-DLL3 CAR comprising (i) an extracellular DLL3 binding domain, (ii) a transmembrane domain, and (iii) an intracellular signaling domain, and an exogenous THP, wherein the an anti-DLL3 CAR comprises a first V_{H}H antibody moiety that comprises a CDR1 comprising the amino acid sequence of SEQ ID NO: 29 or a variant thereof comprising up to about 3 amino acid substitutions, a CDR2 comprising the amino acid sequence of SEQ ID NO:30 or a variant thereof comprising up to about 3 amino acid substitutions, and a CDR3 comprising the amino acid sequence of SEQ ID NO: 31 or a variant thereof comprising up to about 3 amino acid substitutions, and a second V_{H}H antibody moiety that comprises a CDR1 comprising the amino acid sequence of SEQ ID NO: 32 or a variant thereof comprising up to about 3 amino acid substitutions, a CDR2 comprising the amino acid sequence of SEQ ID NO: 33 or a variant thereof comprising up to about 3 amino acid substitutions, and a CDR3 comprising the amino acid sequence of SEQ ID NO: 34 or a variant thereof comprising up to about 3 amino acid substitutions. In some embodiments, the transmembrane domain of the anti-DLL3 CAR is derived from a molecule selected from the group consisting of CD7, CD8, CD8α, CD4, CD28, CD137 (4-1BB), CD80, CD86, CD152, FasL, TR2 and PD1. In some embodiments, the transmembrane domain of the anti-DLL3 CAR is derived from CD7. In some embodiments, the transmembrane domain of the anti-DLL3 CAR is derived from CD8. In some embodiments, the transmembrane domain of the anti-DLL3 CAR is derived from TR2. In some embodiments, the intracellular signaling domain of the anti-DLL3 CAR comprises a primary intracellular signaling domain of an immune effector cell. In some embodiments, the intracellular signaling domain of the anti-DLL3 CAR is from CD3. In some embodiments, the intracellular signaling domain of the anti-DLL3 CAR comprises a co-stimulatory signaling domain. In some embodiments, the co-stimulatory signaling domain is derived from a co-stimulatory molecule selected from the group consisting of CD27, CD28, CD137 (4-1BB), OX40, CD30, CD40, CD3, LFA-1, ICOS, CD2, CD7, LIGHT, NKG2C, B7-H3, ligands of CD83 and combinations thereof. In some embodiments, the anti-DLL3 CAR further comprises a hinge domain located between the C-terminus of the extracellular antigen binding domain and the N-terminus of the transmembrane domain. In some embodiments, the hinge domain is from CD8. In some embodiments, the anti-DLL3 CAR further comprises a signal peptide located at the N-terminus of the extracellular antigen binding domain. In some embodiments, the signal peptide is from CD8. In some embodiments, the THP is a RGD-based THP, a NGR-based THP, or a combination thereof. In some embodiments, the THP is fused with a transmembrane domain. In some embodiments, the THP is fused with a transmembrane domain and a hinge domain. In some embodiments, the THP is fused with a tag sequence, a peptide linker, and/or a membrane anchoring sequence. In some embodiments, the THP is fused with a TGF-β dominant-negative receptor (TGF-β DNR, e.g., dnTGF-βRII) optionally via a peptide linker. In some embodiments, the immune effector cell is selected from the group consisting of T cells, natural killer (NK) cells, B cells, neutrophils, eosinophils, monocytes, macrophages, dendritic cells, peripheral blood mononuclear cells (PBMC), stem cells from which lymphoid cells may be differentiated, and combinations thereof.

In some embodiments, there is provided an immune effector cell comprising an anti-DLL3 CAR comprising (i) an extracellular DLL3 binding domain, (ii) a transmembrane domain, and (iii) an intracellular signaling domain, and an exogenous THP, wherein the an anti-DLL3 CAR comprises a first V_{H}H antibody moiety comprising the amino acid sequences of SEQ ID NO: 27 or an amino acid sequence having at least 75%, 80%, 90%, 91%, 92%, 93%, 94%, 95%, 96%, 97%, 98%, or 99% identity to the amino acid sequence of SEQ ID NO: 27, and a second V_{H}H antibody moiety comprising the amino acid sequences of SEQ ID NO: 28 or an amino acid sequence having at least 75%, 80%, 90%, 91%, 92%, 93%, 94%, 95%, 96%, 97%, 98%, or 99% identity to the amino acid sequence of SEQ ID NO: 28. In some embodiments, the transmembrane domain of the anti-DLL3 CAR is derived from a molecule selected from the group consisting of CD7, CD8, CD8α, CD4, CD28, CD137 (4-1BB), CD80, CD86, CD152, FasL, TR2 and PD1. In some embodiments, the transmembrane domain of the anti-DLL3 CAR is derived from CD7. In some embodiments, the transmembrane domain of the anti-DLL3 CAR is derived from CD8. In some embodiments, the transmembrane domain of the anti-DLL3 CAR is derived from TR2. In some embodiments, the intracellular signaling domain of the anti-DLL3 CAR comprises a primary intracellular signaling domain of an immune effector cell. In some embodiments, the intracellular signaling domain of the anti-DLL3 CAR is from CD3. In some embodiments, the intracellular signaling domain of the anti-DLL3 CAR comprises a co-stimulatory signaling domain. In some embodiments, the co-stimulatory signaling domain is derived from a co-stimulatory molecule selected from the group consisting of CD27, CD28, CD137 (4-1BB), OX40, CD30, CD40, CD3, LFA-1, ICOS, CD2, CD7, LIGHT, NKG2C, B7-H3, ligands of CD83 and combinations thereof. In some embodiments, the anti-DLL3 CAR further comprises a hinge domain located between the C-terminus of the extracellular antigen binding domain and the N-terminus of the transmembrane domain. In some embodiments, the hinge domain is from CD8. In some embodiments, the anti-DLL3 CAR further comprises a signal peptide located at the N-terminus of the extracellular antigen binding domain. In some embodiments, the signal peptide is from CD8. In some embodiments, the THP is a RGD-based THP, a NGR-based THP, or a combination thereof. In some embodiments, the THP is fused with a transmembrane domain. In some embodiments, the THP is fused with a transmembrane domain and a hinge domain. In some embodiments, the THP is fused with a tag sequence, a peptide linker, and/or a membrane anchoring sequence. In some embodiments, the THP is fused with a TGF-β dominant-negative receptor (TGF-β DNR, e.g., dnTGF-βRII) optionally via a peptide linker. In some embodiments, the immune effector cell is selected from the group consisting of T cells, natural killer (NK) cells, B cells, neutrophils, eosinophils, monocytes, macrophages, dendritic cells, peripheral blood mononuclear cells (PBMC), stem cells from which lymphoid cells may be differentiated, and combinations thereof.

In some embodiments, there is provided an immune effector cell comprising an anti-DLL3 CAR comprising (i) an extracellular DLL3 binding domain, (ii) a transmembrane domain, and (iii) an intracellular signaling domain, and an exogenous THP, wherein the an anti-DLL3 CAR comprises the amino acid sequence of SEQ ID NO: 8 or an amino acid sequence having at least 75%, 80%, 90%, 91%, 92%, 93%, 94%, 95%, 96%, 97%, 98%, or 99% identity to the amino acid sequence of SEQ ID NO: 8. In some embodiments, the transmembrane domain of the anti-DLL3 CAR is derived from a molecule selected from the group consisting of CD7, CD8, CD8α, CD4, CD28, CD137 (4-1BB), CD80, CD86, CD152, FasL, TR2 and PD1. In some embodiments, the transmembrane domain of the anti-DLL3 CAR is derived from CD7. In some embodiments, the transmembrane domain of the anti-DLL3 CAR is derived from CD8. In some embodiments, the transmembrane domain of the anti-DLL3 CAR is derived from TR2. In some embodiments, the intracellular signaling domain of the anti-DLL3 CAR comprises a primary intracellular signaling domain of an immune effector cell. In some embodiments, the intracellular signaling domain of the anti-DLL3 CAR is from CD3. In some embodiments, the intracellular signaling domain of the anti-DLL3 CAR comprises a co-stimulatory signaling domain. In some embodiments, the co-stimulatory signaling domain is derived from a co-stimulatory molecule selected from the group consisting of CD27, CD28, CD137 (4-1BB), OX40, CD30, CD40, CD3, LFA-1, ICOS, CD2, CD7, LIGHT, NKG2C, B7-H3, ligands of CD83 and combinations thereof. In some embodiments, the anti-DLL3 CAR further comprises a hinge domain located between the C-terminus of the extracellular antigen binding domain and the N-terminus of the transmembrane domain. In some embodiments, the hinge domain is from CD8. In some embodiments, the anti-DLL3 CAR further comprises a signal peptide located at the N-terminus of the extracellular antigen binding domain. In some embodiments, the signal peptide is from CD8. In some embodiments, the anti-DLL3 CAR further comprises a signal peptide located at the N-terminus of the extracellular antigen binding domain. In some embodiments, the signal peptide is from CD8. In some embodiments, the anti-DLL3 CAR comprises the amino acid sequence of SEQ ID NO: 8. In some embodiments, the THP is a RGD-based THP, a NGR-based THP, or a combination thereof. In some embodiments, the THP is fused with a transmembrane domain. In some embodiments, the THP is fused with a transmembrane domain and a hinge domain. In some embodiments, the THP is fused with a tag sequence, a peptide linker, and/or a membrane anchoring sequence. In some embodiments, the THP is fused with a TGF-β dominant-negative receptor (TGF-β DNR, e.g., dnTGF-βRII) optionally via a peptide linker. In some embodiments, the immune effector cell is selected from the group consisting of T cells, natural killer (NK) cells, B cells, neutrophils, eosinophils, monocytes, macrophages, dendritic cells, peripheral blood mononuclear cells (PBMC), stem cells from which lymphoid cells may be differentiated, and combinations thereof.

In some embodiments, there is provided an immune effector cell comprising an anti-DLL3 CAR and an exogenous membrane-bound RGD. In some embodiments, the immune effector cell comprises a polypeptide comprising, from N-terminus to C-terminus: an anti-DLL3 CAR (SEQ ID NO: 8), RGD-4C (SEQ ID NO: 2), a (G4S)₃ linker, and the hinge domain (SEQ ID NO: 13) and transmembrane domain (SEQ ID NO: 14) from CD7. In some embodiments, the immune effector cell comprises a polypeptide comprising the amino acid sequence of SEQ ID NO: 26, or a variant thereof having, at least about 95% (for example at least about any of 96%, 97%, 98%, or 99%) sequence identity to SEQ ID NO: 26. In some embodiments, the immune effector cell comprises a polypeptide comprising the amino acid sequence of SEQ ID NO: 26. In some embodiments, the immune effector cell is selected from the group consisting of T cells, natural killer (NK) cells, B cells, neutrophils, eosinophils, monocytes, macrophages, dendritic cells, peripheral blood mononuclear cells (PBMC), stem cells from which lymphoid cells may be differentiated, and combinations thereof.

In some embodiments, there is provided an immune effector cell comprising an anti-DLL3 CAR and an exogenous membrane-bound NGR. In some embodiments, the immune effector cell comprises a polypeptide comprising, from N-terminus to C-terminus: an anti-DLL3 CAR (SEQ ID NO: 8), NGR (SEQ ID NO: 38), a (G45)₂ linker, and the hinge domain (SEQ ID NO: 13) and transmembrane domain (SEQ ID NO: 14) from CD7. In some embodiments, the immune effector cell comprises a polypeptide comprising the amino acid sequence of SEQ ID NO: 39, or a variant thereof having, at least about 95% (for example at least about any of 96%, 97%, 98%, or 99%) sequence identity to SEQ ID NO: 39. In some embodiments, the immune effector cell comprises a polypeptide comprising the amino acid sequence of SEQ ID NO: 39. In some embodiments, the immune effector cell is selected from the group consisting of T cells, natural killer (NK) cells, B cells, neutrophils, eosinophils, monocytes, macrophages, dendritic cells, peripheral blood mononuclear cells (PBMC), stem cells from which lymphoid cells may be differentiated, and combinations thereof.

In some embodiments, there is provided an immune effector cell comprising an anti-DLL3 CAR and an exogenous membrane-bound RGD fused with TGF-β DNR, wherein the immune effector cell comprises a polypeptide comprising, from N-terminus to C-terminus: an anti-DLL3 CAR (SEQ ID NO: 8), a CD28 signal peptide, RGD-4C (SEQ ID NO: 2), a G4S linker, and a TGF-β DNR (e.g., dnTGF-βRII). In some embodiments, the TGF-β DNR is anchored to the membrane via a transmembrane domain of TR2. In some embodiments, the immune effector cell comprises a polypeptide comprising the amino acid sequence of SEQ ID NO: 52 or a variant thereof having, at least about 95% (for example at least about any of 96%, 97%, 98%, or 99%) sequence identity to SEQ ID NO: 52. In some embodiments, the immune effector cell comprises a polypeptide comprising the amino acid sequence of SEQ ID NO: 52. In some embodiments, the immune effector cell is selected from the group consisting of T cells, natural killer (NK) cells, B cells, neutrophils, eosinophils, monocytes, macrophages, dendritic cells, peripheral blood mononuclear cells (PBMC), stem cells from which lymphoid cells may be differentiated, and combinations thereof.

In some embodiments, there is provided an immune effector cell comprising an anti-DLL3 CAR and an exogenous membrane-bound NGR fused with TGF-β DNR (e.g., dnTGF-βRII), wherein the immune effector cell comprises a polypeptide comprising, from N-terminus to C-terminus: an anti-DLL3 CAR (SEQ ID NO: 8), a CD28 signal peptide, NGR (SEQ ID NO: 38), a G4S linker, and a TGF-β DNR (e.g., dnTGF-βRII). In some embodiments, the TGF-β DNR is anchored to the membrane via a transmembrane domain of TR2. In some embodiments, the immune effector cell comprises a polypeptide comprising the amino acid sequence of SEQ ID NO: 54 or a variant thereof having, at least about 95% (for example at least about any of 96%, 97%, 98%, or 99%) sequence identity to SEQ ID NO: 54. In some embodiments, the immune effector cell comprises a polypeptide comprising the amino acid sequence of SEQ ID NO: 54. In some embodiments, the immune effector cell is selected from the group consisting of T cells, natural killer (NK) cells, B cells, neutrophils, eosinophils, monocytes, macrophages, dendritic cells, peripheral blood mononuclear cells (PBMC), stem cells from which lymphoid cells may be differentiated, and combinations thereof.

In some embodiments, there is provided an immune effector cell comprising an anti-DLL3 CAR and an exogenous membrane-bound NGR fused with TGF-β DNR (e.g., dnTGF-βRII), wherein the immune effector cell comprises a polypeptide comprising, from N-terminus to C-terminus: an anti-DLL3 CAR (SEQ ID NO: 8), a TGF-β DNR (e.g., dnTGF-βRII), a P2A, NGR (SEQ ID NO: 38), and a hinge domain (SEQ ID NO: 13) and a transmembrane domain from CD7 (SEQ ID NO: 14). In some embodiments, the TGF-β DNR is anchored to the membrane via a transmembrane domain of TR2. In some embodiments, the immune effector cell comprises a polypeptide comprising the amino acid sequence of SEQ ID NO: 55 or a variant thereof having, at least about 95% (for example at least about any of 96%, 97%, 98%, or 99%) sequence identity to SEQ ID NO: 55. In some embodiments, the immune effector cell comprises a polypeptide comprising the amino acid sequence of SEQ ID NO: 55. In some embodiments, the immune effector cell is selected from the group consisting of T cells, natural killer (NK) cells, B cells, neutrophils, eosinophils, monocytes, macrophages, dendritic cells, peripheral blood mononuclear cells (PBMC), stem cells from which lymphoid cells may be differentiated, and combinations thereof.

In some embodiments, there is provided an immune effector cell comprising an anti-DLL3 CAR and an exogenous membrane-bound NGR fused with TGF-β DNR (e.g., dnTGF-βRII), wherein the immune effector cell comprises a polypeptide comprising, from N-terminus to C-terminus: an anti-DLL3 CAR (SEQ ID NO: 8), NGR (SEQ ID NO: 38), a G4S linker, NGR (SEQ ID NO: 38), a hinge domain (SEQ ID NO: 13) and a transmembrane domain from CD7 (SEQ ID NO: 14), a T2A, and a TGF-β DNR (e.g., dnTGF-βRII). In some embodiments, the TGF-DNR is anchored to the membrane via a transmembrane domain of TR2. In some embodiments, the immune effector cell comprises a polypeptide comprising the amino acid sequence of SEQ ID NO: 57 or a variant thereof having, at least about 95% (for example at least about any of 96%, 97%, 98%, or 99%) sequence identity to SEQ ID NO: 57. In some embodiments, the immune effector cell comprises a polypeptide comprising the amino acid sequence of SEQ ID NO: 57. In some embodiments, the immune effector cell is selected from the group consisting of T cells, natural killer (NK) cells, B cells, neutrophils, eosinophils, monocytes, macrophages, dendritic cells, peripheral blood mononuclear cells (PBMC), stem cells from which lymphoid cells may be differentiated, and combinations thereof.

In some embodiments, there is provided an immune effector cell comprising an anti-MSLN CAR comprising (i) an extracellular MSLN binding domain, (ii) a transmembrane domain, and (iii) an intracellular signaling domain, and an exogenous THP. In some embodiments, the extracellular antigen binding domain of the anti-MSLN CAR comprises a tandem V_{H}H antibody moiety. In some embodiments, the transmembrane domain of the anti-MSLN CAR is derived from a molecule selected from the group consisting of CD7, CD8, CD8α, CD4, CD28, CD137 (4-1BB), CD80, CD86, CD152, FasL, TR2 and PD1. In some embodiments, the transmembrane domain of the anti-MSLN CAR is derived from CD7. In some embodiments, the transmembrane domain of the anti-MSLN CAR is derived from CD8. In some embodiments, the transmembrane domain of the anti-MSLN CAR is derived from TR2. In some embodiments, the intracellular signaling domain of the anti-MSLN CAR comprises a primary intracellular signaling domain of an immune effector cell. In some embodiments, the intracellular signaling domain of the anti-MSLN CAR is from CD3ζ. In some embodiments, the intracellular signaling domain of the anti-MSLN CAR comprises a co-stimulatory signaling domain. In some embodiments, the co-stimulatory signaling domain is derived from a co-stimulatory molecule selected from the group consisting of CD27, CD28, CD137 (4-1BB), OX40, CD30, CD40, CD3, LFA-1, ICOS, CD2, CD7, LIGHT, NKG2C, B7-H3, ligands of CD83 and combinations thereof. In some embodiments, the anti-MSLN CAR further comprises a hinge domain located between the C-terminus of the extracellular antigen binding domain and the N-terminus of the transmembrane domain. In some embodiments, the hinge domain is from CD8. In some embodiments, the anti-MSLN CAR further comprises a signal peptide located at the N-terminus of the extracellular antigen binding domain. In some embodiments, the signal peptide is from CD8. In some embodiments, the THP is a RGD-based THP, a NGR-based THP, or a combination thereof. In some embodiments, the THP is fused with a transmembrane domain. In some embodiments, the THP is fused with a transmembrane domain and a hinge domain. In some embodiments, the THP is fused with a tag sequence, a peptide linker, and/or a membrane anchoring sequence. In some embodiments, the THP is fused with a TGF-β dominant-negative receptor (TGF-β DNR, e.g., dnTGF-βRII) optionally via a peptide linker. In some embodiments, the immune effector cell is selected from the group consisting of T cells, natural killer (NK) cells, B cells, neutrophils, eosinophils, monocytes, macrophages, dendritic cells, peripheral blood mononuclear cells (PBMC), stem cells from which lymphoid cells may be differentiated, and combinations thereof.

In some embodiments, there is provided an immune effector cell comprising an anti-MSLN CAR comprising (i) an extracellular MSLN binding domain, (ii) a transmembrane domain, and (iii) an intracellular signaling domain, and an exogenous THP, wherein the an anti-MSLN CAR comprises a first V_{H}H antibody moiety comprising the amino acid sequences of SEQ ID NO: 41, or an amino acid sequence having at least 75%, 80%, 90%, 91%, 92%, 93%, 94%, 95%, 96%, 97%, 98%, or 99% identity to the amino acid sequence of SEQ ID NO: 41. In some embodiments, the extracellular antigen binding domain of the anti-MSLN CAR comprises a tandem V_{H}H antibody moiety. In some embodiments, the transmembrane domain of the anti-MSLN CAR is derived from a molecule selected from the group consisting of CD7, CD8, CD8α, CD4, CD28, CD137 (4-1BB), CD80, CD86, CD152, FasL, TR2 and PD1. In some embodiments, the transmembrane domain of the anti-MSLN CAR is derived from CD7. In some embodiments, the transmembrane domain of the anti-MSLN CAR is derived from CD8. In some embodiments, the transmembrane domain of the anti-MSLN CAR is derived from TR2. In some embodiments, the intracellular signaling domain of the anti-MSLN CAR comprises a primary intracellular signaling domain of an immune effector cell. In some embodiments, the intracellular signaling domain of the anti-MSLN CAR is from CD3. In some embodiments, the intracellular signaling domain of the anti-MSLN CAR comprises a co-stimulatory signaling domain. In some embodiments, the co-stimulatory signaling domain is derived from a co-stimulatory molecule selected from the group consisting of CD27, CD28, CD137 (4-1BB), OX40, CD30, CD40, CD3, LFA-1, ICOS, CD2, CD7, LIGHT, NKG2C, B7-H3, ligands of CD83 and combinations thereof. In some embodiments, the anti-MSLN CAR further comprises a hinge domain located between the C-terminus of the extracellular antigen binding domain and the N-terminus of the transmembrane domain. In some embodiments, the hinge domain is from CD8. In some embodiments, the anti-MSLN CAR further comprises a signal peptide located at the N-terminus of the extracellular antigen binding domain. In some embodiments, the signal peptide is from CD8. In some embodiments, the THP is a RGD-based THP, a NGR-based THP, or a combination thereof. In some embodiments, the THP is fused with a transmembrane domain. In some embodiments, the THP is fused with a transmembrane domain and a hinge domain. In some embodiments, the THP is fused with a tag sequence, a peptide linker, and/or a membrane anchoring sequence. In some embodiments, the THP is fused with a TGF-β dominant-negative receptor (TGF-β DNR, e.g., dnTGF-βRII) optionally via a peptide linker. In some embodiments, the immune effector cell is selected from the group consisting of T cells, natural killer (NK) cells, B cells, neutrophils, eosinophils, monocytes, macrophages, dendritic cells, peripheral blood mononuclear cells (PBMC), stem cells from which lymphoid cells may be differentiated, and combinations thereof.

In some embodiments, there is provided an immune effector cell comprising an anti-MSLN CAR and an exogenous membrane-bound NGR. In some embodiments, the immune effector cell comprises a polypeptide comprising, from N-terminus to C-terminus: an anti-MSLN CAR (SEQ ID NO: 41), NGR (SEQ ID NO: 38), a (G45)₂ linker, and the hinge domain (SEQ ID NO: 13) and transmembrane domain (SEQ ID NO: 14) from CD7. In some embodiments, the immune effector cell comprises a polypeptide comprising the amino acid sequence of SEQ ID NO: 42, or a variant thereof having, at least about 95% (for example at least about any of 96%, 97%, 98%, or 99%) sequence identity to SEQ ID NO: 42. In some embodiments, the immune effector cell comprises a polypeptide comprising the amino acid sequence of SEQ ID NO: 42. In some embodiments, the immune effector cell is selected from the group consisting of T cells, natural killer (NK) cells, B cells, neutrophils, eosinophils, monocytes, macrophages, dendritic cells, peripheral blood mononuclear cells (PBMC), stem cells from which lymphoid cells may be differentiated, and combinations thereof.

In some embodiments, there is provided an immune effector cell comprising an anti-GPC2 CAR comprising (i) an extracellular GPC2 binding domain, (ii) a transmembrane domain, and (iii) an intracellular signaling domain, and an exogenous THP. In some embodiments, the extracellular antigen binding domain of the anti-GPC2 CAR comprises a tandem V_{H}H antibody moiety. In some embodiments, the transmembrane domain of the anti-GPC2 CAR is derived from a molecule selected from the group consisting of CD7, CD8, CD8α, CD4, CD28, CD137 (4-1BB), CD80, CD86, CD152, FasL, TR2 and PD1. In some embodiments, the transmembrane domain of the anti-GPC2 CAR is derived from CD7. In some embodiments, the transmembrane domain of the anti-GPC2 CAR is derived from CD8. In some embodiments, the transmembrane domain of the anti-GPC2 CAR is derived from TR2. In some embodiments, the intracellular signaling domain of the anti-GPC2 CAR comprises a primary intracellular signaling domain of an immune effector cell. In some embodiments, the intracellular signaling domain of the anti-GPC2 CAR is from CD3ζ. In some embodiments, the intracellular signaling domain of the anti-GPC2 CAR comprises a co-stimulatory signaling domain. In some embodiments, the co-stimulatory signaling domain is derived from a co-stimulatory molecule selected from the group consisting of CD27, CD28, CD137 (4-1BB), OX40, CD30, CD40, CD3, LFA-1, ICOS, CD2, CD7, LIGHT, NKG2C, B7-H3, ligands of CD83 and combinations thereof. In some embodiments, the anti-GPC2 CAR further comprises a hinge domain located between the C-terminus of the extracellular antigen binding domain and the N-terminus of the transmembrane domain. In some embodiments, the hinge domain is from CD8. In some embodiments, the anti-GPC2 CAR further comprises a signal peptide located at the N-terminus of the extracellular antigen binding domain. In some embodiments, the signal peptide is from CD8. In some embodiments, the THP is a RGD-based THP, a NGR-based THP, or a combination thereof. In some embodiments, the THP is fused with a transmembrane domain. In some embodiments, the THP is fused with a transmembrane domain and a hinge domain. In some embodiments, the THP is fused with a tag sequence, a peptide linker, and/or a membrane anchoring sequence. In some embodiments, the THP is fused with a TGF-β dominant-negative receptor (TGF-β DNR, e.g., dnTGF-βRII) optionally via a peptide linker. In some embodiments, the immune effector cell is selected from the group consisting of T cells, natural killer (NK) cells, B cells, neutrophils, eosinophils, monocytes, macrophages, dendritic cells, peripheral blood mononuclear cells (PBMC), stem cells from which lymphoid cells may be differentiated, and combinations thereof.

In some embodiments, there is provided an immune effector cell comprising an anti-GPC2 CAR comprising (i) an extracellular GPC2 binding domain, (ii) a transmembrane domain, and (iii) an intracellular signaling domain, and an exogenous THP, wherein the an anti-GPC2 CAR comprises a first V_{H}H antibody moiety comprising the amino acid sequences of SEQ ID NO: 44, or an amino acid sequence having at least 75%, 80%, 90%, 91%, 92%, 93%, 94%, 95%, 96%, 97%, 98%, or 99% identity to the amino acid sequence of SEQ ID NO: 44. In some embodiments, the extracellular antigen binding domain of the anti-GPC2 CAR comprises a tandem V_{H}H antibody moiety. In some embodiments, the transmembrane domain of the anti-GPC2 CAR is derived from a molecule selected from the group consisting of CD7, CD8, CD8α, CD4, CD28, CD137 (4-1BB), CD80, CD86, CD152, FasL, TR2 and PD1. In some embodiments, the transmembrane domain of the anti-GPC2 CAR is derived from CD7. In some embodiments, the transmembrane domain of the anti-GPC2 CAR is derived from CD8. In some embodiments, the transmembrane domain of the anti-GPC2 CAR is derived from TR2. In some embodiments, the intracellular signaling domain of the anti-GPC2 CAR comprises a primary intracellular signaling domain of an immune effector cell. In some embodiments, the intracellular signaling domain of the anti-GPC2 CAR is from CD3ζ. In some embodiments, the intracellular signaling domain of the anti-GPC2 CAR comprises a co-stimulatory signaling domain. In some embodiments, the co-stimulatory signaling domain is derived from a co-stimulatory molecule selected from the group consisting of CD27, CD28, CD137 (4-1BB), OX40, CD30, CD40, CD3, LFA-1, ICOS, CD2, CD7, LIGHT, NKG2C, B7-H3, ligands of CD83 and combinations thereof. In some embodiments, the anti-GPC2 CAR further comprises a hinge domain located between the C-terminus of the extracellular antigen binding domain and the N-terminus of the transmembrane domain. In some embodiments, the hinge domain is from CD8. In some embodiments, the anti-GPC2 CAR further comprises a signal peptide located at the N-terminus of the extracellular antigen binding domain. In some embodiments, the signal peptide is from CD8. In some embodiments, the THP is a RGD-based THP, a NGR-based THP, or a combination thereof. In some embodiments, the THP is fused with a transmembrane domain. In some embodiments, the THP is fused with a transmembrane domain and a hinge domain. In some embodiments, the THP is fused with a tag sequence, a peptide linker, and/or a membrane anchoring sequence. In some embodiments, the THP is fused with a TGF-β dominant-negative receptor (TGF-β DNR, e.g., dnTGF-βRII) optionally via a peptide linker. In some embodiments, the immune effector cell is selected from the group consisting of T cells, natural killer (NK) cells, B cells, neutrophils, eosinophils, monocytes, macrophages, dendritic cells, peripheral blood mononuclear cells (PBMC), stem cells from which lymphoid cells may be differentiated, and combinations thereof.

In some embodiments, there is provided an immune effector cell comprising an anti-GPC2 CAR and an exogenous membrane-bound NGR. In some embodiments, the immune effector cell comprises a polypeptide comprising, from N-terminus to C-terminus: an anti-GPC2 CAR (SEQ ID NO: 44), NGR (SEQ ID NO: 38), a (G45)₂ linker, and the hinge domain (SEQ ID NO: 13) and transmembrane domain (SEQ ID NO: 14) from CD7. In some embodiments, the immune effector cell comprises a polypeptide comprising the amino acid sequence of SEQ ID NO: 45, or a variant thereof having, at least about 95% (for example at least about any of 96%, 97%, 98%, or 99%) sequence identity to SEQ ID NO: 45. In some embodiments, the immune effector cell comprises a polypeptide comprising the amino acid sequence of SEQ ID NO: 45. In some embodiments, the immune effector cell is selected from the group consisting of T cells, natural killer (NK) cells, B cells, neutrophils, eosinophils, monocytes, macrophages, dendritic cells, peripheral blood mononuclear cells (PBMC), stem cells from which lymphoid cells may be differentiated, and combinations thereof.

In some embodiments, there is provided an immune effector cell comprising a cTCR and an exogenous THP. In some embodiments, the cTCR is an ε-T-cell receptor fusion construct (“ε-TruC”). In some embodiments, the cTCR is an anti-DLL3 cTCR, an anti-MSLN cTCR, or an anti-GPC2 cTCR. In some embodiments, the THP is a RGD-based THP, a NGR-based THP, or a combination thereof. In some embodiments, the THP is fused with a transmembrane domain. In some embodiments, the THP is fused with a transmembrane domain and a hinge domain. In some embodiments, the THP is fused with a tag sequence, a peptide linker, and/or a membrane anchoring sequence. In some embodiments, the THP is fused with a TGF-β dominant-negative receptor (TGF-β DNR, e.g., dnTGF-βRII) optionally via a peptide linker. In some embodiments, the immune effector cell is selected from the group consisting of T cells, natural killer (NK) cells, B cells, neutrophils, eosinophils, monocytes, macrophages, dendritic cells, peripheral blood mononuclear cells (PBMC), stem cells from which lymphoid cells may be differentiated, and combinations thereof.

In some embodiments, there is provided an immune effector cell comprising an anti-DLL3 cTCR (i.e., DLL3-TRuC) and an exogenous THP. In some embodiments, the anti-DLL3 cTCR comprises the amino acid sequence of SEQ ID NO: 35, or a variant thereof having, at least about 95% (for example at least about any of 96%, 97%, 98%, or 99%) sequence identity to SEQ ID NO: 35. In some embodiments, the THP is a RGD-based THP, a NGR-based THP, or a combination thereof. In some embodiments, the THP is fused with a transmembrane domain. In some embodiments, the THP is fused with a transmembrane domain and a hinge domain. In some embodiments, the THP is fused with a tag sequence, a peptide linker, and/or a membrane anchoring sequence. In some embodiments, the THP is fused with a TGF-β dominant-negative receptor (TGF-β DNR, e.g., dnTGF-βRII) optionally via a peptide linker. In some embodiments, the immune effector cell is selected from the group consisting of T cells, natural killer (NK) cells, B cells, neutrophils, eosinophils, monocytes, macrophages, dendritic cells, peripheral blood mononuclear cells (PBMC), stem cells from which lymphoid cells may be differentiated, and combinations thereof.

In some embodiments, there is provided an immune effector cell comprising an anti-DLL3 cTCR (i.e., DLL3-TRuC) and an exogenous membrane bound RGD. In some embodiments, the immune effector cell comprises a polypeptide that comprises, from N-terminus to C-terminus: an DLL3-ε-TRuC (SEQ ID NO: 35), a P2A linker (SEQ ID NO: 9), an RGD-4C peptide (SEQ ID NO: 2), a (G45)₃ linker, and a hinge domain (SEQ ID NO: 13) and a transmembrane domain from CD7 (SEQ ID NO: 14). In some embodiments, the immune effector cell comprises a polypeptide comprising the amino acid sequence of SEQ ID NO: 36, or a variant thereof having, at least about 95% (for example at least about any of 96%, 97%, 98%, or 99%) sequence identity to SEQ ID NO: 36. In some embodiments, the immune effector cell comprises a polypeptide comprising the amino acid sequence of SEQ ID NO: 36. In some embodiments, the immune effector cell is selected from the group consisting of T cells, natural killer (NK) cells, B cells, neutrophils, eosinophils, monocytes, macrophages, dendritic cells, peripheral blood mononuclear cells (PBMC), stem cells from which lymphoid cells may be differentiated, and combinations thereof.

In some embodiments, there is provided an immune effector cell comprising a combination of any of the exogenous functional receptors, such as any of the CARs, TCRs, cTCRs, TACs, or TAC-like chimeric receptors, and/or a combination of any of the exogenous THPs, such as a RGD-based peptide or NGR-based peptide, as described herein. For example, in some embodiments, the immune effector cell comprises a CAR and a combination of a RGD and a NGR. In some embodiments, the immune effector cell comprises a cTCR and a combination of a RGD and a NGR. In some embodiments, a first polypeptide comprises a first functional receptor and one or more THPs, and a second polypeptide comprises a second functional receptor and one or more THPs. Both the first polypeptide and the second polypeptide may be expressed in the immune effector cell expressing the combination of functional receptors and/or THPs. In some embodiments, the functional receptor expressed on the first polypeptide is different than the functional receptor expressed on the second polypeptide. In some embodiments, the first polypeptide and the second polypeptide comprise the same type of functional receptor, and the functional receptor on the first polypeptide targets a different antigen than the functional receptor on the second polypeptide. In some embodiments, the first polypeptide and the second polypeptide comprise the same THP. In some embodiments, the first polypeptide and the second polypeptide comprise a different THP. In some embodiments, the first polypeptide comprises a different functional receptor or THP compared with the second polypeptide. In some embodiments, first polypeptide comprises a different functional receptor and THP compared with the second polypeptide.

According to other aspects of the disclosure, there are also provided an engineered immune effector cell expressing or comprising the composition described above; a pharmaceutical composition comprising the composition or the engineered immune effector cell, and a pharmaceutically acceptable carrier; a method of making the engineered immune effector cell by introducing the composition into an immune effector cell; and a method of treating a disease or disorder in a subject, comprising administering to the subject an effective amount of the pharmaceutical composition.

### 2.1 Immune Effector Cells

“Immune effector cells” are immune cells that can perform immune effector functions. In some embodiments, the immune effector cells express at least FcγRIII and perform ADCC effector function.

In some embodiments, the immune effector cell is selected from the group consisting of T cells, natural killer (NK) cells, B cells, neutrophils, eosinophils, monocytes, macrophages, dendritic cells, peripheral blood mononuclear cells (PBMC), stem cells from which lymphoid cells may be differentiated, and combinations thereof.

In some embodiments, the immune effector cells are T cells. In some embodiments, the immune effector cell is selected from the group consisting of a cytotoxic T cell, a helper T cell, a natural killer (NK) T cell, an iNK-T cell, an NK-T like cell, a γδT cell, a tumor-infiltrating T cell and a DC-activated T cell. In some embodiments, the T cells are CD4+/CD8−, CD4−/CD8+, CD4+/CD8+, CD4−/CD8−, or combinations thereof. In some embodiments, the T cells produce IL-2, IFN, and/or TNF upon expressing the CAR and binding to the target cells. In some embodiments, the CD8+ T cells lyse antigen-specific target cells upon expressing the CAR and binding to the target cells.

In some embodiments, the immune effector cells are NK cells. In other embodiments, the immune effector cells can be established cell lines, for example, NK-92 cells.

Subpopulations of immune cells can be defined by the presence or absence of one or more cell surface markers known in the art (e.g., CD3, CD4, CD8, CD19, CD20, CD11c, CD123, CD56, CD34, CD14, CD33, etc.). In some embodiments, the immune effector cells described herein may be present in a composition (e.g., a pharmaceutical composition) that comprises a plurality of engineered immune effector cells. In some embodiments, the engineered immune effector cells can be a specific subpopulation of an immune cell type, a combination of subpopulations of an immune cell type, or a combination of two or more immune cell types. In some embodiments, the immune effector cell is present in a homogenous immune cell population. In some embodiments, the immune effector cell is present in a heterogeneous immune cell population that is enhanced in the immune effector cell. In some embodiments, the engineered immune cell is a lymphocyte. In some embodiments, the engineered immune cell is not a lymphocyte. In some embodiments, the engineered immune cell is suitable for adoptive immunotherapy.

In some embodiments, the immune effector cell is derived from a primary cell. In some embodiments, the immune effector cell is a primary cell isolated from an individual. In some embodiments, the immune effector cell is propagated (such as proliferated and/or differentiated) from a primary cell isolated from an individual. In some embodiments, the primary cell is obtained from the thymus. In some embodiments, the primary cell is obtained from the lymph or lymph nodes (such as tumor draining lymph nodes). In some embodiments, the primary cell is obtained from the spleen. In some embodiments, the primary cell is obtained from the bone marrow. In some embodiments, the primary cell is obtained from the blood, such as the peripheral blood. In some embodiments, the primary cell is a Peripheral Blood Mononuclear Cell (PBMC). In some embodiments, the primary cell is derived from the blood plasma. In some embodiments, the primary cell is derived from a tumor. In some embodiments, the primary cell is obtained from the mucosal immune system. In some embodiments, the primary cell is obtained from a biopsy sample.

In some embodiments, the immune effector cell is derived from a cell line. In some embodiments, the immune effector cell is obtained from a commercial cell line. In some embodiments, the immune effector cell is propagated (such as proliferated and/or differentiated) from a cell line established from a primary cell isolated from an individual. In some embodiments, the cell line is mortal. In some embodiments, the cell line is immortalized. In some embodiments, the cell line is a tumor cell line, such as a leukemia or lymphoma cell line. In some embodiments, the cell line is a cell line derived from the PBMC. In some embodiments, the cell line is a stem cell line. In some embodiments, the cell line is NK-92.

In some embodiments, the immune effector cell is derived from a stem cell. In some embodiments, the stem cell is an embryonic stem cell (ESC). In some embodiments, the stem cell is hematopoietic stem cell (HSC). In some embodiments, the stem cell is a mesenchymal stem cell. In some embodiments, the stem cell is an induced pluripotent stem cell (iPSC).

### 2.2 Chimeric Antigen Receptor (CAR)

In an aspect of the present disclosure, provided herein is a CAR or CAR-T cell. In some embodiments, an immune cell comprising the CAR or CAR-T cell further comprises a THP.

Several “generations” of CARs have been developed. First-generation CAR T-cells utilize an intracellular domain from the CD3ζ-chain of the TCR, which provides so called ‘signal 1,’ and induces cytotoxicity against targeted cells. Engagement and signaling via the CD3ζ chain is required for T-cell stimulation and proliferation but is not often sufficient for sustained proliferation and activity in the absence of a second signal or ‘signal 2.’ Second-generation CARs were developed to enhance efficacy and persistence in vivo after reinfusion into a subject and contain an second costimulatory signaling domain (CD28 or 4-1BB) intracellular domain that functions to provide ‘signal 2’ to mitigate energy and activation-induced cell death seen with first generation CAR T-cells. Third-generation CARs are further optimized by use of two distinct costimulatory domains in tandem, e.g., CD28/4-1BB/CD3ζ or CD28/OX-40/CDK3ζ (see, e.g., Yeku et al., 2016, Armored CAR T-cells: utilizing cytokines and pro-inflammatory ligands to enhance CAR T-cell anti-tumor efficacy. Biochem Soc Trans. 44(2):412). CARs have been further optimized or “armored” to secrete active cytokines or express costimulatory ligands that further improve efficacy and persistence. The CAR used in the present disclosure can be first-generation, second-generation, third-generation, or “armored” CARs.

In some embodiments, the CAR is specific for DLL3, MSLN, or GPC2. In some embodiments, the CAR is specific for DLL3. In some embodiments, the CAR is specific for MSLN. In some embodiments, the CAR is specific for GPC2. In some embodiments, the CAR is specific for DLL3, MSLN, or GPC2, and is armored with a THP. In some embodiments, the CAR is an anti-DLL3 CAR armored with a THP. In some embodiments, the CAR is an anti-MSLN CAR armored with a THP. In some embodiments, the CAR is an anti-GPC2 CAR armored with a THP. In some embodiments, the THP is an RGD-based peptide. In some embodiments, the THP is a NGR-based peptide.

In some embodiments, the CAR is specific for DLL3, i.e. it is an anti-DLL3 CAR. DLL3 protein (delta-like protein 3) has been found to be clinically associated with various proliferative disorders, including tumors exhibiting neuroendocrine features, such as small-cell lung cancer (SCLC), ovarian cancer (OC) or neuroblastoma (NBL). The term DLL3 as used herein includes variants, isoforms, species homologs (for example, from rodents or other non-human mammal) of human DLL3, and analogs having at least one common epitope with DLL3. In some embodiments, the CAR is specific for human DLL3.

An exemplary amino acid sequence of human DLL3 is disclosed at UniProtKB—Q9NYJ7 (DLL3 HUMAN). In some embodiments, a variant of human DLL3 may be at least about 80%, 85%, 90%, 95%, 96%, 97%, 98%, or 99% identical in amino acid sequence to the human DLL3 of UniProtKB—Q9NYJ7. In some embodiments, the CAR is specific for human DLL3 of UniProtKB—Q9NYJ7.

SCLC, originating from neuroendocrine progenitor cells, comprises approximately 15% of all lung cancers, and has one of the lowest 5-year survival rates at 6% (Alvarado-Luna et al., 2016, Transl Lung Cancer Res 5:26-38; Siegel et al., 2017, CA Cancer J Clin 67:7-30). This is because it is highly aggressive, with about two-thirds of patients having metastatic diseases at diagnosis, and is highly refractory to conventional treatment (e.g., platinum-based chemotherapy).

In some embodiments, the anti-DLL3 CAR comprises (a) an extracellular antigen binding domain that binds specifically to DLL3, especially murine or human DLL3; (b) a transmembrane domain; and (c) an intracellular signaling domain.

The anti-DLL3 CAR or a CAR targeting other antigen(s) that can be useful in the present disclosure can have one or more antigen binding moieties in its extracellular antigen binding domain. For example, The CAR can have two tandem V_{H}H moieties in the extracellular antigen binding domain.

In some embodiments, the anti-DLL3 CAR comprises a first V_{H}H antibody moiety that comprises a CDR1 comprising the amino acid sequence of SEQ ID NO: 29 or a variant thereof comprising up to about 3 amino acid substitutions, a CDR2 comprising the amino acid sequence of SEQ ID NO:30 or a variant thereof comprising up to about 3 amino acid substitutions, and a CDR3 comprising the amino acid sequence of SEQ ID NO: 31 or a variant thereof comprising up to about 3 amino acid substitutions, and a second V_{H}H antibody moiety that comprises a CDR1 comprising the amino acid sequence of SEQ ID NO: 32 or a variant thereof comprising up to about 3 amino acid substitutions, a CDR2 comprising the amino acid sequence of SEQ ID NO:33 or a variant thereof comprising up to about 3 amino acid substitutions, and a CDR3 comprising the amino acid sequence of SEQ ID NO: 34 or a variant thereof comprising up to about 3 amino acid substitutions.

In some embodiments, the anti-DLL3 CAR comprises a first V_{H}H antibody moiety that comprises a CDR1 comprising the amino acid sequence of SEQ ID NO: 29, a CDR2 comprising the amino acid sequence of SEQ ID NO: 30, and a CDR3 comprising the amino acid sequence of SEQ ID NO: 31, and a second V_{H}H antibody moiety that comprises a CDR1 comprising the amino acid sequence of SEQ ID NO: 32, a CDR2 comprising the amino acid sequence of SEQ ID NO:33, and a CDR3 comprising the amino acid sequence of SEQ ID NO: 34. In some embodiments, said first V_{H}H antibody moiety and second V_{H}H antibody moiety are linked via a (G4S)n linker, wherein the linker can be any appropriate length, for example, n can be any integer between 1 to 20, preferably between 3 to 12, such as for example 3, 4, 5, 6, 7, 8, 9, 10, 11, 12, or more.

In some embodiments, the anti-DLL3 CAR comprises a first V_{H}H antibody moiety comprising the amino acid sequences of SEQ ID NO: 27 (i.e. AS63997VH5 V_{H}H) or an amino acid sequence having at least 75%, 80%, 90%, 91%, 92%, 93%, 94%, 95%, 96%, 97%, 98%, or 99% identity to the amino acid sequence of SEQ ID NO: 27, and a second V_{H}H antibody moiety comprising the amino acid sequences of SEQ ID NO: 28 (i.e. AS64380VH5 V_{H}H) or an amino acid sequence having at least 75%, 80%, 90%, 91%, 92%, 93%, 94%, 95%, 96%, 97%, 98%, or 99% identity to the amino acid sequence of SEQ ID NO: 28. In some embodiments, the anti-DLL3 CAR comprises a first V_{H}H antibody moiety comprising the amino acid sequences of SEQ ID NO: 27, and a second V_{H}H antibody moiety comprising the amino acid sequences of SEQ ID NO: 28. In some embodiments, said first V_{H}H antibody moiety and second V_{H}H antibody moiety are linked via a (G4S)₃ linker.

In some embodiments, the anti-DLL3 CAR comprises the amino acid sequence of SEQ ID NO: 8 or an amino acid sequence having at least 75%, 80%, 90%, 91%, 92%, 93%, 94%, 95%, 96%, 97%, 98%, or 99% identity to the amino acid sequence of SEQ ID NO: 8. In some embodiments, the anti-DLL3 CAR comprises the amino acid sequence of SEQ ID NO: 8.

In some embodiments, the CAR is specific for MSLN, i.e. it is an anti-MSLN CAR. The term MSLN as used herein includes variants, isoforms, species homologs (for example, from rodents or other non-human mammal) of human MSLN, and analogs having at least one common epitope with MSLN. In some embodiments, the CAR is specific for human MSLN. An exemplary amino acid sequence of human MSLN is disclosed at UniProtKB—H3BR90 (H3BR90_HUMAN). In some embodiments, a variant of human MSLN may be at least about 80%, 85%, 90%, 95%, 96%, 97%, 98%, or 99% identical in amino acid sequence to the human MSLN of UniProtKB—H3BR90. In some embodiments, the CAR is specific for human MSLN of UniProtKB—H3BR90.

In some embodiments, the anti-MSLN CAR comprises (a) an extracellular antigen binding domain that binds specifically to MSLN, especially murine or human MSLN; (b) a transmembrane domain; and (c) an intracellular signaling domain. In some embodiments, the anti-MSLN CAR comprises the amino acid sequence of SEQ ID NO: 41 or an amino acid sequence having at least 75%, 80%, 90%, 91%, 92%, 93%, 94%, 95%, 96%, 97%, 98%, or 99% identity to the amino acid sequence of SEQ ID NO: 41. In some embodiments, the anti-MSLN CAR comprises the amino acid sequence of SEQ ID NO: 41.

In some embodiments, the CAR is specific for GPC2, i.e. it is an anti-GPC2 CAR. The term GPC2 as used herein includes variants, isoforms, species homologs (for example, from rodents or other non-human mammal) of human GPC2, and analogs having at least one common epitope with GPC2. In some embodiments, the CAR is specific for human GPC2. An exemplary amino acid sequence of human GPC2 is disclosed at UniProtKB—Q8N158 (GPC2_HUMAN). In some embodiments, a variant of human GPC2 may be at least about 80%, 85%, 90%, 95%, 96%, 97%, 98%, or 99% identical in amino acid sequence to the human GPC2 of UniProtKB—Q8N158. In some embodiments, the CAR is specific for human GPC2 of UniProtKB—Q8N158.

In some embodiments, the anti-GPC2 CAR comprises (a) an extracellular antigen binding domain that binds specifically to GPC2, especially murine or human GPC2; (b) a transmembrane domain; and (c) an intracellular signaling domain. In some embodiments, the anti-GPC2 CAR comprises the amino acid sequence of SEQ ID NO: 44 or an amino acid sequence having at least 75%, 80%, 90%, 91%, 92%, 93%, 94%, 95%, 96%, 97%, 98%, or 99% identity to the amino acid sequence of SEQ ID NO: 44. In some embodiments, the anti-GPC2 CAR comprises the amino acid sequence of SEQ ID NO: 44.

### 2.2.1 Extracellular Antigen Binding Domain of a CAR

The term “extracellular antigen binding domain”, “antigen binding fragment”, “antigen recognition portion”, and similar expressions are used interchangeably with reference to CARs or TCRs in the present application. Antigen binding domains take many forms. Non-limiting examples include bispecific receptors (Zakaria Grada, et al. TanCAR: A Novel Bispecific Chimeric Antigen Receptor for Cancer Immunotherapy. Molecular Therapy, 2013, 2, e105), single domain V_{H}H based CARs (De Meyer T, et a., V_{H}H-based products as research and diagnostic tools. Trends Biotechnol. 2014 May; 32(5):263-70), and “universal” CARs comprising avidin that binds to any antigen receptor that incorporates biotin (Huan Shi, et al. Chimeric antigen receptor for adoptive immunotherapy of cancer: latest research and future prospects. Molecular Cancer, 2014,13:219).

In some embodiments, the antigen binding domain is selected from the group consisting of Fab, a Fab′, a (Fab′)₂, an Fv, a single chain Fv (scFv), a single domain antibody (sdAb), and a peptide ligand specifically binding to the target molecule.

In some embodiments, the antigen binding domain is an antibody moiety. In some embodiments, the antibody moiety is monospecific. In some embodiments, the antibody moiety is multi-specific. In some embodiments, the antibody moiety is bispecific. In some embodiments, the antibody moiety is a tandem scFv, a diabody (Db), a single chain diabody (scDb), a dual-affinity retargeting (DART) antibody, a dual variable domain (DVD) antibody, a chemically cross-linked antibody, a heteromultimeric antibody, or a heteroconjugate antibody. In some embodiments, the antibody moiety is a scFv. In some embodiments, the antibody moiety is a single domain antibody (sdAb). In some embodiments, the antibody moiety is a V_{H}H. In some embodiments, the antibody moiety comprises a tandem V_{H}H. In some embodiments, the antibody moiety is fully human, semi-synthetic with human antibody framework regions, or humanized.

In certain embodiments, the antigen binding domain is multispecific, such as bispecific or trispecific. The term “multispecific” is used in the present disclosure in its broader sense, which is, an antigen binding domain is multispecific if it can target more than one epitopes on the same antigen or it can target more than one antigens. In some embodiments, the antigen binding domain comprises a tandem V_{H}H domain. A tandem V_{H}H may generated, for example, by fusing two tandem V_{H}H domains to the hinge and Fc domains.

Antigens have been identified in most of the human cancers, including Burkitt lymphoma, neuroblastoma, melanoma, osteosarcoma, renal cell carcinoma, breast cancer, prostate cancer, lung carcinoma, and colon cancer. Tumor associated antigens (TAAs) include, without limitation, GPC2, CD19, CD20, CD22, CD24, CD33, CD38, CD123, CD228, CD138, BCMA, GPC3, CEA, folate receptor (FRα), mesothelin (MSLN), DLL3, CD276, gp100, 5T4, GD2, EGFR, MUC-1, PSMA, EpCAM, MCSP, SM5-1, MICA, MICB, ULBP and HER-2. TAAs further include neoantigens, peptide/MHC complexes, and HSP/peptide complexes.

### 2.2.2 Intracellular Signaling Domain of a CAR

The intracellular signaling domain (ICD) comprises a primary intracellular signaling domain of an immune cell (such as T cell). In certain embodiments, the primary intracellular signaling domain is derived from CD3ζ, FcRγ, FcRβ, CD3γ, CD3δ, CD3ε, CD5, CD22, CD79a, CD79b, or CD66d. In certain embodiments, the primary intracellular signaling domain is derived from CD3ζ (i.e., “a CD3ζ intracellular signaling domain”). In certain embodiments, the intracellular signaling domain comprises an intracellular co-stimulatory sequence. In certain embodiments, the intracellular signaling domain comprises both a primary intracellular signaling domain (e.g., a CD3ζ intracellular signaling domain) and an intracellular co-stimulatory domain. In certain embodiments, the intracellular signaling domain comprises a primary intracellular signaling domain but does not comprise an intracellular co-stimulatory domain. In certain embodiments, the intracellular signaling domain comprises an intracellular co-stimulatory sequence but does not comprise a primary intracellular signaling domain.

### 2.2.3 Co-Stimulatory Domain

“Co-stimulatory domain” (CSD) as used herein refers to the portion of the CAR which enhances the proliferation, survival and/or development of memory cells. The CARs of the disclosure may comprise one or more co-stimulatory domains. Each costimulatory domain comprises a costimulatory domain of any one or more of, for example, members of the TNFR superfamily, CD28, CD137 (4-1BB), CD134 (OX40), Dap10, CD27, CD2, CD5, ICAM-1, LFA-1 (CD11a/CD18), ICOS (CD278), Lck, TNFR-I, TNFR-II, Fas, CD30, CD40 and combinations thereof. Further costimulatory domains used with the disclosure comprise one or more of: 2B4/CD244/SLAMF4, 4-1BB/TNFSF9/CD137, B7-1/CD80, B7-2/CD86, B7-H1/PD-L1, B7-H2, B7-H3, B7-H4, B7-H6, B7-H7, BAFF-R/TNFRSF13C, BAFF/BLyS/TNFSF13B, BLAME/SLAMF8, BTLA/CD272, CD100 (SEMA4D), CD103, CD11a, CD11b, CD11c, CD11d, CD150, CD160 (BY55), CD18, CD19, CD2, CD200, CD229/SLAMF3, CD27 Ligand/TNFSF7, CD27/TNFRSF7, CD28, CD29, CD2F-10/SLAMF9, CD30 Ligand/TNFSF8, CD30/TNFRSF8, CD300a/LMIR1, CD4, CD40 Ligand/TNFSF5, CD40/TNFRSF5, CD48/SLAMF2, CD49a, CD49D, CD49f, CD53, CD58/LFA-3, CD69, CD7, CD8, CD8α, CD8β, CD82/Kai-1, CD84/SLAMF5, CD90/Thy1, CD96, CDS, CEACAM1, CRACC/SLAMF7, CRTAM, CTLA-4, DAP12, Dectin-1/CLEC7A, DNAM1 (CD226), DPPIV/CD26, DR3/TNFRSF25, EphB6, GADS, Gi24/VISTA/B7-H5, GITR Ligand/TNFSF18, GITR/TNFRSF18, HLA Class I, HLA-DR, HVEM/TNFRSF14, IA4, ICAM-1, ICOS/CD278, Ikaros, IL2Rβ, IL2Rγ, IL7Rα, Integrin α4/CD49d, Integrin α4β1, Integrin α4β7/LPAM-1, IPO-3, ITGA4, ITGA6, ITGAD, ITGAE, ITGAL, ITGAM, ITGAX, ITGB1, ITGB2, ITGB7, KIRDS2, LAG-3, LAT, LIGHT/TNFSF14, LTBR, Ly108, Ly9 (CD229), lymphocyte function associated antigen-1 (LFA-1), Lymphotoxin-α/TNF-β, NKG2C, NKG2D, NKp30, NKp44, NKp46, NKp80 (KLRF1), NTB-A/SLAMF6, OX40 Ligand/TNFSF4, OX40/TNFRSF4, PAG/Cbp, PD-1, PDCD6, PD-L2/B7-DC, PSGL1, RELT/TNFRSF19L, SELPLG (CD162), SLAM (SLAMF1), SLAM/CD150, SLAMF4 (CD244), SLAMF6 (NTB-A), SLAMF7, SLP-76, TACI/TNFRSF13B, TCL1A, TCL1B, TIM-1/KIM-1/HAVCR, TIM-4, TL1A/TNFSF15, TNF RIFTNFRSF1B, TNF-α, TRANCE/RANKL, TSLP, TSLPR, VLA1, TR2, and VLA-6.

In certain embodiments, the intracellular signaling domain comprises an intracellular co-stimulatory domain derived from a co-stimulatory molecule. In some embodiments, the co-stimulatory molecule selected from the group consisting of CD27, CD28, 4-1BB, OX40, CD40, PD-1, LFA-1, ICOS, CD2, CD7, LIGHT, NKG2C, B7-H3, TNFRSF9, TNFRSF4, TNFRSF8, CD40LG, ITGB2, KLRC2, TNFRSF18, TNFRSF14, HAVCR1, LGALS9, DAP10, DAP12, CD83, ligands of CD83, and combinations thereof. In some embodiments, the co-stimulatory molecule comprises CD7. In some embodiments, the co-stimulatory molecule comprises CD28.

### 2.2.4 Transmembrane Domain

In some embodiments, the CARs of the present disclosure comprise a transmembrane domain. In CARs, “transmembrane domain” (TMD or TM) as used herein refers to the region of the CAR which crosses the plasma membrane. The transmembrane domain of the CAR of the disclosure is the transmembrane region of a transmembrane protein (for example Type I transmembrane proteins), an artificial hydrophobic sequence or a combination thereof. Although the main function of the transmembrane is to anchor the CAR in the T cell membrane, in certain embodiments, the transmembrane domain influences CAR function. In some embodiments, the transmembrane domain of the CAR comprises a transmembrane domain chosen from the transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL-2R beta, IL-2R gamma, IL-7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C.

In certain embodiments, the transmembrane domain is from CD7, CD8, CD8α, CD4, CD28, CD137, CD80, CD86, CD152, FasL, TR2 and PD1 or ICOS. Gueden et al. associated use of the ICOS transmembrane domain with increased CAR T cell persistence and overall anti-tumor efficacy (Guedan S. et al., Enhancing CAR T cell persistence through ICOS and 4-1BB costimulation. JCI Insight. 2018; 3:96976). In an embodiment, the transmembrane domain comprises a hydrophobic a helix that spans the cell membrane. Other transmembrane domains will be apparent to those of skill in the art and may be used in connection with alternate embodiments of the disclosure. In certain embodiments, the transmembrane domain is a human transmembrane domain. In certain embodiments, the transmembrane domain comprises a CD8 transmembrane domain In certain embodiments, the transmembrane domain comprises human CD8a transmembrane domain. In certain embodiments, the transmembrane domain comprises human CD28 transmembrane domain. In certain embodiments, the transmembrane domain comprises a CD7 transmembrane domain. In certain embodiments, the transmembrane domain comprises a TR2 transmembrane domain. In certain embodiments, the transmembrane domain comprises a FasL transmembrane domain.

### 2.2.5 Hinge Region

The chimeric receptors of the present application may comprise a hinge domain that is located between the extracellular domain and the transmembrane domain. A hinge domain is an amino acid segment that is generally found between two domains of a protein and may allow for flexibility of the protein and movement of one or both of the domains relative to one another. Any amino acid sequence that provides such flexibility and movement of the extracellular domain relative to the transmembrane domain of the effector molecule can be used. The hinge domain may contain about 10-100 amino acids, e.g., about any one of 15-75 amino acids, 20-50 amino acids, or 30-60 amino acids. In some embodiments, the hinge domain may be at least about any one of 10, 11, 12, 13, 14, 15, 16, 17, 18, 19, 20, 21, 22, 23, 24, 25, 26, 27, 28, 29, 30, 35, 40, 45, 50, 55, 60, 65, 70, or 75 amino acids in length.

In certain embodiments, the hinge domain is a hinge domain of a naturally occurring protein. Hinge domains of any protein known in the art to comprise a hinge domain are compatible for use in the chimeric receptors described herein. In certain embodiments, the hinge domain is at least a portion of a hinge domain of a naturally occurring protein and confers flexibility to the chimeric receptor. In certain embodiments, the hinge domain is derived from CD8, such as CD8α. In certain embodiments, the hinge domain is a portion of the hinge domain of CD8α, e.g., a fragment containing at least 15 (e.g., 20, 25, 30, 35, or 40) consecutive amino acids of the hinge domain of CD8α. In certain embodiments, the hinge domain is derived from CD28. In certain embodiments, the hinge domain is derived from CD7.

Hinge domains of antibodies, such as an IgG, IgA, IgM, IgE, or IgD antibodies, are also compatible for use in the chimeric receptor systems described herein. In certain embodiments, the hinge domain is the hinge domain that joins the constant domains CH1 and CH2 of an antibody. In certain embodiments, the hinge domain is of an antibody and comprises the hinge domain of the antibody and one or more constant regions of the antibody. In certain embodiments, the hinge domain comprises the hinge domain of an antibody and the CH3 constant region of the antibody. In certain embodiments, the hinge domain comprises the hinge domain of an antibody and the CH2 and CH3 constant regions of the antibody. In certain embodiments, the antibody is an IgG, IgA, IgM, IgE, or IgD antibody. In certain embodiments, the antibody is an IgG antibody. In some embodiments, the antibody is an IgG1, IgG2, IgG3, or IgG4 antibody. In certain embodiments, the hinge region comprises the hinge region and the CH2 and CH3 constant regions of an IgG1 antibody. In certain embodiments, the hinge region comprises the hinge region and the CH3 constant region of an IgG1 antibody.

Non-naturally occurring peptides may also be used as hinge domains for the chimeric receptors described herein. In certain embodiments, the hinge domain between the C-terminus of the extracellular ligand-binding domain of an Fc receptor and the N-terminus of the transmembrane domain is a peptide linker, such as a (GxS)n linker, wherein x and n, independently can be an integer between 1 and 20, preferably between 3 and 12, including 3, 4, 5, 6, 7, 8, 9, 10, 11, 12, or more.

### 2.2.6 Signal Peptide

The chimeric receptors of the present application may comprise a signal peptide (also known as a leading peptide) at the N-terminus of the polypeptide. In general, leading peptides are peptide sequences that target a polypeptide to the desired site in a cell. Leading peptides including signal sequences of naturally occurring proteins or synthetic, non-naturally occurring signal sequences may be compatible for use in the chimeric receptors described herein. In some embodiments, the leading peptide is derived from a molecule selected from the group consisting of CD8, GM-CSF receptorα, and IgG1 heavy chain. In some embodiments, the signal peptide is derived from CD8, such as CD8α. In some embodiments, the signal peptide is derived from CD28.

### 2.3 T-Cell Receptors (TCRs) and Chimeric TCRs (cTCRs)

In some embodiments, the functional exogenous receptor is a recombinant T-cell receptor (TCR). In some embodiments, the recombinant TCR is specific for a tumor antigen. In some embodiments, the tumor antigen is DLL3, MSLN, GPC2, CD19, BCMA, NY-ESO-1, VEGFR2, MAGE-A3, VEGFR2, MAGE-A3, CD20, CD22, CD33, CD38, CEA, EGFR (such as EGFRvIII), GD2, HER2, IGF1R, PSMA, ROR1, WT1, or another tumor antigen with clinical significance. In some embodiments, the tumor antigen is derived from an intracellular protein of tumor cells. In some embodiments, the TCR is specific for DLL3. In some embodiments, the TCR is specific for MSLN. In some embodiments, the TCR is specific for GPC2. Any of the TCRs known in the art may be used in the present application. In some embodiments, the TCR has an enhanced affinity to the tumor antigen. Exemplary TCRs and methods for introducing the TCRs to immune effector cells have been described, for example, in U.S. Pat. No. 5,830,755, and Kessels et al. Immunotherapy through TCR gene transfer. Nat. Immunol. 2, 957-961 (2001). In some embodiments, the immune effector cell is a TCR-T cell or a chimeric TCR (cTCR)-T cell.

The TCR receptor complex is an octomeric complex formed by variable TCR receptor α and β chains (γ and δ chains on case of γδ T cells) with three dimeric signaling modules CD3δ/ε, CD3γ/ε and CD247 (T-cell surface glycoprotein CD3 zeta chain) ζ/ζ or ζ/η. Ionizable residues in the transmembrane domain of each subunit form a polar network of interactions that hold the complex together. TCR complex has the function of activating signaling cascades in T cells.

In some embodiments, the functional exogenous receptor is a chimeric TCR (“cTCR”). In some embodiments, the functional exogenous receptor is an engineered TCR comprising one or more cTCRs. Exemplary cTCRs have been described, for example, in US20170166622A1, which is incorporated herein by reference in its entirety. cTCRs typically comprise a chimeric receptor (CR) antigen binding domain linked (e.g., fused) directly or indirectly to the full-length or a portion of a TCR subunit, such as TCRα, TCRβ, TCRγ, TCRδ, CD3γ, CD3ε, and CD3δ. The fusion polypeptide can be incorporated into a functional TCR complex along with other TCR subunits and confers antigen specificity to the TCR complex. In some embodiments, the binding domain is linked (e.g., fused) directly or indirectly to the full-length or a portion of the CD3ε subunit (referred to as “εTCR”). The intracellular signaling domain of the cTCR can be derived from the intracellular signaling domain of a TCR subunit. The transmembrane domain of the cTCR can also be derived from a TCR subunit. In some embodiments, the intracellular signaling domain and the transmembrane domain of the cTCR are derived from the same TCR subunit. In some embodiments, the intracellular signaling domain and the transmembrane domain of the cTCR are derived from CD3ε. In some embodiments, the binding domain and the TCR subunit (or a portion thereof) can be fused via a linker (such as a GS linker). In some embodiments, the cTCR further comprises an extracellular domain of a TCR subunit or a portion thereof, which can be the same or different from the TCR subunit from which the intracellular signaling domain and/or transmembrane domain are derived from.

In some embodiments, the transmembrane domain of the cTCR is derived from the transmembrane domain of a TCR subunit selected from the group consisting of TCRα, TCRβ, TCRγ, TCRδ, CD3γ, CD3ε, and CD3δ. In some embodiments, the cTCR comprises a transmembrane domain that comprises a transmembrane domain of a CD3ε TCR subunit (e.g., εTCR, or ε-TRuC).

In some embodiments, the transmembrane domain and intracellular signaling domain of the cTCR are derived from the same TCR subunit. In some embodiments, the cTCR further comprises at least a portion of an extracellular sequence of a TCR subunit, and the TCR extracellular sequence in some embodiments may be derived from the same TCR subunit as the transmembrane domain and/or intracellular signaling domain. In some embodiments, the cTCR comprises a full-length TCR subunit. For example, in some embodiments, the cTCR comprises a binding domain fused (directly or indirectly) to the N-terminus of a TCR subunit (e.g., CD3ε).

In some embodiments, the intracellular signaling domain of the cTCR is derived from the intracellular signaling domain of a TCR subunit selected from the group consisting of TCRα, TCRβ, TCRγ, TCRδ, CD3γ, CD3ε, and CD3δ. In some embodiments, the intracellular signaling domain of the cTCR is derived from the intracellular signaling domain of CD3ε.

In some embodiments, the cTCR comprises a TCR subunit comprising at least a portion of a TCR extracellular domain, and a TCR intracellular domain comprising a stimulatory domain from an intracellular signaling domain of CD3ε; and an antigen binding domain, wherein the TCR subunit and the antigen binding domain are operatively linked, and wherein the cTCR incorporates into a TCR when expressed in an immune effector cell (such as a T cell). In some embodiments, the antigen binding domain is specific for DLL3, MSLN, GPC2, or a combination thereof. In some embodiments, the antigen binding domain is specific for DLL3.

In some embodiments, the cTCR is an ε-T-cell receptor fusion construct (“ε-TruC”). In some embodiments, the ε-TruC comprises a TCR subunit comprising at least a portion of a TCR extracellular domain, and a TCR intracellular domain comprising a stimulatory domain from an intracellular signaling domain of CD3ε; and an antigen binding domain, wherein the TCR subunit and the antigen binding domain are operatively linked, and wherein the cTCR incorporates into a TCR when expressed in an immune effector cell (such as a T cell). In some embodiments, the ε-TruC is armored with an exogenous THP. In some embodiments, In some embodiments, the cTCR is an anti-DLL3 cTCR, an anti-MSLN cTCR, or an anti-GPC2 cTCR. In some embodiments, the THP is a RGD-based THP, a NGR-based THP, or a combination thereof. In some embodiments, the THP is fused with a transmembrane domain. In some embodiments, the THP is fused with a transmembrane domain and a hinge domain. In some embodiments, the THP is fused with a tag sequence, a peptide linker, and/or a membrane anchoring sequence.

In some embodiments, the cTCR is an anti-DLL3 cTCR (i.e., DLL3-TRuC). In some embodiments, the anti-DLL3 cTCR comprises the amino acid sequence of SEQ ID NO: 35, or a variant thereof having, at least about 95% (for example at least about any of 96%, 97%, 98%, or 99%) sequence identity to SEQ ID NO: 35.

In some embodiments, the cTCR is an anti-DLL3 cTCR (i.e., DLL3-TRuC) armored with and an exogenous membrane bound RGD. In some embodiments, the cTCR comprises, from N-terminus to C-terminus: a DLL3-ε-TRuC (SEQ ID NO: 35), a P2A linker (SEQ ID NO: 9), an RGD-4C peptide (SEQ ID NO: 2), a (G4S)₃ linker, and a hinge domain (SEQ ID NO: 13) and a transmembrane domain from CD7 (SEQ ID NO: 14). In some embodiments, the immune effector cell comprises the amino acid sequence of SEQ ID NO:36, or a variant thereof having, at least about 95% (for example at least about any of 96%, 97%, 98%, or 99%) sequence identity to SEQ ID NO: 36.

### 2.4 T-Cell Antigen Coupler (TAC) and TAC-Like Chimeric Receptors

In some embodiments, the functional exogenous receptor is a T-cell antigen coupler (TAC) receptor. Exemplary TAC receptors have been described, for example, in US20160368964A1, which is incorporated herein by reference. In some embodiments, the TAC comprises a targeting domain, a TCR-binding domain that specifically binds a protein associated with the TCR complex, and a TCR-signaling domain. In some embodiments, the targeting domain is an antibody fragment, such as scFv or V_{H}H, which specifically binds to a tumor antigen. In some embodiments, the targeting domain is a designed Ankyrin repeat (DARPin) polypeptide. In some embodiments, the tumor antigen is selected from the group consisting of In some embodiments, the recombinant TCR is specific for a tumor antigen. In some embodiments, the tumor antigen is DLL3, MSLN, GPC2, CD19, BCMA, NY-ESO-1, VEGFR2, MAGE-A3, VEGFR2, MAGE-A3, CD20, CD22, CD33, CD38, CEA, EGFR (such as EGFRvIII), GD2, HER2, IGF1R, PSMA, ROR1, WT1, or another tumor antigen with clinical significance. In some embodiments, the tumor antigen is derived from an intracellular protein of tumor cells. In some embodiments, the targeting domain of the TAC is specific for DLL3. In some embodiments, the targeting domain of the TAC is specific for MSLN. In some embodiments, the targeting domain of the TAC is specific for GPC2.

In some embodiments, the protein associated with the TCR complex is CD3, such as CD3ε. In some embodiments, the TCR-binding domain is a single chain antibody, such as scFv, or a V_{H}H. In some embodiments, the TCR-binding domain is derived from UCHT1. In some embodiments, the TAC receptor comprises a cytosolic domain and a transmembrane domain. In some embodiments, the TCR-signaling domain comprises a cytosolic domain derived from a TCR co-receptor. Exemplary TCR co-receptors include, but are not limited to, CD4, CD7, CD8, CD28, CD45, CD4, CD5, CD9, CD16, CD22, CD28, CD33, CD37, CD64, CD80, CD86, CD134, CD137 (4-1BB) and CD154. In some embodiments, the TAC receptor comprises a transmembrane domain and a cytosolic domain derived from CD4. In some embodiments, the TAC receptor comprises a transmembrane domain and a cytosolic domain derived from CD8 (such as CD8a).

T cell co-receptors are expressed as membrane proteins on T cells. They can provide stabilization of the TCR: peptide: MHC complex and facilitate signal transduction. The two subtypes of T cell co-receptor, CD4 and CD8, display strong specificity for particular MEW classes. The CD4 co-receptor can only stabilize TCR: MEW II complexes while the CD8 co-receptor can only stabilize the TCR: MEW I complex. The differential expression of CD4 and CD8 on different T cell types results in distinct T cell functional subpopulations. CD8⁺ T cells are cytotoxic T cells.

CD4 is a glycoprotein expressed on the surface of immune cells such as T helper cells, monocytes, macrophages, and dendritic cells. CD4 has four immunoglobulin domains (D₁ to D₄) exposed on the extracellular cell surface. CD4 contains a special sequence of amino acids on its short cytoplasmic/intracellular tail, which allow CD4 tail to recruit and interact with the tyrosine kinase Lck. When the TCR complex and CD4 each bind to distinct regions of the MHC II molecule, the close proximity between the TCR complex and CD4 allows Lck bound to the cytoplasmic tail of CD4 to tyrosine-phosphorylate the Immunoreceptor Tyrosine Activation Motifs (ITAM) on the cytoplasmic domains of CD3, thus amplifying TCR generated signal.

CD8 is a glycoprotein of either a homodimer composed of two a chains (less common), or a heterodimer composed of one α and one β chain (more common), each comprising an immunoglobulin variable (IgV)-like extracellular domain connected to the membrane by a thin stalk, and an intracellular tail. CD8 is predominantly expressed on the surface of cytotoxic T cells, but can also be found on natural killer cells, cortical thymocytes, and dendritic cells. The CD8 cytoplasmic tail interacts with Lck, which phosphorylates the cytoplasmic CD3 and ζ-chains of the TCR complex once TCR binds its specific antigen. Tyrosine-phosphorylation on the cytoplasmic CD3 and ζ-chains initiates a cascade of phosphorylation, eventually leading to gene transcription.

### 2.5 Tumor Homing Peptides (THPs)

“Tumor homing peptide (THP)” is a kind of peptides specifically targeting tumor stroma, especially tumor vasculature, possibly via specific ligands expressed on tumor vessels. Arginine-glycine-aspartic (RGD) peptides and asparagine-glycine-arginine (NGR) peptides are two of the well-known THPs. In some embodiments, a RGD peptide may comprise one or more RGD motifs, such as any of 1, 2, 3, or more RGD motifs. In some embodiments, a NGR peptide may comprise one or more NGR motifs, such as any of 1, 2, 3, or more NGR motifs. In some embodiments, the THP comprises both a RGD peptide motif and a NGR peptide motif. In some embodiments, the THP comprises one or more RGD peptide motifs and one or more NGR peptide motifs. In some embodiments, the THPs described herein may comprise any suitable THP known in the art. In some embodiments, the THP is a combination of THPs described herein.

In some embodiments, the THP is membrane bound. In some embodiments, the THP is a membrane bound RGD (mbRGD). In some embodiments, the THP is membrane bound NGR (mbNGR).

Solid tumors are composed of two distinct but interdependent compartments: the malignant cells themselves (parenchyma) and the supporting connective tissue (stroma) that they induce and in which they are dispersed. Tumor stroma differs strikingly from normal connective tissue. Blood vessels offer one example. Tumor vessels differ from their normal counterparts with respect to organization, structure and function. Unlike the normal vasculature, tumor vessels are not arranged in a hierarchical pattern but are instead irregularly spaced and structurally heterogeneous. They are also hyperpermeable to plasma and plasma proteins, may lack pericytes, and are lined by actively dividing endothelial cells. See, Dvorak H F. Rous-Whipple Award Lecture. How tumors make bad blood vessels and stroma. Am J Pathol. 2003 June; 162(6):1747-57. doi: 10.1016/50002-9440(10)64309-x. PMID: 12759232; PMCID: PMC1868128.

Tumor stroma mainly consists of the basement membrane, fibroblasts, extracellular matrix, immune cells, and vasculature. See, Roy M. Bremnes, et al. The Role of Tumor Stroma in Cancer Progression and Prognosis: Emphasis on Carcinoma-Associated Fibroblasts and Non-small Cell Lung Cancer. Journal of Thoracic Oncology, Volume 6, Issue 1, 2011, Pages 209-217.

THPs are usually short peptides in sequence lengths of 3 to 15 amino acids. Since the introduction of tumor homing concept in 1998, a large number of THPs have been identified by in vitro and in vivo phage display technology. THPs have some common motifs like RGD, NGR, which specifically bind to a surface molecule on tumor cells or tumor vasculature. For example, RGD peptide binds to a integrins and NGR binds to a receptor aminopeptidase N, which is present on the surface of tumor endothelial cells. Due to their tumor homing capability, THPs are being used in cancer diagnosis and treatment. See, Sharma, A., Kapoor, P., Gautam, A. et al. Computational approach for designing tumor homing peptides. Sci Rep 3, 1607 (2013). https://doi.org/10.1038/srep01607.

Tumor-homing is a complex, multistep process used by many cells to travel from a distant location to a tumor. The term “tumor homing” for the purpose of the present application refers to any action that involves travel from a distant location to a tumor. A tumor homing peptide used in the present disclosure can guide a molecule or cell population to be enriched or infiltrated into tumor. A THP does not have to have a target on the surface of tumor cells per se, but may leverage other processes that are required in the enrichment. For instance, we utilized the high expression of integrin αvβ5, a feature of tumor angiogenic endothelial cells, to restore the capability of CAR-T cells to complete the trans-endothelial migration which is required for CAR-T to function in solid tumors. As our previous test showed, integrin αvβ5 is not expressed in the tumor cells we used. In some embodiments, a tumor homing peptide used in the present disclosure is not a tumor targeting peptide that leads to direct contact with tumor cells and therefore requires to have a target directly exposed on tumor cells.

THPs useful in the present disclosure comprise but are not limited to RGD-based peptides, i.e. a THP comprising a RGD motif, and NGR-based peptides, i.e. a THP comprising a NGR motif. RGD-based peptides are also called RGD peptides, and similarly to NGR-based peptides. In some embodiments, the THP is selected from the group consisting of a RGD-based peptide, a NGR-based peptide, and combinations thereof. In some embodiments, the THP is selected from the group consisting of RGD, RGD-4C, and iRGD. In some embodiments, the THP is RGD-4C. In some embodiments, the THP comprises at least 90% sequence identity to the amino acid sequence of SEQ ID NO: 2, such as at least any of about 91%, 92%, 93%, 94%, 95%, 96%, 97%, 98%, 99%, or greater sequence identity to the amino acid sequence of SEQ ID NO: 2. In some embodiments, the THP has the amino acid sequence of SEQ ID NO: 2. In some embodiments, the THP is NGR. In some embodiments, the THP comprises at least 90% sequence identity to the amino acid sequence of SEQ ID NO: 38, such as at least any of about 91%, 92%, 93%, 94%, 95%, 96%, 97%, 98%, 99%, or greater sequence identity to the amino acid sequence of SEQ ID NO: 38. In some embodiments, the THP has the amino acid sequence of SEQ ID NO: 38.

RGD peptides could bind multiple integrins, and the binding specificity is determined by the flanked sequence of RGD. RGD-4C is a RGD peptide flanked on both sides by cysteine-aspartate/phenylalanine-cysteine residues, and showed specificity for integrin αvβ5, which are exclusively expressed on tumor cells or neo-vasculature endothelium. See, Nagel H, Maag S, Tassis A, Nestlé F O, Greber U F, Hemmi S. The αvβ5 integrin of hematopoietic and nonhematopoietic cells is a transduction receptor of RGD-4C fiber-modified adenoviruses. Gene Ther. 2003 September; 10(19):1643-53. PMID: 12923563.

The iRGD (CRGDKGPDC) peptide has been developed on the basis of RGD peptides and is composed of nine amino acids. It first binds to αv integrins, which are expressed on tumor cells and vessels. Subsequently, it is cleaved by proteases to expose the neuropilin-1 (NRP-1)-binding CRGDK/R, which effectively triggers the tumor penetration process. The scientific interest in iRGD has resulted from its binding to NRP-1 in particular, as this triggers extravasation. Furthermore, iRGD specifically penetrates into angiogenic vessels and tumor tissues. Due to this novel delivery system and the low toxicity to normal cells, iRGD has attracted significant attention. See, Yin, H., Yang, J., Zhang, Q., Wang, H., Xu, J., Zheng, J. “iRGD as a tumor-penetrating peptide for cancer therapy (Review)”. Molecular Medicine Reports 15.5 (2017): 2925-2930.

Among various homing devices, peptides containing the NGR tripeptide sequence represent a promising approach to selectively recognize CD13 receptor isoforms on the surface of tumor cells. Several peptides containing the NGR tripeptide motif that specifically recognize the CD13 receptor isoform on tumor cells have been successfully used for the delivery of various compounds and chemotherapeutic drugs to tumor vessels. See, Tripodi, A. A. P., Randelović, I., Biri-Kovács, B. et al. In Vivo Tumor Growth Inhibition and Antiangiogenic Effect of Cyclic NGR Peptide-Daunorubicin Conjugates Developed for Targeted Drug Delivery. Pathol. Oncol. Res. 26, 1879-1892 (2020).

### 2.6 TGF-β Dominant-Negative Receptor (TGF-β DNR)

TGF-β has been targeted in several studies seeking to boost anti-tumor immunity. In particular, TGF-β signaling within the tumor microenvironment may be targeted with a TGF-β dominant-negative receptor (TGF-β DNR, e.g., dnTGF-βRII). TGF-β DNR may render immune effector cells (such as CAR, TCR, cTCR or TAC-transduced T cells armored with THPs) unresponsive to TGF-β. Immune effector cells homing and infiltration into tumor may be increased while protecting neighboring immune cells from the suppressive effects of TGF-β. In some embodiments, the immune effector cells provided herein are engineered to retain cytolytic activity in the presence of TGF-β.

In some embodiments, the functional exogenous receptors provided herein are armored with TGF-β DNR (e.g., dnTGF-βRII). In some embodiments, the functional exogenous receptor is selected from the group consisting of a CAR, TCR, cTCR, TAC, and a TAC-like chimeric receptor. In some embodiments, the functional exogenous receptor is a CAR, such as any of the CARs provided herein. In some embodiments, the functional exogenous receptor is a cTCR, such as any of the cTCRs provided herein. In some embodiments, the TGF-β DNR is directly fused with the functional exogenous receptor. In some embodiments, the TGF-β DNR is indirectly fused with the functional exogenous receptor. In some embodiments, the TGF-β DNR is fused with the N-terminus of the functional exogenous receptor. In some embodiments, the TGF-β DNR is fused with the C-terminus of the functional exogenous receptor.

In some embodiments, the functional exogenous receptors provided herein are armored with TGF-β DNR (e.g., dnTGF-βRII) and a THP. In some embodiments, the TGF-β DNR is directly fused with the THP. In some embodiments, the TGF-β DNR is indirectly fused with the THP. In some embodiments, the TGF-β DNR is fused with the N-terminus of the THP. In some embodiments, the TGF-β DNR is fused with the C-terminus of the THP. In some embodiments, the functional exogenous receptor is selected from the group consisting of a CAR, TCR, cTCR, TAC, and a TAC-like chimeric receptor. In some embodiments, the functional exogenous receptor is a CAR, such as any of the CARs provided herein. In some embodiments, the functional exogenous receptor is a cTCR, such as any of the cTCRs provided herein. In some embodiments, the THP is an RGD-based peptide, an NGR-based peptide, or a combination thereof. In some embodiments, provided herein is a CAR armored with TGF-β DNR and a THP. In some embodiments, the CAR is an anti-DLL3 CAR.

In some embodiments, the anti-DLL3 CAR is armored with TGF-β DNR (e.g., dnTGF-βRII) and RGD. In some embodiments, the anti-DLL3 CAR armored with TGF-β DNR and RGD comprises, from N-terminus to C-terminus: an anti-DLL3 CAR (SEQ ID NO: 8), a CD28 signal peptide, RGD-4C (SEQ ID NO: 2), a G4S linker, and a TGF-β DNR. In some embodiments, the anti-DLL3 CAR armored with TGF-β DNR and RGD comprises the amino acid sequence of SEQ ID NO: 52 or a variant thereof having, at least about 95% (for example at least about any of 96%, 97%, 98%, or 99%) sequence identity to SEQ ID NO: 52.

In some embodiments, the anti-DLL3 CAR is armored with TGF-β DNR (e.g., dnTGF-βRII) and RGD. In some embodiments, the anti-DLL3 CAR armored with TGF-β DNR and RGD comprises, from N-terminus to C-terminus: an anti-DLL3 CAR (SEQ ID NO: 8), a CD5 signal peptide, RGD-4C (SEQ ID NO: 2), a G4S linker, and a TGF-β DNR. In some embodiments, the anti-DLL3 CAR armored with TGF-β DNR and RGD comprises the amino acid sequence of SEQ ID NO: 53 or a variant thereof having, at least about 95% (for example at least about any of 96%, 97%, 98%, or 99%) sequence identity to SEQ ID NO: 53.

In some embodiments, the anti-DLL3 CAR is armored with TGF-β DNR (e.g., dnTGF-βRII) and NGR. In some embodiments, the anti-DLL3 CAR armored with TGF-β DNR and NGR comprises, from N-terminus to C-terminus: an anti-DLL3 CAR (SEQ ID NO: 8), a CD28 signal peptide, NGR, a G4S linker, and a TGF-β DNR. In some embodiments, the anti-DLL3 CAR armored with TGF-β DNR and NGR comprises the amino acid sequence of SEQ ID NO: 54 or a variant thereof having, at least about 95% (for example at least about any of 96%, 97%, 98%, or 99%) sequence identity to SEQ ID NO: 54.

In some embodiments, the anti-DLL3 CAR armored with TGF-β DNR and NGR comprises, from N-terminus to C-terminus: an anti-DLL3 CAR (SEQ ID NO: 8), a TGF-β DNR, a P2A, NGR (SEQ ID NO: 38), and a hinge domain (SEQ ID NO: 13) and a transmembrane domain from CD7 (SEQ ID NO: 14). In some embodiments, the anti-DLL3 CAR armored with TGF-β DNR and NGR comprises the amino acid sequence of SEQ ID NO: 55 or a variant thereof having, at least about 95% (for example at least about any of 96%, 97%, 98%, or 99%) sequence identity to SEQ ID NO: 55.

In some embodiments, the anti-DLL3 CAR armored with TGF-β DNR and NGR comprises, from N-terminus to C-terminus: an anti-DLL3 CAR (SEQ ID NO: 8), a TGF-β DNR, a P2A, NGR (SEQ ID NO: 38), Flag (SEQ ID NO: 3) and a hinge domain (SEQ ID NO: 13) and a transmembrane domain from CD7 (SEQ ID NO: 14). In some embodiments, the anti-DLL3 CAR armored with TGF-β DNR and NGR comprises the amino acid sequence of SEQ ID NO: 56 or a variant thereof having, at least about 95% (for example at least about any of 96%, 97%, 98%, or 99%) sequence identity to SEQ ID NO: 56.

In some embodiments, the anti-DLL3 CAR armored with TGF-β DNR and NGR comprises, from N-terminus to C-terminus: an anti-DLL3 CAR (SEQ ID NO: 8), NGR (SEQ ID NO: 38), a G4S linker, NGR (SEQ ID NO: 38), a hinge domain (SEQ ID NO: 13) and a transmembrane domain from CD7 (SEQ ID NO: 14), a T2A, and a TGF-β DNR. In some embodiments, the anti-DLL3 CAR armored with TGF-β DNR and NGR comprises the amino acid sequence of SEQ ID NO: 57 or a variant thereof having, at least about 95% (for example at least about any of 96%, 97%, 98%, or 99%) sequence identity to SEQ ID NO: 57.

In some embodiments, the anti-DLL3 CAR armored with TGF-β DNR and NGR comprises, from N-terminus to C-terminus: an anti-DLL3 CAR (SEQ ID NO: 8), NGR (SEQ ID NO: 38), a G4S linker, NGR (SEQ ID NO: 38), Flag (SEQ ID NO: 3) a hinge domain (SEQ ID NO: 13) and a transmembrane domain from CD7 (SEQ ID NO: 14), a T2A, and a TGF-β DNR. In some embodiments, the anti-DLL3 CAR armored with TGF-β DNR and NGR comprises the amino acid sequence of SEQ ID NO: 58 or a variant thereof having, at least about 95% (for example at least about any of 96%, 97%, 98%, or 99%) sequence identity to SEQ ID NO: 58.

### 2.7 Polypeptides Comprising the Functional Exogenous Receptor and the Exogenous Tumor Homing Peptide

In an aspect of the present disclosure, there is provided a polypeptide comprising a functional exogenous receptor and an exogenous THP. In some embodiments, the functional exogenous receptor is a CAR, a TCR, a cTCR, a TAC, or a TAC-like chimeric receptor. In some embodiments, the THP is an RGD-peptide, and NGR-peptide, or a combination thereof. In some embodiments, the THP is membrane bound.

In some embodiments, the functional exogenous receptor of the polypeptide comprising a functional exogenous receptor and a THP is a CAR. In some embodiments, the functional exogenous receptor of the polypeptide is a CAR comprising (i) an extracellular antigen binding domain, (ii) a transmembrane domain, and (iii) an intracellular signaling domain. In some embodiments, the CAR is an anti-DLL3 CAR, an anti-MSLN CAR, or an anti-GPC2 CAR.

In some embodiments, the anti-DLL3 CAR of the polypeptide comprising an anti-DLL3 CAR and a THP comprises a first V_{H}H antibody moiety that comprises a CDR1 comprising the amino acid sequence of SEQ ID NO: 29 or a variant thereof comprising up to about 3 amino acid substitutions, a CDR2 comprising the amino acid sequence of SEQ ID NO:30 or a variant thereof comprising up to about 3 amino acid substitutions, and a CDR3 comprising the amino acid sequence of SEQ ID NO: 31 or a variant thereof comprising up to about 3 amino acid substitutions, and a second V_{H}H antibody moiety that comprises a CDR1 comprising the amino acid sequence of SEQ ID NO: 32 or a variant thereof comprising up to about 3 amino acid substitutions, a CDR2 comprising the amino acid sequence of SEQ ID NO:33 or a variant thereof comprising up to about 3 amino acid substitutions, and a CDR3 comprising the amino acid sequence of SEQ ID NO: 34 or a variant thereof comprising up to about 3 amino acid substitutions. In some embodiments, said first V_{H}H antibody moiety and second V_{H}H antibody moiety are linked via a (G4S)n linker, wherein the linker can be any appropriate length, for example, n can be any integer between 1 to 20, preferably between 3 to 12, such as for example 3, 4, 5, 6, 7, 8, 9, 10, and more.

In some embodiments, the anti-DLL3 CAR of the polypeptide comprising an anti-DLL3 CAR and a THP comprises a first V_{H}H antibody moiety comprising the amino acid sequences of SEQ ID NO: 27 (i.e. AS63997VH5 V_{H}H) or an amino acid sequence having at least 75%, 80%, 90%, 91%, 92%, 93%, 94%, 95%, 96%, 97%, 98%, or 99% identity to the amino acid sequence of SEQ ID NO: 27, and a second V_{H}H antibody moiety comprising the amino acid sequences of SEQ ID NO: 28 (i.e. AS64380VH5 V_{H}H) or an amino acid sequence having at least 75%, 80%, 90%, 91%, 92%, 93%, 94%, 95%, 96%, 97%, 98%, or 99% identity to the amino acid sequence of SEQ ID NO: 28.

In some embodiments, the anti-DLL3 CAR of the polypeptide comprising an anti-DLL3 CAR and a THP comprises the amino acid sequence of SEQ ID NO: 8 or an amino acid sequence having at least 75%, 80%, 90%, 91%, 92%, 93%, 94%, 95%, 96%, 97%, 98%, or 99% identity to the amino acid sequence of SEQ ID NO: 8.

In some embodiments, the CAR of the polypeptide comprising a functional exogenous receptor and a THP is an anti-MSLN CAR. In some embodiments, the anti-MSLN CAR comprises the amino acid sequence of SEQ ID NO: 41 or an amino acid sequence having at least 75%, 80%, 90%, 91%, 92%, 93%, 94%, 95%, 96%, 97%, 98%, or 99% identity to the amino acid sequence of SEQ ID NO: 41.

In some embodiments, the CAR of the polypeptide comprising a functional exogenous receptor and a THP is an anti-GPC2 CAR. In some embodiments, the anti-GPC2 CAR comprises the amino acid sequence of SEQ ID NO: 44 or an amino acid sequence having at least 75%, 80%, 90%, 91%, 92%, 93%, 94%, 95%, 96%, 97%, 98%, or 99% identity to the amino acid sequence of SEQ ID NO: 44.

In some embodiments, the exogenous THP is selected from the group consisting of arginine-glycine-aspartic (RGD)-based peptides, asparagine-glycine-arginine (NGR)-based peptides, and combinations thereof. In some embodiments, the THP comprises the RGD-4C peptide having the amino acid sequence of SEQ ID NO: 2.

In some embodiments, the THP is fused with a transmembrane domain. In some embodiments, the THP is fused with a transmembrane domain and a hinge domain. In some embodiments, the transmembrane domain and/or the hinge domain is from CD7, CD80, CD28, TR2, or FasL. In some embodiments, the transmembrane domain and/or the hinge domain is from CD7. In some embodiments, the transmembrane domain comprises the amino acid sequence of SEQ ID NO: 14, and the hinge domain comprises the amino acid sequence of SEQ ID NO: 13.

In some embodiments, the THP is fused with a peptide linker, such as a (GxS)n linker, wherein x and n, independently can be an integer between 1 and 20, preferably between 3 and 12, such as, for example 3, 4, 5, 6, 7, 8, 9, 10, 11, 12, or more. In some embodiments, the THP is fused with a (G4S)n linker, wherein n is as defined above. For example, in some embodiments, the THP is fused with a (G4S)₃ linker. In some embodiments, the THP is fused with a tag sequence such as a Flag tag. In some embodiments, the THP such as the RGD-4C peptide is fused, sequentially from N terminus to C terminus, with the Flag tag, the (G4S)₃ linker, a hinge region, and a transmembrane domain. The transmembrane domain and the hinge domain are each independently selected from the group consisting of CD7, CD8, CD80, CD28, TR2, and FasL. Preferably, the transmembrane domain and the hinge domain are from CD7.

In some embodiments, the polypeptide of the present disclosure comprises, from N terminus to C terminus, an anti-DLL3 CAR, the P2A self-cleaving linker, leader peptide (e.g. from CD28), RGD-4C, Flag, (G45)₃ linker, hinge region, and transmembrane domain. In some embodiments, the transmembrane domain and the hinge domain are each independently selected from the group consisting of CD7, CD8, CD8α, CD80, TR2, CD28, and FasL. Preferably, the transmembrane domain and the hinge domain are from CD7.

In some embodiments, the polypeptide of the present disclosure comprises, from N terminus to C terminus, an anti-DLL3 CAR, the P2A self-cleaving linker, transmembrane domain, hinge region, Flag tag, (G45)₃ linker, and RGD-4C. In some embodiments, the transmembrane domain and the hinge domain are each independently selected from the group consisting of CD7, CD8, CD8α, CD80, CD28, TR2, and FasL.

In some embodiments, the polypeptide comprises the amino acid sequence set forth in any one of SEQ ID NOs: 10, 11, 19, 21-26, 35, 36, 37, 39-40, 42, 43, 45, 46, 49, 50, and 52-58, or an amino acid sequence having at least 75%, 80%, 90%, 91%, 92%, 93%, 94%, 95%, 96%, 97%, 98%, or 99% identity to the amino acid sequence of any one of SEQ ID NOs: 10, 11, 19, 21-26, 35, 36, 37, 39-40, 42, 43, 45, 46, 49, 50, and 52-58.

In some embodiments, the polypeptide comprises an amino acid sequence encoding an anti-DLL3 CAR. In some embodiments, the anti-DLL3 CAR comprises the amino acid sequence set forth in SEQ ID NO: 8, or an amino acid sequence having at least 75%, 80%, 90%, 91%, 92%, 93%, 94%, 95%, 96%, 97%, 98%, or 99% identity to the amino acid sequence of SEQ ID NO: 8. In some embodiments, the polypeptide comprises an amino acid sequence encoding RGD-4C. In some embodiments, the RGD-4C comprises the amino acid sequence set forth in SEQ ID NO: 2. In some embodiments, the polypeptide comprises the amino acid sequence set forth in SEQ ID NO: 26, or an amino acid sequence having at least 75%, 80%, 90%, 91%, 92%, 93%, 94%, 95%, 96%, 97%, 98%, or 99% identity to the amino acid sequence of SEQ ID NO: 26. In some embodiments, the polypeptide comprises an amino acid sequence encoding NGR. In some embodiments, the NGR comprises the amino acid sequence set forth in SEQ ID NO: 38. In some embodiments, the polypeptide comprises the amino acid sequence set forth in SEQ ID NO: 39, or an amino acid sequence having at least 75%, 80%, 90%, 91%, 92%, 93%, 94%, 95%, 96%, 97%, 98%, or 99% identity to the amino acid sequence of SEQ ID NO: 39.

In some embodiments, the polypeptide comprises an amino acid sequence encoding TGF-β (e.g., TGF-β DNR, such as dnTGF-βRII). In some embodiments, the polypeptide further comprises an amino acid sequence encoding an anti-DLL3 CAR. In some embodiments, the anti-DLL3 CAR comprises the amino acid sequence set forth in SEQ ID NO: 8, or an amino acid sequence having at least 75%, 80%, 90%, 91%, 92%, 93%, 94%, 95%, 96%, 97%, 98%, or 99% identity to the amino acid sequence of SEQ ID NO: 8. In some embodiments, the polypeptide comprises an amino acid sequence encoding NGR. In some embodiments, the NGR comprises the amino acid sequence set forth in SEQ ID NO: 38. In some embodiments, the polypeptide comprises the amino acid sequence set forth in SEQ ID NO: 54, or an amino acid sequence having at least 75%, 80%, 90%, 91%, 92%, 93%, 94%, 95%, 96%, 97%, 98%, or 99% identity to the amino acid sequence of SEQ ID NO: 54. In some embodiments, the polypeptide comprises the amino acid sequence set forth in SEQ ID NO: 55, or an amino acid sequence having at least 75%, 80%, 90%, 91%, 92%, 93%, 94%, 95%, 96%, 97%, 98%, or 99% identity to the amino acid sequence of SEQ ID NO: 55. In some embodiments, the polypeptide comprises the amino acid sequence set forth in SEQ ID NO: 56, or an amino acid sequence having at least 75%, 80%, 90%, 91%, 92%, 93%, 94%, 95%, 96%, 97%, 98%, or 99% identity to the amino acid sequence of SEQ ID NO: 56. In some embodiments, the polypeptide comprises the amino acid sequence set forth in SEQ ID NO: 57, or an amino acid sequence having at least 75%, 80%, 90%, 91%, 92%, 93%, 94%, 95%, 96%, 97%, 98%, or 99% identity to the amino acid sequence of SEQ ID NO: 57. In some embodiments, the polypeptide comprises the amino acid sequence set forth in SEQ ID NO: 58, or an amino acid sequence having at least 75%, 80%, 90%, 91%, 92%, 93%, 94%, 95%, 96%, 97%, 98%, or 99% identity to the amino acid sequence of SEQ ID NO: 58. In some embodiments, the polypeptide comprises an amino acid sequence encoding RGD-4C. In some embodiments, the RGD-4C comprises the amino acid sequence set forth in SEQ ID NO: 2. In some embodiments, the polypeptide comprises the amino acid sequence set forth in SEQ ID NO: 52, or an amino acid sequence having at least 75%, 80%, 90%, 91%, 92%, 93%, 94%, 95%, 96%, 97%, 98%, or 99% identity to the amino acid sequence of SEQ ID NO: 52. In some embodiments, the polypeptide comprises the amino acid sequence set forth in SEQ ID NO: 53, or an amino acid sequence having at least 75%, 80%, 90%, 91%, 92%, 93%, 94%, 95%, 96%, 97%, 98%, or 99% identity to the amino acid sequence of SEQ ID NO: 53.

In some embodiments, the polypeptide comprises an amino acid sequence encoding an anti-MSLN CAR. In some embodiments, the anti-MSLN CAR comprises the amino acid sequence set forth in SEQ ID NO: 41, or an amino acid sequence having at least 75%, 80%, 90%, 91%, 92%, 93%, 94%, 95%, 96%, 97%, 98%, or 99% identity to the amino acid sequence of SEQ ID NO: 41. In some embodiments, the polypeptide comprises an amino acid sequence encoding RGD-4C. In some embodiments, the RGD-4C comprises the amino acid sequence set forth in SEQ ID NO: 2. In some embodiments, the polypeptide comprises an amino acid sequence encoding NGR. In some embodiments, the polypeptide comprises the amino acid sequence set forth in SEQ ID NO: 42, or an amino acid sequence having at least 75%, 80%, 90%, 91%, 92%, 93%, 94%, 95%, 96%, 97%, 98%, or 99% identity to the amino acid sequence of SEQ ID NO: 42. In some embodiments, the polypeptide comprises the amino acid sequence set forth in SEQ ID NO: 43, or an amino acid sequence having at least 75%, 80%, 90%, 91%, 92%, 93%, 94%, 95%, 96%, 97%, 98%, or 99% identity to the amino acid sequence of SEQ ID NO: 43.

In some embodiments, the polypeptide comprises an amino acid sequence encoding anti-GPC2 CAR. In some embodiments, the anti-GPC2 CAR comprises the amino acid sequence set forth in SEQ ID NO: 44, or an amino acid sequence having at least 75%, 80%, 90%, 91%, 92%, 93%, 94%, 95%, 96%, 97%, 98%, or 99% identity to the amino acid sequence of SEQ ID NO: 44. In some embodiments, the polypeptide comprises an amino acid sequence encoding RGD-4C. In some embodiments, the RGD-4C comprises the amino acid sequence set forth in SEQ ID NO: 2. In some embodiments, the polypeptide comprises an amino acid sequence encoding NGR. In some embodiments, the NGR comprises the amino acid sequence set forth in SEQ ID NO: 38. In some embodiments, the polypeptide comprises the amino acid sequence set forth in SEQ ID NO: 45, or an amino acid sequence having at least 75%, 80%, 90%, 91%, 92%, 93%, 94%, 95%, 96%, 97%, 98%, or 99% identity to the amino acid sequence of SEQ ID NO: 45. In some embodiments, the polypeptide comprises the amino acid sequence set forth in SEQ ID NO: 46, or an amino acid sequence having at least 75%, 80%, 90%, 91%, 92%, 93%, 94%, 95%, 96%, 97%, 98%, or 99% identity to the amino acid sequence of SEQ ID NO: 46.

In some embodiments, the polypeptide comprises an amino acid sequence encoding a cTCR. In some embodiments, the cTCR is an anti-DLL3, an anti-MSLN, or an anti-GPC2 cTCR. In some embodiments, the cTCR is a εTCR (i.e., ε-TRuC). In some embodiments, the ε-TRuC is an anti-DLL3, an anti-MSLN, or an anti-GPC2 ε-TRuC. In some embodiments, the polypeptide further comprises an amino acid sequence encoding a THP. In some embodiments, the THP is a RGD-based peptide, an NGR-based peptide, or a combination thereof.

In some embodiments, the polypeptide comprises an amino acid sequence encoding an anti-DLL3-ε-TRuC (i.e., an anti-DLL3 cTCR, i.e., an anti-DLL3 εTCR). In some embodiments, the anti-DLL3-ε-TRuC comprises the amino acid sequence set forth in SEQ ID NO: 35, or an amino acid sequence having at least 75%, 80%, 90%, 91%, 92%, 93%, 94%, 95%, 96%, 97%, 98%, or 99% identity to the amino acid sequence of SEQ ID NO: 35. In some embodiments, the polypeptide comprises the amino acid sequence set forth in SEQ ID NO: 36, or an amino acid sequence having at least 75%, 80%, 90%, 91%, 92%, 93%, 94%, 95%, 96%, 97%, 98%, or 99% identity to the amino acid sequence of SEQ ID NO: 36. In some embodiments, the polypeptide comprises the amino acid sequence set forth in SEQ ID NO: 37, or an amino acid sequence having at least 75%, 80%, 90%, 91%, 92%, 93%, 94%, 95%, 96%, 97%, 98%, or 99% identity to the amino acid sequence of SEQ ID NO: 37.

In some embodiments, the polypeptide comprises an amino acid sequence encoding a single domain antibody (sdAb). In some embodiments, the sdAb is an anti-DLL3 sdAb, an anti-MSLN sdAb, or an anti-GPC2 sdAb. In some embodiments, the polypeptide comprises an amino acid sequence encoding a CD8α hinge domain (e.g., SEQ ID NO: 47). In some embodiments, the polypeptide comprises an amino acid sequence encoding a CD8α transmembrane domain (e.g., SEQ ID NO: 48). In some embodiments, the polypeptide further comprises an amino acid sequence encoding a THP. In some embodiments, the THP is a RGD-based peptide, an NGR-based peptide, or a combination thereof. In some embodiments, the polypeptide comprises an amino acid sequence encoding RGD-4C. In some embodiments, the RGD-4C comprises the amino acid sequence set forth in SEQ ID NO: 2. In some embodiments, the polypeptide comprises an amino acid sequence encoding NGR. In some embodiments, the NGR comprises the amino acid sequence set forth in SEQ ID NO: 38.

In some embodiments, the polypeptide comprises an amino acid sequence encoding an anti-DLL3 sdAb. In some embodiments, the polypeptide comprises the amino acid sequence set forth in SEQ ID NO: 49, or an amino acid sequence having at least 75%, 80%, 90%, 91%, 92%, 93%, 94%, 95%, 96%, 97%, 98%, or 99% identity to the amino acid sequence of SEQ ID NO: 49. In some embodiments, the polypeptide comprises the amino acid sequence set forth in SEQ ID NO: 50, or an amino acid sequence having at least 75%, 80%, 90%, 91%, 92%, 93%, 94%, 95%, 96%, 97%, 98%, or 99% identity to the amino acid sequence of SEQ ID NO: 50.

Various codon optimization techniques can be used to obtain an optimized amino acid sequence from the polypeptides discussed herein. For example, it may be desirable to improve the binding affinity and/or other biological properties of an antigen binding domain or other moieties. Amino acid sequence variants may be prepared by introducing appropriate modifications into the nucleotide sequence encoding a polypeptide, or by peptide synthesis. Such modifications include, for example, deletions from, and/or insertions into and/or substitutions of residues within an amino acid sequence. Any combination of deletion, insertion, and substitution can be made to arrive at the final construct, provided that the final construct possesses the desired characteristics, e.g., antigen-binding or signal conversion.

In some embodiments, antibody binding domain moieties or other polypeptide moieties comprising one or more amino acid substitutions, deletions, or insertions are contemplated. Sites of interest for mutational changes include the antibody binding domain heavy and light chain variable regions (VRs) and frameworks (FRs). Amino acid substitutions may be introduced into a binding domain of interest and the products screened for a desired activity, e.g., retained/improved antigen binding or decreased immunogenicity. In certain embodiments, amino acid substitutions may be introduced into one or more of the primary co-stimulatory receptor domain (extracellular or intracellular), secondary costimulatory receptor domain, or extracellular co-receptor domain.

Accordingly, the disclosure encompasses the polypeptides particularly discussed herein as well as polypeptides having at least 80%, at least 85%, at least 87%, at least 90%, at least 91%, at least 92%, at least 93%, at least 94%, at least 95%, at least 96%, at least 97%, at least 98%, or at least 99% sequence identity to the amino acid sequences particularly discussed herein. The terms “percent similarity,” “percent identity,” and “percent homology” when referring to a particular sequence are used as set forth in the University of Wisconsin GCG software program BestFit. Other algorithms may be used, e.g. BLAST, psiBLAST or TBLASTN (which use the method of Altschul et al. (1990) J. Mol. Biol. 215: 405-410), FASTA (which uses the method of Pearson and Lipman (1988) PNAS USA 85: 2444-2448).

### 3. Nucleic Acid

In an aspect, the present disclosure provides an isolated nucleic acid encoding the inventive polypeptide, such as any of the polypeptides described herein.

As used herein, the terms “polynucleotide”, “nucleotide”, and “nucleic acid” are intended to be synonymous with each other. It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed, e.g. codon optimization. Nucleic acids according to the disclosure may comprise DNA or RNA. They may be single stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the present disclosure, it is to be understood that the polynucleotides may be modified by any method available in the art. Such modifications may be carried out in order to enhance the in vivo activity or life span of polynucleotides of interest.

The nucleic acid sequences may be joined by a sequence allowing co-expression of two or more nucleic acid sequences. For example, the construct may comprise an internal promoter. There can be expression of multiple polypeptides by one polynucleotide, using for example, an additional promoter, an internal ribosome entry sequence (IRES) sequence or a sequence encoding a cleavage site. The cleavage site may be self-cleaving, such that when the polypeptide is produced, it is immediately cleaved into the discrete proteins without the need for any external cleavage activity. Various self-cleaving sites are known, including the Foot-and Mouth disease virus (FMDV) and the 2A self-cleaving peptide (e.g. P2A, T2A). The co-expressing sequence may be an internal ribosome entry sequence (IRES). The co-expressing sequence may be an internal promoter.

As used herein, the term “operatively linked,” and similar phrases, when used in reference to nucleic acids or amino acids, refer to the operational linkage of nucleic acid sequences or amino acid sequence, respectively, placed in functional relationships with each other. For example, an operatively linked promoter, enhancer elements, open reading frame, 5′ and 3′ UTR, and terminator sequences result in the accurate production of a nucleic acid molecule (e.g., RNA). In some embodiments, operatively linked nucleic acid elements result in the transcription of an open reading frame and ultimately the production of a polypeptide (i.e., expression of the open reading frame).

### 4. Vectors

In another aspect of the present disclosure, there is provided a vector comprising the isolated nucleic acid according to the present disclosure.

Vectors may be used to introduce the nucleic acid sequence(s) or nucleic acid construct(s) into a host cell so that it expresses one or more polypeptides according to an aspect of the disclosure and, optionally, one or more other proteins of interest (POI). The vector may, for example, be a plasmid or a viral vector, such as a retroviral vector or a lentiviral vector, or a transposon-based vector or synthetic mRNA.

Vectors derived from retroviruses, such as the lentivirus, are suitable tools to achieve long-term gene transfer since they allow long-term, stable integration of a transgene or transgenes and its propagation in daughter cells. The vector may be capable of transfecting or transducing a lymphocyte.

In some embodiments, a nucleic acid discussed in the present disclosure is inserted into a vector. Two nucleic acids can be inserted into one vector or two separate vectors. The expression of the combination according to the present disclosure can be achieved by operably linking a nucleic acid encoding one element of the combination to a promoter and operably linking other nucleic acids encoding the other elements of the combination to other promoters, and incorporating the constructs into one or more expression vectors. In some embodiments, genetic modification strategies of the expression vectors are used to render engineered immune cells expressing said vectors resistant to hostile cellular environments. For example, the nucleic acid encoding a signal converter or a dominant negative receptor can be operably linked to a promoter, the nucleic acid encoding the functional exogenous receptor, such as a CAR, TCR, cTCR, TAC, or TAC-like chimeric receptor can be linked to another promoter, then the two constructs are incorporated into one or two expression vectors, and the vector(s) are introduced into immune cells such as T cells for expression. As used herein the term “signal converter” refers to a sequence that converts immune cell inhibitory signals into stimulatory signals. Exemplary dominant negative receptors include TGF-β, which is a widely used immune evasion strategy by tumors since it promotes tumor growth while drastically inhibiting tumor-specific cellular immunity. Another way to achieve such expression is to put two or more than two nucleic acids under the control of one promoter. Various methods for the expression of proteins of interest are within the knowledge of a skilled artisan in the field.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline.

The vectors can be suitable for replication and integration in eukaryotic cells. Typical cloning vectors contain transcription and translation terminators, initiation sequences, and promoters useful for regulation of the expression of the desired nucleic acid sequence. Viral vector technology is well known in the art and is described, for example, in Sambrook et al. (2001, Molecular Cloning: A Laboratory Manual, Cold Spring Harbor Laboratory, New York), and in other virology and molecular biology manuals, see also, WO 01/96584; WO 01/29058; and U.S. Pat. No. 6,326,193). In some embodiments, the nucleic acid construct of the disclosure is a multi-cistronic construct comprising two promoters. In some embodiments, the dual promoter constructs of the disclosure are uni-directional. In other embodiments, the dual promoter constructs of the disclosure are bi-directional. In order to assess the expression of the polypeptides, the expression vector to be introduced into a cell can also contain either a selectable marker gene or a reporter gene or both to facilitate identification and selection of expressing cells from the population of cells sought to be transfected or transduced through viral vectors.

### 5. Pharmaceutical Composition

Also provided herein are immune effector cell compositions (such as pharmaceutical compositions, also referred to herein as formulations) comprising an immune effector cell (such as a T cell) described herein. In one aspect, the present disclosure further provides pharmaceutical compositions comprising the immune effector cell, the polypeptide, the nucleic acid, or the vector according to the present disclosure.

In some embodiments, there is provided an immune effector cell composition comprising a homogeneous cell population of immune effector cells (such as engineered T cells) of the same cell type and expressing the same functional exogenous receptor and optionally the same THP. In some embodiments, the immune effector cell is selected from the group consisting of T cells, natural killer (NK) cells, B cells, neutrophils, eosinophils, monocytes, macrophages, dendritic cells, peripheral blood mononuclear cells (PBMC), stem cells from which lymphoid cells may be differentiated, and combinations thereof. In some embodiments, the immune effector cell is a T cell. In some embodiments, the immune effector cell is selected from the group consisting of a cytotoxic T cell, a helper T cell, a natural killer T cell, and a γδT cell. In some embodiments, the immune effector cell composition is a pharmaceutical composition.

In some embodiments, the pharmaceutical composition comprises a therapeutically effective amount of the immune effector cell, the polypeptide, the nucleic acid, or the vector according to the present disclosure and a pharmaceutically acceptable excipient.

In a specific embodiment, the term “excipient” can also refer to a diluent, adjuvant (e.g., Freunds' adjuvant (complete or incomplete), carrier or vehicle. Pharmaceutical excipients can be sterile liquids, such as water and oils, including those of petroleum, animal, vegetable or synthetic origin, such as peanut oil, soybean oil, mineral oil, sesame oil and the like. Saline solutions and aqueous dextrose and glycerol solutions can also be employed as liquid excipients. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Examples of suitable pharmaceutical excipients are described in Remington's Pharmaceutical Sciences (1990) Mack Publishing Co., Easton, PA. Such compositions will contain a prophylactically or therapeutically effective amount of the active ingredient provided herein, such as in purified form, together with a suitable amount of excipient so as to provide the form for proper administration to the patient. The formulation should suit the mode of administration.

In some embodiments, the choice of excipient is determined in part by the particular cell, and/or by the method of administration. Accordingly, there are a variety of suitable formulations. Typically, acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers, stabilizers, metal complexes (e.g. Zn-protein complexes); chelating agents such as EDTA and/or non-ionic surfactants.

The route of administration is in accordance with known and accepted methods, such as by single or multiple bolus or infusion over a long period of time in a suitable manner, e.g., injection or infusion by subcutaneous, intravenous, intraperitoneal, intramuscular, intraarterial, intralesional or intraarticular routes, topical administration, inhalation or by sustained release or extended-release means.

In some embodiments, the pharmaceutical composition provided herein contains the binding molecules and/or cells in amounts effective to treat or prevent the disease or disorder, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined.

In some embodiments, the pharmaceutical composition is suitable for administration to an individual, such as a human individual. In some embodiments, the pharmaceutical composition is suitable for injection. In some embodiments, the pharmaceutical composition is suitable for infusion. In some embodiments, the pharmaceutical composition is substantially free of cell culture medium. In some embodiments, the pharmaceutical composition is substantially free of endotoxins or allergenic proteins. In some embodiments, “substantially free” is less than about any of 10%, 5%, 1%, 0.1%, 0.01%, 0.001%, 1 ppm or less of total volume or weight of the pharmaceutical composition. In some embodiments, the pharmaceutical composition is free of mycoplasma, microbial agents, and/or communicable disease agents.

The pharmaceutical composition of the present applicant may comprise any number of the immune effector cells. In some embodiments, the pharmaceutical composition comprises a single copy of the immune effector cell. In some embodiments, the pharmaceutical composition comprises at least about any of 1, 10, 100, 1000, 10⁴, 10⁵, 10⁶, 10⁷, 10⁸ or more copies of the immune effector cells. In some embodiments, the pharmaceutical composition comprises a single type of immune effector cell. In some embodiments, the pharmaceutical composition comprises at least two types of immune effector cells, wherein the different types of immune effector cells differ by their cell sources, cell types, expressed functional exogenous receptors, THPs and/or promoters, etc.

At various points during preparation of a composition, it can be necessary or beneficial to cryopreserve a cell. The terms “frozen/freezing” and “cryopreserved/cryopreserving” can be used interchangeably. Freezing includes freeze-drying.

In some embodiments, cells can be harvested from a culture medium, and washed and concentrated into a carrier in a therapeutically effective amount. Exemplary carriers include saline, buffered saline, physiological saline, water, Hanks' solution, Ringer's solution, Nonnosol-R (Abbott Labs), Plasma-Lyte A® (Baxter Laboratories, Inc., Morton Grove, IL), glycerol, ethanol, and combinations thereof.

In some embodiments, carriers can be supplemented with human serum albumin (HSA) or other human serum components or fetal bovine serum. In particular embodiments, a carrier for infusion includes buffered saline with 5% HAS or dextrose. Additional isotonic agents include polyhydric sugar alcohols including trihydric or higher sugar alcohols, such as glycerin, erythritol, arabitol, xylitol, sorbitol, or mannitol.

Carriers can include buffering agents, such as citrate buffers, succinate buffers, tartrate buffers, fumarate buffers, gluconate buffers, oxalate buffers, lactate buffers, acetate buffers, phosphate buffers, histidine buffers, and/or trimethylamine salts.

Stabilizers refer to a broad category of excipients, which can range in function from a bulking agent to an additive, which helps to prevent cell adherence to container walls. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, alpha-monothioglycerol, and sodium thiosulfate; low molecular weight polypeptides (i.e., <10 residues); proteins such as HSA, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran.

Where necessary or beneficial, compositions can include a local anesthetic such as lidocaine to ease pain at a site of injection.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Therapeutically effective amounts of cells within compositions can be greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹ cells, including any values and ranges in between these values.

In compositions and formulations disclosed herein, cells are generally in a volume of a liter or less, 500 ml or less, 250 ml or less or 100 ml or less. Hence the density of administered cells is typically greater than 10⁴ cells/ml, 10⁷ cells/ml or 10⁸ cells/ml.

Also provided herein are nucleic acid compositions (such as pharmaceutical compositions, also referred to herein as formulations) comprising any of the polypeptide encoding an functional exogenous receptor, a THP, and/or TGF-β (e.g., TGF-β DNR, such as dnTGF-βRII) described herein. In some embodiments, the nucleic acid composition is a pharmaceutical composition. In some embodiments, the nucleic acid composition further comprises any of an isotonizing agent, an excipient, a diluent, a thickener, a stabilizer, a buffer, and/or a preservative; and/or an aqueous vehicle, such as purified water, an aqueous sugar solution, a buffer solution, physiological saline, an aqueous polymer solution, or RNase free water. The amounts of such additives and aqueous vehicles to be added can be suitably selected according to the form of use of the nucleic acid composition.

The compositions and formulations disclosed herein can be prepared for administration by, for example, injection, infusion, perfusion, or lavage. The compositions and formulations can further be formulated for bone marrow, intravenous, intradermal, intraarterial, intranodal, intralymphatic, intraperitoneal, intralesional, intraprostatic, intravaginal, intrarectal, topical, intrathecal, intratumoral, intramuscular, intravesicular, and/or subcutaneous injection.

The formulations to be used for in vivo administration must be sterile. This is readily accomplished by, e.g., filtration through sterile filtration membranes.

### 6. Excipient

The pharmaceutical compositions of the present application are useful for therapeutic purposes. Thus, different from other compositions comprising immune effector cells, such as production cells that express the functional exogenous receptor, a THP, and/or TGF-β (e.g., TGF-βDNR, such as dnTGF-βRII), the pharmaceutical compositions of the present application comprises a pharmaceutically acceptable excipient suitable for administration to an individual.

Suitable pharmaceutically acceptable excipient may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. In some embodiments, the pharmaceutically acceptable excipient comprises autologous serum. In some embodiments, the pharmaceutically acceptable excipient comprises human serum. In some embodiments, the pharmaceutically acceptable excipient is non-toxic, biocompatible, non-immunogenic, biodegradable, and can avoid recognition by the host's defense mechanism. The excipient may also contain adjuvants such as preserving stabilizing, wetting, emulsifying agents and the like. In some embodiments, the pharmaceutically acceptable excipient enhances the stability of the immune effector cell or the antibody or other therapeutic proteins secreted thereof. In some embodiments, the pharmaceutically acceptable excipient reduces aggregation of the antibody or other therapeutic proteins secreted by the immune effector cell. The final form may be sterile and may also be able to pass readily through an injection device such as a hollow needle. The proper viscosity may be achieved and maintained by the proper choice of excipients.

In some embodiments, the pharmaceutical composition is formulated to have a pH in the range of about 4.5 to about 9.0, including for example pH ranges of about any one of 5.0 to about 8.0, about 6.5 to about 7.5, or about 6.5 to about 7.0. In some embodiments, the pharmaceutical composition can also be made to be isotonic with blood by the addition of a suitable tonicity modifier, such as glycerol.

In some embodiments, the pharmaceutical composition is suitable for administration to a human. In some embodiments, the pharmaceutical composition is suitable for administration to a human by parenteral administration. Formulations suitable for parenteral administration include aqueous and non-aqueous, isotonic sterile injection solutions, which can contain anti-oxidants, buffers, bacteriostats, and solutes that render the formulation compatible with the blood of the intended recipient, and aqueous and non-aqueous sterile suspensions that can include suspending agents, solubilizers, thickening agents, stabilizing agents, and preservatives. The formulations can be presented in unit-dose or multi-dose sealed containers, such as ampules and vials, and can be stored in a condition requiring only the addition of the sterile liquid excipient methods of treatment, methods of administration, and dosage regimens described herein (i.e., water) for injection, immediately prior to use. In some embodiments, the pharmaceutical composition is contained in a single-use vial, such as a single-use sealed vial. In some embodiments, the pharmaceutical composition is contained in a multi-use vial. In some embodiments, the pharmaceutical composition is contained in bulk in a container. In some embodiments, the pharmaceutical composition is cryopreserved.

In some embodiments, the pharmaceutical composition is formulated for intravenous administration. In some embodiments, the pharmaceutical composition is formulated for subcutaneous administration. In some embodiments, the pharmaceutical composition is formulated for local administration to a tumor site. In some embodiments, the pharmaceutical composition is formulated for intratumoral injection.

In some embodiments, the pharmaceutical composition must meet certain standards for administration to an individual. For example, the United States Food and Drug Administration has issued regulatory guidelines setting standards for cell-based immunotherapeutic products, including 21 CFR 610 and 21 CFR 610.13. Methods are known in the art to assess the appearance, identity, purity, safety, and/or potency of pharmaceutical compositions. In some embodiments, the pharmaceutical composition is substantially free of extraneous protein capable of producing allergenic effects, such as proteins of an animal source used in cell culture other than the engineered mammalian immune cells. In some embodiments, “substantially free” is less than about any of 10%, 5%, 1%, 0.1%, 0.01%, 0.001%, 1 ppm or less of total volume or weight of the pharmaceutical composition. In some embodiments, the pharmaceutical composition is prepared in a GMP-level workshop. In some embodiments, the pharmaceutical composition comprises less than about 5 EU/kg body weight/hr of endotoxin for parenteral administration. In some embodiments, at least about 70% of the engineered immune cells in the pharmaceutical composition are alive for intravenous administration. In some embodiments, the pharmaceutical composition has a “no growth” result when assessed using a 14-day direct inoculation test method as described in the United States Pharmacopoeia (USP). In some embodiments, prior to administration of the pharmaceutical composition, a sample including both the engineered immune cells and the pharmaceutically acceptable excipient should be taken for sterility testing approximately about 48-72 hours prior to the final harvest (or coincident with the last re-feeding of the culture). In some embodiments, the pharmaceutical composition is free of mycoplasma contamination. In some embodiments, the pharmaceutical composition is free of detectable microbial agents. In some embodiments, the pharmaceutical composition is free of communicable disease agents, such as HIV type I, HIV type II, HBV, HCV, Human T-lymphotropic virus, type I; and Human T-lymphotropic virus, type II.

### 7. Method of Preparation

The engineered immune effector cells can be prepared by introducing the polypeptide(s) provided herein into the immune effector cells, such as T cells. In some embodiments, the polypeptide is introduced to the immune effector cells by transferring any one of the isolated nucleic acids or any one of the vectors described herein. Methods of introducing vectors or isolated nucleic acids into a mammalian cell are well known in the art. For example, the vectors described can be transferred into an immune effector cell by physical, chemical, or biological methods.

Therefore, the present disclosure according to one aspect provides a method of making the inventive engineered immune effector cell, comprising introducing into an immune effector cell: the nucleic acid or the vector of the present disclosure, or a composition comprising two nucleic acids each encoding a functional exogenous receptor and an exogenous tumor homing peptide (THP).

### 7.1 Sources of T Cells

In some embodiments, prior to expansion and genetic modification of the T cells, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments, any number of T cell lines available in the art, may be used. In some embodiments, T cells can be obtained from a unit of blood collected from a subject using any number of techniques known to the skilled artisan, such as Ficoll™ separation. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing steps. In some embodiments, the cells are washed with phosphate buffered saline (PBS). In some embodiments, the wash solution lacks calcium and may lack magnesium or may lack many if not all divalent cations. Initial activation steps in the absence of calcium may lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca²⁺-free, Mg²⁺-free PBS, PlasmaLyte A, or other saline solution with or without buffer. Alternatively, the undesirable components of the apheresis sample may be removed and the cells directly resuspended in culture media.

In some embodiments, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3+, CD28+, CD4+, CD8+, CD45RA+, and CD45R0+ T cells, can be further isolated by positive or negative selection techniques. For example, in some embodiments, T cells are isolated by incubation with anti-CD3/anti-CD28 (i.e., 3×28)-conjugated beads, such as DYNABEADS® M-450 CD3/CD28 T, for a time period sufficient for positive selection of the desired T cells. In some embodiments, the time period is about 30 minutes. In a further embodiment, the time period ranges from 30 minutes to 36 hours or longer and all integer values there between. In a further embodiment, the time period is at least 1, 2, 3, 4, 5, or 6 hours. In some embodiments, the time period is 10 to 24 hours. In some embodiments, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immune-compromised individuals. Further, use of longer incubation times can increase the efficiency of capture of CD8+ T cells. Thus, in some embodiments, by simply shortening or lengthening the time T cells are allowed to bind to the CD3/CD28 beads and/or by increasing or decreasing the ratio of beads to T cells, subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used. In some embodiments, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative immuno-magnetic adherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. In certain embodiments, it may be desirable to enrich for or positively select for regulatory T cells which typically express CD4+, CD25+, CD62Lhi, GITR+, and FoxP3+. Alternatively, in certain embodiments, T regulatory cells are depleted by anti-C25 conjugated beads or other similar method of selection.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/mL is used. In one embodiment, a concentration of 1 billion cells/mL is used. In a further embodiment, greater than 100 million cells/mL is used. In a further embodiment, a concentration of cells of 10, 15, 20, 25, 30, 35, 40, 45, or 50 million cells/mL is used. In yet another embodiment, a concentration of cells from 75, 80, 85, 90, 95, or 100 million cells/mL is used. In further embodiments, concentrations of 125 or 150 million cells/mL can be used. Using high concentrations may result in increased cell yield, cell activation, and cell expansion. Further, use of high cell concentrations may allow more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. In some embodiments, using high concentration of cells allows more efficient selection of CD8+ T cells that normally have weaker CD28 expression.

In some embodiments, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In some embodiments, the concentration of cells used is 5×10⁶/mL. In some embodiments, the concentration used can be from about 1×10⁵/mL to 1×10⁶/mL, and any integer value in between.

In some embodiments, the cells may be incubated on a rotator for varying lengths of time at varying speeds at either 2-10° C., or at room temperature.

T cells for stimulation can also be frozen after a washing step. Without being bound by theory, the freeze and subsequent thaw step may provide a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% dextran 40 and 5% dextrose, 20% human serum albumin and 7.5% DMSO, or 31.25% plasmalyte-A, 31.25% dextrose 5%, 0.45% NaCl, 10% dextran 40 and 5% dextrose, 20% human serum albumin, and 7.5% DMSO or other suitable cell freezing media containing for example, Hespan and PlasmaLyte A. The cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In some embodiments, cryopreserved cells are thawed and washed as described herein and allowed to rest for one hour at room temperature prior to activation.

Also contemplated in the present disclosure is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In one embodiment, a blood sample or an apheresis is taken from a generally healthy subject. In certain embodiments, a blood sample or an apheresis is taken from a generally healthy subject who is at risk of developing a disease, but who has not yet developed a disease, and the cells of interest are isolated and frozen for later use. In certain embodiments, the T cells may be expanded, frozen, and used at a later time. In certain embodiments, samples are collected from a patient shortly after diagnosis of a particular disease as described herein but prior to any treatments. In a further embodiment, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66:807-815 (1991); Henderson et al., Immun 73:316-321 (1991); Bierer et al., Curr. Opin. Immun. 5:763-773 (1993)). In a further embodiment, the cells are isolated for a patient and frozen for later use in conjunction with (e.g., before, simultaneously or following) bone marrow or stem cell transplantation, T cell ablative therapy using either chemotherapy agents such as, fludarabine, external-beam radiation therapy (XRT), cyclophosphamide, or antibodies such as OKT3 or CAMPATH. In another embodiment, the cells are isolated prior to and can be frozen for later use for treatment following B-cell ablative therapy such as agents that react with CD20, e.g., Rituxan.

In some embodiments, T cells are obtained from a patient directly following treatment. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present disclosure to collect blood cells, including T cells, dendritic cells, or other cells of the hematopoietic lineage, during this recovery phase. Further, in certain embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

### 7.2 Activation and Expansion of T Cells

In some embodiments, prior to or after genetic modification of the T cells with the CARs, TCRs, cTCRs, TACs, TAC-like chimeric receptors, CARs and THP(s), TCRs and THP(s), cTCRs and THP(s), TACs and THP(s), TAC-like chimeric receptors and THP(s), or combinations thereof, described herein, the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, T cells can be expanded by contact with a surface having attached thereto an agent that stimulates a CD3/TCR complex associated signal and a ligand that stimulates a co-stimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody. Examples of an anti-CD3 antibody include UCHT1, OKT3, HIT3a (BioLegend, San Diego, US) can be used as can other methods commonly known in the art (Graves J, et al., J. Immunol. 146:2102 (1991); Li B, et al., Immunology 116:487 (2005); Rivollier A, et al., Blood 104:4029 (2004)). Examples of an anti-CD28 antibody include 9.3, B-T3, XR-CD28 (Diaclone, Besancon, France) can be used as can other methods commonly known in the art (Berg et al., Transplant Proc. 30(8):3975-3977 (1998); Haanen et al., J. Exp. Med. 190(9):13191328 (1999); Garland et al., J. Immunol Meth. 227(1-2):53-63 (1999)).

In some embodiments, the primary stimulatory signal and the co-stimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one embodiment, the agent providing the co-stimulatory signal is bound to a cell surface and the agent providing the primary activation signal is in solution or coupled to a surface. In certain embodiments, both agents can be in solution. In another embodiment, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in certain embodiments in the present disclosure.

In some embodiments, the T cells, are combined with agent-coated beads, the beads and the cells are subsequently separated, and then the cells are cultured. In an alternative embodiment, prior to culture, the agent-coated beads and cells are not separated but are cultured together. In a further embodiment, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached (3×28 beads) to contact the T cells. In one embodiment, the cells (for example, 10⁴ to 4×10⁸ T cells) and beads (for example, anti-CD3/CD28 MACSiBead particles at a recommended titer of 1:100) are combined in a buffer, preferably PBS (without divalent cations such as, calcium and magnesium). Those of ordinary skill in the art can readily appreciate any cell concentration may be used. For example, the target cell may be very rare in the sample and comprise only 0.01% of the sample or the entire sample (i.e., 100%) may comprise the target cell of interest. Accordingly, any cell number is within the context of the present disclosure. In certain embodiments, it may be desirable to significantly decrease the volume in which particles and cells are mixed together (i.e., increase the concentration of cells), to ensure maximum contact of cells and particles. For example, in one embodiment, a concentration of about 2 billion cells/mL is used. In another embodiment, greater than 100 million cells/mL is used. In a further embodiment, a concentration of cells of 10, 15, 20, 25, 30, 35, 40, 45, or 50 million cells/mL is used. In yet another embodiment, a concentration of cells from 75, 80, 85, 90, 95, or 100 million cells/mL is used. In further embodiments, concentrations of 125 or 150 million cells/mL can be used. Using high concentrations may result in increased cell yield, cell activation, and cell expansion. Further, use of high cell concentrations may allow more efficient capture of cells that may weakly express target antigens of interest, such as CD28-negative T cells. Such populations of cells may have therapeutic value and would be desirable to obtain in certain embodiments. For example, using high concentration of cells allows more efficient selection of CD8+ T cells that normally have weaker CD28 expression.

In some embodiments, the mixture may be cultured for several hours (about 3 hours) to about 14 days or any hourly integer value in between. In another embodiment, the mixture may be cultured for 21 days. In one embodiment, the beads and the T cells are cultured together for about eight days. In another embodiment, the beads and T cells are cultured together for 2-3 days. Several cycles of stimulation may also be desired such that culture time of T cells can be 60 days or more. Conditions appropriate for T cell culture include an appropriate media (e.g., Minimal Essential Media, RPMI Media 1640 or X-vivo 15 (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGF-β, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂). T cells that have been exposed to varied stimulation times may exhibit different characteristics. For example, typical blood or apheresed peripheral blood mononuclear cell products have a helper T cell population (TH, CD4+) that is greater than the cytotoxic or suppressor T cell population (TC, CD8). Ex vivo expansion of T cells by stimulating CD3 and CD28 receptors produces a population of T cells that prior to about days 8-9 consists predominately of TH cells, while after about days 8-9, the population of T cells comprises an increasingly greater population of TC cells. Accordingly, depending on the purpose of treatment, infusing a subject with a T cell population comprising predominately of TH cells may be advantageous. Similarly, if an antigen-specific subset of TC cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

Viral- and non-viral-based genetic engineering tools can be used to generate CAR-T cells, such as any of the CAR-T cells described herein (e.g., T cells expressing a CAR and a THP), T cells expressing other functional exogenous receptors described herein (e.g., TCR, cTCR, TAC, TAC-like chimeric receptor expressing T cells, T cells expressing a TCR and a THP, T cells expressing a cTCR and a THP, T cells expressing a TAC and a THP, T cells expressing a TAC-like chimeric receptor and a THP, or combinations thereof), resulting in permanent or transient expression of therapeutic genes. Retrovirus-based gene delivery is a mature, well-characterized technology, which has been used to permanently integrate CARs into the host cell genome (Scholler J., e.g. Decade-long safety and function of retroviral-modified chimeric antigen receptor T cells. Sci. Transl. Med. 2012; 4:132ra53; Rosenberg S. A. et al., Gene transfer into humans—immunotherapy of patients with advanced melanoma, using tumor-infiltrating lymphocytes modified by retroviral gene transduction. N. Engl. J. Med. 1990; 323:570-578).

Non-viral DNA transfection methods can also be used. For example, Singh et al describes use of the Sleeping Beauty (SB) transposon system developed to engineer CAR T cells (Singh H., et al., Redirecting specificity of T-cell populations for CD19 using the Sleeping Beauty system. Cancer Res. 2008; 68:2961-2971) and is being used in clinical trials (see e.g., ClinicalTrials.gov: NCT00968760 and NCT01653717). The same technology is applicable to engineer T-cells and the like according to the disclosure.

### 8 Method of Treatment and Uses

The present disclosure, in an aspect, provides a method for treating a disease in a subject, the method comprising administering to the subject the engineered immune effector cell or the pharmaceutical composition described herein, wherein the disease is selected from the group consisting of cancer, infectious disease, inflammation, immune-related disease, and the combinations thereof.

In another aspect, provided herein are methods for using and uses of the engineered cells provided herein (e.g., CAR-T cells, such as any of the CAR-T cells described herein (e.g., T cells expressing a CAR and a THP), T cells expressing other functional exogenous receptors described herein (e.g., TCR, cTCR, TAC, TAC-like chimeric receptor expressing T cells, T cells expressing a TCR and a THP, T cells expressing a cTCR and a THP, T cells expressing a TAC and a THP, T cells expressing a TAC-like chimeric receptor and a THP, or combinations thereof). Such methods and uses include therapeutic methods and uses, for example, involving administration of the cells, or compositions containing the same, to a subject having a disease or disorder. In some embodiments, the cell is administered in an effective amount to effect treatment of the disease or disorder. Uses include uses of the cells in such methods and treatments, and in the preparation of a medicament in order to carry out such therapeutic methods. In some embodiments, the methods are carried out by administering the cells, or compositions comprising the same, to the subject having or suspected of having the disease or condition. In some embodiments, the methods thereby treat the disease or disorder in the subject.

In some embodiments, the treatment provided herein cause complete or partial amelioration or reduction of a disease or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms include, but do not imply, complete curing of a disease or complete elimination of any symptom or effect(s) on all symptoms or outcomes.

As used herein, in some embodiments, the treatment provided herein delay development of a disease or disorder, e.g., defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease or disorder. For example, a late stage cancer, such as development of metastasis, may be delayed. In other embodiments, the method or the use provided herein prevents a disease or disorder.

In some embodiments, the present cell therapies are used for treating solid tumor cancer. In other embodiments, the present cell therapies are used for treating hematological cancer. In other embodiments, the disease or disorder is an autoimmune and inflammatory disease. In other embodiments, the disease or disorder is an infectious disease. In some embodiments, the present cell therapies comprise therapies comprising T cells expressing the various CARs, TCRs, cTCRs, TACs, TAC-like chimeric receptors, CARs and THP(s), TCRs and THP(s), cTCRs and THP(s), TACs and THP(s), TAC-like chimeric receptors and THP(s), or combinations thereof, described herein.

In some embodiments, the disease or disorder is selected from the group consisting of bladder cancer, brain cancer, breast cancer, bone marrow cancer, cervical cancer, chronic lymphocytic leukemia, acute lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, neuroblastoma, oral cancer, ovarian cancer, non-small-cell lung cancer, prostate cancer, small-cell lung cancer and spleen cancer. In some embodiments, the disease or disorder is a tumor that exhibiting neuroendocrine features, such as small-cell lung cancer (SCLC), ovarian cancer (OC), or neuroblastoma (NBL).

In some embodiments, the disease or disorder is a cancer that is related to DLL3, such as a cancer that expresses DLL3. In some embodiments, an anti-DLL3 functional exogenous receptor (e.g., a CAR, TCR, cTCR, TAC, TAC-like receptor, or combinations thereof) specifically binds with a cell (e.g., a cancer cell) that expresses DLL3. In some embodiments, an anti-DLL3 CAR, such as any of the anti-DLL3 CARs described herein, specifically binds with a cell (e.g., a cancer cell) that expresses DLL3. In some embodiments, an anti-DLL3 cTCR, such as any of the anti-DLL3 cTCRs described herein, specifically binds with a cell (e.g., a cancer cell) that expresses DLL3.

In some embodiments, the disease or disorder is a cancer that is related to MSLN, such as a cancer that expresses MSLN. In some embodiments, an anti-MSLN functional exogenous receptor (e.g., a CAR, TCR, cTCR, TAC, TAC-like receptor, or combinations thereof) specifically binds with a cell (e.g., a cancer cell) that expresses MSLN. In some embodiments, an anti-MSLN CAR, such as any of the anti-MSLN CARs described herein, specifically binds with a cell (e.g., a cancer cell) that expresses MSLN.

In some embodiments, the disease or disorder is a cancer that is related to GPC2, such as a cancer that expresses GPC2. In some embodiments, an anti-GPC2 functional exogenous receptor (e.g., a CAR, TCR, cTCR, TAC, TAC-like receptor, or combinations thereof) specifically binds with a cell (e.g., a cancer cell) that expresses GPC2. In some embodiments, an anti-GPC2 CAR, such as any of the anti-GPC2 CARs described herein, specifically binds with a cell (e.g., a cancer cell) that expresses GPC2.

In some embodiments, the disease or disorder is a hematological cancer, such as leukemia, lymphoma, or myeloma. In some embodiments, the cancer is selected from a group consisting of Hodgkin's lymphoma, non-Hodgkin's lymphoma (NHL), cutaneous B-cell lymphoma, activated B-cell lymphoma, diffuse large B-cell lymphoma (DLBCL), mantle cell lymphoma (MCL), follicular center lymphoma, transformed lymphoma, lymphocytic lymphoma of intermediate differentiation, intermediate lymphocytic lymphoma (ILL), diffuse poorly differentiated lymphocytic lymphoma (PDL), centrocytic lymphoma, diffuse small-cleaved cell lymphoma (DSCCL), peripheral T-cell lymphomas (PTCL), cutaneous T-Cell lymphoma, mantle zone lymphoma, low grade follicular lymphoma, multiple myeloma (MM), chronic lymphocytic leukemia (CLL), diffuse large B-cell lymphoma (DLBCL), myelodysplastic syndrome (MDS), acute T cell leukemia, acute myeloid leukemia (AML), acute promyelocytic leukemia, acute myeloblastic leukemia, acute megakaryoblastic leukemia, precursor B acute lymphoblastic leukemia, precursor T acute lymphoblastic leukemia, Burkitt's leukemia (Burkitt's lymphoma), acute biphenotypic leukemia, chronic myeloid lymphoma, chronic myelogenous leukemia (CIVIL), and chronic monocytic leukemia. In a specific embodiment, the disease or disorder is myelodysplastic syndromes (MDS). In another specific embodiment, the disease or disorder is acute myeloid leukemia (AML). In another specific embodiment, the disease or disorder is chronic lymphocytic leukemia (CLL). In yet another specific embodiment, the disease or disorder is multiple myeloma (MM).

In other embodiments, the disease or disorder is a solid tumor cancer. In some embodiments, the solid tumor cancer is selected from a group consisting of a carcinoma, an adenocarcinoma, an adrenocortical carcinoma, a colon adenocarcinoma, a colorectal adenocarcinoma, a colorectal carcinoma, a ductal cell carcinoma, a lung carcinoma, a thyroid carcinoma, a nasopharyngeal carcinoma, a melanoma, a non-melanoma skin carcinoma, a liver cancer and a lung cancer.

In some embodiments, the disease or disorder is caused by a pathogen. In some embodiments, the pathogen causes an infectious disease. In some embodiments, the pathogen is a bacteria. In some embodiments, the pathogen is a parasite. In some embodiments, the pathogen is a virus.

In other embodiments, the disease or disorder is an immune or autoimmune disorder. In some embodiments, the disease or disorder is an inflammatory disease. Inflammation plays a fundamental role in host defenses and the progression of immune-mediated diseases. The inflammatory response is initiated in response to injury (e.g., trauma, ischemia, and foreign particles) and infection (e.g., bacterial or viral infection) by a complex cascade of events, including chemical mediators (e.g., cytokines and prostaglandins) and inflammatory cells (e.g., leukocytes). The inflammatory response is characterized by increased blood flow, increased capillary permeability, and the influx of phagocytic cells. These events result in swelling, redness, warmth (altered heat patterns), and pus formation at the site of injury or infection.

Methods for administration of cells for adoptive cell therapy are known, as described, e.g., in US Patent Application Publication No. 2003/0170238; U.S. Pat. No. 4,690,915; Rosenberg, Nat Rev Clin Oncol. 8 (10):577-85 (2011); Themeli et al., Nat Biotechnol. 31(10): 928-933 (2013); Tsukahara et al., Biochem Biophys Res Commun 438(1): 84-9 (2013); and Davila et al., PLoS ONE 8(4): e61338 (2013). These methods may be used in connection with the methods and compositions provided herein.

In some embodiments, the cell therapy (e.g., adoptive T cell therapy) is carried out by autologous transfer, in which the cells are isolated and/or otherwise prepared from the subject who is to receive the cell therapy, or from a sample derived from such a subject. Thus, in some aspects, the cells are derived from a subject in need of a treatment and the cells, following isolation and processing are administered to the same subject. In other embodiments, the cell therapy (e.g., adoptive T cell therapy) is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the subject, to whom the cells, cell populations, or compositions are administered is a primate, such as a human. The subject can be male or female and can be any suitable age, including infant, juvenile, adolescent, adult, and geriatric subjects. In some examples, the subject is a validated animal model for disease, adoptive cell therapy, and/or for assessing toxic outcomes.

The composition provided herein can be administered by any suitable means, for example, by injection, e.g., intravenous or subcutaneous injections. In some embodiments, they are administered by parenteral, intrapulmonary, and intranasal, and, if desired for local treatment, intralesional administration.

The amount of a prophylactic or therapeutic agent provided herein that will be effective in the prevention and/or treatment of a disease or condition can be determined by standard clinical techniques. Effective doses may be extrapolated from dose-response curves derived from in vitro or animal model test systems. For the prevention or treatment of disease, the appropriate dosage of the binding molecule or cell may depend on the type of disease or disorder to be treated, the type of binding molecule, the severity and course of the disease or disorder, whether the therapeutic agent is administered for preventive or therapeutic purposes, previous therapy, the patient's clinical history and response to the agent, and the discretion of the attending physician. The compositions, molecules and cells are in some embodiments suitably administered to the patient at one time or over a series of treatments. Multiple doses may be administered intermittently. An initial higher loading dose, followed by one or more lower doses may be administered.

In the context of genetically engineered cells, in some embodiments, a subject may be administered the range of about one million to about 100 billion cells and/or that amount of cells per kilogram of body weight. In some embodiments, wherein the pharmaceutical composition comprises any one of the engineered immune cells described herein, the pharmaceutical composition is administered at a dosage of at least about any of 10⁴, 10⁵, 10⁶, 10⁷, 10⁸, or 10⁹ cells/kg of body weight of the individual. Dosages may vary depending on attributes particular to the disease or disorder and/or patient and/or other treatments.

In some embodiments, the pharmaceutical composition is administered for a single time. In some embodiments, the pharmaceutical composition is administered for multiple times (such as any of 2, 3, 4, 5, 6, or more times). In some embodiments, the pharmaceutical composition is administered once or multiple times during a dosing cycle. A dosing cycle can be, e.g., 1, 2, 3, 4, 5 or more week(s), or 1, 2, 3, 4, 5, or more month(s). The optimal dosage and treatment regime for a particular patient can be determined by one skilled in the art of medicine by monitoring the patient for signs of disease and adjusting the treatment accordingly.

In some embodiments, the compositions provided herein are administered as part of a combination treatment, such as simultaneously with or sequentially with, in any order, another therapeutic intervention, such as another antibody or engineered cell or receptor or agent, such as a cytotoxic or therapeutic agent.

In certain embodiments, once the cells are administered to a mammal (e.g., a human), the biological activity of the engineered cell populations is measured by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells also can be measured by assaying expression and/or secretion of certain cytokines, such as CD107a, IFNγ, IL-2, and TNF. In some aspects the biological activity is measured by assessing clinical outcome, such as reduction in tumor burden or load.

### 9 Kits and Articles of Manufacture

Further provided are kits, unit dosages, and articles of manufacture comprising any of the engineered immune effector cells described herein. In some embodiments, a kit is provided which contains any one of the pharmaceutical compositions described herein and preferably provides instructions for its use.

The kits of the present application are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder (such as cancer) described herein, and may have a sterile access port (for example the container may be an intravenous solution bag or a vial having a stopper pierceable by a hypodermic injection needle). The label or package insert indicates that the composition is used for treating the particular condition in an individual. The label or package insert will further comprise instructions for administering the composition to the individual. The label may indicate directions for reconstitution and/or use. The container holding the pharmaceutical composition may be a multi-use vial, which allows for repeat administrations (e.g. from 2-6 administrations) of the reconstituted formulation. Package insert refers to instructions customarily included in commercial packages of therapeutic products that contain information about the indications, usage, dosage, administration, contraindications and/or warnings concerning the use of such therapeutic products. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kits or article of manufacture may include multiple unit doses of the pharmaceutical composition and instructions for use, packaged in quantities sufficient for storage and use in pharmacies, for example, hospital pharmacies and compounding pharmacies.

For the sake of conciseness, certain abbreviations are used herein. One example is the single letter abbreviation to represent amino acid residues. The amino acids and their corresponding three letter and single letter abbreviations are as follows:

| | | |
|---|---|---|
| alanine | Ala | (A) |
| arginine | Arg | (R) |
| asparagine | Asn | (N) |
| aspartic acid | Asp | (D) |
| cysteine | Cys | (C) |
| glutamic acid | Glu | (E) |
| glutamine | Gln | (Q) |
| glycine | Gly | (G) |
| histidine | His | (H) |
| isoleucine | Ile | (I) |
| leucine | Leu | (L) |
| lysine | Lys | (K) |
| methionine | Met | (M) |
| phenylalanine | Phe | (F) |
| proline | Pro | (P) |
| serine | Ser | (S) |
| threonine | Thr | (T) |
| tryptophan | Trp | (W) |
| tyrosine | Tyr | (Y) |
| valine | Val | (V) |

A number of embodiments of the disclosure have been described. Nevertheless, it will be understood that various modifications may be made without departing from the spirit and scope of the disclosure. Accordingly, the following examples are intended to illustrate but not limit the scope of disclosure described in the claims.

### EXAMPLES

The examples described herein are not intended to represent that the experiments below are all or the only experiments performed. Efforts have been made to ensure accuracy with respect to numbers used (for example, amounts, temperature, etc.), but some experimental errors and deviations should be accounted for. Unless indicated otherwise, parts are parts by weight, molecular weight is weight average molecular weight, temperature is in degree Celsius, and pressure is at or near atmospheric.

### Example 1: Generation of CARs and CAR-T Cells Armored with Membrane Bound Arginine-Glycine-Aspartic (RGD) Fusion Protein

Cloning of mbRGDs Armored CAR Constructs

In order to enhance T cells homing and infiltration into tumor, tumor homing peptides were fused with transmembrane domain, and functioned as membrane bound form, which were able to anchor to membrane of T cells.

Firstly we designed several membrane bound forms of RGD (mbRGD) comprising the hinge and transmembrane domain of CD28, and used as armor with DLL3 specific CAR-T cells. One of the mbRGDs armored CARs consisted of a DLL3-CAR (SEQ ID NO: 8), P2A (SEQ ID NO: 9), leader peptide of CD28 (SEQ ID NO: 1), RGD-4C peptide (SEQ ID NO: 2), Flag tag (SEQ ID NO: 3), (G45)₃ linker, a CD28 hinge region (SEQ ID NO: 4), and the transmembrane domain of CD28 (SEQ ID NO: 5). This construct was designated as DLL3-RGD-Flag-CD28 (SEQ ID NO: 10). Other one of the mbRGD armored CARs consisted of a DLL3-CAR (SEQ ID NO: 8), P2A (SEQ ID NO: 9), leader peptide of CD28 (SEQ ID NO: 1), RGD-4C peptide (SEQ ID NO: 2), Flag tag (SEQ ID NO: 3), (G45)₃ linker, a FasL hinge region (SEQ ID NO: 6), and the transmembrane domain of FasL (SEQ ID NO: 7). This construct was designated as DLL3-FasL-Flag-RGD (SEQ ID NO: 11). Sequences of the related elements and CARs are shown in Table 1.

TABLE 1
Modalities and sequences of mbRGD armored CARs

| SEQ ID NO | Modality | Sequence |
|---|---|---|
| 1 | CD28 leader | MLRLLLALNLFPSIQVTG |
| 2 | RGD-4C | CDCRGDCFC |
| 3 | Flag tag | DYKDDDDK |
| 4 | CD28 Hinge | IEVMYPPPYLDNEKSNGTIIHVKGKHLCPSPLFPGPSKP |
| 5 | CD28 TM | FWVLVVVGGVLACYSLLVTVAFIIFWVRS |
| 6 | FasL Hinge | QLFHLQK |
| 7 | FasL TM | LKKRGNHSTGLCLLVMFFMVLVALVGLGLGMF |
| 8 | DLL3-CAR | MALPVTALLLPLALLLHAARPQVQLVESGGGVVQPGGSLRLSCAASFS |
| | | GYGVSTMAWFRQAPGKGLEGVAAITVGSGNTYYADSVKGRFTISRDN |
| | | SKNTVYLQMNSLRAEDTAMYYCAVGYLSGGSWDVPGRYNYWGQGT |
| | | LVTVSSGGGGSGGGGSGGGGSEVQLVESGGGLVQPGGSLRLSCAASG |
| | | NTYSSNYMGWFRQAPGKGLEEVAVIYTRGGHTYYVDSVRGRFTISQD |
| | | NAKNSLYLQMNSLRAEDTAVYYCAASSRHRLGLNNPRDYDYWGQGT |
| | | LVTVSSTTTPAPRPPTPAPTIASQPLSLRPEACRPAAGGAVHTRGLDFAC |
| | | DIYIWAPLAGTCGVLLLSLVITLYCKRGRKKLLYIFKQPFMRPVQTTQE |
| | | EDGCSCRFPEEEEGGCELRVKFSRSADAPAYKQGQNQLYNELNLGRRE |
| | | EYDVLDKRRGRDPEMGGKPRRKNPQEGLYNELQKDKMAEAYSEIGM |
| | | KGERRRGKGHDGLYQGLSTATKDTYDALHMQALPPR |
| 9 | P2A | GSGATNFSLLKQAGDVEENPGP |
| 10 | DLL3-RGD- | MALPVTALLLPLALLLHAARPQVQLVESGGGVVQPGGSLRLSCAASFS |
| | Flag-CD28 | GYGVSTMAWFRQAPGKGLEGVAAITVGSGNTYYADSVKGRFTISRDN |
| | | SKNTVYLQMNSLRAEDTAMYYCAVGYLSGGSWDVPGRYNYWGQGT |
| | | LVTVSSGGGGSGGGGSGGGGSEVQLVESGGGLVQPGGSLRLSCAASG |
| | | NTYSSNYMGWFRQAPGKGLEEVAVIYTRGGHTYYVDSVRGRFTISQD |
| | | NAKNSLYLQMNSLRAEDTAVYYCAASSRHRLGLNNPRDYDYWGQGT |
| | | LVTVSSTTTPAPRPPTPAPTIASQPLSLRPEACRPAAGGAVHTRGLDFAC |
| | | DIYIWAPLAGTCGVLLLSLVITLYCKRGRKKLLYIFKQPFMRPVQTTQE |
| | | EDGCSCRFPEEEEGGCELRVKFSRSADAPAYKQGQNQLYNELNLGRRE |
| | | EYDVLDKRRGRDPEMGGKPRRKNPQEGLYNELQKDKMAEAYSEIGM |
| | | KGERRRGKGHDGLYQGLSTATKDTYDALHMQALPPRGSGATNFSLLK |
| | | QAGDVEENPGPMLRLLLALNLFPSIQVTGCDCRGDCFCGGGGSGGGGS |
| | | GGGGSDYKDDDDKIEVMYPPPYLDNEKSNGTIIHVKGKHLCPSPLFPG |
| | | PSKPFWVLVVVGGVLACYSLLVTVAFIIFWVRS |
| 11 | DLL3-FasL- | MALPVTALLLPLALLLHAARPQVQLVESGGGVVQPGGSLRLSCAASFS |
| | Flag-RGD | GYGVSTMAWFRQAPGKGLEGVAAITVGSGNTYYADSVKGRFTISRDN |
| | | SKNTVYLQMNSLRAEDTAMYYCAVGYLSGGSWDVPGRYNYWGQGT |
| | | LVTVSSGGGGSGGGGSGGGGSEVQLVESGGGLVQPGGSLRLSCAASG |
| | | NTYSSNYMGWFRQAPGKGLEEVAVIYTRGGHTYYVDSVRGRFTISQD |
| | | NAKNSLYLQMNSLRAEDTAVYYCAASSRHRLGLNNPRDYDYWGQGT |
| | | LVTVSSTTTPAPRPPTPAPTIASQPLSLRPEACRPAAGGAVHTRGLDFAC |
| | | DIYIWAPLAGTCGVLLLSLVITLYCKRGRKKLLYIFKQPFMRPVQTTQE |
| | | EDGCSCRFPEEEEGGCELRVKFSRSADAPAYKQGQNQLYNELNLGRRE |
| | | EYDVLDKRRGRDPEMGGKPRRKNPQEGLYNELQKDKMAEAYSEIGM |
| | | KGERRRGKGHDGLYQGLSTATKDTYDALHMQALPPRGSGATNFSLLK |
| | | QAGDVEENPGPLKKRGNHSTGLCLLVMFFMVLVALVGLGLGMFQLF |
| | | HLQKDYKDDDDKGGGGSGGGGSGGGGSCDCRGDCFC |

Generation of mbRGD Armored CAR-T Cell

### 1. Preparation of Lentivirus

The sequences encoding the RGD armored DLL3-CARs as described above were codon optimized and synthesized by GenScript. Then the sequences were incorporated into 2n d generation lentivirus vectors via standard molecular clone methods. All plasmid sequences were verified via sequencing. Then the plasmids and lentiviral packaging plasmids were co-transfected into HEK293 cells to produce lentivirus. Virus particles were collected from the supernatant, and concentrated by PEG precipitation or ultracentrifugation. The concentrated lentiviral particles were used to transduce T cells.

### 2. T Cell Transduction

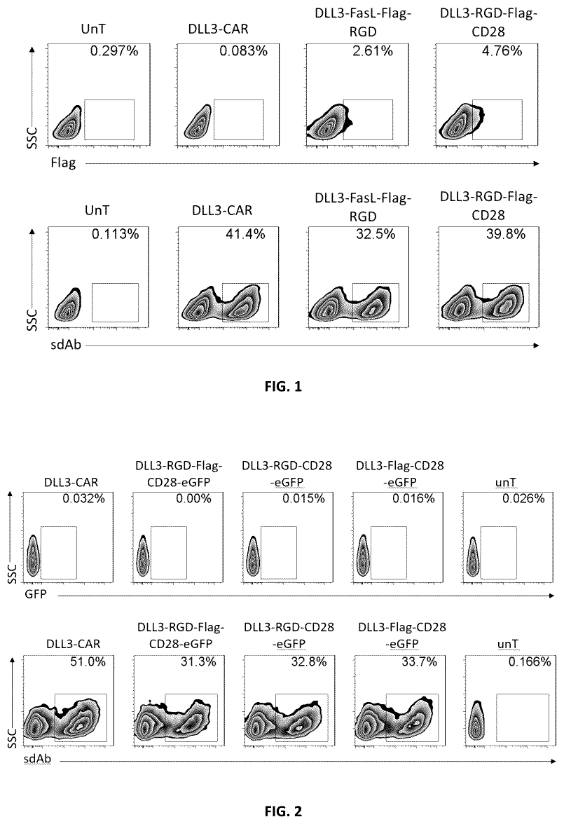

T cells were isolated from healthy donor PBMCs (HemaCare) using pan T cell isolation kit (Miltenyi Biotec, 130096535). Isolated T cells were cultured under AIMV (Gibco, 31035025) medium with 5% FBS (Gibco, 10099141) and further activated by CD3/CD28 activation beads (Miltenyi Biotec, 130091442) at a 2:1 ratio in the 37° C., 5% CO₂ incubator. 48 or 72 hours after initial activation, T cells were transduced with lentivirus expressing a DLL3 targeting CAR at proper multiplicity of infection (MOI) in the presence of 8 μg/ml polybrene (SIGMA-ALDRICH, H9268-10G). Additional IL-2 was supplemented to a final concentration of 300 IU/ml. Fresh medium was replaced 24 hours post lentiviral infection. Infected T cells were maintained under AIMV medium with 5% FBS and 300 IU/ml IL-2 at a cell density between 5×10⁵ to 1×10⁶ cells/ml. 4 days after transduction, the expression of CAR and RGD on the cell surface were determined by FACS. Specifically, 1×10⁵ transduced or untransduced T cells (unT) were collected by centrifugation at 300 g for 5 min. The cells were then re-suspended with 100 μl PBS containing 2 μl BV421-conjugated anti-Flag antibody (Biolegend, US) or iFlour488-Anti-sdAb (GenScript, China). Flag positive ratio represents RGD ratio on T cells, and sdAb positive ratio represents CAR ratio. As shown in FIG. 1, CAR positive ratio on DLL3-CAR, DLL3-FasL-Flag-RGD, and DLL3-RGD-Flag-CD28 were 41.4%, 32.5% and 39.8%. While the mbRGDs expressed on the surface of DLL3-FasL-Flag-RGD and DLL3-RGD-Flag-CD28 were only 2.6% and 4.8%. These results indicated that these mbRGD structures were poorly expressed on T cells.

### 3. Optimization of the CAR Structures

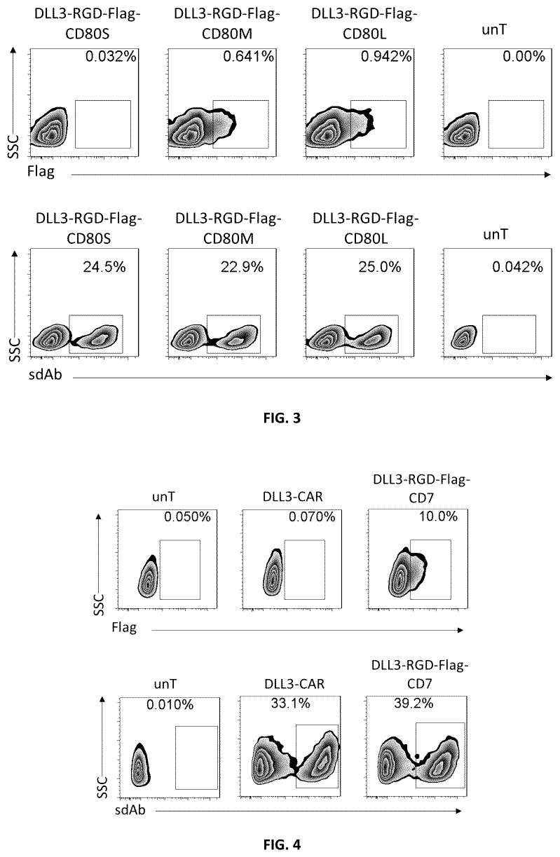

Then we sought to determine which module(s) account for the low expression of mbRGD. Several constructs were designed by replacing the hinge, intracellular, or transmembrane domain of mbRGD. The sequences and structures are shown in Table 2. Firstly, to determine whether addition of an intracellular domain could stabilize the mbRGD, eGFP (SEQ ID NO: 12) sequence was conjugated to transmembrane domain of mbRGD, which was named as DLL3-RGD-Flag-CD28-eGFP (SEQ ID NO: 19). Then mbRGD lacking of Flag (DLL3-RGD-CD28-eGFP (SEQ ID NO: 21) or RGD (DLL3-Flag-CD28-eGFP (SEQ ID NO: 20) was constructed to determine whether these module influences expression of mbRGD. CAR-T cells were generated as described in Example 1. As shown in FIG. 2, none of these mbRGD successfully expressed on CAR-T cells. These results suggested the transmembrane domain of CD28 may contribute to poor expression of mbRGD. Then we sought to determine whether replacing CD28 transmembrane of mbRGD with CD7 or CD80 may enhance mbRGD expression on CAR-T. Specifically, DLL3-RGD-Flag-CD7 (SEQ ID NO: 22) and DLL3-RGD-CD7 (SEQ ID NO: 26) were constructed comprising hinge (SEQ ID NO: 13) and transmembrane domains (SEQ ID NO: 14) of CD7, DLL3-RGD-Flag-CD80S (SEQ ID NO: 23), DLL3-RGD-Flag-CD80M (SEQ ID NO: 24), and DLL3-RGD-Flag-CD80L (SEQ ID NO: 25) were constructed comprising a transmembrane domain (SEQ ID NO:18) of CD80, with short (SEQ ID NO:15), medium (SEQ ID NO:16), or long (SEQ ID NO:17) hinge domains of CD80. The relevant sequences are shown in Table 2. These constructs were packaged into lentivirus, and transduced into primary T cells. As shown in FIG. 3, DLL3-RGD-Flag-CD80S, DLL3-RGD-Flag-CD80M, and DLL3-RGD-Flag-CD80L showed minimal level of mbRGD expression. As shown in FIG. 4, DLL3-RGD-Flag-CD7 showed relative higher expression of mbRGD than other constructs. These results indicated that expression of mbRGD may be facilitated by CD7 transmembrane and hinge domains. Therefore, we then utilized DLL3-RGD-CD7 (SEQ ID NO: 26) for cell function assays.

TABLE 2
Modalities and sequences of mbRGD armored CARs

| SEQ ID NO | Modality | Sequence |
|---|---|---|
| 12 | eGFP | MVSKGEELFTGVVPILVELDGDVNGHKFSVSGEGEGDATYGKLTLKFI |
| | | CTTGKLPVPWPTLVTTLTYGVQCFSRYPDHMKQHDFFKSAMPEGYVQ |
| | | ERTIFFKDDGNYKTRAEVKFEGDTLVNRIELKGIDFKEDGNILGHKLEY |
| | | NYNSHNVYIMADKQKNGIKVNFKIRHNIEDGSVQLADHYQQNTPIGD |
| | | GPVLLPDNHYLSTQSALSKDPNEKRDHMVLLEFVTAAGITLGMDELY |
| | | K |
| 13 | CD7 hinge | PPRASALPAPPTGSALPDPQTASALPDPPAASALP |
| 14 | CD7 | AALAVISFLLGLGLGVACVLA |
| | transmembrane | |
| | domain | |
| 15 | CD80 short | DN |
| | Hinge | |
| 16 | CD80 medium | WNTTKQEHFPDN |
| | Hinge | |
| 17 | CD80 long | PSISDFEIPTSNIRRIICSTSGGFPEPHLSWLENGEELNAINTTVSQDPETE |
| | Hinge | LYAVSSKLDFNMTTNHSFMCLIKYGHLRVNQTFNWNTTKQEHFPDN |
| 18 | CD80 | LLPSWAITLISVNGIFVICCLTYCFAPRCRERRRNERLRRESVRPV |
| | transmembrane | |
| | domain | |
| 19 | DLL3-RGD- | MALPVTALLLPLALLLHAARPQVQLVESGGGVVQPGGSLRLSCAASFS |
| | Flag-CD28- | GYGVSTMAWFRQAPGKGLEGVAAITVGSGNTYYADSVKGRFTISRDN |
| | eGFP | SKNTVYLQMNSLRAEDTAMYYCAVGYLSGGSWDVPGRYNYWGQGT |
| | | LVTVSSGGGGSGGGGSGGGGSEVQLVESGGGLVQPGGSLRLSCAASG |
| | | NTYSSNYMGWFRQAPGKGLEEVAVIYTRGGHTYYVDSVRGRFTISQD |
| | | NAKNSLYLQMNSLRAEDTAVYYCAASSRHRLGLNNPRDYDYWGQGT |
| | | LVTVSSTTTPAPRPPTPAPTIASQPLSLRPEACRPAAGGAVHTRGLDFAC |
| | | DIYIWAPLAGTCGVLLLSLVITLYCKRGRKKLLYIFKQPFMRPVQTTQE |
| | | EDGCSCRFPEEEEGGCELRVKFSRSADAPAYKQGQNQLYNELNLGRRE |
| | | EYDVLDKRRGRDPEMGGKPRRKNPQEGLYNELQKDKMAEAYSEIGM |
| | | KGERRRGKGHDGLYQGLSTATKDTYDALHMQALPPRGSGATNFSLLK |
| | | QAGDVEENPGPMLRLLLALNLFPSIQVTGCDCRGDCFCGGGGSGGGGS |
| | | GGGGSDYKDDDDKIEVMYPPPYLDNEKSNGTIIHVKGKHLCPSPLFPG |
| | | PSKPFWVLVVVGGVLACYSLLVTVAFIIFWVRSMVSKGEELFTGVVPIL |
| | | VELDGDVNGHKFSVSGEGEGDATYGKLTLKFICTTGKLPVPWPTLVTT |
| | | LTYGVQCFSRYPDHMKQHDFFKSAMPEGYVQERTIFFKDDGNYKTRA |
| | | EVKFEGDTLVNRIELKGIDFKEDGNILGHKLEYNYNSHNVYIMADKQK |
| | | NGIKVNFKIRHNIEDGSVQLADHYQQNTPIGDGPVLLPDNHYLSTQSAL |
| | | SKDPNEKRDHMVLLEFVTAAGITLGMDELYK |
| 20 | DLL3-Flag- | MALPVTALLLPLALLLHAARPQVQLVESGGGVVQPGGSLRLSCAASFS |
| | CD28-eGFP | GYGVSTMAWFRQAPGKGLEGVAAITVGSGNTYYADSVKGRFTISRDN |
| | | SKNTVYLQMNSLRAEDTAMYYCAVGYLSGGSWDVPGRYNYWGQGT |
| | | LVTVSSGGGGSGGGGSGGGGSEVQLVESGGGLVQPGGSLRLSCAASG |
| | | NTYSSNYMGWFRQAPGKGLEEVAVIYTRGGHTYYVDSVRGRFTISQD |
| | | NAKNSLYLQMNSLRAEDTAVYYCAASSRHRLGLNNPRDYDYWGQGT |
| | | LVTVSSTTTPAPRPPTPAPTIASQPLSLRPEACRPAAGGAVHTRGLDFAC |
| | | DIYIWAPLAGTCGVLLLSLVITLYCKRGRKKLLYIFKQPFMRPVQTTQE |
| | | EDGCSCRFPEEEEGGCELRVKFSRSADAPAYKQGQNQLYNELNLGRRE |
| | | EYDVLDKRRGRDPEMGGKPRRKNPQEGLYNELQKDKMAEAYSEIGM |
| | | KGERRRGKGHDGLYQGLSTATKDTYDALHMQALPPRGSGATNFSLLK |
| | | QAGDVEENPGPMLRLLLALNLFPSIQVTGGGGGSGGGGSGGGGSDYK |
| | | DDDDKIEVMYPPPYLDNEKSNGTIIHVKGKHLCPSPLFPGPSKPFWVLV |
| | | VVGGVLACYSLLVTVAFIIFWVRSMVSKGEELFTGVVPILVELDGDVN |
| | | GHKFSVSGEGEGDATYGKLTLKFICTTGKLPVPWPTLVTTLTYGVQCF |
| | | SRYPDHMKQHDFFKSAMPEGYVQERTIFFKDDGNYKTRAEVKFEGDT |
| | | LVNRIELKGIDFKEDGNILGHKLEYNYNSHNVYIMADKQKNGIKVNFK |
| | | IRHNIEDGSVQLADHYQQNTPIGDGPVLLPDNHYLSTQSALSKDPNEKR |
| | | DHMVLLEFVTAAGITLGMDELYK |
| 21 | DLL3-RGD- | MALPVTALLLPLALLLHAARPQVQLVESGGGVVQPGGSLRLSCAASFS |
| | CD28-eGFP | GYGVSTMAWFRQAPGKGLEGVAAITVGSGNTYYADSVKGRFTISRDN |
| | | SKNTVYLQMNSLRAEDTAMYYCAVGYLSGGSWDVPGRYNYWGQGT |
| | | LVTVSSGGGGSGGGGSGGGGSEVQLVESGGGLVQPGGSLRLSCAASG |
| | | NTYSSNYMGWFRQAPGKGLEEVAVIYTRGGHTYYVDSVRGRFTISQD |
| | | NAKNSLYLQMNSLRAEDTAVYYCAASSRHRLGLNNPRDYDYWGQGT |
| | | LVTVSSTTTPAPRPPTPAPTIASQPLSLRPEACRPAAGGAVHTRGLDFAC |
| | | DIYIWAPLAGTCGVLLLSLVITLYCKRGRKKLLYIFKQPFMRPVQTTQE |
| | | EDGCSCRFPEEEEGGCELRVKFSRSADAPAYKQGQNQLYNELNLGRRE |
| | | EYDVLDKRRGRDPEMGGKPRRKNPQEGLYNELQKDKMAEAYSEIGM |
| | | KGERRRGKGHDGLYQGLSTATKDTYDALHMQALPPRGSGATNFSLLK |
| | | QAGDVEENPGPMLRLLLALNLFPSIQVTGCDCRGDCFCGGGGSGGGGS |
| | | GGGGSIEVMYPPPYLDNEKSNGTIIHVKGKHLCPSPLFPGPSKPFWVLV |
| | | VVGGVLACYSLLVTVAFIIFWVRSMVSKGEELFTGVVPILVELDGDVN |
| | | GHKFSVSGEGEGDATYGKLTLKFICTTGKLPVPWPTLVTTLTYGVQCF |
| | | SRYPDHMKQHDFFKSAMPEGYVQERTIFFKDDGNYKTRAEVKFEGDT |
| | | LVNRIELKGIDFKEDGNILGHKLEYNYNSHNVYIMADKQKNGIKVNFK |
| | | IRHNIEDGSVQLADHYQQNTPIGDGPVLLPDNHYLSTQSALSKDPNEKR |
| | | DHMVLLEFVTAAGITLGMDELYK |
| 22 | DLL3-RGD- | MALPVTALLLPLALLLHAARPQVQLVESGGGVVQPGGSLRLSCAASFS |
| | Flag-CD7 | GYGVSTMAWFRQAPGKGLEGVAAITVGSGNTYYADSVKGRFTISRDN |
| | | SKNTVYLQMNSLRAEDTAMYYCAVGYLSGGSWDVPGRYNYWGQGT |
| | | LVTVSSGGGGSGGGGSGGGGSEVQLVESGGGLVQPGGSLRLSCAASG |
| | | NTYSSNYMGWFRQAPGKGLEEVAVIYTRGGHTYYVDSVRGRFTISQD |
| | | NAKNSLYLQMNSLRAEDTAVYYCAASSRHRLGLNNPRDYDYWGQGT |
| | | LVTVSSTTTPAPRPPTPAPTIASQPLSLRPEACRPAAGGAVHTRGLDFAC |
| | | DIYIWAPLAGTCGVLLLSLVITLYCKRGRKKLLYIFKQPFMRPVQTTQE |
| | | EDGCSCRFPEEEEGGCELRVKFSRSADAPAYKQGQNQLYNELNLGRRE |
| | | EYDVLDKRRGRDPEMGGKPRRKNPQEGLYNELQKDKMAEAYSEIGM |
| | | KGERRRGKGHDGLYQGLSTATKDTYDALHMQALPPRGSGATNFSLLK |
| | | QAGDVEENPGPMPMGSLQPLATLYLLGMLVASCLGCDCRGDCFCGG |
| | | GGSGGGGSGGGGSDYKDDDDKPPRASALPAPPTGSALPDPQTASALPD |
| | | PPAASALPAALAVISFLLGLGLGVACVLA |
| 23 | DLL3-RGD- | MALPVTALLLPLALLLHAARPQVQLVESGGGVVQPGGSLRLSCAASFS |
| | Flag-CD80S | GYGVSTMAWFRQAPGKGLEGVAAITVGSGNTYYADSVKGRFTISRDN |
| | | SKNTVYLQMNSLRAEDTAMYYCAVGYLSGGSWDVPGRYNYWGQGT |
| | | LVTVSSGGGGSGGGGSGGGGSEVQLVESGGGLVQPGGSLRLSCAASG |
| | | NTYSSNYMGWFRQAPGKGLEEVAVIYTRGGHTYYVDSVRGRFTISQD |
| | | NAKNSLYLQMNSLRAEDTAVYYCAASSRHRLGLNNPRDYDYWGQGT |
| | | LVTVSSTTTPAPRPPTPAPTIASQPLSLRPEACRPAAGGAVHTRGLDFAC |
| | | DIYIWAPLAGTCGVLLLSLVITLYCKRGRKKLLYIFKQPFMRPVQTTQE |
| | | EDGCSCRFPEEEEGGCELRVKFSRSADAPAYKQGQNQLYNELNLGRRE |
| | | EYDVLDKRRGRDPEMGGKPRRKNPQEGLYNELQKDKMAEAYSEIGM |
| | | KGERRRGKGHDGLYQGLSTATKDTYDALHMQALPPRGSGATNFSLLK |
| | | QAGDVEENPGPMGHTRRQGTSPSKCPYLNFFQLLVLAGLSHFCSGCDC |
| | | RGDCFCGGGGSDYKDDDDKDNLLPSWAITLISVNGIFVICCLTYCFAPR |
| | | CRERRRNERLRRESVRPV |
| 24 | DLL3-RGD- | MALPVTALLLPLALLLHAARPQVQLVESGGGVVQPGGSLRLSCAASFS |
| | Flag-CD80M | GYGVSTMAWFRQAPGKGLEGVAAITVGSGNTYYADSVKGRFTISRDN |
| | DLL3-RGD- | SKNTVYLQMNSLRAEDTAMYYCAVGYLSGGSWDVPGRYNYWGQGT |
| | | LVTVSSGGGGSGGGGSGGGGSEVQLVESGGGLVQPGGSLRLSCAASG |
| | | NTYSSNYMGWFRQAPGKGLEEVAVIYTRGGHTYYVDSVRGRFTISQD |
| | | NAKNSLYLQMNSLRAEDTAVYYCAASSRHRLGLNNPRDYDYWGQGT |
| | | LVTVSSTTTPAPRPPTPAPTIASQPLSLRPEACRPAAGGAVHTRGLDFAC |
| | | DIYIWAPLAGTCGVLLLSLVITLYCKRGRKKLLYIFKQPFMRPVQTTQE |
| | | EDGCSCRFPEEEEGGCELRVKFSRSADAPAYKQGQNQLYNELNLGRRE |
| | | EYDVLDKRRGRDPEMGGKPRRKNPQEGLYNELQKDKMAEAYSEIGM |
| | | KGERRRGKGHDGLYQGLSTATKDTYDALHMQALPPRGSGATNFSLLK |
| | | QAGDVEENPGPMGHTRRQGTSPSKCPYLNFFQLLVLAGLSHFCSGCDC |
| | | RGDCFCGGGGSDYKDDDDKWNTTKQEHFPDNLLPSWAITLISVNGIFV |
| | | ICCLTYCFAPRCRERRRNERLRRESVRPV |
| 25 | Flag-CD80L | MALPVTALLLPLALLLHAARPQVQLVESGGGVVQPGGSLRLSCAASFS |
| | | GYGVSTMAWFRQAPGKGLEGVAAITVGSGNTYYADSVKGRFTISRDN |
| | | SKNTVYLQMNSLRAEDTAMYYCAVGYLSGGSWDVPGRYNYWGQGT |
| | | LVTVSSGGGGSGGGGSGGGGSEVQLVESGGGLVQPGGSLRLSCAASG |
| | | NTYSSNYMGWFRQAPGKGLEEVAVIYTRGGHTYYVDSVRGRFTISQD |
| | | NAKNSLYLQMNSLRAEDTAVYYCAASSRHRLGLNNPRDYDYWGQGT |
| | | LVTVSSTTTPAPRPPTPAPTIASQPLSLRPEACRPAAGGAVHTRGLDFAC |
| | | DIYIWAPLAGTCGVLLLSLVITLYCKRGRKKLLYIFKQPFMRPVQTTQE |
| | | EDGCSCRFPEEEEGGCELRVKFSRSADAPAYKQGQNQLYNELNLGRRE |
| | | EYDVLDKRRGRDPEMGGKPRRKNPQEGLYNELQKDKMAEAYSEIGM |
| | | KGERRRGKGHDGLYQGLSTATKDTYDALHMQALPPRGSGATNFSLLK |
| | | QAGDVEENPGPMGHTRRQGTSPSKCPYLNFFQLLVLAGLSHFCSGCDC |
| | | RGDCFCGGGGSDYKDDDDKPSISDFEIPTSNIRRIICSTSGGFPEPHLSWL |
| | | ENGEELNAINTTVSQDPETELYAVSSKLDFNMTTNHSFMCLIKYGHLR |
| | | VNQTFNWNTTKQEHFPDNLLPSWAITLISVNGIFVICCLTYCFAPRCRE |
| | | RRRNERLRRESVRPV |
| 26 | DLL3-RGD- | MALPVTALLLPLALLLHAARPQVQLVESGGGVVQPGGSLRLSCAASFS |
| | CD7 | GYGVSTMAWFRQAPGKGLEGVAAITVGSGNTYYADSVKGRFTISRDN |
| | | SKNTVYLQMNSLRAEDTAMYYCAVGYLSGGSWDVPGRYNYWGQGT |
| | | LVTVSSGGGGSGGGGSGGGGSEVQLVESGGGLVQPGGSLRLSCAASG |
| | | NTYSSNYMGWFRQAPGKGLEEVAVIYTRGGHTYYVDSVRGRFTISQD |
| | | NAKNSLYLQMNSLRAEDTAVYYCAASSRHRLGLNNPRDYDYWGQGT |
| | | LVTVSSTTTPAPRPPTPAPTIASQPLSLRPEACRPAAGGAVHTRGLDFAC |
| | | DIYIWAPLAGTCGVLLLSLVITLYCKRGRKKLLYIFKQPFMRPVQTTQE |
| | | EDGCSCRFPEEEEGGCELRVKFSRSADAPAYKQGQNQLYNELNLGRRE |
| | | EYDVLDKRRGRDPEMGGKPRRKNPQEGLYNELQKDKMAEAYSEIGM |
| | | KGERRRGKGHDGLYQGLSTATKDTYDALHMQALPPRGSGATNFSLLK |
| | | QAGDVEENPGPMPMGSLQPLATLYLLGMLVASCLGCDCRGDCFCGG |
| | | GGSGGGGSGGGGSPPRASALPAPPTGSALPDPQTASALPDPPAASALPA |
| | | ALAVISFLLGLGLGVACVLA |
| 27 | AS63997VH5 | QVQLVESGGGVVQPGGSLRLSCAASFSGYGVSTMAWFRQAPGKGLEG |
| | V_{H}H | VAAITVGSGNTYYADSVKGRFTISRDNSKNTVYLQMNSLRAEDTAMY |
| | | YCAVGYLSGGSWDVPGRYNYWGQGTLVTVSS |
| 28 | AS64380VH5 | EVQLVESGGGLVQPGGSLRLSCAASGNTYSSNYMGWFRQAPGKGLEE |
| | V_{H}H | VAVIYTRGGHTYYVDSVRGRFTISQDNAKNSLYLQMNSLRAEDTAVY |
| | | YCAASSRHRLGLNNPRDYDYWGQGTLVTVSS |
| 29 | AS63997VH5 | FSGYGVSTMA |
| | V_{H}H CDR1 | |
| 30 | AS63997VH5 | AITVGSGNTYYADSVKG |
| | V_{H}H CDR2 | |
| 31 | AS63997VH5 | GYLSGGSWDVPGRYNY |
| | V_{H}H CDR3 | |
| 32 | AS64380VH5 | GNTYSSNYMG |
| | V_{H}H CDR1 | |
| 33 | AS64380VH5 | VIYTRGGHTYYVDSVRG |
| | V_{H}H CDR2 | |
| 34 | AS64380VH5 | SSRHRLGLNNPRDYDY |
| | V_{H}H CDR3 | |

### Example 2: mbRGD Enhances CAR-T Cells Adhesion to Endothelial Cells

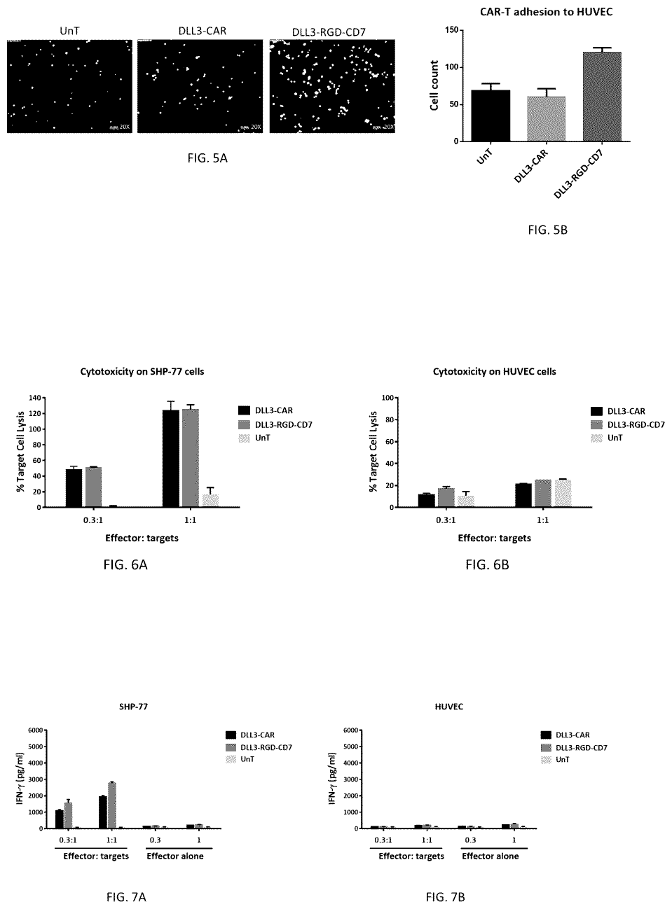

To investigate whether mbRGD can improve T cell adhesion to neo-vascular endothelial cells, endothelial cell adhesion assay was performed. In this assay, human umbilical vein endothelial cells (HUVEC) were used to represent neo-vascular endothelial cells. Firstly, expression of integrin αv and integrin β5 (both of which are receptors for RGD peptides) on HUVEC surface were detected. Then RGD armored DLL3 CARs, specifically DLL3-RGD-CD7, were prepared and their adhesion to HUVEC were investigated. Briefly, 1×10⁵ HUVEC cells were seeded onto the bottom of a 24-well-plate to form a single layer. The next day, 3×10⁵ DLL3-RGD-CD7, DLL3-CAR, and untransduced T cells (unT) were labeled with CF SE and added to the single HUVEC layer respectively. After 30 min incubation, the unbounded T cells were carefully rinsed out with fresh medium. The bounded cell were imaged on a fluorescence microscopy and counted. As shown in FIGS. 5A and 5B, DLL3-RGD-CD7 had remarkably more cell adhesion to HUVEC than DLL3-CAR or unT cells. These results suggested that mbRGD can enhance T cell adhesion to neo-vascular endothelial cells.

### Example 3: Influence of the mbRGD on Cytotoxicity and Cytokine Secretion of CAR-T Cells Against Target Cells and Endothelial Cells

To investigate whether mbRGD influences the cytotoxicity of CAR-T cells against target cells or endothelial cells, HUVEC and DLL3 positive cells SHP77 were used for evaluating the cytotoxicity and cytokine secretion of DLL3-CAR-T cells with or without mbRGD. Specifically, SHP77 cells were co-incubated with DLL3-RGD-CD7, DLL3-CAR, and unT for 22 h. Then, LDH (lactate dehydrogenase) levels were measured. As shown in FIGS. 6A-6B, cytotoxicity levels of mbRGD armored CAR-T against both SHP77 and HUVEC were comparable to the conventional CAR-T cells. These results demonstrated that mbRGD has no influence on the cytotoxicity of CAR-T cells against target cells; meanwhile the mbRGD armored CAR-T cells did not cause cytotoxicity to endothelial cells. The supernatants of the co-cultures were then taken for IFN-γ measurement by HTFR methods. As shown in FIGS. 7A-7B, IFN-γ secretion of all the CAR-T cells is highly activated by SHP77 co-culture. And DLL3-RGD-CD7 secreted higher levels of IFN-γ than DLL3-CAR-T cells upon co-culture with SHP77 (FIG. 7A). Although the basal level IFN-γ of DLL3-RGD-CD7 is higher than DLL3-CAR-T cells, none of the CAR-T cells showed increased IFN-γ secretion upon co-culture with HUVEC compared to basal level (CAR-T cells co-cultured with HUVEC). These results demonstrated that mbRGD armored CAR-T cells are safe to endothelial cells.

### Example 4: mbRGD Armored CAR-T Cells Demonstrated Superior Anti-Tumor Efficacy in Vivo

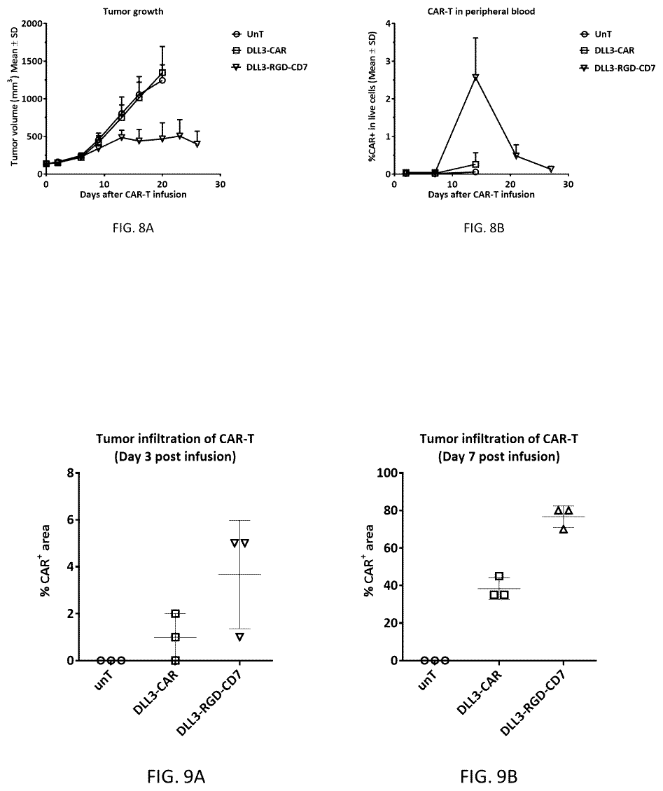

To investigate whether the mbRGD could enhance anti-tumor efficacy of CAR-T cells, an in vivo study was conducted on a SHP77 xenograft NCG mice model. Specifically, SHP77 cells were implanted subcutaneously to NCG mice, and SHP77 tumors were allowed to grow to 100-160 mm³. In order to compare antitumor efficacy, a suboptimal dose of DLL3-CAR-T was used, which showed little antitumor effect in this model. Specifically, 0.15 M of DLL3-RGD-CD7, DLL3-CAR, and untransduced T cells (“unT”) were infused into mice intravenously. The tumor volumes were monitored twice a week, and percentage of CAR-T in peripheral blood were monitored once a week. As shown in FIGS. 8A-8B, DLL3-RGD-CD7 showed much better antitumor efficacy (FIG. 8A) and CAR-T cell expansion (FIG. 8B) than the unarmored CAR-T cells. These results demonstrated that the mbRGD could enhance anti-tumor efficacy of CAR-T cells in vivo.

### Example 5: mbRGD Armored CAR-T Cells Demonstrated Superior Tumor Infiltration in Vivo

To investigate whether the enhanced antitumor efficacy of mbRGD armored CAR-T is at least partly due to enhanced tumor infiltration, we performed an in vivo assay to determine tumor infiltration of different CAR-T. Firstly, SHP77 cells were implanted subcutaneously to NCG mice. The tumors were allowed to grow to 250-300 mm³. Then 3 M of DLL3-CAR or DLL3-RGD-CD7 CAR-T cells were infused intravenously. The mice were euthanized and tumors resected 3 days and 7 days after infusion. The resected tumors were used for anti-DLL3 sdAb staining by immunohistochemistry (IHC) to observe the level of CAR-T infiltration in the tumor.

As shown in FIGS. 9A-9B, DLL3-RGD-CD7 CAR-T cells showed higher tumor infiltration of DLL3 positive cells than DLL3-CAR both at Day 3 and Day 7. These results indicated that mbRGD could enhance tumor infiltration of CAR-T cells.

### Example 6: The Expression of mbRGD was Positively Correlated with the Adhesion of CAR-T Cells to Endothelial Cells

To investigate whether the expression of mbRGD would affect CAR-T cells adhesion to endothelial cells, endothelial cell adhesion assay was performed. In this assay, human umbilical vein endothelial cells (HUVEC) were used to represent neo-vascular endothelial cells. The RGD armored DLL3 CAR-T were prepared and their adhesion to HUVEC were investigated. Briefly, 1×10⁵ HUVEC cells were seeded onto the bottom of a 24-well-plate to form a single layer. The next day, 3×10⁵ DLL3-RGD-CD7, DLL3-RGD-Flag-CD7, DLL3-CAR, DLL3-RGD-Flag-CD28, DLL3-FasL-Flag-RGD, DLL3-RGD-Flag-CD80S, DLL3-RGD-Flag-CD80M, DLL3-RGD-Flag-CD80L and untransduced T cells (unT) were labeled with CFSE and added to the single HUVEC layer respectively. After 30 min incubation, the unbounded T cells were carefully rinsed out with fresh medium. The bounded cell were imaged on a fluorescence microscopy and counted. As shown in FIGS. 10A and 10B, DLL3-RGD-CD7 and DLL3-RGD-Flag-CD7 had remarkably more cell adhesion to HUVEC than other CAR or unT cells. These results suggested that the expression of mbRGD was positively correlated with the adhesion of CAR-T cells to endothelial cells.

### Example 7: Generation of ε-TRuC and ε-TRuC-T Cells Armored with Membrane Bound Arginine-Glycine-Aspartic (RGD) Fusion Protein

Cloning of mbRGDs Armored ε-TRuC Constructs

In order to enhance T cells homing and infiltration into tumor, tumor homing peptides were fused with transmembrane domain, and functioned as membrane bound form, which were able to anchor to membrane of T cells.

We designed several membrane bound form of RGD (mbRGD) based on hinge and transmembrane domain of CD7, and used as armor with DLL3 specific ε-T-cell receptor fusion construct (i.e., ε-TruC, also referred to as cTCR) cells. One of the mbRGDs armored ε-TRuC consisted of DLL3-ε-TRuC (SEQ ID NO: 35), P2A (SEQ ID NO: 9), RGD-4C peptide (SEQ ID NO: 2), (G45)₃ linker, CD7 hinge (SEQ ID NO: 13), and transmembrane domain of CD7 (SEQ ID NO: 14). This construct was designated as DLL3-ε-TRuC-RGD-CD7 (SEQ ID NO: 36). Other one of the mbRGD armored ε-TRuC consisted of DLL3-ε-TRuC (SEQ ID NO: 35), P2A (SEQ ID NO: 9), RGD-4C peptide (SEQ ID NO: 2), Flag tag (SEQ ID NO: 3), (G45)₃ linker, CD7 hinge (SEQ ID NO: 13), and transmembrane domain of CD7 (SEQ ID NO: 14). This construct was designated as DLL3-ε-TRuC-RGD-Flag-CD7 (SEQ ID NO: 37). Sequences of the related elements and ε-TRuC are shown in Tables 1, 2 and 3.

TABLE 3
Modalities and sequences of mbRGD armored &-TRUC

| SEQ ID NO | Modality | Sequence |
|---|---|---|
| 35 | DLL3-8- | MQSGTHWRVLGLCLLSVGVWGQQVQLVESGGGVVQPGGSLRLSCAASF |
| | TRUC | SGYGVSTMAWFRQAPGKGLEGVAAITVGSGNTYYADSVKGRFTISRDNS |
| | | KNTVYLQMNSLRAEDTAMYYCAVGYLSGGSWDVPGRYNYWGQGTLVT |
| | | VSSGGGGSGGGGSGGGGSEVQLVESGGGLVQPGGSLRLSCAASGNTYSS |
| | | NYMGWFRQAPGKGLEEVAVIYTRGGHTYYVDSVRGRFTISQDNAKNSLY |
| | | LQMNSLRAEDTAVYYCAASSRHRLGLNNPRDYDYWGQGTLVTVSSGGG |
| | | GSGGGGSGGGGSDGNEEMGGITQTPYKVSISGTTVILTCPQYPGSEILWQH |
| | | NDKNIGGDEDDKNIGSDEDHLSLKEFSELEQSGYYVCYPRGSKPEDANFY |
| | | LYLRARVCENCMEMDVMSVATIVIVDICITGGLLLLVYYWSKNRKAKAK |
| | | PVTRGAGAGGRQRGQNKERPPPVPNPDYEPIRKGQRDLYSGLNQRRI |
| 36 | DLL3-8- | MQSGTHWRVLGLCLLSVGVWGQQVQLVESGGGVVQPGGSLRLSCAASF |
| | TRUC- | SGYGVSTMAWFRQAPGKGLEGVAAITVGSGNTYYADSVKGRFTISRDNS |
| | RGD-CD7 | KNTVYLQMNSLRAEDTAMYYCAVGYLSGGSWDVPGRYNYWGQGTLVT |
| | | VSSGGGGSGGGGSGGGGSEVQLVESGGGLVQPGGSLRLSCAASGNTYSS |
| | | NYMGWFRQAPGKGLEEVAVIYTRGGHTYYVDSVRGRFTISQDNAKNSLY |
| | | LQMNSLRAEDTAVYYCAASSRHRLGLNNPRDYDYWGQGTLVTVSSGGG |
| | | GSGGGGSGGGGSDGNEEMGGITQTPYKVSISGTTVILTCPQYPGSEILWQH |
| | | NDKNIGGDEDDKNIGSDEDHLSLKEFSELEQSGYYVCYPRGSKPEDANFY |
| | | LYLRARVCENCMEMDVMSVATIVIVDICITGGLLLLVYYWSKNRKAKAK |
| | | PVTRGAGAGGRQRGQNKERPPPVPNPDYEPIRKGQRDLYSGLNQRRIGSG |
| | | ATNFSLLKQAGDVEENPGPMPMGSLQPLATLYLLGMLVASCLGCDCRGD |
| | | CFCGGGGSGGGGSGGGGSPPRASALPAPPTGSALPDPQTASALPDPPAASA |
| | | LPAALAVISFLLGLGLGVACVLA |
| 37 | DLL3-8- | MQSGTHWRVLGLCLLSVGVWGQQVQLVESGGGVVQPGGSLRLSCAASF |
| | TRUC- | SGYGVSTMAWFRQAPGKGLEGVAAITVGSGNTYYADSVKGRFTISRDNS |
| | RGD-Flag- | KNTVYLQMNSLRAEDTAMYYCAVGYLSGGSWDVPGRYNYWGQGTLVT |
| | CD7 | VSSGGGGSGGGGSGGGGSEVQLVESGGGLVQPGGSLRLSCAASGNTYSS |
| | | NYMGWFRQAPGKGLEEVAVIYTRGGHTYYVDSVRGRFTISQDNAKNSLY |
| | | LQMNSLRAEDTAVYYCAASSRHRLGLNNPRDYDYWGQGTLVTVSSGGG |
| | | GSGGGGSGGGGSDGNEEMGGITQTPYKVSISGTTVILTCPQYPGSEILWQH |
| | | NDKNIGGDEDDKNIGSDEDHLSLKEFSELEQSGYYVCYPRGSKPEDANFY |
| | | LYLRARVCENCMEMDVMSVATIVIVDICITGGLLLLVYYWSKNRKAKAK |
| | | PVTRGAGAGGRQRGQNKERPPPVPNPDYEPIRKGQRDLYSGLNQRRIGSG |
| | | ATNFSLLKQAGDVEENPGPMPMGSLQPLATLYLLGMLVASCLGCDCRGD |
| | | CFCGGGGSGGGGSGGGGSDYKDDDDKPPRASALPAPPTGSALPDPQTASA |
| | | LPDPPAASALPAALAVISFLLGLGLGVACVLA |

Generation of mbRGD Armored ε-TRuC-T Cell

### 1. Preparation of Lentivirus

The sequences encoding the RGD armored DLL3-ε-TRuC as described above were codon optimized and synthesized by GenScript. Then the sequences were incorporated into 2^{nd} generation lentivirus vectors via standard molecular clone methods. All plasmid sequences were verified via sequencing. Then the plasmids and lentiviral packaging plasmids were co-transfected into HEK293 cells to produce lentivirus. Virus particles were collected from the supernatant, and concentrated by PEG precipitation or ultracentrifugation. The concentrated lentiviral particles were used to transduce T cells.

### 2. T Cell Transduction

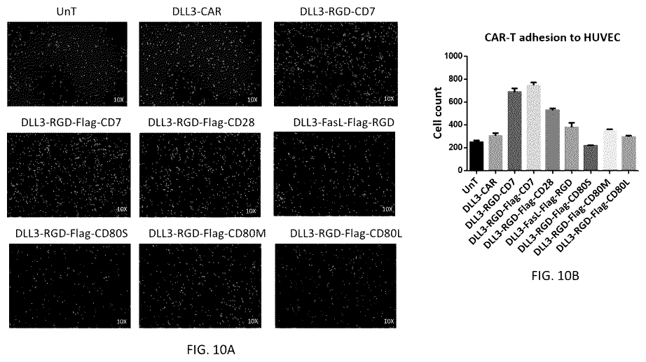

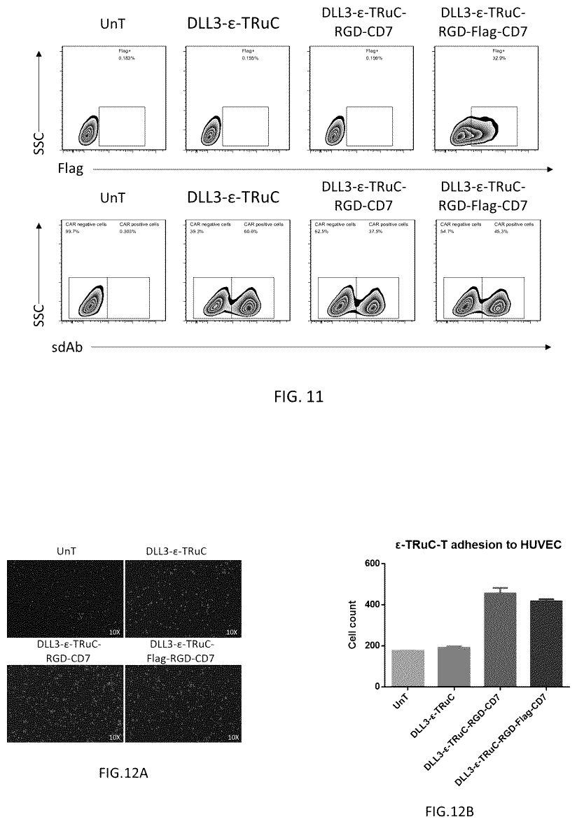

T cells were isolated from healthy donor PBMCs (HemaCare) using pan T cell isolation kit (Miltenyi Biotec, 130096535). Isolated T cells were cultured under AIMV (Gibco, 31035025) medium with 5% FBS (Gibco, 10099141) and further activated by CD3/CD28 activation beads (Miltenyi Biotec, 130091442) at a 2:1 ratio in the 37° C., 5% CO₂ incubator. 48 or 72 hours after initial activation, T cells were transduced with lentivirus expressing a DLL3 targeting ε-TRuC at proper multiplicity of infection (MOI) in the presence of 8 μg/ml polybrene (SIGMA-ALDRICH, H9268-10G). Additional IL-2 was supplemented to a final concentration of 300 IU/ml. Fresh medium was replaced 24 hours post lentiviral infection. Infected T cells were maintained under AIMV medium with 5% FBS and 300 IU/ml IL-2 at a cell density between 5×10⁵ to 1×10⁶ cells/ml. 4 days after transduction, the expression of ε-TRuC and RGD on the cell surface were determined by FACS. Specifically, 1×10⁵ transduced or untransduced T cells (unT) were collected by centrifugation at 300 g for 5 min. The cells were then re-suspended with 100 μl PBS containing PE-conjugated anti-Flag antibody (Biolegend, US) or iFlour488-Anti-sdAb (GenScript, China). Flag positive ratio represents RGD ratio on T cells, and sdAb positive ratio represents ε-TRuC ratio. As shown in FIG. 11, ε-TRuC positive ratio on DLL3-ε-TRuC, DLL3-ε-TRuC-RGD-CD7 and DLL3-ε-TRuC-RGD-Flag-CD7 were 60.6%, 37.5% and 45.3%. The mbRGDs expressed on the surface of DLL3-ε-TRuC-RGD-Flag-CD7 was 32.9%.

### Example 8: mbRGD Enhances ε-TRuC-T Cells Adhesion to Endothelial Cells

To investigate whether mbRGD can improve T cell adhesion to neo-vascular endothelial cells, endothelial cell adhesion assay was performed. In this assay, human umbilical vein endothelial cells (HUVEC) were used to represent neo-vascular endothelial cells. DLL3-ε-TRuC-RGD-CD7 and DLL3-ε-TRuC-RGD-Flag-CD7 were prepared and their adhesion to HUVEC were investigated. Briefly, 1×10⁵ HUVEC cells were seeded onto the bottom of a 24-well-plate to form a single layer. The next day, 3×10⁵ DLL3-ε-TRuC-RGD-CD7 and DLL3-ε-TRuC-RGD-Flag-CD7, DLL3-ε-TRuC, and untransduced T cells (unT) were labeled with CFSE and added to the single HUVEC layer respectively. After 30 min incubation, the unbounded T cells were carefully rinsed out with fresh medium. The bounded cell were imaged on a fluorescence microscopy and counted. As shown in FIGS. 12A and 12B, DLL3-ε-TRuC-RGD-CD7 and DLL3-ε-TRuC-RGD-Flag-CD7 had remarkably more cell adhesion to HUVEC than DLL3-ε-TRuC or unT cells. These results suggested that mbRGD can enhance DLL3-ε-TRuC-T cell adhesion to neo-vascular endothelial cells.

### Example 9: Influence of the mbRGD on Cytotoxicity and Cytokine Secretion of DLL3-ε-TRuC-T Cells Against Target Cells and Endothelial Cells

To investigate whether mbRGD influences the cytotoxicity of DLL3-ε-TRuC-T cells against target cells or endothelial cells, HUVEC and DLL3 positive cells SHP77 were used for evaluating the cytotoxicity and cytokine secretion of DLL3-ε-TruC-T cells with or without mbRGD. Specifically, SHP77 cells were co-incubated with DLL3-ε-TRuC-RGD-CD7 and DLL3-ε-TRuC-RGD-Flag-CD7, DLL3-ε-TRuC, and unT for 22 h. Then, LDH (lactate dehydrogenase) levels were measured. As shown in FIGS. 13A-13B, cytotoxicity levels of mbRGD armored ε-TRuC against both SHP77 and HUVEC were comparable to the conventional ε-TRuC-T cells. These results demonstrated that mbRGD has no influence on the cytotoxicity of ε-TRuC-T cells against target cells; meanwhile the mbRGD armored ε-TRuC-T cells did not cause cytotoxicity to endothelial cells. The supernatants of the co-cultures were then taken for IFN-γ measurement by HTFR methods. As shown in FIGS. 14A-14B, IFN-γ secretion of all the ε-TRuC-T cells is highly activated by SHP77 co-culture (FIG. 14A). None of the ε-TRuC-T cells showed increased IFN-γ secretion upon co-culture with HUVEC compared to ε-TRuC-T cells. These results demonstrated that mbRGD armored ε-TRuC-T cells are safe to endothelial cells.

### Example 10: Generation of CARs and CAR-T Cells Armored with Membrane Bound Asparagine-Glycine-Arginine (NGR)-Fusion Protein

Cloning of mbNGRs Armored CAR Constructs

In order to enhance T cells homing and infiltration into tumor, tumor homing peptides were fused with transmembrane domain, and functioned as membrane bound form, which were able to anchor to membrane of T cells.

We designed several membrane bound form of NGR (mbNGR) based on hinge and transmembrane domain of CD7, and used as armor with DLL3 specific CAR-T cells. One of the mbNGRs armored DLL3 CARs consisted of DLL3-CAR (SEQ ID NO: 8), P2A (SEQ ID NO: 9), NGR peptide (SEQ ID NO: 38), (G4S)₃ linker, CD7 hinge (SEQ ID NO: 13), and the transmembrane domain of CD7 (SEQ ID NO: 14). And this construct was designated as DLL3-CAR-NGR-CD7 (SEQ ID NO: 39). Other one of the mbNGR armored DLL3 CARs consisted of DLL3 CAR (SEQ ID NO: 8), P2A (SEQ ID NO: 9), NGR peptide (SEQ ID NO: 38), Flag tag (SEQ ID NO: 3), (G45)₃ linker, CD7 hinge (SEQ ID NO: 13), and the transmembrane domain of CD7 (SEQ ID NO: 14). This construct was designated as DLL3-CAR-NGR-Flag-CD7 (SEQ ID NO: 40). Sequences of the related elements and CARs are shown in Tables 1, 2 and 4.

TABLE 4
Modalities and sequences of mbNGR armored DLL3 CARS

| SEQ ID NO | Modality | Sequence |
|---|---|---|
| 38 | NGR | CNGRCG |
| 39 | DLL3- | MALPVTALLLPLALLLHAARPQVQLVESGGGVVQPGGSLRLSCAASFSGY |
| | CAR- | GVSTMAWFRQAPGKGLEGVAAITVGSGNTYYADSVKGRFTISRDNSKNT |
| | NGR-CD7 | VYLQMNSLRAEDTAMYYCAVGYLSGGSWDVPGRYNYWGQGTLVTVSS |
| | | GGGGSGGGGSGGGGSEVQLVESGGGLVQPGGSLRLSCAASGNTYSSNYM |
| | | GWFRQAPGKGLEEVAVIYTRGGHTYYVDSVRGRFTISQDNAKNSLYLQM |
| | | NSLRAEDTAVYYCAASSRHRLGLNNPRDYDYWGQGTLVTVSSTTTPAPR |
| | | PPTPAPTIASQPLSLRPEACRPAAGGAVHTRGLDFACDIYIWAPLAGTCGV |
| | | LLLSLVITLYCKRGRKKLLYIFKQPFMRPVQTTQEEDGCSCRFPEEEEGGC |
| | | ELRVKFSRSADAPAYKQGQNQLYNELNLGRREEYDVLDKRRGRDPEMG |
| | | GKPRRKNPQEGLYNELQKDKMAEAYSEIGMKGERRRGKGHDGLYQGLS |
| | | TATKDTYDALHMQALPPRGSGATNFSLLKQAGDVEENPGPMPMGSLQPL |
| | | ATLYLLGMLVASCLGCNGRCGGGGGSGGGGSPPRASALPAPPTGSALPDP |
| | | QTASALPDPPAASALPAALAVISFLLGLGLGVACVLA |
| 40 | DLL3- | MALPVTALLLPLALLLHAARPQVQLVESGGGVVQPGGSLRLSCAASFSGY |
| | CAR- | GVSTMAWFRQAPGKGLEGVAAITVGSGNTYYADSVKGRFTISRDNSKNT |
| | NGR-Flag- | VYLQMNSLRAEDTAMYYCAVGYLSGGSWDVPGRYNYWGQGTLVTVSS |
| | CD7 | GGGGSGGGGSGGGGSEVQLVESGGGLVQPGGSLRLSCAASGNTYSSNYM |
| | | GWFRQAPGKGLEEVAVIYTRGGHTYYVDSVRGRFTISQDNAKNSLYLQM |
| | | NSLRAEDTAVYYCAASSRHRLGLNNPRDYDYWGQGTLVTVSSTTTPAPR |
| | | PPTPAPTIASQPLSLRPEACRPAAGGAVHTRGLDFACDIYIWAPLAGTCGV |
| | | LLLSLVITLYCKRGRKKLLYIFKQPFMRPVQTTQEEDGCSCRFPEEEEGGC |
| | | ELRVKFSRSADAPAYKQGQNQLYNELNLGRREEYDVLDKRRGRDPEMG |
| | | GKPRRKNPQEGLYNELQKDKMAEAYSEIGMKGERRRGKGHDGLYQGLS |
| | | TATKDTYDALHMQALPPRGSGATNFSLLKQAGDVEENPGPMPMGSLQPL |
| | | ATLYLLGMLVASCLGCNGRCGGGGGSGGGGSDYKDDDDKPPRASALPAP |
| | | PTGSALPDPQTASALPDPPAASALPAALAVISFLLGLGLGVACVLA |

To demonstrate that mbNGRs armor is applicable to different tumor models, we designed several membrane bound form of NGR (mbNGR) comprising the hinge and transmembrane domains of CD7, and used as armor with MSLN/GPC2 specific CAR-T cells. One of the mbNGRs armored CARs consisted of MSLN-CAR (SEQ ID NO: 41), P2A (SEQ ID NO: 9), NGR peptide (SEQ ID NO: 38), (G4S)₃ linker, CD7 hinge (SEQ ID NO: 13), the and transmembrane domain of CD7 (SEQ ID NO: 14). This construct was designated as MSLN-CAR-NGR-CD7 (SEQ ID NO: 42). Another of the mbNGR armored CARs consisted of MSLN CAR (SEQ ID NO: 41), P2A (SEQ ID NO: 9), NGR peptide (SEQ ID NO: 38), Flag tag (SEQ ID NO: 3), (G45)₃ linker, CD7 hinge (SEQ ID NO: 13), and the transmembrane domain of CD7 (SEQ ID NO: 14). This construct was designated as MSLN-CAR-NGR-Flag-CD7 (SEQ ID NO: 43). Another of the mbNGRs armored CARs consisted of GPC2-CAR (SEQ ID NO: 44), P2A (SEQ ID NO: 9), NGR peptide (SEQ ID NO: 38), (G4S)₃ linker, CD7 hinge (SEQ ID NO: 13), and the transmembrane domain of CD7 (SEQ ID NO: 14). This construct was designated as GPC2-CAR-NGR-CD7 (SEQ ID NO: 45). Another of the mbNGR armored CARs consisted of GPC2 CAR (SEQ ID NO: 43), P2A (SEQ ID NO: 9), NGR peptide (SEQ ID NO: 38), Flag tag (SEQ ID NO: 3), (G45)₃ linker, CD7 hinge (SEQ ID NO: 13), and the transmembrane domain of CD7 (SEQ ID NO: 14). This construct was designated as GPC2-CAR-NGR-Flag-CD7 (SEQ ID NO: 46). Sequences of the related elements and CARs are shown in Tables 1, 2, 4 and 5.

TABLE 5
Modalities and sequences of mbNGR armored MSLN/GPC2 CARS

| SEQ ID NO | Modality | Sequence |
|---|---|---|
| 41 | MSLN- | MALPVTALLLPLALLLHAARPQVQLVESGGGVVQPGGSLRLSCAASGLSF |
| | CAR | STYTVAWFRQAPGKGLEGVAAIPYTSQHMVYTDSVKGRFTISRDNSKNTL |
| | | YLQMNSLRAEDTAVYYCATDRRPGTSMLAINGYNRWGQGTTVTVSSTTT |
| | | PAPRPPTPAPTIASQPLSLRPEACRPAAGGAVHTRGLDFACDIYIWAPLAGT |
| | | CGVLLLSLVITLYCKRGRKKLLYIFKQPFMRPVQTTQEEDGCSCRFPEEEE |
| | | GGCELRVKFSRSADAPAYKQGQNQLYNELNLGRREEYDVLDKRRGRDPE |
| | | MGGKPRRKNPQEGLYNELQKDKMAEAYSEIGMKGERRRGKGHDGLYQG |
| | | LSTATKDTYDALHMQALPPR |
| 42 | MSLN- | MALPVTALLLPLALLLHAARPQVQLVESGGGVVQPGGSLRLSCAASGLSF |
| | CAR- | STYTVAWFRQAPGKGLEGVAAIPYTSQHMVYTDSVKGRFTISRDNSKNTL |
| | NGR-CD7 | YLQMNSLRAEDTAVYYCATDRRPGTSMLAINGYNRWGQGTTVTVSSTTT |
| | | PAPRPPTPAPTIASQPLSLRPEACRPAAGGAVHTRGLDFACDIYIWAPLAGT |
| | | CGVLLLSLVITLYCKRGRKKLLYIFKQPFMRPVQTTQEEDGCSCRFPEEEE |
| | | GGCELRVKFSRSADAPAYKQGQNQLYNELNLGRREEYDVLDKRRGRDPE |
| | | MGGKPRRKNPQEGLYNELQKDKMAEAYSEIGMKGERRRGKGHDGLYQG |
| | | LSTATKDTYDALHMQALPPRGSGATNFSLLKQAGDVEENPGPMPMGSLQ |
| | | PLATLYLLGMLVASCLGCNGRCGGGGGSGGGGSGGGGSPPRASALPAPPT |
| | | GSALPDPQTASALPDPPAASALPAALAVISFLLGLGLGVACVLA |
| 43 | MSLN- | MALPVTALLLPLALLLHAARPQVQLVESGGGVVQPGGSLRLSCAASGLSF |
| | CAR- | STYTVAWFRQAPGKGLEGVAAIPYTSQHMVYTDSVKGRFTISRDNSKNTL |
| | NGR-Flag- | YLQMNSLRAEDTAVYYCATDRRPGTSMLAINGYNRWGQGTTVTVSSTTT |
| | CD7 | PAPRPPTPAPTIASQPLSLRPEACRPAAGGAVHTRGLDFACDIYIWAPLAGT |
| | | CGVLLLSLVITLYCKRGRKKLLYIFKQPFMRPVQTTQEEDGCSCRFPEEEE |
| | | GGCELRVKFSRSADAPAYKQGQNQLYNELNLGRREEYDVLDKRRGRDPE |
| | | MGGKPRRKNPQEGLYNELQKDKMAEAYSEIGMKGERRRGKGHDGLYQG |
| | | LSTATKDTYDALHMQALPPRGSGATNFSLLKQAGDVEENPGPMPMGSLQ |
| | | PLATLYLLGMLVASCLGCNGRCGGGGGSGGGGSGGGGSDYKDDDDKPP |
| | | RASALPAPPTGSALPDPQTASALPDPPAASALPAALAVISFLLGLGLGVAC |
| | | VLA |
| 44 | GPC2- | MALPVTALLLPLALLLHAARPQVQLVESGGGSAQSGGSLRLSCAISEFTYK |
| | CAR | NTCVGWFRQAPGKEREGVAAIDSDGNTNYVDSVKGRFTISQGNAKNTVY |
| | | LQMNSLKPEDTAMYYCAAGAYCGRLLLWIGNYAYWGQGTQVTVSSTTT |
| | | PAPRPPTPAPTIASQPLSLRPEACRPAAGGAVHTRGLDFACDIYIWAPLAGT |
| | | CGVLLLSLVITLYCKRGRKKLLYIFKQPFMRPVQTTQEEDGCSCRFPEEEE |
| | | GGCELRVKFSRSADAPAYKQGQNQLYNELNLGRREEYDVLDKRRGRDPE |
| | | MGGKPRRKNPQEGLYNELQKDKMAEAYSEIGMKGERRRGKGHDGLYQG |
| | | LSTATKDTYDALHMQALPPR |
| 45 | GPC2- | MALPVTALLLPLALLLHAARPQVQLVESGGGSAQSGGSLRLSCAISEFTYK |
| | CAR- | NTCVGWFRQAPGKEREGVAAIDSDGNTNYVDSVKGRFTISQGNAKNTVY |
| | NGR-CD7 | LQMNSLKPEDTAMYYCAAGAYCGRLLLWIGNYAYWGQGTQVTVSSTTT |
| | | PAPRPPTPAPTIASQPLSLRPEACRPAAGGAVHTRGLDFACDIYIWAPLAGT |
| | | CGVLLLSLVITLYCKRGRKKLLYIFKQPFMRPVQTTQEEDGCSCRFPEEEE |
| | | GGCELRVKFSRSADAPAYKQGQNQLYNELNLGRREEYDVLDKRRGRDPE |
| | | MGGKPRRKNPQEGLYNELQKDKMAEAYSEIGMKGERRRGKGHDGLYQG |
| | | LSTATKDTYDALHMQALPPRGSGATNFSLLKQAGDVEENPGPMPMGSLQ |
| | | PLATLYLLGMLVASCLGCNGRCGGGGGSGGGGSGGGGSPPRASALPAPPT |
| | | GSALPDPQTASALPDPPAASALPAALAVISFLLGLGLGVACVLA |
| 46 | GPC2- | MALPVTALLLPLALLLHAARPQVQLVESGGGSAQSGGSLRLSCAISEFTYK |
| | CAR- | NTCVGWFRQAPGKEREGVAAIDSDGNTNYVDSVKGRFTISQGNAKNTVY |
| | NGR-Flag- | LQMNSLKPEDTAMYYCAAGAYCGRLLLWIGNYAYWGQGTQVTVSSTTT |
| | CD7 | PAPRPPTPAPTIASQPLSLRPEACRPAAGGAVHTRGLDFACDIYIWAPLAGT |
| | | CGVLLLSLVITLYCKRGRKKLLYIFKQPFMRPVQTTQEEDGCSCRFPEEEE |
| | | GGCELRVKFSRSADAPAYKQGQNQLYNELNLGRREEYDVLDKRRGRDPE |
| | | MGGKPRRKNPQEGLYNELQKDKMAEAYSEIGMKGERRRGKGHDGLYQG |
| | | LSTATKDTYDALHMQALPPRGSGATNFSLLKQAGDVEENPGPMPMGSLQ |
| | | PLATLYLLGMLVASCLGCNGRCGGGGGSGGGGSGGGGSDYKDDDDKPP |
| | | RASALPAPPTGSALPDPQTASALPDPPAASALPAALAVISFLLGLGLGVAC |
| | | VLA |

Generation of mbNGR Armored CAR-T Cell

### 1. Preparation of Lentivirus

The sequences encoding the NGR armored DLL3/MSLN/GPC2 CARs as described above were codon optimized and synthesized by GenScript. Then the sequences were incorporated into 2n d generation lentivirus vectors via standard molecular clone methods. All plasmid sequences were verified via sequencing. Then the plasmids and lentiviral packaging plasmids were co-transfected into HEK293 cells to produce lentivirus. Virus particles were collected from the supernatant, and concentrated by PEG precipitation or ultracentrifugation. The concentrated lentiviral particles were used to transduce T cells.

### 2. T Cell Transduction

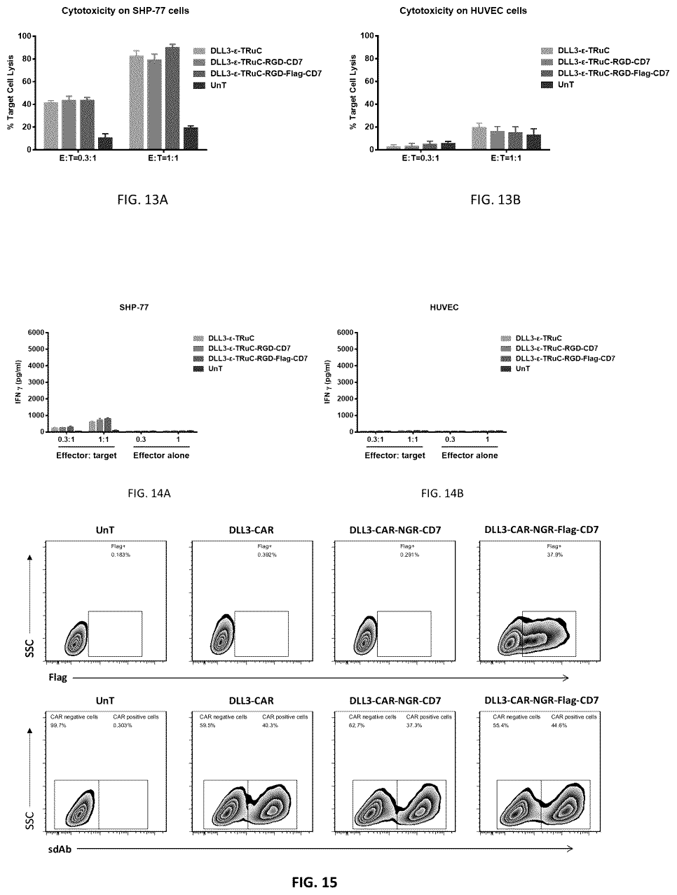

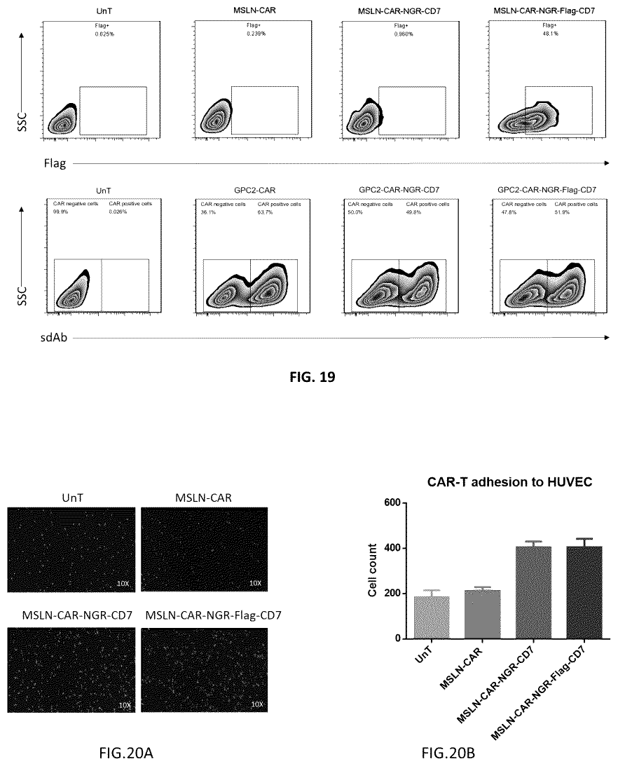

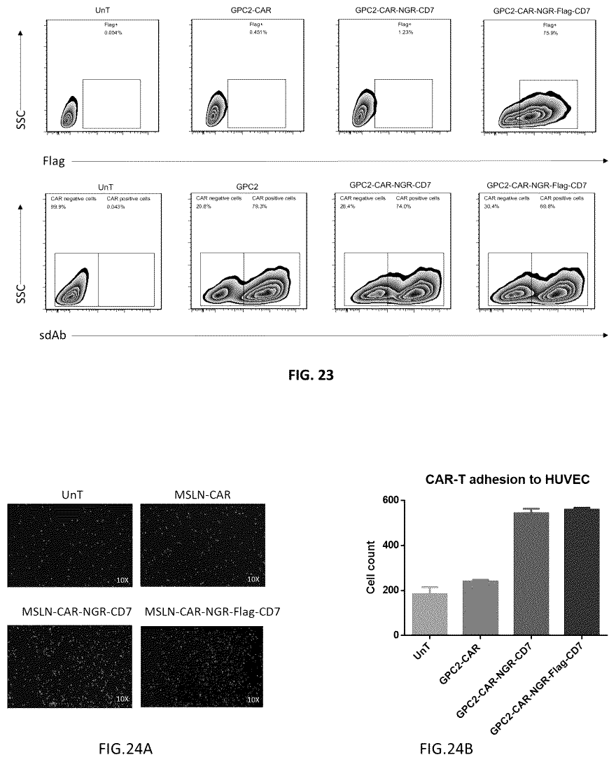

T cells were isolated from healthy donor PBMCs (HemaCare) using pan T cell isolation kit (Miltenyi Biotec, 130096535). Isolated T cells were cultured under AIMV (Gibco, 31035025) medium with 5% FBS (Gibco, 10099141) and further activated by CD3/CD28 activation beads (Miltenyi Biotec, 130091442) at a 2:1 ratio in the 37° C., 5% CO₂ incubator. 48 or 72 hours after initial activation, T cells were transduced with lentivirus expressing a DLL3/MSLN/GPC2 targeting CAR at proper multiplicity of infection (MOI) in the presence of 8 μg/ml polybrene (SIGMA-ALDRICH, H9268-10G). Additional IL-2 was supplemented to a final concentration of 300 IU/ml. Fresh medium was replaced 24 hours post lentiviral infection. Infected T cells were maintained under AIMV medium with 5% FBS and 300 IU/ml IL-2 at a cell density between 5×10⁵ to 1×10⁶ cells/ml. 4 days after transduction, the expression of CAR and NGR on the cell surface were determined by FACS. Specifically, 1×10⁵ transduced or untransduced T cells (unT) were collected by centrifugation at 300 g for 5 min. The cells were then re-suspended with 100 μl PBS containing PE-conjugated anti-Flag antibody (Biolegend, US) or iFlour488-Anti-sdAb (GenScript, China). Flag positive ratio represents NGR ratio on T cells, and sdAb positive ratio represents CAR ratio. As shown in FIG. 15, CAR positive ratio on DLL3-CAR, DLL3-CAR-NGR-CD7 and DLL3-CAR-NGR-Flag-CD7 were 41.3%, 37.3% and 44.6%. The mbNGRs expressed on the surface of DLL3-CAR-NGR-Flag-CD7 was 37.9%. As shown in FIG. 19, CAR positive ratio on MSLN-CAR, MSLN-CAR-NGR-CD7 and MSLN-CAR-NGR-Flag-CD7 were 63.7%, 49.8% and 51.9%. The mbNGRs expressed on the surface of MSLN-CAR-NGR-Flag-CD7 was 48.1%. As shown in FIG. 23, CAR positive ratio on GPC2-CAR, GPC2-CAR-NGR-CD7 and GPC2-CAR-NGR-Flag-CD7 were 79.3%, 74% and 69.8%. The mbNGRs expressed on the surface of GPC2-CAR-NGR-Flag-CD7 was 75.9%.

### Example 11: mbNGR Enhances DLL3/MSLN/GPC2 CAR-T Cells Adhesion to Endothelial Cells

To investigate whether mbNGR can improve MSLN CAR-T cell adhesion to neo-vascular endothelial cells, endothelial cell adhesion assay was performed. In this assay, human umbilical vein endothelial cells (HUVEC) were used to represent neo-vascular endothelial cells. MSLN-CAR-NGR-CD7 and MSLN-CAR-NGR-Flag-CD7 were prepared and their adhesion to HUVEC were investigated. Briefly, 1×10⁵ HUVEC cells were seeded onto the bottom of a 24-well-plate to form a single layer. The next day, 3×10⁵ MSLN-CAR-NGR-CD7, MSLN-CAR-NGR-Flag-CD7, MSLN-CAR, and untransduced T cells (unT) were labeled with CF SE and added to the single HUVEC layer respectively. After 30 min incubation, the unbounded T cells were carefully rinsed out with fresh medium. The bounded cell were imaged on a fluorescence microscopy and counted. As shown in FIGS. 20A and 20B, MSLN-CAR-NGR-CD7 and MSLN-CAR-NGR-Flag-CD7 had remarkably more cell adhesion to HUVEC than MSLN CAR or unT cells. These results suggested that mbNGR can enhance MSLN CAR-T cell adhesion to neo-vascular endothelial cells.

To investigate whether mbNGR can improve GPC2 CAR-T cell adhesion to neo-vascular endothelial cells, endothelial cell adhesion assay was performed. In this assay, human umbilical vein endothelial cells (HUVEC) were used to represent neo-vascular endothelial cells. GPC2-CAR-NGR-CD7 and GPC2-CAR-NGR-Flag-CD7 were prepared and their adhesion to HUVEC were investigated. Briefly, 1×10⁵ HUVEC cells were seeded onto the bottom of a 24-well-plate to form a single layer. The next day, 3×10⁵ GPC2-CAR-NGR-CD7, GPC2-CAR-NGR-Flag-CD7, GPC2-CAR, and untransduced T cells (unT) were labeled with CFSE and added to the single HUVEC layer respectively. After 30 min incubation, the unbounded T cells were carefully rinsed out with fresh medium. The bounded cell were imaged on a fluorescence microscopy and counted. As shown in FIGS. 24A and 24B, GPC2-CAR-NGR-CD7 and GPC2-CAR-NGR-Flag-CD7 had remarkably more cell adhesion to HUVEC than GPC2 CAR or unT cells. These results suggested that mbNGR can enhance GPC2 CAR-T cell adhesion to neo-vascular endothelial cells.

### Example 12: Influence of the mbNGR on Cytotoxicity and Cytokine Secretion of DLL3/MSLN/GPC2 CAR-T Cells Against Target Cells and Endothelial Cells

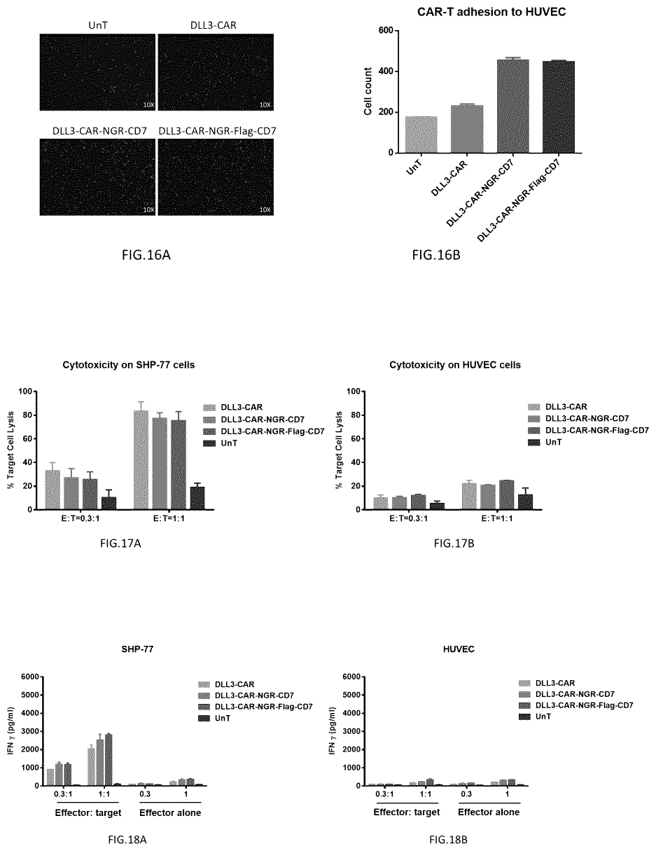

To investigate whether mbNGR influences the cytotoxicity of DLL3 CAR-T cells against target cells or endothelial cells, HUVEC and DLL3 positive cells SHP77 were used for evaluating the cytotoxicity and cytokine secretion of DLL3-CAR-T cells with or without mbNGR. Specifically, SHP77 cells were co-incubated with DLL3-CAR-NGR-CD7, DLL3-CAR-NGR-Flag-CD7, DLL3-CAR, and unT for 22 h. Then, LDH (lactate dehydrogenase) levels were measured. As shown in FIGS. 17A-17B, cytotoxicity levels of mbNGR armored CAR-T against both SHP77 and HUVEC were comparable to the conventional CAR-T cells. These results demonstrated that mbNGR has no influence on the cytotoxicity of CAR-T cells against target cells; meanwhile the mbNGR armored CAR-T cells did not cause cytotoxicity to endothelial cells. The supernatants of the co-cultures were then taken for IFN-γ measurement by HTFR methods. As shown in FIGS. 18A-18B, IFN-γ secretion of all DLL3 CAR-T cells is highly activated by SHP77 co-culture (FIG. 18A). None of the DLL3 CAR-T cells showed increased IFN-γ secretion upon co-culture with HUVEC compared to DLL3 CAR-T cells only. These results demonstrated that mbNGR armored DLL3 CAR-T cells are safe to endothelial cells. To investigate whether mbNGR can improve DLL3 CAR-T cell adhesion to neo-vascular endothelial cells, endothelial cell adhesion assay was performed. In this assay, human umbilical vein endothelial cells (HUVEC) were used to represent neo-vascular endothelial cells. DLL3-CAR-NGR-CD7 and DLL3-CAR-NGR-Flag-CD7 were prepared and their adhesion to HUVEC were investigated. Briefly, 1×10⁵ HUVEC cells were seeded onto the bottom of a 24-well-plate to form a single layer. The next day, 3×10⁵ DLL3-CAR-NGR-CD7, DLL3-CAR-NGR-Flag-CD7, DLL3-CAR, and untransduced T cells (unT) were labeled with CFSE and added to the single HUVEC layer respectively. After 30 min incubation, the unbounded T cells were carefully rinsed out with fresh medium. The bounded cell were imaged on a fluorescence microscopy and counted. As shown in FIGS. 16A and 16B, DLL3-CAR-NGR-CD7 and DLL3-CAR-NGR-Flag-CD7 had remarkably more cell adhesion to HUVEC than DLL3 CAR or unT cells. These results suggested that mbNGR can enhance DLL3 CAR-T cell adhesion to neo-vascular endothelial cells.

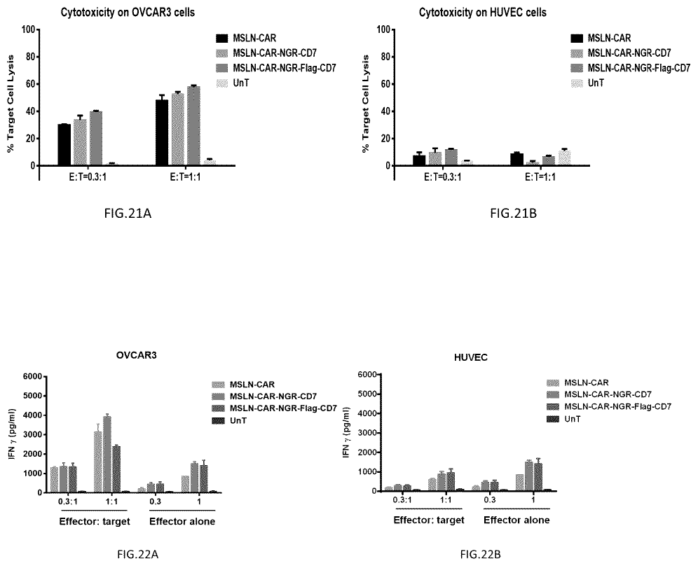

To investigate whether mbNGR influences the cytotoxicity of MSLN CAR-T cells against target cells or endothelial cells, HUVEC and MSLN positive cells OVCAR3 were used for evaluating the cytotoxicity and cytokine secretion of MSLN-CAR-T cells with or without mbNGR. Specifically, OVCAR3 cells were co-incubated with MSLN-CAR-NGR-CD7, MSLN-CAR-NGR-Flag-CD7, MSLN-CAR, and unT for 22 h. Then, LDH (lactate dehydrogenase) levels were measured. As shown in FIGS. 21A-21B, cytotoxicity levels of mbNGR armored CAR-T against both OVCAR3 and HUVEC were comparable to the conventional CAR-T cells. These results demonstrated that mbNGR has no influence on the cytotoxicity of CAR-T cells against target cells; meanwhile the mbNGR armored CAR-T cells did not cause cytotoxicity to endothelial cells. The supernatants of the co-cultures were then taken for IFN-γ measurement by HTFR methods. As shown in FIGS. 22A-22B, IFN-γ secretion of all MSLN CAR-T cells is highly activated by OVCAR3 co-culture (FIG. 22A). None of the MSLN CAR-T cells showed increased IFN-γ secretion upon co-culture with HUVEC compared to MSLN CAR-T cells only. These results demonstrated that mbNGR armored MSLN CAR-T cells are safe to endothelial cells.

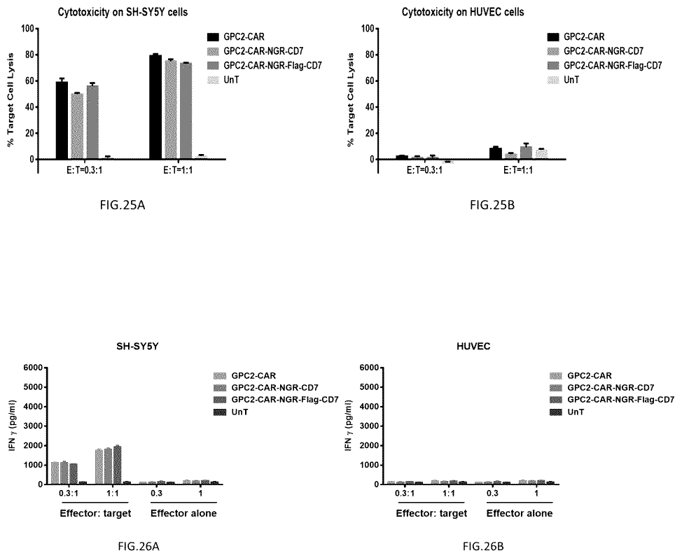

To investigate whether mbNGR influences the cytotoxicity of GPC2 CAR-T cells against target cells or endothelial cells, HUVEC and GPC2 positive cells SH-SY5Y were used for evaluating the cytotoxicity and cytokine secretion of GPC2-CAR-T cells with or without mbNGR. Specifically, SH-SY5Y cells were co-incubated with GPC2-CAR-NGR-CD7, GPC2-CAR-NGR-Flag-CD7, GPC2-CAR, and unT for 22 h. Then, LDH (lactate dehydrogenase) levels were measured. As shown in FIGS. 25A-25B, cytotoxicity levels of mbNGR armored CAR-T against both SH-SY5Y and HUVEC were comparable to the conventional CAR-T cells. These results demonstrated that mbNGR has no influence on the cytotoxicity of CAR-T cells against target cells; meanwhile the mbNGR armored CAR-T cells did not cause cytotoxicity to endothelial cells. The supernatants of the co-cultures were then taken for IFN-γ measurement by HTFR methods. As shown in FIGS. 26A-26B, IFN-γ secretion of all GPC2 CAR-T cells is highly activated by SH-SY5Y co-culture (FIG. 26A). None of the GPC2 CAR-T cells showed increased IFN-γ secretion upon co-culture with HUVEC compared to GPC2 CAR-T cells only. These results demonstrated that mbNGR armored GPC2 CAR-T cells are safe to endothelial cells.

### Example 13: Generation of RGD Embedding CARs and CAR-T Cells

### Cloning of RGD Embedding CAR Constructs

In order to analyze the similarities and differences between RGD embedding CAR-T and mbRGD armored CAR-T cellular functions, we designed several RGD embedding DLL3 specific CAR-T cells. They were the RGD-(G4S)₃-DLL3 sdAb-CD8α hinge-CD8α TM-4-1BB-CD3z (SEQ ID NO: 49) and the DLL3 sdAb-(G4S)₃-RGD-CD8α hinge-CD8α TM-4-1BB-CD3z (SEQ ID NO: 50). Sequences of the related CARs were shown in Table 6.

TABLE 6
Modalities and sequences of RGD embedding CARs

| SEQ ID NO | Modality | Sequence |
|---|---|---|
| 47 | CD8α hinge | TTTPAPRPPTPAPTIASQPLSLRPEACRPAAGGAVHTRGLDFACD |
| 48 | CD8α TM | IYIWAPLAGTCGVLLLSLVIT |
| 49 | RGD- | MALPVTALLLPLALLLHAARPCDCRGDCFCGGGGSGGGGSGGGGSQVQ |
| | (G4S)₃- | LVESGGGVVQPGGSLRLSCAASFSGYGVSTMAWFRQAPGKGLEGVAAI |
| | DLL3 sdAb- | TVGSGNTYYADSVKGRFTISRDNSKNTVYLQMNSLRAEDTAMYYCAVG |
| | CD8α hinge- | YLSGGSWDVPGRYNYWGQGTLVTVSSGGGGSGGGGSGGGGSEVQLVE |
| | CD8 TM-4- | SGGGLVQPGGSLRLSCAASGNTYSSNYMGWFRQAPGKGLEEVAVIYTR |
| | 1BB-CD3z | GGHTYYVDSVRGRFTISQDNAKNSLYLQMNSLRAEDTAVYYCAASSRH |
| | | RLGLNNPRDYDYWGQGTLVTVSSTTTPAPRPPTPAPTIASQPLSLRPEAC |
| | | RPAAGGAVHTRGLDFACDIYIWAPLAGTCGVLLLSLVITLYCKRGRKKL |
| | | LYIFKQPFMRPVQTTQEEDGCSCRFPEEEEGGCELRVKFSRSADAPAYKQ |
| | | GQNQLYNELNLGRREEYDVLDKRRGRDPEMGGKPRRKNPQEGLYNEL |
| | | QKDKMAEAYSEIGMKGERRRGKGHDGLYQGLSTATKDTYDALHMQAL |
| | | PPR |
| 50 | DLL3 sdAb- | MALPVTALLLPLALLLHAARPQVQLVESGGGVVQPGGSLRLSCAASFSG |
| | (G4S)₃- | YGVSTMAWFRQAPGKGLEGVAAITVGSGNTYYADSVKGRFTISRDNSK |
| | RGD-CD8α | NTVYLQMNSLRAEDTAMYYCAVGYLSGGSWDVPGRYNYWGQGTLVT |
| | hinge-CD8 | VSSGGGGSGGGGSGGGGSEVQLVESGGGLVQPGGSLRLSCAASGNTYSS |
| | TM-4-1BB- | NYMGWFRQAPGKGLEEVAVIYTRGGHTYYVDSVRGRFTISQDNAKNSL |
| | CD3z | YLQMNSLRAEDTAVYYCAASSRHRLGLNNPRDYDYWGQGTLVTVSSG |
| | | GGGSGGGGSGGGGSCDCRGDCFCTTTPAPRPPTPAPTIASQPLSLRPEAC |
| | | RPAAGGAVHTRGLDFACDIYIWAPLAGTCGVLLLSLVITLYCKRGRKKL |
| | | LYIFKQPFMRPVQTTQEEDGCSCRFPEEEEGGCELRVKFSRSADAPAYKQ |
| | | GQNQLYNELNLGRREEYDVLDKRRGRDPEMGGKPRRKNPQEGLYNEL |
| | | QKDKMAEAYSEIGMKGERRRGKGHDGLYQGLSTATKDTYDALHMQAL |
| | | PPR |

### Generation of RGD Embedding CAR-T Cell

### 1. Preparation of Lentivirus

The sequences encoding the RGD embedding DLL3-CARs as described above were codon optimized and synthesized by GenScript. Then the sequences were incorporated into 3^{rd} generation lentivirus vectors via standard molecular clone methods. All plasmid sequences were verified via sequencing. Then the plasmids and lentiviral packaging plasmids were co-transfected into HEK293 cells to produce lentivirus. Virus particles were collected from the supernatant, and concentrated by PEG precipitation or ultracentrifugation. The concentrated lentiviral particles were used to transduce T cells.

### 2. T Cell Transduction

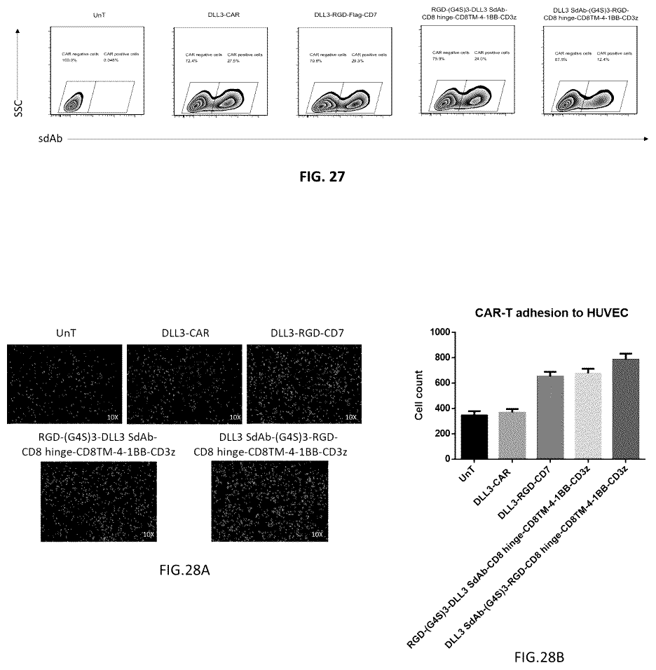

T cells were isolated from healthy donor PBMCs (HemaCare) using pan T cell isolation kit (Miltenyi Biotec, 130096535). Isolated T cells were cultured under AIMV (Gibco, 31035025) medium with 5% FBS (Gibco, 10099141) and further activated by CD3/CD28 activation beads (Miltenyi Biotec, 130091442) at a 2:1 ratio in the 37° C., 5% CO₂ incubator. 48 or 72 hours after initial activation, T cells were transduced with lentivirus expressing a DLL3 targeting CAR at proper multiplicity of infection (MOI) in the presence of 8 μg/ml polybrene (SIGMA-ALDRICH, H9268-10G). Additional IL-2 was supplemented to a final concentration of 300 IU/ml. Fresh medium was replaced 24 hours post lentiviral infection. Infected T cells were maintained under AIMV medium with 5% FBS and 300 IU/ml IL-2 at a cell density between 5×10⁵ to 1×10⁶ cells/ml. 4 days after transduction, the expression of CAR on the cell surface were determined by FACS. Specifically, 1×10⁵ transduced or untransduced T cells (unT) were collected by centrifugation at 300 g for 5 min. The cells were then re-suspended with 100 μl PBS containing iFlour488-Anti-sdAb (GenScript, China). SdAb positive ratio represents CAR ratio. As shown in FIG. 27, CAR positive ratio on DLL3-CAR, DLL3-RGD-CD7, RGD-(G4S)₃-DLL3 sdAb-CD8 hinge-CD8TM-4-1BB-CD3z and DLL3 sdAb-(G4S)₃-RGD-CD8 hinge-CD8 TM-4-1BB-CD3z were 27.5%, 29.3%, 24.0% and 12.4%.

### Example 14: RGD Embedding CAR Enhances CAR-T Cells Adhesion to Endothelial Cells

To investigate whether RGD embedding CAR can improve T cell adhesion to neo-vascular endothelial cells, endothelial cell adhesion assay was performed. In this assay, human umbilical vein endothelial cells (HUVEC) were used to represent neo-vascular endothelial cells. DLL3-RGD-CD7, RGD-(G4S)₃-DLL3 sdAb-CD8 hinge-CD8 TM-4-1BB-CD3z and DLL3 sdAb-(G4S)₃-RGD-CD8 hinge-CD8 TM-4-1BB-CD3z were prepared and their adhesion to HUVEC were investigated. Briefly, 1×10⁵ HUVEC cells were seeded onto the bottom of a 24-well-plate to form a single layer. The next day, 3×10⁵ DLL3-CAR, DLL3-RGD-CD7, RGD-(G4S)₃-DLL3 sdAb-CD8 hinge-CD8TM-4-1BB-CD3z, DLL3 sdAb-(G4S)₃-RGD-CD8 hinge-CD8 TM-4-1BB-CD3z and untransduced T cells (unT) were labeled with CFSE and added to the single HUVEC layer respectively. After 30 min incubation, the unbounded T cells were carefully rinsed out with fresh medium. The bounded cell were imaged on a fluorescence microscopy and counted. As shown in FIGS. 28A and 28B, RGD-(G4S)₃-DLL3 sdAb-CD8 hinge-CD8TM-4-1BB-CD3z, DLL3 sdAb-(G4S)₃-RGD-CD8 hinge-CD8TM-4-1BB-CD3z and DLL3-RGD-CD7 had remarkably more cell adhesion to HUVEC than DLL3 CAR or unT cells. These results suggested that both RGD embedding CAR and mbRGD armored CAR can enhance CAR-T cell adhesion to neo-vascular endothelial cells.

### Example 15: Influence of the RGD Embedding CAR on Cytotoxicity and Cytokine Secretion of CAR-T Cells Against Target Cells and Endothelial Cells

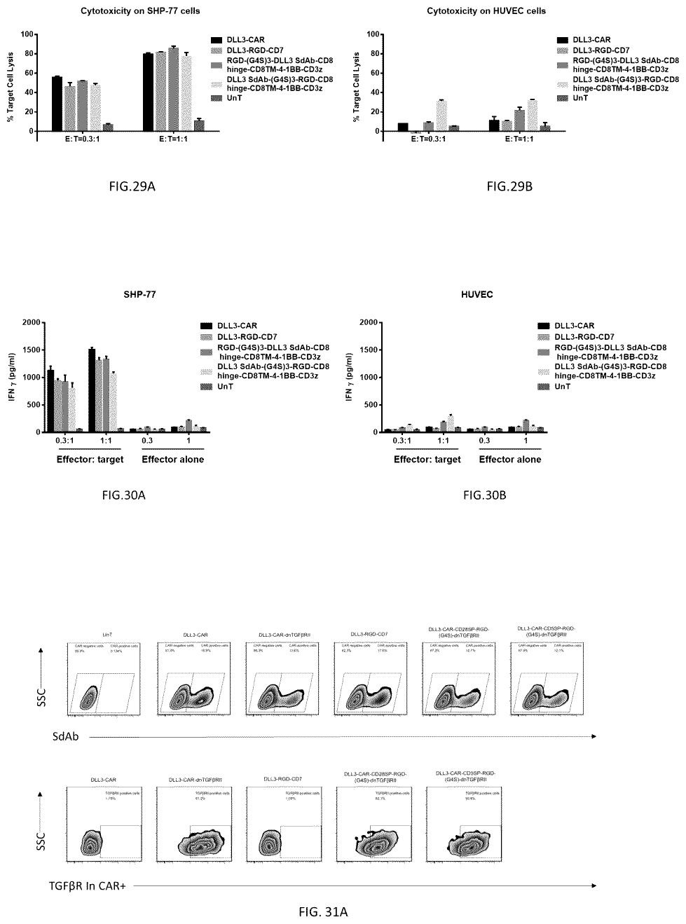

To investigate whether RGD embedding CAR influences the cytotoxicity of CAR-T cells against target cells or endothelial cells, HUVEC and DLL3 positive cells SHP77 were used for evaluating the cytotoxicity and cytokine secretion of DLL3-CAR-T, RGD embedding CAR-T and mbRGD armored CAR-T cells. Specifically, SHP77 cells were co-incubated with RGD-(G4S)₃-DLL3 sdAb-CD8 hinge-CD8 TM-4-1BB-CD3z, DLL3 sdAb-(G4S)₃-RGD-CD8 hinge-CD8 TM-4-1BB-CD3z, DLL3-RGD-CD7, DLL3-CAR, and unT for 22 h. Then, LDH (lactate dehydrogenase) levels were measured. As shown in FIGS. 29A-29B, cytotoxicity levels of RGD embedding CAR-T against SHP77 were comparable to the mbRGD armored CAR-T and conventional CAR-T cells. Cytotoxicity levels of RGD embedding CAR-T to HUVEC was higher than that of mbRGD armored CAR-T and conventional CAR-T cells. These results demonstrated that RGD embedding CAR has no influence on the cytotoxicity of CAR-T cells against target cells; but the RGD embedding CAR-T cells caused cytotoxicity to endothelial cells. The supernatants of the co-cultures were then taken for IFN-γ measurement by HTFR methods. As shown in FIGS. 30A-30B, IFN-γ secretion of all DLL3 CAR-T cells is highly activated by SHP77 co-culture (FIG. 30A). The RGD embedding CAR-T cells showed increased IFN-γ secretion upon co-culture with HUVEC compared to DLL3 CAR-T cells only. These results demonstrated that RGD embedding CAR-T cells aren't safe to endothelial cells.

### Example 16: Generation of mbRGD/mbNGR Combined TGF-β DNR Armored CARs and CAR-T Cells

Cloning of mbRGD/mbNGR Combined TGF-β DNR Armored CAR Constructs

Because of its suppressive role in the tumor microenvironment, TGF-β has been targeted in several studies seeking to boost anti-tumor immunity. We had more specifically targeted TGF-β signaling within the tumor microenvironment with a TGF-β dominant-negative receptor (TGF-β DNR), which renders transduced tumor-specific T cells unresponsive to TGF-β. In order to enhance CAR-T cells homing and infiltration into tumor while protect neighboring immune cells from the suppressive effects of TGF-β by enabling CAR-T cells to retain cytolytic activity in the presence of TGF-β, we designed several mbRGD/mbNGR combined TGF-β DNR armored CAR-T cells. They were the DLL3-CAR-CD28SP-RGD-(G4S)-dnTGFβRII (SEQ ID NO: 52), DLL3-CAR-CD5SP-RGD-(G4S)-dnTGFβRII (SEQ ID NO: 53), DLL3-CAR-CD28SP-NGR-(G4S)-dnTGFβRII (SEQ ID NO: 54), DLL3-CAR-dnTGFβRII-P2A-NGR-CD7 (SEQ ID NO: 55), DLL3-CAR-dnTGFβRII-P2A-NGR-Flag-CD7 (SEQ ID NO: 56), DLL3-CAR-NGR-(G4S)-NGR-CD7-T2A-dnTGFβRII (SEQ ID NO: 57) and DLL3-CAR-NGR-(G45)-NGR-Flag-CD7-T2A-dnTGFβRII (SEQ ID NO: 58). Sequences of the related CARs are shown in Table 7.

TABLE 7
Modalities and sequences of mbRGD/mbNGR combined TGF-β DNR armor CARs

| SEQ ID NO | Modality | Sequence |
|---|---|---|
| 51 | DLL3-CAR- | MALPVTALLLPLALLLHAARPQVQLVESGGGVVQPGGSLRLSCAASFSG |
| | dnTGFβRII | YGVSTMAWFRQAPGKGLEGVAAITVGSGNTYYADSVKGRFTISRDNSK |
| | | NTVYLQMNSLRAEDTAMYYCAVGYLSGGSWDVPGRYNYWGQGTLVT |
| | | VSSGGGGSGGGGSGGGGSEVQLVESGGGLVQPGGSLRLSCAASGNTYSS |
| | | NYMGWFRQAPGKGLEEVAVIYTRGGHTYYVDSVRGRFTISQDNAKNSL |
| | | YLQMNSLRAEDTAVYYCAASSRHRLGLNNPRDYDYWGQGTLVTVSST |
| | | TTPAPRPPTPAPTIASQPLSLRPEACRPAAGGAVHTRGLDFACDIYIWAPL |
| | | AGTCGVLLLSLVITLYCKRGRKKLLYIFKQPFMRPVQTTQEEDGCSCRFP |
| | | EEEEGGCELRVKFSRSADAPAYKQGQNQLYNELNLGRREEYDVLDKRR |
| | | GRDPEMGGKPRRKNPQEGLYNELQKDKMAEAYSEIGMKGERRRGKGH |
| | | DGLYQGLSTATKDTYDALHMQALPPRGSGEGRGSLLTCGDVEENPGPM |
| | | GRGLLRGLWPLHIVLWTRIASTIPPHVQKSVNNDMIVTDNNGAVKFPQL |
| | | CKFCDVRFSTCDNQKSCMSNCSITSICEKPQEVCVAVWRKNDENITLETV |
| | | CHDPKLPYHDFILEDAASPKCIMKEKKKPGETFFMCSCSSDECNDNIIFSE |
| | | EYNTSNPDLLLVIFQVTGISLLPPLGVAISVIIIFYCYRVNRQQKLSS |
| 52 | DLL3-CAR- | MALPVTALLLPLALLLHAARPQVQLVESGGGVVQPGGSLRLSCAASFSG |
| | CD28SP- | YGVSTMAWFRQAPGKGLEGVAAITVGSGNTYYADSVKGRFTISRDNSK |
| | RGD-(G4S)- | NTVYLQMNSLRAEDTAMYYCAVGYLSGGSWDVPGRYNYWGQGTLVT |
| | dnTGFβRII | VSSGGGGSGGGGSGGGGSEVQLVESGGGLVQPGGSLRLSCAASGNTYSS |
| | | NYMGWFRQAPGKGLEEVAVIYTRGGHTYYVDSVRGRFTISQDNAKNSL |
| | | YLQMNSLRAEDTAVYYCAASSRHRLGLNNPRDYDYWGQGTLVTVSST |
| | | TTPAPRPPTPAPTIASQPLSLRPEACRPAAGGAVHTRGLDFACDIYIWAPL |
| | | AGTCGVLLLSLVITLYCKRGRKKLLYIFKQPFMRPVQTTQEEDGCSCRFP |
| | | EEEEGGCELRVKFSRSADAPAYKQGQNQLYNELNLGRREEYDVLDKRR |
| | | GRDPEMGGKPRRKNPQEGLYNELQKDKMAEAYSEIGMKGERRRGKGH |
| | | DGLYQGLSTATKDTYDALHMQALPPRGSGEGRGSLLTCGDVEENPGPM |
| | | LRLLLALNLFPSIQVTGCDCRGDCFCGGGGSTIPPHVQKSVNNDMIVTDN |
| | | NGAVKFPQLCKFCDVRFSTCDNQKSCMSNCSITSICEKPQEVCVAVWRK |
| | | NDENITLETVCHDPKLPYHDFILEDAASPKCIMKEKKKPGETFFMCSCSS |
| | | DECNDNIIFSEEYNTSNPDLLLVIFQVTGISLLPPLGVAISVIIIFYCYRVNR |
| | | QQKLSS |
| 53 | DLL3-CAR- | MALPVTALLLPLALLLHAARPQVQLVESGGGVVQPGGSLRLSCAASFSG |
| | CD5SP- | YGVSTMAWFRQAPGKGLEGVAAITVGSGNTYYADSVKGRFTISRDNSK |
| | RGD-(G4S)- | NTVYLQMNSLRAEDTAMYYCAVGYLSGGSWDVPGRYNYWGQGTLVT |
| | dnTGFβRII | VSSGGGGSGGGGSGGGGSEVQLVESGGGLVQPGGSLRLSCAASGNTYSS |
| | | NYMGWFRQAPGKGLEEVAVIYTRGGHTYYVDSVRGRFTISQDNAKNSL |
| | | YLQMNSLRAEDTAVYYCAASSRHRLGLNNPRDYDYWGQGTLVTVSST |
| | | TTPAPRPPTPAPTIASQPLSLRPEACRPAAGGAVHTRGLDFACDIYIWAPL |
| | | AGTCGVLLLSLVITLYCKRGRKKLLYIFKQPFMRPVQTTQEEDGCSCRFP |
| | | EEEEGGCELRVKFSRSADAPAYKQGQNQLYNELNLGRREEYDVLDKRR |
| | | GRDPEMGGKPRRKNPQEGLYNELQKDKMAEAYSEIGMKGERRRGKGH |
| | | DGLYQGLSTATKDTYDALHMQALPPRGSGEGRGSLLTCGDVEENPGPM |
| | | PMGSLQPLATLYLLGMLVASCLGCDCRGDCFCGGGGSTIPPHVQKSVNN |
| | | DMIVTDNNGAVKFPQLCKFCDVRFSTCDNQKSCMSNCSITSICEKPQEVC |
| | | VAVWRKNDENITLETVCHDPKLPYHDFILEDAASPKCIMKEKKKPGETF |
| | | FMCSCSSDECNDNIIFSEEYNTSNPDLLLVIFQVTGISLLPPLGVAISVIIIFY |
| | | CYRVNRQQKLSS |
| 54 | DLL3-CAR- | MALPVTALLLPLALLLHAARPQVQLVESGGGVVQPGGSLRLSCAASFSG |
| | CD28SP- | YGVSTMAWFRQAPGKGLEGVAAITVGSGNTYYADSVKGRFTISRDNSK |
| | NGR-(G4S)- | NTVYLQMNSLRAEDTAMYYCAVGYLSGGSWDVPGRYNYWGQGTLVT |
| | dnTGFβRII | VSSGGGGSGGGGSGGGGSEVQLVESGGGLVQPGGSLRLSCAASGNTYSS |
| | | NYMGWFRQAPGKGLEEVAVIYTRGGHTYYVDSVRGRFTISQDNAKNSL |
| | | YLQMNSLRAEDTAVYYCAASSRHRLGLNNPRDYDYWGQGTLVTVSST |
| | | TTPAPRPPTPAPTIASQPLSLRPEACRPAAGGAVHTRGLDFACDIYIWAPL |
| | | AGTCGVLLLSLVITLYCKRGRKKLLYIFKQPFMRPVQTTQEEDGCSCRFP |
| | | EEEEGGCELRVKFSRSADAPAYKQGQNQLYNELNLGRREEYDVLDKRR |
| | | GRDPEMGGKPRRKNPQEGLYNELQKDKMAEAYSEIGMKGERRRGKGH |
| | | DGLYQGLSTATKDTYDALHMQALPPRGSGEGRGSLLTCGDVEENPGPM |
| | | LRLLLALNLFPSIQVTGCNGRCGGGGGSTIPPHVQKSVNNDMIVTDNNG |
| | | AVKFPQLCKFCDVRFSTCDNQKSCMSNCSITSICEKPQEVCVAVWRKND |
| | | ENITLETVCHDPKLPYHDFILEDAASPKCIMKEKKKPGETFFMCSCSSDE |
| | | CNDNIIFSEEYNTSNPDLLLVIFQVTGISLLPPLGVAISVIIIFYCYRVNRQQ |
| | | KLSS |
| 55 | DLL3-CAR- | MALPVTALLLPLALLLHAARPQVQLVESGGGVVQPGGSLRLSCAASFSG |
| | dnTGFβRII- | YGVSTMAWFRQAPGKGLEGVAAITVGSGNTYYADSVKGRFTISRDNSK |
| | P2A-NGR- | NTVYLQMNSLRAEDTAMYYCAVGYLSGGSWDVPGRYNYWGQGTLVT |
| | CD7 | VSSGGGGSGGGGSGGGGSEVQLVESGGGLVQPGGSLRLSCAASGNTYSS |
| | | NYMGWFRQAPGKGLEEVAVIYTRGGHTYYVDSVRGRFTISQDNAKNSL |
| | | YLQMNSLRAEDTAVYYCAASSRHRLGLNNPRDYDYWGQGTLVTVSST |
| | | TTPAPRPPTPAPTIASQPLSLRPEACRPAAGGAVHTRGLDFACDIYIWAPL |
| | | AGTCGVLLLSLVITLYCKRGRKKLLYIFKQPFMRPVQTTQEEDGCSCRFP |
| | | EEEEGGCELRVKFSRSADAPAYKQGQNQLYNELNLGRREEYDVLDKRR |
| | | GRDPEMGGKPRRKNPQEGLYNELQKDKMAEAYSEIGMKGERRRGKGH |
| | | DGLYQGLSTATKDTYDALHMQALPPRGSGEGRGSLLTCGDVEENPGPM |
| | | GRGLLRGLWPLHIVLWTRIASTIPPHVQKSVNNDMIVTDNNGAVKFPQL |
| | | CKFCDVRFSTCDNQKSCMSNCSITSICEKPQEVCVAVWRKNDENITLETV |
| | | CHDPKLPYHDFILEDAASPKCIMKEKKKPGETFFMCSCSSDECNDNIIFSE |
| | | EYNTSNPDLLLVIFQVTGISLLPPLGVAISVIIIFYCYRVNRQQKLSSGSGA |
| | | TNFSLLKQAGDVEENPGPMPMGSLQPLATLYLLGMLVASCLGCNGRCG |
| | | GGGGSGGGGSGGGGSPPRASALPAPPTGSALPDPQTASALPDPPAASALP |
| | | AALAVISFLLGLGLGVACVLA |
| 56 | DLL3-CAR- | MALPVTALLLPLALLLHAARPQVQLVESGGGVVQPGGSLRLSCAASFSG |
| | dnTGFßRII- | YGVSTMAWFRQAPGKGLEGVAAITVGSGNTYYADSVKGRFTISRDNSK |
| | P2A-NGR- | NTVYLQMNSLRAEDTAMYYCAVGYLSGGSWDVPGRYNYWGQGTLVT |
| | Flag-CD7 | VSSGGGGSGGGGGGGGSEVQLVESGGGLVQPGGSLRLSCAASGNTYSS |
| | | NYMGWFRQAPGKGLEEVAVIYTRGGHTYYVDSVRGRFTISQDNAKNSL |
| | | YLQMNSLRAEDTAVYYCAASSRHRLGLNNPRDYDYWGQGTLVTVSST |
| | | TTPAPRPPTPAPTIASQPLSLRPEACRPAAGGAVHTRGLDFACDIYIWAPL |
| | | AGTCGVLLLSLVITLYCKRGRKKLLYIFKQPFMRPVQTTQEEDGCSCRFP |
| | | EEEEGGCELRVKFSRSADAPAYKQGQNQLYNELNLGRREEYDVLDKRR |
| | | GRDPEMGGKPRRKNPQEGLYNELQKDKMAEAYSEIGMKGERRRGKGH |
| | | DGLYQGLSTATKDTYDALHMQALPPRGSGEGRGSLLTCGDVEENPGPM |
| | | GRGLLRGLWPLHIVLWTRIASTIPPHVQKSVNNDMIVTDNNGAVKFPQL |
| | | CKFCDVRFSTCDNQKSCMSNCSITSICEKPQEVCVAVWRKNDENITLETV |
| | | CHDPKLPYHDFILEDAASPKCIMKEKKKPGETFFMCSCSSDECNDNIIFSE |
| | | EYNTSNPDLLLVIFQVTGISLLPPLGVAISVIIIFYCYRVNRQQKLSSGSGA |
| | | TNFSLLKQAGDVEENPGPMPMGSLQPLATLYLLGMLVASCLGCNGRCG |
| | | GGGGSGGGGSGGGGSDYKDDDDKPPRASALPAPPTGSALPDPQTASALP |
| | | DPPAASALPAALAVISFLLGLGLGVACVLA |
| 57 | DLL3-CAR- | MALPVTALLLPLALLLHAARPQVQLVESGGGVVQPGGSLRLSCAASFSG |
| | NGR-(G4S)- | YGVSTMAWFRQAPGKGLEGVAAITVGSGNTYYADSVKGRFTISRDNSK |
| | NGR-CD7- | NTVYLQMNSLRAEDTAMYYCAVGYLSGGSWDVPGRYNYWGQGTLVT |
| | T2A- | VSSGGGGSGGGGSGGGGSEVQLVESGGGLVQPGGSLRLSCAASGNTYSS |
| | dnTGFβRII | NYMGWFRQAPGKGLEEVAVIYTRGGHTYYVDSVRGRFTISQDNAKNSL |
| | | YLQMNSLRAEDTAVYYCAASSRHRLGLNNPRDYDYWGQGTLVTVSST |
| | | TTPAPRPPTPAPTIASQPLSLRPEACRPAAGGAVHTRGLDFACDIYIWAPL |
| | | AGTCGVLLLSLVITLYCKRGRKKLLYIFKQPFMRPVQTTQEEDGCSCRFP |
| | | EEEEGGCELRVKFSRSADAPAYKQGQNQLYNELNLGRREEYDVLDKRR |
| | | GRDPEMGGKPRRKNPQEGLYNELQKDKMAEAYSEIGMKGERRRGKGH |
| | | DGLYQGLSTATKDTYDALHMQALPPRGSGATNFSLLKQAGDVEENPGP |
| | | MPMGSLQPLATLYLLGMLVASCLGCNGRCGGGGGSCNGRCGGGGGSG |
| | | GGGSGGGGSPPRASALPAPPTGSALPDPQTASALPDPPAASALPAALAVI |
| | | SFLLGLGLGVACVLAGSGEGRGSLLTCGDVEENPGPMGRGLLRGLWPL |
| | | HIVLWTRIASTIPPHVQKSVNNDMIVTDNNGAVKFPQLCKFCDVRFSTCD |
| | | NQKSCMSNCSITSICEKPQEVCVAVWRKNDENITLETVCHDPKLPYHDFI |
| | | LEDAASPKCIMKEKKKPGETFFMCSCSSDECNDNIIFSEEYNTSNPDLLLV |
| | | IFQVTGISLLPPLGVAISVIIIFYCYRVNRQQKLSS |
| 58 | DLL3-CAR- | MALPVTALLLPLALLLHAARPQVQLVESGGGVVQPGGSLRLSCAASFSG |
| | NGR-(G4S)- | YGVSTMAWFRQAPGKGLEGVAAITVGSGNTYYADSVKGRFTISRDNSK |
| | NGR-Flag- | NTVYLQMNSLRAEDTAMYYCAVGYLSGGSWDVPGRYNYWGQGTLVT |
| | CD7-T2A- | VSSGGGGSGGGGSGGGGSEVQLVESGGGLVQPGGSLRLSCAASGNTYSS |
| | dnTGFβRII | NYMGWFRQAPGKGLEEVAVIYTRGGHTYYVDSVRGRFTISQDNAKNSL |
| | | YLQMNSLRAEDTAVYYCAASSRHRLGLNNPRDYDYWGQGTLVTVSST |
| | | TTPAPRPPTPAPTIASQPLSLRPEACRPAAGGAVHTRGLDFACDIYIWAPL |
| | | AGTCGVLLLSLVITLYCKRGRKKLLYIFKQPFMRPVQTTQEEDGCSCRFP |
| | | EEEEGGCELRVKFSRSADAPAYKQGQNQLYNELNLGRREEYDVLDKRR |
| | | GRDPEMGGKPRRKNPQEGLYNELQKDKMAEAYSEIGMKGERRRGKGH |
| | | DGLYQGLSTATKDTYDALHMQALPPRGSGATNFSLLKQAGDVEENPGP |
| | | MPMGSLQPLATLYLLGMLVASCLGCNGRCGGGGGSCNGRCGGGGGSG |
| | | GGGSGGGGSDYKDDDDKPPRASALPAPPTGSALPDPQTASALPDPPAAS |
| | | ALPAALAVISFLLGLGLGVACVLAGSGEGRGSLLTCGDVEENPGPMGRG |
| | | LLRGLWPLHIVLWTRIASTIPPHVQKSVNNDMIVTDNNGAVKFPQLCKF |
| | | CDVRFSTCDNQKSCMSNCSITSICEKPQEVCVAVWRKNDENITLETVCH |
| | | DPKLPYHDFILEDAASPKCIMKEKKKPGETFFMCSCSSDECNDNIIFSEEY |
| | | NTSNPDLLLVIFQVTGISLLPPLGVAISVIIIFYCYRVNRQQKLSS |

Generation of mbRGD/mbNGR Combined TGF-β DNR Armored CAR-T Cell

### 1. Preparation of Lentivirus

The sequences encoding the mbRGD/mbNGR combined TGF-β DNR armored CARs as described above were codon optimized and synthesized by GenScript. Then the sequences were incorporated into 3^{rd} generation lentivirus vectors via standard molecular clone methods. All plasmid sequences were verified via sequencing. Then the plasmids and lentiviral packaging plasmids were co-transfected into HEK293 cells to produce lentivirus. Virus particles were collected from the supernatant, and concentrated by PEG precipitation or ultracentrifugation. The concentrated lentiviral particles were used to transduce T cells.

### 2. T Cell Transduction

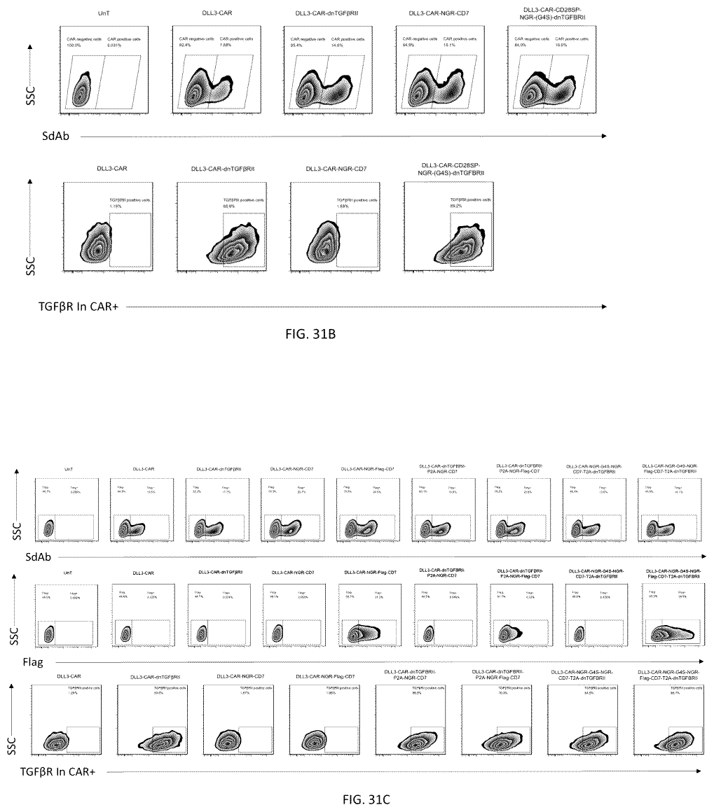

T cells were isolated from healthy donor PBMCs (HemaCare) using pan T cell isolation kit (Miltenyi Biotec, 130096535). Isolated T cells were cultured under AIMV (Gibco, 31035025) medium with 5% FBS (Gibco, 10099141) and further activated by CD3/CD28 activation beads (Miltenyi Biotec, 130091442) at a 2:1 ratio in the 37° C., 5% CO₂ incubator. 48 or 72 hours after initial activation, T cells were transduced with lentivirus expressing a DLL3 targeting CAR at proper multiplicity of infection (MOI) in the presence of 8 μg/ml polybrene (SIGMA-ALDRICH, H9268-10G). Additional IL-2 was supplemented to a final concentration of 300 IU/ml. Fresh medium was replaced 24 hours post lentiviral infection. Infected T cells were maintained under AIMV medium with 5% FBS and 300 IU/ml IL-2 at a cell density between 5×10⁵ to 1×10⁶ cells/ml. 4 days after transduction, the expression of CAR on the cell surface were determined by FACS. Specifically, 1×10⁵ transduced or untransduced T cells (unT) were collected by centrifugation at 300 g for 5 min. The cells were then re-suspended with 100 μl PBS containing iFlour488-Anti-sdAb (GenScript, China), PE-Anti-Flag (Biolegend, US) or APC-Anti-TGF-βRII (Abeam, UK). SdAb positive ratio represents CAR ratio on T cells, Flag positive ratio represents mbNGR ratio on T cells, and TGF-βRII positive ratio represents TGF-β DNR ratio on CAR positive T cells. As shown in FIG. 31A, CAR positive ratio on DLL3-CAR, DLL3-CAR-dnTGFβRII, DLL3-RGD-CD7, DLL3-CAR-CD28SP-RGD-(G4S)-dnTGFβRII and DLL3-CAR-CD28SP-RGD-(G45)-dnTGFβRII were 18.9%, 13.6%, 17.6%, 12.7% and 12.1%. The TGFβRII expressed on the surface of DLL3-CAR-dnTGFβRII, DLL3-CAR-CD28SP-RGD-(G4S)-dnTGFβRII and DLL3-CAR-CD28SP-RGD-(G4S)-dnTGFβRII CAR positive T cells were 91.2%, 82.1% and 90.6%. As shown in FIG. 31B, CAR positive ratio on DLL3-CAR, DLL3-CAR-dnTGFβRII, DLL3-CAR-NGR-CD7 and DLL3-CAR-CD28SP-NGR-(G4S)-dnTGFβRII were 7.58%, 14.8%, 15.1% and 15.9%. The TGFβRII expressed on the surface of DLL3-CAR-dnTGFβRII and DLL3-CAR-CD28SP-NGR-(G4S)-dnTGFβRII CAR positive T cells were 88.6% and 89.2%. As shown in FIG. 31C, CAR positive ratio on DLL3-CAR, DLL3-CAR-dnTGFβRII, DLL3-CAR-NGR-CD7, DLL3-CAR-NGR-Flag-CD7, DLL3-CAR-dnTGFβRII-P2A-NGR-CD7, DLL3-CAR-dnTGFβRII-P2A-NGR-Flag-CD7, DLL3-CAR-NGR-(G4S)-NGR-CD7-T2A-dnTGFβRII and DLL3-CAR-NGR-(G4S)-NGR-Flag-CD7-T2A-dnTGFβRII were 15.5%, 17.7%, 20.7%, 24.5%, 19.9% 23.8% 13.6% and 14.1%. The mbNGRs expressed on the surface of DLL3-CAR-NGR-Flag-CD7, DLL3-CAR-dnTGFβRII-P2A-NGR-Flag-CD7 and DLL3-CAR-NGR-(G45)-NGR-Flag-CD7-T2A-dnTGFβRII were 31.3%, 8.3% and 39.5%. The TGFβRII expressed on the surface of DLL3-CAR-dnTGFβRII, DLL3-CAR-dnTGFβRII-P2A-NGR-CD7, DLL3-CAR-dnTGFβRII-P2A-NGR-Flag-CD7, DLL3-CAR-NGR-(G4S)-NGR-CD7-T2A-dnTGFβRII and DLL3-CAR-NGR-(G45)-NGR-Flag-CD7-T2A-dnTGFβRII CAR positive T cells were 90.6%, 86.8%, 76.3%, 84.9% and 86.7%.

### Example 17: mbRGD/mbNGR Enhances mbRGD/mbNGR Combined TGF-β DNR Armored CAR-T Cells Adhesion to Endothelial Cells

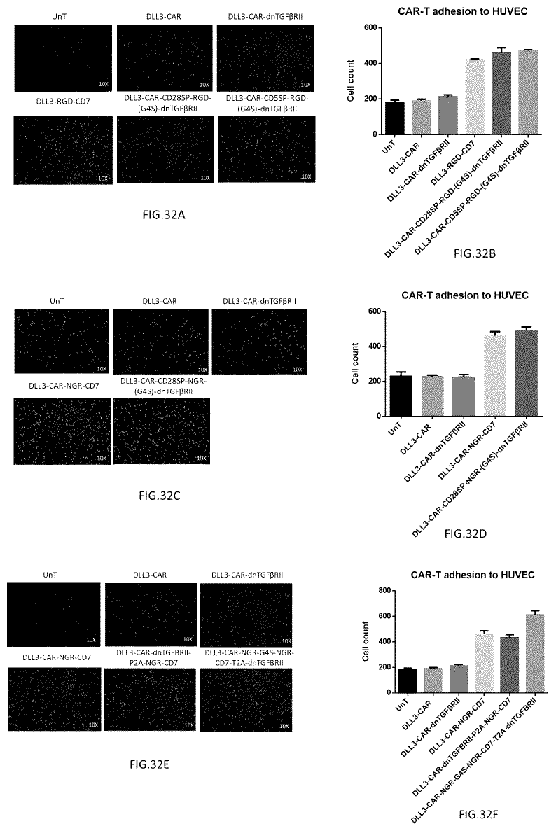

To investigate whether mbRGD can improve mbRGD combined TGF-β DNR armored CAR-T cell adhesion to neo-vascular endothelial cells, endothelial cell adhesion assay was performed. In this assay, human umbilical vein endothelial cells (HUVEC) were used to represent neo-vascular endothelial cells. DLL3-CAR-CD28SP-RGD-(G4S)-dnTGFβRII and DLL3-CAR-CD28SP-RGD-(G4S)-dnTGFβRII were prepared and their adhesion to HUVEC were investigated. Briefly, 1×10⁵ HUVEC cells were seeded onto the bottom of a 24-well-plate to form a single layer. The next day, 3×10⁵ DLL3-CAR-CD28SP-RGD-(G4S)-dnTGFβRII, DLL3-CAR-CD28SP-RGD-(G4S)-dnTGFβRII, DLL3-CAR-dnTGFβRII, DLL3-RGD-CD7, DLL3-CAR, and untransduced T cells (unT) were labeled with CFSE and added to the single HUVEC layer respectively. After 30 min incubation, the unbounded T cells were carefully rinsed out with fresh medium. The bounded cell were imaged on a fluorescence microscopy and counted. As shown in FIGS. 32A and 32B, DLL3-CAR-CD28SP-RGD-(G4S)-dnTGFβRII, DLL3-CAR-CD28SP-RGD-(G4S)-dnTGFβRII and DLL3-RGD-CD7 had remarkably more cell adhesion to HUVEC than DLL3-CAR-dnTGFβRII, DLL3 CAR or unT cells. These results suggested that mbRGD can enhance mbRGD combined TGF-β DNR armored CAR-T cell adhesion to neo-vascular endothelial cells.

To investigate whether mbNGR can improve mbNGR combined TGF-β DNR armored CAR-T cell adhesion to neo-vascular endothelial cells, endothelial cell adhesion assay was performed. In this assay, human umbilical vein endothelial cells (HUVEC) were used to represent neo-vascular endothelial cells. DLL3-CAR-CD28SP-NGR-(G4S)-dnTGFβRII, DLL3-CAR-dnTGFβRII-P2A-NGR-CD7 and DLL3-CAR-NGR-(G4S)-NGR-CD7-T2A-dnTGFβRII were prepared and their adhesion to HUVEC were investigated. Briefly, 1×10⁵ HUVEC cells were seeded onto the bottom of a 24-well-plate to form a single layer. The next day, 3×10⁵ DLL3-CAR-CD28SP-NGR-(G4S)-dnTGFβRII, DLL3-CAR-dnTGFβRII-P2A-NGR-CD7, DLL3-CAR-NGR-(G4S)-NGR-CD7-T2A-dnTGFβRII, DLL3-CAR-dnTGFβRII, DLL3-CAR-NGR-CD7, DLL3-CAR and untransduced T cells (unT) were labeled with CFSE and added to the single HUVEC layer respectively. After 30 min incubation, the unbounded T cells were carefully rinsed out with fresh medium. The bounded cell were imaged on a fluorescence microscopy and counted. As shown in FIGS. 32C-F, DLL3-CAR-CD28SP-NGR-(G4S)-dnTGFβRII, DLL3-CAR-dnTGFβRII-P2A-NGR-CD7, DLL3-CAR-NGR-(G4S)-NGR-CD7-T2A-dnTGFβRII and DLL3-CAR-NGR-CD7 had remarkably more cell adhesion to HUVEC than DLL3-CAR-dnTGFβRII, DLL3 CAR or unT cells. These results suggested that mbNGR can enhance mbNGR combined TGF-β DNR armored CAR-T cell adhesion to neo-vascular endothelial cells.

### Example 18: Expression of dnTGF-βRII on mbRGD/mbNGR Combined TGF-β DNR Armored CAR-T Cells Prevents TGF-β Signal Induction Through SMAD2

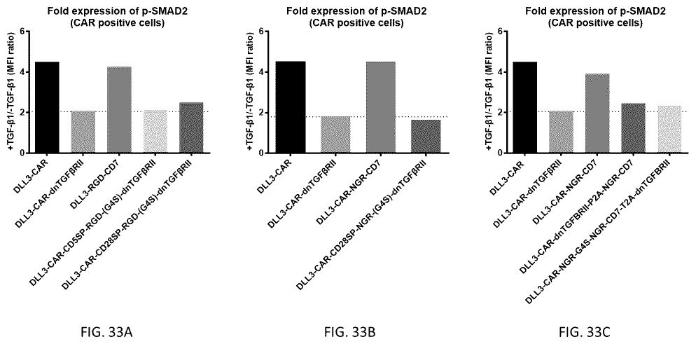

To investigate whether dnTGF-βRII can prevents mbRGD combined TGF-β DNR armored CAR-T cell TGF-β signal induction through SMAD2, analysis of phosphoproteins by intracellular flow cytometry was performed. Specifically, DLL3-CAR-CD28SP-RGD-(G45)-dnTGFβRII, DLL3-CAR-CD28SP-RGD-(G4S)-dnTGFβRII, DLL3-CAR-dnTGFβRII, DLL3-RGD-CD7 and DLL3-CAR were rested in cytokine-free media overnight, Stained with iFlour488-Anti-sdAb antibodies, the cells were fixed with 10% formalin, followed by permeabilization by ice-cold methanol, and added Recombinant Human TGF-β1 protein (R&D, US) to stimulate for 30 minutes, Stained with PE Phospho-SMAD2 mAb antibodies (CST, US). Then, the phosphorylation of SMAD2 within the mbRGD combined TGF-β DNR armored CAR-T cell population was analyzed by flow cytometry. As shown in FIG. 33A, DLL3-CAR-CD28SP-RGD-(G4S)-dnTGFβRII, DLL3-CAR-CD28SP-RGD-(G4S)-dnTGFβRII and DLL3-CAR-dnTGFβRII had remarkably more prevents to Phospho-SMAD2 than DLL3-RGD-CD7 and DLL3-CAR. These results suggested that dnTGFβRII can prevents mbRGD combined TGF-β DNR armored CAR-T cell TGF-β signal induction through SMAD2.

To investigate whether dnTGF-βRII can prevent mbNGR combined TGF-β DNR armored CAR-T cell TGF-β signal induction through SMAD2, analysis of phosphoproteins by intracellular flow cytometry was performed. Specifically, DLL3-CAR-CD28SP-NGR-(G45)-dnTGFβRII, DLL3-CAR-dnTGFβRII-P2A-NGR-CD7, DLL3-CAR-NGR-(G4S)-NGR-CD7-T2A-dnTGFβRII, DLL3-CAR-dnTGFβRII, DLL3-CAR-NGR-CD7 and DLL3-CAR were rested in cytokine-free media overnight, Stained with iFlour488-Anti-sdAb antibodies, the cells were fixed with 10% formalin, followed by permeabilization by ice-cold methanol, and added Recombinant Human TGF-β1 protein (R&D, US) to stimulate for 30 minutes, Stained with PE Phospho-SMAD2 mAb antibodies (CST, US). Then, The phosphorylation of SMAD2 within the mbNGR combined TGF-β DNR armored CAR-T cell population was analyzed by flow cytometry. As shown in FIGS. 33B-33C, DLL3-CAR-CD28SP-NGR-(G4S)-dnTGFβRII, DLL3-CAR-dnTGFβRII-P2A-NGR-CD7, DLL3-CAR-NGR-(G4S)-NGR-CD7-T2A-dnTGFβRII and DLL3-CAR-dnTGFβRII had remarkably more prevents to Phospho-SMAD2 than DLL3-CAR-NGR-CD7 and DLL3-CAR. These results suggested that dnTGFβRII can prevents mbNGR combined TGF-β DNR armored CAR-T cell TGF-β signal induction through SMAD2.

### Example 19: Influence of the mbRGD/mbNGR Combined TGF-β DNR Armored CAR on Cytotoxicity and Cytokine Secretion of CAR-T Cells Against Target Cells and Endothelial Cells

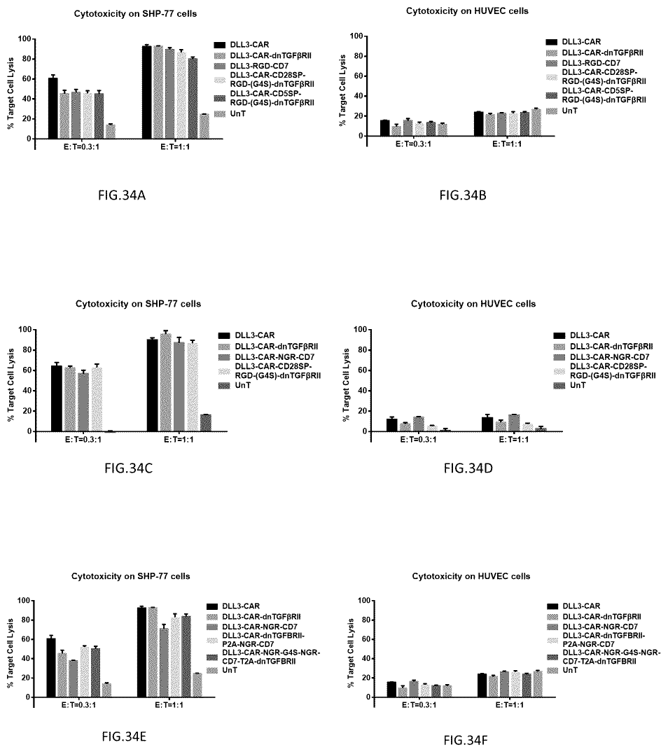

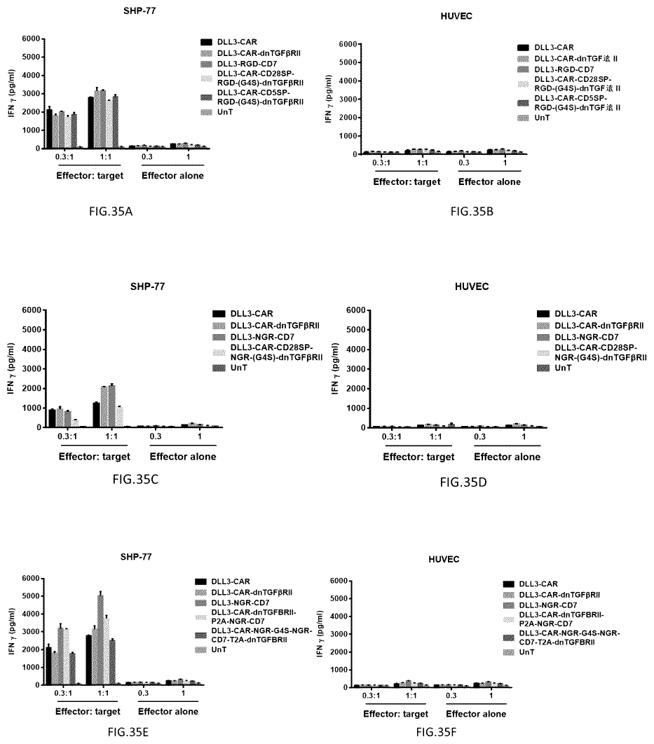

To investigate whether mbRGD combined TGF-β DNR armored CAR influences the cytotoxicity of CAR-T cells against target cells or endothelial cells, HUVEC and DLL3 positive cells SHP77 were used for evaluating the cytotoxicity and cytokine secretion of DLL3-CAR-CD28SP-RGD-(G4S)-dnTGFβRII, DLL3-CAR-CD28SP-RGD-(G4S)-dnTGFβRII, DLL3-CAR-dnTGFβRII, DLL3-RGD-CD7 and DLL3-CAR. Specifically, SHP77 cells were co-incubated with CAR-T and unT for 22 h. Then, LDH (lactate dehydrogenase) levels were measured. As shown in FIGS. 34A-34B, cytotoxicity levels of mbRGD combined TGF-β DNR armored CAR against both SHP77 and HUVEC were comparable to the conventional CAR-T cells. These results demonstrated that mbRGD combined TGF-β DNR armor has no influence on the cytotoxicity of CAR-T cells against target cells; meanwhile the mbRGD combined TGF-β DNR armored CAR-T cells did not cause cytotoxicity to endothelial cells. The supernatants of the co-cultures were then taken for IFN-γ measurement by HTFR methods. As shown in FIG. 35A-35B, IFN-γ secretion of all CAR-T cells is highly activated by SHP77 co-culture. None of the mbRGD combined TGF-0 DNR armored CAR-T cells showed increased IFN-γ secretion upon co-culture with HUVEC compared to CAR-T cells only. These results demonstrated that mbRGD combined TGF-β DNR armored CAR-T cells are safe to endothelial cells.

To investigate whether mbNGR combined TGF-β DNR armored CAR influences the cytotoxicity of CAR-T cells against target cells or endothelial cells, HUVEC and DLL3 positive cells SHP77 were used for evaluating the cytotoxicity and cytokine secretion of DLL3-CAR-CD28SP-NGR-(G4S)-dnTGFβRII, DLL3-CAR-dnTGFβRII-P2A-NGR-CD7, DLL3-CAR-NGR-(G4S)-NGR-CD7-T2A-dnTGFβRII, DLL3-CAR-dnTGFβRII, DLL3-CAR-NGR-CD7 and DLL3-CAR. Specifically, SHP77 cells were co-incubated with CAR-T and unT for 22 h. Then, LDH (lactate dehydrogenase) levels were measured. As shown in FIGS. 34C-34F, cytotoxicity levels of mbNGR combined TGF-β DNR armored CAR against both SHP77 and HUVEC were comparable to the conventional CAR-T cells. These results demonstrated that mbNGR combined TGF-β DNR armor has no influence on the cytotoxicity of CAR-T cells against target cells; meanwhile the mbNGR combined TGF-β DNR armored CAR-T cells did not cause cytotoxicity to endothelial cells. The supernatants of the co-cultures were then taken for IFN-γ measurement by HTFR methods. As shown in FIGS. 35C-35F, IFN-γ secretion of all CAR-T cells is highly activated by SHP77 co-culture. None of the mbNGR combined TGF-β DNR armored CAR-T cells showed increased IFN-γ secretion upon co-culture with HUVEC compared to CAR-T cells only. These results demonstrated that mbNGR combined TGF-β DNR armored CAR-T cells are safe to endothelial cells.

### Example 20: mbRGD/mbNGR Combined TGF-β DNR Armored CAR-T Cells Demonstrated Superior Tumor Infiltration In Vivo

To investigate whether the enhanced tumor infiltration of mbRGD/mbNGR combined TGF-β DNR armored CAR-T, we performed an in vivo assay to determine tumor infiltration of different CAR-T. Firstly, NCI-H82 cells were implanted subcutaneously to NCG mice. The tumors were allowed to grow to 280-330 mm³. Then 2 M of DLL3-CAR-dnTGFβRII, DLL3-CAR-CD28SP-RGD-(G4S)-dnTGFβRII, DLL3-CAR-CD28SP-NGR-(G4S)-dnTGFβRII or DLL3-CAR-NGR-(G4S)-NGR-CD7-T2A-dnTGFβRII CAR-T cells were infused intravenously. The mice were euthanized and tumors resected 5 days after infusion. The resected tumors were used for sdAb staining by immunohistochemistry (IHC) to observe the level of CAR-T infiltration in the tumor.

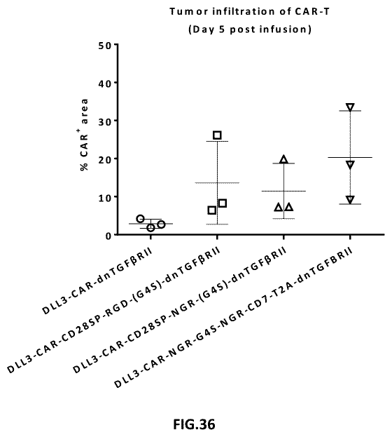

As shown in FIG. 36, DLL3-CAR-CD28SP-RGD-(G4S)-dnTGFβRII, DLL3-CAR-CD28SP-NGR-(G4S)-dnTGFβRII and DLL3-CAR-NGR-(G4S)-NGR-CD7-T2A-dnTGFβRII CAR-T cells showed higher tumor infiltration of sdAb positive cells than DLL3-CAR-dnTGFβRII both at Day 5. These results indicated that mbRGD/mbNGR combined TGF-β DNR could enhance tumor infiltration of CAR-T cells.

Having thus described in detail preferred embodiments of the present disclosure, it is to be understood that the disclosure defined by the above paragraphs is not to be limited to particular details set forth in the above description as many apparent variations thereof are possible without departing from the spirit or scope of the present disclosure.

What is claimed:

1. An immune effector cell expressing:
(a) a functional exogenous receptor, and
(b) an exogenous tumor homing peptide (THP).

2. The immune effector cell of claim 1, wherein the functional exogenous receptor is selected from the group consisting of a chimeric antigen receptor (CAR), an engineered T cell receptor (TCR), a chimeric TCR (cTCR), a T cell antigen coupler (TAC), a TAC-like chimeric receptor, and combinations thereof.

3. The immune effector cell of claim 2, wherein the functional exogenous receptor is a CAR and wherein the CAR comprises (i) an extracellular antigen binding domain, (ii) a transmembrane domain, and (iii) an intracellular signaling domain, optionally, the CAR further comprises a hinge domain.

4. The immune effector cell of claim 3, wherein the intracellular signaling domain comprises a primary intracellular signaling domain of an immune effector cell.

5. The immune effector cell of claim 4, wherein the primary intracellular signaling domain is from CD3.

6. The immune effector cell of any one of claims 3 to 5, wherein the intracellular signaling domain comprises a co-stimulatory signaling domain.

7. The immune effector cell of any one of claims 3 to 6, further comprising a signal peptide, wherein the signal peptide is from CD8 or CD28.

8. The immune effector cell of any one of claims 1 to 7, wherein the immune effector cell is selected from the group consisting of T cells, natural killer (NK) cells, B cells, neutrophils, eosinophils, monocytes, macrophages, dendritic cells, peripheral blood mononuclear cells (PBMC), stem cells from which lymphoid cells may be differentiated, and combinations thereof.

9. The immune effector cell of claim 8, wherein the immune effector cell is a T cell.

10. The immune effector cell of any one of claims 3 to 9, wherein the CAR is an anti-DLL3 CAR.

11. The immune effector cell of claim 10, wherein the anti-DLL3 CAR comprises a first V_{H}H antibody moiety that comprises a CDR1 comprising the amino acid sequence of SEQ ID NO: 29 or a variant thereof comprising up to about 3 amino acid substitutions, a CDR2 comprising the amino acid sequence of SEQ ID NO:30 or a variant thereof comprising up to about 3 amino acid substitutions, and a CDR3 comprising the amino acid sequence of SEQ ID NO: 31 or a variant thereof comprising up to about 3 amino acid substitutions, and a second V_{H}H antibody moiety that comprises a CDR1 comprising the amino acid sequence of SEQ ID NO: 32 or a variant thereof comprising up to about 3 amino acid substitutions, a CDR2 comprising the amino acid sequence of SEQ ID NO:33 or a variant thereof comprising up to about 3 amino acid substitutions, and a CDR3 comprising the amino acid sequence of SEQ ID NO: 34 or a variant thereof comprising up to about 3 amino acid substitutions.

12. The immune effector cell of claim 10 or claim 11, wherein the anti-DLL3 CAR comprises a first V_{H}H antibody moiety comprising the amino acid sequences of SEQ ID NO: 27 or an amino acid sequence having at least 75%, 80%, 90%, 91%, 92%, 93%, 94%, 95%, 96%, 97%, 98%, or 99% identity to the amino acid sequence of SEQ ID NO: 27, and a second V_{H}H antibody moiety comprising the amino acid sequences of SEQ ID NO: 28 or an amino acid sequence having at least 75%, 80%, 90%, 91%, 92%, 93%, 94%, 95%, 96%, 97%, 98%, or 99% identity to the amino acid sequence of SEQ ID NO: 28.

13. The immune effector cell of claim 10, wherein the anti-DLL3 CAR comprises the amino acid sequence of SEQ ID NO: 8 or an amino acid sequence having at least 75%, 80%, 90%, 91%, 92%, 93%, 94%, 95%, 96%, 97%, 98%, or 99% identity to the amino acid sequence of SEQ ID NO: 8.

14. The immune effector cell of any one of claims 3 to 9, wherein the CAR is an anti-MSLN CAR.

15. The immune effector cell of claim 14, wherein the anti-MSLN CAR comprises the amino acid sequence of SEQ ID NO: 41 or an amino acid sequence having at least 75%, 80%, 90%, 91%, 92%, 93%, 94%, 95%, 96%, 97%, 98%, or 99% identity to the amino acid sequence of SEQ ID NO: 41.

16. The immune effector cell of any one of claims 3 to 9, wherein the CAR is an anti-GPC2 CAR.

17. The immune effector cell of claim 16, wherein the anti-GPC2 CAR comprises the amino acid sequence of SEQ ID NO: 44 or an amino acid sequence having at least 75%, 80%, 90%, 91%, 92%, 93%, 94%, 95%, 96%, 97%, 98%, or 99% identity to the amino acid sequence of SEQ ID NO: 44.

18. The immune effector cell of any one of claims 1 to 17, wherein the THP is selected from the group consisting of arginine-glycine-aspartic (RGD)-based peptides, asparagine-glycine-arginine (NGR)-based peptides, and combinations thereof.

19. The immune effector cell of any one of claims 1 to 18, wherein the THP comprises the RGD-4C peptide having the amino acid sequence of SEQ ID NO: 2.

20. The immune effector cell of any one of claims 1 to 18, wherein the THP comprises the NGR peptide having the amino acid sequence of SEQ ID NO: 38.

21. The immune effector cell of any one of claims 3 to 20, wherein the THP is fused with a transmembrane domain and/or a hinge domain.

22. The immune effector cell of claim 21, wherein the transmembrane domain and/or the hinge domain is from CD7 or TR2.

23. The immune effector cell of claim 22, wherein the transmembrane domain comprises the amino acid sequence of SEQ ID NO: 14, and the hinge domain comprises the amino acid sequence of SEQ ID NO: 13.

24. The immune effector cell of any one of claims 1 to 23, wherein the THP is fused with a TGF-β dominant-negative receptor (TGF-β DNR).

25. The immune effector cell of claim 24, comprising a polypeptide comprising:
i) the amino acid sequence of SEQ ID NO: 55 or an amino acid sequence having at least 75%, 80%, 90%, 91%, 92%, 93%, 94%, 95%, 96%, 97%, 98%, or 99% identity to the amino acid sequence of SEQ ID NO: 55;
ii) the amino acid sequence of SEQ ID NO: 57 or an amino acid sequence having at least 75%, 80%, 90%, 91%, 92%, 93%, 94%, 95%, 96%, 97%, 98%, or 99% identity to the amino acid sequence of SEQ ID NO: 57;
iii) the amino acid sequence of SEQ ID NO: 54 or an amino acid sequence having at least 75%, 80%, 90%, 91%, 92%, 93%, 94%, 95%, 96%, 97%, 98%, or 99% identity to the amino acid sequence of SEQ ID NO: 54;
iv) the amino acid sequence of SEQ ID NO: 56 or an amino acid sequence having at least 75%, 80%, 90%, 91%, 92%, 93%, 94%, 95%, 96%, 97%, 98%, or 99% identity to the amino acid sequence of SEQ ID NO: 56;
v) the amino acid sequence of SEQ ID NO: 58 or an amino acid sequence having at least 75%, 80%, 90%, 91%, 92%, 93%, 94%, 95%, 96%, 97%, 98%, or 99% identity to the amino acid sequence of SEQ ID NO: 58;
vi) the amino acid sequence of SEQ ID NO: 53 or an amino acid sequence having at least 75%, 80%, 90%, 91%, 92%, 93%, 94%, 95%, 96%, 97%, 98%, or 99% identity to the amino acid sequence of SEQ ID NO: 53; or
vii) the amino acid sequence of SEQ ID NO: 52 or an amino acid sequence having at least 75%, 80%, 90%, 91%, 92%, 93%, 94%, 95%, 96%, 97%, 98%, or 99% identity to the amino acid sequence of SEQ ID NO: 52.

26. The immune effector cell of any one of claims 1 to 25, wherein the THP is fused with a GPI linkage.

27. The immune effector cell of claim 1 or 2, wherein the functional exogenous receptor is a cTCR.

28. The immune effector cell of claim 27, wherein the cTCR is an anti-DLL3 cTCR.

29. The immune effector cell of claim 28, wherein the anti-DLL3 cTCR comprises the amino acid sequence of SEQ ID NO: 35 or an amino acid sequence having at least 75%, 80%, 90%, 91%, 92%, 93%, 94%, 95%, 96%, 97%, 98%, or 99% identity to the amino acid sequence of SEQ ID NO: 35.

30. The immune effector cell of any one of claims 1-29, comprising
(i) a polypeptide having the amino acid sequence set forth in any one of SEQ ID NOs: 10, 11, 19, 21-26, 36, 37, 39-40, 42, 43, 45, 46, 49, 50, and 52-58; or
(ii) a polypeptide having at least 75%, 80%, 90%, 91%, 92%, 93%, 94%, 95%, 96%, 97%, 98%, or 99% identity to any one of SEQ ID NOs: 10, 11, 19, 21-26, 36, 37, 39-40, 42, 43, 45, 46, 49, 50, and 52-58.

31. A polypeptide comprising:
(a) a functional exogenous receptor, and
(b) an exogenous THP.

32. The polypeptide of claim 31, wherein the functional exogenous receptor is selected from the group consisting of a CAR, an engineered TCR, a cTCR, a TAC, a TAC-like chimeric receptor, and combinations thereof.

33. The polypeptide of claim 32, wherein the functional exogenous receptor is a CAR and wherein the CAR comprises (i) an extracellular antigen binding domain, (ii) a transmembrane domain, and (iii) an intracellular signaling domain, optionally, the CAR further comprises a hinge domain.

34. The polypeptide of claim 33, wherein the intracellular signaling domain comprises a primary intracellular signaling domain of an immune effector cell.

35. The polypeptide of claim 34, wherein the primary intracellular signaling domain is from CD3.

36. The polypeptide of any one of claims 33 to 35, wherein the intracellular signaling domain comprises a co-stimulatory signaling domain.

37. The polypeptide of any one of claims 33 to 36, further comprising a signal peptide, wherein the signal peptide is from CD28 or CD8.

38. The polypeptide of any one of claims 33 to 37, wherein the CAR is an anti-DLL3 CAR.

39. The polypeptide of claim 38, wherein the anti-DLL3 CAR comprises a first V_{H}H antibody moiety that comprises a CDR1 comprising the amino acid sequence of SEQ ID NO: 29 or a variant thereof comprising up to about 3 amino acid substitutions, a CDR2 comprising the amino acid sequence of SEQ ID NO: 30 or a variant thereof comprising up to about 3 amino acid substitutions, and a CDR3 comprising the amino acid sequence of SEQ ID NO: 31 or a variant thereof comprising up to about 3 amino acid substitutions, and a second V_{H}H antibody moiety that comprises a CDR1 comprising the amino acid sequence of SEQ ID NO: 32 or a variant thereof comprising up to about 3 amino acid substitutions, a CDR2 comprising the amino acid sequence of SEQ ID NO:33 or a variant thereof comprising up to about 3 amino acid substitutions, and a CDR3 comprising the amino acid sequence of SEQ ID NO: 34 or a variant thereof comprising up to about 3 amino acid substitutions.

40. The polypeptide of claim 38 or 39, wherein the anti-DLL3 CAR comprises a first V_{H}H antibody moiety comprising the amino acid sequences of SEQ ID NO: 27 or an amino acid sequence having at least 75%, 80%, 90%, 91%, 92%, 93%, 94%, 95%, 96%, 97%, 98%, or 99% identity to the amino acid sequence of SEQ ID NO: 27, and a second V_{H}H antibody moiety comprising the amino acid sequences of SEQ ID NO: 28 or an amino acid sequence having at least 75%, 80%, 90%, 91%, 92%, 93%, 94%, 95%, 96%, 97%, 98%, or 99% identity to the amino acid sequence of SEQ ID NO: 28.

41. The polypeptide of claim 38, wherein the anti-DLL3 CAR comprises the amino acid sequence of SEQ ID NO: 8 or an amino acid sequence having at least 75%, 80%, 90%, 91%, 92%, 93%, 94%, 95%, 96%, 97%, 98%, or 99% identity to the amino acid sequence of SEQ ID NO: 8.

42. The polypeptide of any one of claims 33 to 37, wherein the CAR is an anti-MSLN CAR.

43. The polypeptide of claim 42, wherein the anti-MSLN CAR comprises the amino acid sequence of SEQ ID NO: 41 or an amino acid sequence having at least 75%, 80%, 90%, 91%, 92%, 93%, 94%, 95%, 96%, 97%, 98%, or 99% identity to the amino acid sequence of SEQ ID NO: 41.

44. The polypeptide of any one of claims 33 to 37, wherein the CAR is an anti-GPC2 CAR.

45. The polypeptide of claim 44, wherein the anti-GPC2 CAR comprises the amino acid sequence of SEQ ID NO: 44 or an amino acid sequence having at least 75%, 80%, 90%, 91%, 92%, 93%, 94%, 95%, 96%, 97%, 98%, or 99% identity to the amino acid sequence of SEQ ID NO: 44.

46. The polypeptide of any one of claims 31 to 45, wherein the exogenous THP is selected from the group consisting of RGD-based peptides, NGR-based peptides, and combinations thereof.

47. The polypeptide of any one of claims 31 to 46, wherein the THP comprises the RGD-4C peptide having the amino acid sequence of SEQ ID NO: 2.

48. The polypeptide of any one of claims 31 to 46, wherein the THP comprises the NGR peptide having the amino acid sequence of SEQ ID NO: 38.

49. The polypeptide of any one of claims 31 to 48, wherein the THP is fused with a transmembrane domain and/or a hinge domain.

50. The polypeptide of claim 49, wherein the transmembrane domain and/or the hinge domain is from CD7.

51. The polypeptide of claim 50, wherein the transmembrane domain comprises the amino acid sequence of SEQ ID NO: 14, and the hinge domain comprises the amino acid sequence of SEQ ID NO: 13.

52. The polypeptide of claim 51, wherein the transmembrane domain and/or the hinge domain is from TR2.

53. The polypeptide of any one of claims 31 to 52, wherein the THP is fused with a TGF-β DNR.

54. The polypeptide of claim 53, comprising:
i) the amino acid sequence of SEQ ID NO: 55 or an amino acid sequence having at least 75%, 80%, 90%, 91%, 92%, 93%, 94%, 95%, 96%, 97%, 98%, or 99% identity to the amino acid sequence of SEQ ID NO: 55;
ii) the amino acid sequence of SEQ ID NO: 57 or an amino acid sequence having at least 75%, 80%, 90%, 91%, 92%, 93%, 94%, 95%, 96%, 97%, 98%, or 99% identity to the amino acid sequence of SEQ ID NO: 57;
iii) the amino acid sequence of SEQ ID NO: 54 or an amino acid sequence having at least 75%, 80%, 90%, 91%, 92%, 93%, 94%, 95%, 96%, 97%, 98%, or 99% identity to the amino acid sequence of SEQ ID NO: 54;
iv) the amino acid sequence of SEQ ID NO: 56 or an amino acid sequence having at least 75%, 80%, 90%, 91%, 92%, 93%, 94%, 95%, 96%, 97%, 98%, or 99% identity to the amino acid sequence of SEQ ID NO: 56;
v) the amino acid sequence of SEQ ID NO: 58 or an amino acid sequence having at least 75%, 80%, 90%, 91%, 92%, 93%, 94%, 95%, 96%, 97%, 98%, or 99% identity to the amino acid sequence of SEQ ID NO: 58;
vi) the amino acid sequence of SEQ ID NO: 53 or an amino acid sequence having at least 75%, 80%, 90%, 91%, 92%, 93%, 94%, 95%, 96%, 97%, 98%, or 99% identity to the amino acid sequence of SEQ ID NO: 53; or
vii) the amino acid sequence of SEQ ID NO: 52 or an amino acid sequence having at least 75%, 80%, 90%, 91%, 92%, 93%, 94%, 95%, 96%, 97%, 98%, or 99% identity to the amino acid sequence of SEQ ID NO: 52.

55. The polypeptide of any one of claims 31 to 54, wherein the functional exogenous receptor is at the N terminus or C terminus of the exogenous THP.

56. The polypeptide of any one of claims 31 to 32, wherein the functional exogenous receptor is a cTCR.

57. The polypeptide of claim 56, wherein the cTCR is an anti-DLL3 cTCR, and the anti-DLL3 cTCR comprises the amino acid sequence of SEQ ID NO: 35 or an amino acid sequence having at least 75%, 80%, 90%, 91%, 92%, 93%, 94%, 95%, 96%, 97%, 98%, or 99% identity to the amino acid sequence of SEQ ID NO: 35.

58. The polypeptide of any one of claims 31 to 57, comprising (i) the amino acid sequence set forth in any one of SEQ ID NOs: 10, 11, 19, 21-26, 36, 37, 39-40, 42, 43, 45, 46, 49, 50, and 52-58; or
(ii) the amino acid sequence having at least 75%, 80%, 90%, 91%, 92%, 93%, 94%, 95%, 96%, 97%, 98%, or 99% identity to any one of SEQ ID NOs: 10, 11, 19, 21-26, 36, 37, 39-40, 42, 43, 45, 46, 49, 50, and 52-58.

59. An isolated nucleic acid comprising a nucleic acid sequence encoding the polypeptide of any one of claims 31 to 58.

60. A vector comprising the isolated nucleic acid of claim 59.

61. A host cell comprising the vector of claim 60.

62. A method of making an immune effector cell of any one of claims 1 to 30 comprising introducing into an immune effector cell:
(a) the nucleic acid of claim 59 or the vector of claim 60; or
(b) a composition comprising two nucleic acids each encoding:
(i) a functional exogenous receptor, and
(ii) an exogenous THP.

63. An immune effector cell produced according to the method of claim 62.

64. A pharmaceutical composition, comprising the immune effector cell of any one of claims 1 to 30, the polypeptide of any one of claims 31 to 58, the nucleic acid of claim 59, or the vector of claim 60, and a pharmaceutically acceptable carrier.

65. A method of treating a disease or disorder in a subject, comprising administering to the subject an effective amount of the pharmaceutical composition of claim 64.

66. The method of claim 65, wherein the disease or disorder is selected from the group consisting of cancer, infectious disease, inflammatory disease, autoimmune disease, and the combinations thereof.

67. The method of claim 66, wherein the cancer is a solid tumor cancer or hematological cancer.

68. The method of claim 66 or 67, wherein the cancer is small-cell lung cancer (SCLC), ovarian cancer (OC) or neuroblastoma (NBL).
